(19) United States
(12) Patent Application Publication
(10) Pub. No.: US 2026/0217845 A1
(43) Pub. Date: Jul. 30, 2026

(54) ANTI-TNFR2 ANTIGEN-BINDING PROTEINS AND USES THEREOF

(71) Applicant: Odyssey Therapeutics, Inc., Boston, MA (US)

(72) Inventors: Luigi FRANCHI, Boston, MA (US); Anthony W. OPIPARI, Boston, MA (US); Lee Kim SWEE, Frankfurt am Main (DE); Paul-Albert KÖNIG, Frankfurt am Main (DE); Laura PREISS, Frankfurt am Main (DE); Ferdinand HUBER, Frankfurt am Main (DE); Annegrit SEIFRIED, Frankfurt am Main (DE); Julia KNOPF, Frankfurt am Main (DE); Tomasz PRÓCHNICKI, Frankfurt am Main (DE)

(73) Assignee: Odyssey Therapeutics, Inc., Boston, MA (US)

(21) Appl. No.: 19/146,347

(22) PCT Filed: Jan. 8, 2024
(86) PCT No.: PCT/US2024/010660

§ 371 Date: Jul. 8, 2025

Related U.S. Application Data
(60) Provisional application No. 63/437,877, filed on Jan. 9, 2023.
(60) Provisional application No. 63/472,175, filed on Jun. 9, 2023.

(51) Int. Cl. C07K 16/28; A61K 39/00; A61P 19/02; C07K 14/55

(52) U.S. Cl. CPC C07K 16/2878; A61P 19/02; C07K 14/55; A61K 2039/505; C07K 2317/22; C07K 2317/24; C07K 2317/52; C07K 2317/565; C07K 2317/569; C07K 2317/74; C07K 2317/92; C07K 2319/30

(57) ABSTRACT

The present application provides antigen-binding proteins (e.g., antibodies such as single-domain antibodies) that specifically bind tumor necrosis factor receptor 2 (TNFR2). The application also provides fusion proteins and conjugates comprising the antigen-binding proteins, polynucleotides and recombinant vectors encoding the antigen-binding proteins, as well as host cells and methods for preparing the antigen-binding proteins. The application further provides pharmaceutical compositions comprising the antigen-binding proteins and methods for treating a disease or disorder using the antigen-binding proteins.

### CROSS-REFERENCE TO RELATED APPLICATIONS

This application claims priority to U.S. Provisional Application No. 63/437,877, filed Jan. 9, 2023, and U.S. Provisional Application No. 63/472,175, filed Jun. 9, 2023, the disclosure of both of which is herein incorporated by reference in its entirety.

### SEQUENCE LISTING

The instant application contains a Sequence Listing which has been submitted electronically in XML format and is hereby incorporated by reference in its entirety. Said XML copy, created on Jan. 5, 2024, is named 260525_000029_SL.xml and is 4,669,862 bytes in size.

### FIELD OF THE INVENTION

The present application relates to antigen-binding proteins (e.g., antibodies such as single-domain antibodies) that specifically bind tumor necrosis factor receptor 2 (TNFR2), methods for their preparation, and uses thereof.

### BACKGROUND OF THE INVENTION

Regulatory T cells (Tregs) are a subset of T cells that play a crucial role in peripheral self-tolerance and the prevention of autoimmunity. Due to their potent immunosuppressive function, Tregs can be targeted for the treatment of autoimmunity. Current strategies seeking to increase or modulate Tregs in autoimmune patients are based on the ex vivo expansion of Tregs prior to autologous transfer. However, a major limitation of the current strategies is their inability to stabilize Tregs phenotype to ensure long-lasting immunoregulation.

Tumor necrosis factor receptor 2 (TNFR2) signaling has been shown to induce proliferation, sustained suppressive function and FOXP3 promoter demethylation in Tregs (Tseng et al., 2019). TNFR2 signaling also induces the expression of EZH2 (Urbano et al., 2018), a histone methyl transferase involved in the repression of the effector transcriptomic program and stabilization of the Treg phenotype (DuPage et al., 2015). Because of its role in Tregs biology and FOXP3 promoter demethylation, TNFR2 signaling can be leveraged to induce a stable immunosuppressive phenotype and enhance their function to the benefit of autoimmune diseases. Accordingly, there is a need in the art to develop therapeutic molecules that can effectively activate TNFR2 signaling.

### SUMMARY OF THE INVENTION

As mentioned in the background section above, there is an unmet need in the art to develop therapeutic molecules that can effectively activate TNFR2 signaling. This application provides compositions and methods to address this and other related needs.

In one aspect, the present disclosure provides an antigen-binding protein that specifically binds tumor necrosis factor receptor 2 (TNFR2), comprising a complementarity determining region 3 (CDR3) comprising an amino acid sequence selected from

a).
(SEQ ID NO: 60)
(Y/F)YQ(S/A)LS(T/S)(P/A)N(Y/F)GQ(V/T)F;
b).
(SEQ ID NO: 61)
AADSDL(S/R)TV(V/T)VGPHDY;
c).
(SEQ ID NO: 62)
AKDAG(S/G)WG(T/R)GPFG(Y/F)(E/D)YDY;
d).
(SEQ ID NO: 63)
AA(T/A)PSGKAY(T/S)Y;
e).
(SEQ ID NO: 64)
ATPGPY(T/S/M)YCAPYGSSWSRGYDY;
f).
(SEQ ID NO: 65)
ARV(R/G)G(T/S/A)PY(E/D)Y(N/G)Y;
g).
(SEQ ID NO: 66)
(T/A/V)A(S/A)PTGRAF(T/N/A)Y;
h).
(SEQ ID NO: 42)
AGSAFDF;
i).
(SEQ ID NO: 67)
S(V/M)(V/L)GRDM(M/V)TY;
j).
(SEQ ID NO: 4063)
AVGDFEGELVLKGDY;
k).
(SEQ ID NO: 4517)
AAD(L/V)G(F/V/Y)LY(A/T/V)DYV(P/R)LH(M/T)HHFGS;
l).
(SEQ ID NO: 4771)
A(A/G)(T/A)(P/L)(S/T)GKAY(T/S)Y.

In some embodiments, the CDR3 comprises an amino acid sequence selected from SEQ ID NOs: 3, 6, 10, 14, 18, 22, 26, 30, 34, 38, 42, 46, 4063, 4067, 4071, 4524, 4530, and 4727-4730.

In some embodiments, the antigen-binding protein further comprises a CDR1 comprising an amino acid sequence selected from

a).
(SEQ ID NO: 68)
GSI(V/F)(R/S)(T/A)(N/D)(S/G/A);
b).
(SEQ ID NO: 69)
GFT(F/L)DD(I/Y)A;
c).
(SEQ ID NO: 70)
GFTFS(S/R/G)YA;
d).
(SEQ ID NO: 16)
GRTFSDYG;
e).
(SEQ ID NO: 71)
G(L/F)TLDYYA;
f).
(SEQ ID NO: 72)
GF(T/N)FSMYS;
g).
(SEQ ID NO: 73)
GRTF(G/R/S)(N/S)(Y/L)(T/F);
h).
(SEQ ID NO: 40)
GASLSRNA;
i).
(SEQ ID NO: 74)
GS(I/T)FRFPP;
j).
(SEQ ID NO: 4061)
GFTLDDYA;
and
k).
(SEQ ID NO: 4519)
G(F/V)(S/T)LD(D/Y)(H/Y)T.

In some embodiments, the antigen-binding protein comprises a CDR1 comprising an amino acid sequence selected from SEQ ID NOs: 1, 5, 8, 12, 16, 20, 24, 28, 32, 36, 40, 44, 4061, 4065, 4069, 4520, and 4719-4722.

In some embodiments, the antigen-binding protein comprises a CDR2 comprising an amino acid sequence selected from

a).
(SEQ ID NO: 75)
IRSDGF(T/I);
b).
(SEQ ID NO: 76)
I(Y/F)SY(S/G)(S/P)NT;
c).
(SEQ ID NO: 77)
I(Y/S)(S/D)DGS(E/D)T;
d).
(SEQ ID NO: 4699)
INWSN(G/A)RT;
e).
(SEQ ID NO: 78)
I(S/N)(V/T)(S/G)DGST;
f).
(SEQ ID NO: 79)
IDT(R/G)GST;
g).
(SEQ ID NO: 80)
IR(W/R/Y)(T/P)G(G/L)(S/I)T;
h).
(SEQ ID NO: 41)
IYDDGET;
i).
(SEQ ID NO: 45)
LTSGGST;
j).
(SEQ ID NO: 4062)
IFSYSSNT;
and
k).
(SEQ ID NO: 4518)
I(N/S)SNDG(S/T)(T/V).

In some embodiments, the antigen-binding protein comprises a CDR2 comprises an amino acid sequence selected from SEQ ID NOs: 2, 9, 13, 17, 21, 25, 29, 33, 37, 41, 45, 4062, 4066, 4070, 4527, and 4723-4726.

In some embodiments, the antigen-binding protein comprise
i) a CDR1 comprising an amino acid sequence of SEQ ID NO: 68, a CDR2 comprising an amino acid sequence of SEQ ID NO: 75, and a CDR3 comprising an amino acid sequence of SEQ ID NO: 60;
ii) a CDR1 comprising an amino acid sequence of SEQ ID NO: 69, a CDR2 comprising an amino acid sequence of SEQ ID NO: 76, a CDR3 comprising an amino acid sequence of SEQ ID NO: 61;
iii) a CDR1 comprising an amino acid sequence of SEQ ID NO: 70, a CDR2 comprising an amino acid sequence of SEQ ID NO: 77, a CDR3 comprising an amino acid sequence of SEQ ID NO: 62;
iv) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 63;
v) a CDR1 comprising an amino acid sequence of SEQ ID NO: 71, a CDR2 comprising an amino acid sequence of SEQ ID NO: 78, a CDR3 comprising an amino acid sequence of SEQ ID NO: 64;
vi) a CDR1 comprising an amino acid sequence of SEQ ID NO: 72, a CDR2 comprising an amino acid sequence of SEQ ID NO: 79, a CDR3 comprising an amino acid sequence of SEQ ID NO: 65;
vii) a CDR1 comprising an amino acid sequence of SEQ ID NO: 73, a CDR2 comprising an amino acid sequence of SEQ ID NO: 80, a CDR3 comprising an amino acid sequence of SEQ ID NO: 66;
viii) a CDR1 comprising an amino acid sequence of SEQ ID NO: 40, a CDR2 comprising an amino acid sequence of SEQ ID NO: 41, a CDR3 comprising an amino acid sequence of SEQ ID NO: 42; or
ix) a CDR1 comprising an amino acid sequence of SEQ ID NO: 74, a CDR2 comprising an amino acid sequence of SEQ ID NO: 45, a CDR3 comprising an amino acid sequence of SEQ ID NO: 67;
x) a CDR1 comprising an amino acid sequence of SEQ ID NO: 4061, a CDR2 comprising an amino acid sequence of SEQ ID NO: 4062, a CDR3 comprising an amino acid sequence of SEQ ID NO: 4063;
xi). a CDR1 comprising an amino acid sequence of SEQ ID NO: 4519, a CDR2 comprising an amino acid sequence of SEQ ID NO: 4518, a CDR3 comprising an amino acid sequence of SEQ ID NO: 4517; or
xii). a CDR1 comprising an amino acid sequence of SEQ ID NO: 16, a CDR2 comprising an amino acid sequence of SEQ ID NO: 4699, a CDR3 comprising an amino acid sequence of SEQ ID NO: 4771.

In some embodiments, the antigen-binding protein comprise
a) a CDR1 comprising an amino acid sequence of SEQ ID NO: 69, a CDR2 comprising an amino acid sequence of SEQ ID NO: 76, a CDR3 comprising an amino acid sequence of SEQ ID NO: 61;
b) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 63;
c) a CDR1 comprising an amino acid sequence of SEQ ID NO: 73, a CDR2 comprising an amino acid sequence of SEQ ID NO: 80, a CDR3 comprising an amino acid sequence of SEQ ID NO: 66; or
d) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 4771.

In some embodiments, the antigen-binding protein comprise comprises
i) a CDR1 with an amino acid sequence of GSI(V/F)(R/S)(A/T)(N/D)(G/A) (SEQ ID NO: 4700), a CDR2 comprising an amino acid sequence of IRSDGFT (SEQ ID NO: 2), and a CDR3 comprising an amino acid sequence of YYQ(S/A)LSSPNYGQ(V/T)F (SEQ ID NO: 4701);
ii) a CDR1 with an amino acid sequence of GFTFDDIA (SEQ ID NO: 8), a CDR2 comprising an amino acid sequence of IYSYGPNT (SEQ ID NO: 9), and a CDR3 comprising an amino acid sequence of AADSDLSTW(V/T)GPHDY (SEQ ID NO: 4702);
iii) a CDR1 with an amino acid sequence of GFTFSRYA (SEQ ID NO: 12), a CDR2 comprising an amino acid sequence of ISDDGSDT (SEQ ID NO: 13), and a CDR3 comprising an amino acid sequence of AKDAGSWGTGPFGYEYDY (SEQ ID NO: 14);
iv) a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of AA(T/A)PSGKAYSY (SEQ ID NO: 4703);
v) a CDR1 with an amino acid sequence of GLTLDYYA (SEQ ID NO: 20), a CDR2 comprising an amino acid sequence of ISTSDGST (SEQ ID NO: 21), and a CDR3 comprising an amino acid sequence of ATPGPYTYCAPYGSSWSRGYDY (SEQ ID NO: 22);
vi) a CDR1 with an amino acid sequence of GF(T/N)FSMYS (SEQ ID NO: 72), a CDR2 comprising an amino acid sequence of IDT(R/G)GST (SEQ ID NO: 79), and a CDR3 comprising an amino acid sequence of ARV(G/R)G(T/A)PYEY(N/G)Y (SEQ ID NO: 4704);
vii) a CDR1 with an amino acid sequence of GRTF(G/S)S(Y/L)(T/F) (SEQ ID NO: 4705), a CDR2 comprising an amino acid sequence of IR(W/R/Y)(T/P)G(G/L)(S/I)T (SEQ ID NO: 80), and a CDR3 comprising an amino acid sequence of (A/V)A(A/S)PTGRAF(T/N)Y (SEQ ID NO: 4707);
viii) a CDR1 with an amino acid sequence of GASLSRNA (SEQ ID NO: 40), a CDR2 comprising an amino acid sequence of IYDDGET (SEQ ID NO: 41), and a CDR3 comprising an amino acid sequence of AGSAFDF (SEQ ID NO: 42);
ix) a CDR1 with an amino acid sequence of GS(T/I)FRFPP (SEQ ID NO: 4708), a CDR2 comprising an amino acid sequence of LTSGGST (SEQ ID NO: 45), and a CDR3 comprising an amino acid sequence of SVLGRDM(M/V)TY (SEQ ID NO: 4706);
x) a CDR1 with an amino acid sequence of GFTLDDYA (SEQ ID NO: 4061), a CDR2 comprising an amino acid sequence of IFSYSSNT (SEQ ID NO: 4062), and a CDR3 comprising an amino acid sequence of AVGDFEGELVLKGDY (SEQ ID NO: 4063);
xi) a CDR1 with an amino acid sequence of GFTLDYYT (SEQ ID NO: 4065), a CDR2 comprising an amino acid sequence of ISSNDGSV (SEQ ID NO: 4066), and a CDR3 comprising an amino acid sequence of AADLGYLYVDYVRLHTHHFGS (SEQ ID NO: 4067); or
xii). a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of A(A/G)(T/A)(P/L)(S/T)GKAY(T/S)Y (SEQ ID NO: 4771).

In some embodiments, the antigen-binding protein comprises
a) a CDR1 with an amino acid sequence of GFTFDDIA (SEQ ID NO: 8), a CDR2 comprising an amino acid sequence of IYSYGPNT (SEQ ID NO: 9), and a CDR3 comprising an amino acid sequence of AADSDLSTW(V/T)GPHDY (SEQ ID NO: 4702);
b) a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of AA(T/A)PSGKAYSY (SEQ ID NO: 4703);
c) a CDR1 with an amino acid sequence of GRTF(G/S)S(Y/L)(T/F) (SEQ ID NO: 4705), a CDR2 comprising an amino acid sequence of IR(W/R/Y)(T/P)G(G/L)(S/I)T (SEQ ID NO: 80), and a CDR3 comprising an amino acid sequence of (A/V)A(A/S)PTGRAF(T/N)Y (SEQ ID NO: 4707); or
d). a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of A(A/G)(T/A)(P/L)(S/T)GKAY(T/S)Y (SEQ ID NO: 4771).

In some embodiments, the antigen-binding protein comprises
i) a CDR1 comprising an amino acid sequence of SEQ ID NO: 1, a CDR2 comprising an amino acid sequence of SEQ ID NO: 2, and a CDR3 comprising an amino acid sequence of SEQ ID NO: 3;
ii) a CDR1 comprising an amino acid sequence of SEQ ID NO: 5, a CDR2 comprising an amino acid sequence of SEQ ID NO: 2, a CDR3 comprising an amino acid sequence of SEQ ID NO: 6;
iii) a CDR1 comprising an amino acid sequence of SEQ ID NO: 8, a CDR2 comprising an amino acid sequence of SEQ ID NO: 9, a CDR3 comprising an amino acid sequence of SEQ ID NO: 10;
iv) a CDR1 comprising an amino acid sequence of SEQ ID NO: 12, a CDR2 comprising an amino acid sequence of SEQ ID NO: 13, a CDR3 comprising an amino acid sequence of SEQ ID NO: 14;
v) a CDR1 comprising an amino acid sequence of SEQ ID NO: 16, a CDR2 comprising an amino acid sequence of SEQ ID NO: 17, a CDR3 comprising an amino acid sequence of SEQ ID NO: 18;
vi) a CDR1 comprising an amino acid sequence of SEQ ID NO: 20, a CDR2 comprising an amino acid sequence of SEQ ID NO: 21, a CDR3 comprising an amino acid sequence of SEQ ID NO: 22;
vii) a CDR1 comprising an amino acid sequence of SEQ ID NO: 24, a CDR2 comprising an amino acid sequence of SEQ ID NO: 25, a CDR3 comprising an amino acid sequence of SEQ ID NO: 26;
viii) a CDR1 comprising an amino acid sequence of SEQ ID NO: 28, a CDR2 comprising an amino acid sequence of SEQ ID NO: 29, a CDR3 comprising an amino acid sequence of SEQ ID NO: 30;
ix) a CDR1 comprising an amino acid sequence of SEQ ID NO: 32, a CDR2 comprising an amino acid sequence of SEQ ID NO: 33, a CDR3 comprising an amino acid sequence of SEQ ID NO: 34;
x) a CDR1 comprising an amino acid sequence of SEQ ID NO: 36, a CDR2 comprising an amino acid sequence of SEQ ID NO: 37, a CDR3 comprising an amino acid sequence of SEQ ID NO: 38;
xi) a CDR1 comprising an amino acid sequence of SEQ ID NO: 40, a CDR2 comprising an amino acid sequence of SEQ ID NO: 41, a CDR3 comprising an amino acid sequence of SEQ ID NO: 42;
xii) a CDR1 comprising an amino acid sequence of SEQ ID NO: 44, a CDR2 comprising an amino acid sequence of SEQ ID NO: 45, a CDR3 comprising an amino acid sequence of SEQ ID NO: 46;
xiii) a CDR1 comprising an amino acid sequence of SEQ ID NO: 4061, a CDR2 comprising an amino acid sequence of SEQ ID NO: 4062, a CDR3 comprising an amino acid sequence of SEQ ID NO: 4063;
xiv). a CDR1 comprising an amino acid sequence of SEQ ID NO: 4065, a CDR2 comprising an amino acid sequence of SEQ ID NO: 4066, a CDR3 comprising an amino acid sequence of SEQ ID NO: 4067;
xv). a CDR1 comprising an amino acid sequence of SEQ ID NO: 4069, a CDR2 comprising an amino acid sequence of SEQ ID NO: 4070, a CDR3 comprising an amino acid sequence of SEQ ID NO: 4071;
xvi). a CDR1 comprising an amino acid sequence of SEQ ID NO: 4520, a CDR2 comprising an amino acid sequence of SEQ ID NO: 45, a CDR3 comprising an amino acid sequence of SEQ ID NO: 46;
xvii). a CDR1 comprising an amino acid sequence of SEQ ID NO: 8, a CDR2 comprising an amino acid sequence of SEQ ID NO: 9, a CDR3 comprising an amino acid sequence of SEQ ID NO: 4524;
xviii). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 18;
xix). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 4530;
xx). a CDR1 comprising an amino acid sequence of SEQ ID NO: 4719, a CDR2 comprising an amino acid sequence of SEQ ID NO: 4723, a CDR3 comprising an amino acid sequence of SEQ ID NO: 4727;
xxl). a CDR1 comprising an amino acid sequence of SEQ ID NO: 4720, a CDR2 comprising an amino acid sequence of SEQ ID NO: 4724, a CDR3 comprising an amino acid sequence of SEQ ID NO: 4728;
xxii). a CDR1 comprising an amino acid sequence of SEQ ID NO: 4721, a CDR2 comprising an amino acid sequence of SEQ ID NO: 4725, a CDR3 comprising an amino acid sequence of SEQ ID NO: 4729; or
xxiii). a CDR1 comprising an amino acid sequence of SEQ ID NO: 4722, a CDR2 comprising an amino acid sequence of SEQ ID NO: 4726, a CDR3 comprising an amino acid sequence of SEQ ID NO: 4730.

In some embodiments, the antigen-binding protein comprises
a) a CDR1 comprising an amino acid sequence of SEQ ID NO: 16, a CDR2 comprising an amino acid sequence of SEQ ID NO: 17, a CDR3 comprising an amino acid sequence of SEQ ID NO: 18;
b). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 18;
c). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 4530;
d) a CDR1 comprising an amino acid sequence of SEQ ID NO: 8, a CDR2 comprising an amino acid sequence of SEQ ID NO: 9, a CDR3 comprising an amino acid sequence of SEQ ID NO: 10;
e). a CDR1 comprising an amino acid sequence of SEQ ID NO: 8, a CDR2 comprising an amino acid sequence of SEQ ID NO: 9, a CDR3 comprising an amino acid sequence of SEQ ID NO: 4524;
f). a CDR1 comprising an amino acid sequence of SEQ ID NO: 4069, a CDR2 comprising an amino acid sequence of SEQ ID NO: 4070, a CDR3 comprising an amino acid sequence of SEQ ID NO: 4071;
g). a CDR1 comprising an amino acid sequence of SEQ ID NO: 4719, a CDR2 comprising an amino acid sequence of SEQ ID NO: 4723, a CDR3 comprising an amino acid sequence of SEQ ID NO: 4727;
h). a CDR1 comprising an amino acid sequence of SEQ ID NO: 4720, a CDR2 comprising an amino acid sequence of SEQ ID NO: 4724, a CDR3 comprising an amino acid sequence of SEQ ID NO: 4728;
i). a CDR1 comprising an amino acid sequence of SEQ ID NO: 4721, a CDR2 comprising an amino acid sequence of SEQ ID NO: 4725, a CDR3 comprising an amino acid sequence of SEQ ID NO: 4729; or
j). a CDR1 comprising an amino acid sequence of SEQ ID NO: 4722, a CDR2 comprising an amino acid sequence of SEQ ID NO: 4726, a CDR3 comprising an amino acid sequence of SEQ ID NO: 4730.

In some embodiments, the antigen-binding protein is a single-domain antibody. In some embodiments, the single-domain antibody is a VHH, a VNAR, or an engineered VH domain.

In some embodiments, the VHH is a camelid VHH. In some embodiments, the VHH comprises an amino acid sequence selected from any one of SEQ ID NOs: 4, 7, 11, 15, 19, 23, 27, 31, 35, 39, 43, 47, 4064, 4068, 4072, 4521, 93-640, 4079-4125 2805-3363, 4359-4420, and 4605-4628, or a sequence having at least 75% identity thereto. In some embodiments, the VHH comprises an amino acid sequence selected from any one of SEQ ID NOs: 4, 7, 11, 15, 19, 23, 27, 31, 35, 39, 43, 47, 4064, 4068, 4072, and 4521 or a sequence having at least 75% identity thereto.

In some embodiments, the VHH is a humanized VHH. In some embodiments, the humanized VHH comprises an amino acid sequence selected from any one of SEQ ID NOs: 81-92, 4076-, 4078, 4523, 4526, 4529, 4532, 4731-4734, 641-1127, and 4126-4172, or a sequence having at least 75% Identity thereto. In some embodiments, the humanized VHH comprises an amino acid sequence selected from any one of SEQ ID NOs: 81-92, 4076-4078, 4523, 4526, 4529, 4731-4734, and 4532, or a sequence having at least 75% identity thereto.

In some embodiments, the antigen-binding protein has an agonist effect upon binding to TNFR2,

In some embodiments, the antigen-binding protein binds to human TNFR2. In some embodiments, the antigen-binding protein binds to human TNFR2 with a K_{D} of less than about 3×10⁻⁷ M. In some embodiments, the antigen-binding protein binds to human TNFR2 with a K_{D} of about 1×10⁻¹⁰ to 5×10⁻⁸ M.

In some embodiments, the antigen-binding protein binds to cyno TNFR2. In some embodiments, the antigen-binding protein binds to cyno TNFR2 with a K_{D} of less than about 3×10⁻⁷ M. In some embodiments, the antigen-binding protein binds to cyno TNFR2 with a K_{D} of about 1×10⁻³ to 2×10⁻⁷ M.

In some embodiments, the antigen-binding protein binds to the same epitope(s) as antibody clone MR2-1. In some embodiments, the antigen-binding protein does not bind to the same epitope(s) as antibody clone MR2-1.

In some embodiments, the antigen-binding protein increases expression of one or more proteins selected from a protein in the NF-kB pathway, FOXP3, HELIOS, EZH2, HLA-DR, ICAM-1, OX-40, ICOS, and CCR8.

In some embodiments, the antigen-binding protein comprises one or more modifications that reduce binding of said antigen-binding protein by pre-existing antibodies found in human blood or serum.

In another aspect, provided herein is a fusion protein that specifically binds tumor necrosis factor receptor 2 (TNFR2), comprising one or more of the antigen-binding proteins described herein.

In some embodiments, the fusion protein comprises two of the antigen-binding proteins described herein. In some embodiments, the fusion protein comprises three of the antigen-binding proteins described herein. In some embodiments, the fusion protein comprises four of the antigen-binding proteins described herein. In some embodiments, the fusion protein comprises five of the antigen-binding proteins described herein. In some embodiments, the fusion protein comprises six of the antigen-binding proteins described herein.

In some embodiments of the fusion protein described herein, the one or more antigen-binding proteins may bind to the same epitope on TNFR2. In other embodiments, the one or more antigen-binding proteins may bind to different epitopes on TNFR2.

In some embodiments of the fusion protein described herein, the one or more antigen-binding proteins are one or more single-domain antibodies. In some embodiments, the one or more single-domain antibodies are one or more VHHs.

In some embodiments of the fusion protein described herein, the fusion protein further comprises an immunoglobulin Fc region. In some embodiments, the immunoglobulin Fc region is an Fc region of a human immunoglobulin. In some embodiments, the immunoglobulin Fc region is an Fc region of human IgG1, IgG2, IgG3 or IgG4, or a variant thereof.

In some embodiments, the immunoglobulin Fc region is an Fc region of human IgG1, or a variant thereof. In some embodiments, the Fc region of human IgG1 comprises one or more mutations selected from L234A, L235A, G237A, D265A, N297A, and/or P329A according to EU numbering. In some embodiments, the Fc region of human IgG1 comprises a set of mutations selected from
1). L234A and L235A;
2). L234A, L235A, and P329A;
3). D265A, N297A and P329A; and
4). L234A, L235A, and G237A.

In some embodiments, the immunoglobulin Fc region is an Fc region of human IgG1 comprising L234A, L235A, and P329A.

In some embodiments, the immunoglobulin Fc region is an Fc region of human IgG4, or a variant thereof. In some embodiments, the Fc region of human IgG4 comprises one or more mutations selected from S228P, L235E, L235A, and/or F234A according to EU numbering. In some embodiments, the Fc region of human IgG4 comprises a set of mutations selected from
1). S228P and L235E;
2). S228P and L235A;
3). S228P, F234A, and L235E; and
4). S228P, F234A, and L235A.

In some embodiments, the immunoglobulin Fc region is an Fc region of human IgG4 comprising S228P and L235E.

In some embodiments of the fusion protein described herein, the fusion protein further comprises a cytokine. In some embodiments, the cytokine is IL-2, or a variant thereof. In one embodiment, the cytokine is an IL-2 variant comprising a N88D mutation.

In some embodiments of the fusion protein described herein, the fusion protein further comprises a moiety that binds to serum albumin.

In some embodiments of the fusion protein described herein, the fusion protein comprises an amino acid sequence of any one of SEQ ID Nos: 3933-3964, 4483-4513, 4686-4696, 4709-4716, and 4735-4770, or a sequence having at least 75% identity thereto.

In some embodiments of the fusion protein described herein, the fusion protein comprises an amino acid sequence of SEQ ID No: 4483, or a sequence having at least 75% identity thereto.

In some embodiments of the fusion protein described herein, the fusion protein comprises an amino acid sequence of SEQ ID No: 4489, or a sequence having at least 75% identity thereto.

In another aspect, provided herein is a conjugate comprising the antigen-binding protein described herein or the fusion protein described herein, wherein the antigen-binding protein or fusion protein is conjugated to a second moiety. In some embodiments, the second moiety is selected from a detectable label, a drug, a toxin, a radionuclide, an enzyme, an immunomodulatory agent, a cytokine, a cytotoxic agent, a chemotherapeutic agent, a diagnostic agent, or a combination thereof.

In some embodiments of the conjugate described herein, wherein the second moiety is a cytokine. In some embodiments, the cytokine is IL-2, or a variant thereof. In one embodiment, the cytokine is IL-2 variant comprising a N88D mutation.

In another aspect, provided herein is a polynucleotide molecule encoding the antigen-binding protein described herein or the fusion protein described herein. In some embodiments, the polynucleotide molecule comprises the nucleotide sequence of any one of SEQ ID NOs: 48-59, 4073-4075, 4522, 4525, 4528, 4531, 3364-3922, 4421-4482, and 4629-4652, or a sequence having at least 70% identity thereto. In some embodiments, the polynucleotide molecule comprises the nucleotide sequence of any one of SEQ ID NOs: 48-59, 4073-4075, 4522, 4525, 4528, and 4531, or a sequence having at least 70% identity thereto.

In another aspect, provided herein is a recombinant vector comprising the polynucleotide molecule described herein.

In another aspect, provided herein is a host cell comprising polynucleotide molecule described herein, or the expression vector described herein.

In another aspect, provided herein is a kit comprising the antigen-binding protein, the fusion protein, the conjugate, the polynucleotide molecule, or the recombinant vector described herein, and optionally, instructions and/or packaging for the same.

In another aspect, provided herein is a pharmaceutical composition comprising the antigen-binding protein, the fusion protein, the conjugate, the polynucleotide molecule, or the recombinant vector described herein, and a pharmaceutically acceptable carrier and/or excipient.

In another aspect, provided herein is a method for preparing an antigen-binding protein or a fusion protein that specifically binds tumor necrosis factor receptor 2 (TNFR2), comprising the steps of:
(a) culturing the host cell described herein in a culture medium under conditions suitable for expression of the antigen-binding protein or fusion protein, and
(b) isolating the antigen-binding protein or fusion protein from the host cell and/or culture medium,

In another aspect, provided herein is a method for promoting proliferation, activating and/or enhancing suppressive function, and/or stabilizing immunosuppressive phenotype of a population of regulatory T cells (Treg) comprising contacting the population of regulatory T cells with the antigen-binding protein, the fusion protein, or the conjugate described herein. In some embodiments, said contacting occurs in vitro. In some embodiments, said contacting occurs in vivo. In some embodiments wherein the method occurs in vivo, the method further comprises administering the antigen-binding protein, the fusion protein, or the conjugate into a subject in need thereof.

In another aspect, provided herein is a method of treating or preventing a disease or disorder in a subject in need thereof, said method comprising administering to the subject the antigen-binding protein, the fusion protein, or the conjugate described herein. In some embodiments, the disease or disorder is an Immunological disease, inflammatory disease, cancer, cardiovascular disease, or an infertility and pregnancy-associated disease.

In some embodiments, the immunological disease is selected from an autoimmune disease, a neurological condition, an allergy, asthma, macular degeneration, muscular atrophy, a disease related to miscarriage, atherosclerosis, bone loss, a musculoskeletal disease, obesity, a graft-versus-host disease, and an allograft rejection.

In some embodiments, the autoimmune disease is selected from lupus, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, essential mixed cryoglobulinemia, fibromyalgia-fibromyositis, Goodpastures disease, Graves' disease, Guillain-Barré, Hashimoto's thyroiditis, hypothyroidism, idiopathic pulmonary fibrosis, idiopathic thrombocytopenia purpura (ITP), IgA nephropathy, juvenile arthritis, lichen planus, lichen sclerosis, IgG4-related disease, Meniere's disease, mixed connective tissue disease, multiple sclerosis, myasthenia gravis, neuromyelitis optica spectrum disease, pemphigus vulgaris, or related blistering skin disease, pernicious anemia, polyarteritis nodosa, polychondritis, polyglandular syndromes, polymyalgia rheumatica, polymyositis and dermatomyositis, premature ovarian failure, primary agammaglobulinemia, primary biliary cirrhosis, psoriasis, primary ovarian insufficiency, Raynaud's phenomenon, Reiter's syndrome, rheumatic fever, rheumatoid arthritis, sarcoidosis, scleroderma, Sjögren's syndrome, spondyloarthritis, stiff-man syndrome, type I diabetes, Takayasu arteritis, temporal arteritis/giant cell arteritis, ulcerative colitis, uveitis, vasculitis, vitiligo, and Wegener's granulomatosis (Granulomatosis with polyangiitis) or other immune vasculitis.

In some embodiments, the lupus is systemic lupus erythematosus (SLE), cutaneous lupus, lupus nephritis, neonatal lupus, or drug-induced lupus. In some embodiments, the cutaneous lupus is acute cutaneous lupus, chronic cutaneous lupus erythematosus, discoid lupus erythematosus (DLE), or subacute cutaneous lupus erythematosus.

In some embodiments, the autoimmune disease is atopic dermatitis, psoriasis, systemic lupus erythematosus, or arthritis.

In some embodiments, the neurological condition is selected from a brain tumor, a brain metastasis, a spinal cord injury, schizophrenia, epilepsy, Amyotrophic lateral sclerosis (ALS), Alzheimer's disease, Huntington's disease, Parkinson's disease, and stroke.

In some embodiments, the allergy is selected from food allergy, seasonal allergy, pet allergy, hives, hay fever, allergic conjunctivitis, poison ivy allergy oak allergy, mold allergy, drug allergy, dust allergy, cosmetic allergy, and chemical allergy.

In some embodiments, the allograft rejection is selected from skin graft rejection, bone graft rejection, vascular tissue graft rejection, ligament graft rejection, and organ graft rejection.

In some embodiments, the ligament graft rejection is selected from cricothyroid ligament graft rejection, caudal cruciate ligament graft rejection, periodontal ligament graft rejection, suspensory ligament of the lens graft rejection, palmar radiocarpal ligament graft rejection, dorsal radiocarpal ligament graft rejection, ulnar collateral ligament graft rejection, radial collateral ligament graft rejection, suspensory ligament of the breast graft rejection, anterior sacroiliac ligament graft rejection, posterior sacroiliac ligament graft rejection, sacrotuberous ligament graft rejection, sacrospinous ligament graft rejection, inferior pubic ligament graft rejection, superior pubic ligament graft rejection, anterior cruciate ligament graft rejection, lateral collateral ligament graft rejection, posterior cruciate ligament graft rejection, medial collateral ligament graft rejection, cranial cruciate ligament graft rejection, and patellar ligament graft rejection.

In some embodiments, the organ graft rejection is selected from heart graft rejection, lung graft rejection, kidney graft rejection, liver graft rejection, pancreas graft rejection, intestine graft rejection, and thymus graft rejection.

In some embodiments, the graft-versus-host disease arises from a bone marrow transplant or one or more blood cells selected from B-cells, T-cells, basophils, common myeloid progenitor cells, common lymphoid progenitor cells, dendritic cells, eosinophils, hematopoietic stem cells, neutrophils, natural killer cells, megakaryocytes, monocytes, or macrophages.

In some embodiments, the inflammatory disease is acute or chronic inflammation.

In some embodiments, the inflammatory disease is selected from osteoarthritis, atopic dermatitis, endometriosis, polycystic ovarian syndrome, inflammatory bowel disease, fibrotic lung disease, and cardiac inflammation.

In some embodiments, the cancer is selected from adenoid cystic carcinoma, adrenal gland tumor, amyloidosis, anal cancer, appendix cancer, astrocytoma, ataxia-telangiectasia, Beckwith-Wiedemann syndrome, bile duct cancer (cholangiocarcinoma), Birt-Hogg-Dubé syndrome, bladder cancer, bone cancer (sarcoma of bone), brain stem glioma, brain tumor, breast cancer, inflammatory breast cancer, metastatic breast cancer, male breast cancer, Carney complex, central nervous system tumors (brain and spinal cord), cervical cancer, childhood cancer, colorectal cancer, Cowden syndrome, craniopharyngioma, desmoid tumor, desmoplastic infantile ganglioglioma, childhood tumor, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor (GIST), germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS-related cancer, juvenile polyposis syndrome, kidney cancer, lacrimal gland tumor, laryngeal and hypopharyngeal cancer, acute lymphoblastic leukemia (ALL), acute myeloid leukemia (AML), B-cell prolymphocytic leukemia and hairy cell leukemia, chronic lymphocytic leukemia (CLL), chronic myeloid leukemia (CML), chronic T-cell lymphocytic leukemia, eosinophilic leukemia, Li-Fraumeni syndrome, liver cancer, lung cancer, non-small cell lung cancer, small cell lung cancer, hodgkin lymphoma, non-hodgkin lymphoma, lynch syndrome, mastocytosis, medulloblastoma, melanoma, meningioma, mesothelioma, multiple endocrine neoplasia type 1, multiple endocrine neoplasia type 2, multiple myeloma, MUTYH (or MYH)-associated polyposis, myelodysplastic syndromes (MDS), nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neuroendocrine tumors, neurofibromatosis type 1, neurofibromatosis type 2, nevoid basal cell carcinoma syndrome, oral and oropharyngeal cancer, osteosarcoma, ovarian, fallopian tube, and peritoneal cancer, pancreatic cancer, parathyroid cancer, penile cancer, Peutz-Jeghers syndrome, pheochromocytoma and paraganglioma, pituitary gland tumor, pleuropulmonary blastoma, prostate cancer, retinoblastoma, rhabdomyosarcoma, salivary gland cancer, Kaposi sarcoma, soft tissue sarcomas, skin cancer (non-melanoma), small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom macroglobulinemia (lymphoplasmacytic lymphoma), Werner syndrome, Wilms tumor, or xeroderma pigmentosum.

In some embodiments, the cardiovascular disease is selected from atherosclerosis, heart failure, left heart failure with reduced ejection fraction, left heart failure with preserved ejection fraction, right ventricular failure, congestive heart failure, restrictive cardiomyopathy, dilated cardiomyopathy, hypertrophic cardiomyopathy, ischemic cardiomyopathy, idiopathic cardiomyopathy, and hypertension.

In some embodiments, the infertility and pregnancy-associated diseases is selected from recurrent pregnancy loss, pre-eclampsia, preterm labor, fetal growth restriction, or intrauterine growth restriction.

In another aspect, provided herein is a method of regenerating a tissue or organ comprising one or more TNFR2+ cells, said method comprising contacting the tissue or organ with an effective amount of the antigen-binding protein, the fusion protein, or the conjugate described herein. In some embodiments, the tissue or organ is selected from pancreas, salivary gland, pituitary gland, kidney, heart, lung, hematopoietic system, cranial nerves, heart, aorta, olfactory gland, ear, nerve, eye, thymus, tongue, bone, liver, small intestine, large intestine, gastrointestinal, lung, brain, skin, peripheral nervous system, central nervous system, spinal cord, breast, embryonic structures, embryo, and testes tissue. In some embodiments, said contacting occurs in vitro. In some embodiments, said contacting occurs in vivo. In some embodiments wherein the method occurs in vivo, the method further comprises administering the antigen-binding protein, the fusion protein, or the conjugate into a subject in need thereof.

In various embodiments of the above-described methods, the subject is a mammal. In some embodiments, the mammal is human.

In another aspect, provided herein is a method for inducing tolerance to a foreign agent and/or preventing or reducing immune response to a foreign agent in a subject in need thereof, comprising an administering to the subject the antigen-binding protein, the fusion protein, or the conjugate described herein.

In some embodiments, the foreign agent is a therapeutic protein, a peptide, a vector, a biochemical vector, a lipid, a carbohydrate, a nucleic acid, a sperm, an oocyte, or an embryo. In some embodiments, the vector is a viral vector, a bacterial vector, or a fungal vector. In some embodiments, the viral vector is a DNA or RNA vector.

### BRIEF DESCRIPTION OF DRAWINGS

FIG. 1 depicts an exemplary general panning strategy for isolation of tumor necrosis factor (TNF) receptor type 2 (TNFR2)-specific variable domain of heavy chain (VHH) antibodies, also referred to herein as V-bodies (Vbs). Binders to human and rodent TNFR2 were enriched from VHH immune libraries by two rounds of phage display. BM, bone marrow.

FIG. 2 shows VHH immune library selection for next-generation sequencing (NGS) across the phage display process. Three initial libraries, 12 samples of the first panning round, and 36 samples of the second panning round, were sequenced with 20 million, 2 million, and 2 million reads, respectively. Comparison of V-body enrichment from the initial library to the first and second round of panning enabled identification of potential V-body candidates.

FIG. 3 shows a schematic diagram of an exemplary NGS workflow. Following phage display, the VHH region of the phage eluate was amplified via polymerase chain reaction (PCR). Unique and sample-specific barcodes were then fused, and NGS was subsequently performed using the Illumina NovaSeq platform (Genewiz). The raw data were de-multiplexed, and then processed by the NGS analysis pipeline. Forward and reverse sequence pairs were merged via overlapping regions and the VHHs, Including complementarity determining regions (CDRs) were annotated. Based on CDR3 identity, V-body sequences were clustered, thereby allowing for detailed analysis of, e.g., V-body enrichment during phage display, sequence diversity, CDR3 length distribution, and cluster abundance. Based on such analyses, more than 600 candidates were selected for DNA synthesis (Twist) and further characterization.

FIGS. 4A-4B illustrate human TNFR2 (hTNFR2) V-body binding validation at a fixed concentration of 1 μM V-body. The bar histogram (FIG. 4A) and table (FIG. 4B) show the percentage of Alexa488-positive cells for all VHHs tested. For the bar histogram, the black dotted line indicates background staining (~5%), and the gray dotted line indicates two times the background level. A V-body having a signal-to-noise ratio greater than 2 is considered as a “binder”. Gray shading within the table indicates binders to hTNFR2. Italicized text within the table indicates different cell passage(s) and date(s) of experiment.

FIGS. 5A-5B illustrate human TNFR2 (hTNFR2) V-body binding validation at a fixed concentration of 100 nM V-body. The bar histogram (FIG. 5A) and table (FIG. 5B) show the percentage of Alexa488-positive cells for all VHHs tested. For the bar histogram, the black dotted line indicates background staining (~5%), and the gray dotted line indicates two times the background level. A V-body having a signal-to-noise ratio greater than 2 is considered as a “binder”. Gray shading within the table indicates binders to hTNFR2.

FIGS. 6A-6C depict cross-specificity of V-body binding to mouse TNFR2 (mTNFR2) (FIG. 6A) and cynomolgus TNFR2 (CTNFR2) (FIG. 6B) at a fixed concentration of 100 nM. The bar histograms (FIGS. 6A-6B) and table (FIG. 6C) show the percentage of Alexa488-positive cells for all VHHs tested. For the bar histogram, the black dotted line indicates background staining, and the gray dotted line indicates two times the background level.

FIG. 7 shows testing of human TNFR2 V-body binding across a range of concentrations for V-bodies ODY-31D6, ODY-35A10, ODY-31G3, ODY-31G11, ODY-33D4, ODY-37C7, and V-bodies. V-bodies were tested at molar concentrations of 100 nM, 50 nM, 12.5 nM, 6.25 nM, 3.12 nM, and 1.55 nM.

FIG. 8 shows a schematic diagram of an exemplary experimental setup for determination of binding affinities of the V-bodies for their respective target via surface plasmon resonance (SPR) (left panel) and a corresponding table describing the V-body candidates analyzed (right panel). Figure discloses SEQ ID NO: 4717.

FIGS. 9A-9F depict surface plasmon resonance (SPR) sensorograms of VHH binding to human, cynomolgus, and mouse TNFR2. Fitted binding curves and calculated dissociation constants (K_{D}) are included.

FIG. 10 shows a summary of binding affinities of 16 selected anti-TNFR2 V-bodies to human, cynomolgus and mouse TNFR2. Cyno, cynomolgus.

FIG. 11 demonstrates that some humanized anti-TNFR2 V-bodies targeted the epitope recognized by a MR2-1 bivalent agonist. N1365hu1 and N1409hu1 may recognize the same epitope as MR2-1. MR2-1 binding enhanced binding of N1402hu1, N1425hu1 and N1277hu1 to TNFR2.

FIGS. 12A-12E show TNFR2 agonism by multivalent V-body fusion constructs. Agonism of bivalent (FIG. 12A), tetravalent (FIGS. 12B-12C), and IL-2 N88D fusion (FIG. 12D) anti-TNFR2 constructs were characterized on NF-κB reporter HEK293 cells stably expressing TNFR2. Dot plots show a dose-dependent response of anti-TNFR2 VHHs compared to a control VHH (Ctrl). FIG. 12E shows that activity of WIL_33D4_2×VHH-Fc, and an IL-2 mutein, was demonstrated in reporter cell lines specific for each signaling pathway. RLU, relative luminescence unit.

FIG. 13 depicts HEK293 TNFR2 NF-κB (Luc) reporter gene assay controls. Anti-hTNFR2 agonist MR2-1 monoclonal antibodies were tested on NF-κB reporter (Luc) HEK293 reporter cell-line stably expressing TNFR2 (clone 25) versus parental cell line (PCL), RLU, relative luminescence unit,

FIGS. 14A-14C shows HEK293 TNFR2 NF-κB (Luc) reporter gene assay samples and assay controls. A description of reporter gene assay samples and assay controls is shown in FIG. 14A. A bar graph showing protein concentration (mg/mL) for the V-body constructs and respective controls is depicted in FIG. 14B. A bar graph showing RLUs for V-body constructs and respective controls tested on a PCL control is depicted in FIG. 14C.

FIGS. 15A-15B depict concentration range curve data generated using MR2-1 (FIG. 15A) and TNFα (FIG. 15B) controls for four assay plates.

FIGS. 16A-16C show exemplary dot plots of RLUs measured across increasing concentrations (mol/L) of control (control 12) and tetravalent V-body fusion constructs comprising four V-bodies mounted onto the fragment crystallizable (Fc) region of a IgG4 variant comprising S228P, L235E and P329G mutations.

FIGS. 17A-17F show exemplary dot plots of RLUs measured across increasing concentrations (mol/L) of control (control 10) and an alternative design of tetravalent V-body fusion constructs comprising four V-bodies mounted onto the Fc region of a IgG4 variant comprising S228P, L235E and P329G mutations.

FIGS. 18A-18C show exemplary dot plots of RLUs measured across increasing concentrations (mol/L) of control (control 2) and bivalent V-body fusion constructs. Limit of detection, LOD.

FIGS. 19A-19C show exemplary dot plots of RLUs measured across increasing concentrations (mol/L) of control (control 13) and IL-2 N88D V-body fusion constructs. Limit of detection, LOD.

FIG. 20 depicts a comparison of RLUs measured across increasing concentrations (mol/L) of monospecific construct 10 (tetravalent Fc) and construct 12 (Vb-Fc-Vb) tested on NF-κB reporter (Luc) HEK293 reporter cell-line stably expressing TNFR2 (clone 8).

FIG. 21 depicts an exemplary experimental timeline of TNFR2 stimulation by multivalent V-body fusion constructs (e.g., tetravalent Fc, Vb-Fc-Vb, rigid bivalent no Fc) on primary human peripheral blood mononuclear cells (PBMCs) and cluster of differentiation 4 positive (CD4⁺) CD25⁺ CD127dim regulatory T cells (Tregs).

FIG. 22 shows a bar graph of an overview of in-assay concentrations (nM) of multivalent V-body fusion constructs first wave binders. Concentrations (nM) of the VHH constructs is also shown.

FIG. 23 illustrates an exemplary gating strategy applied for Treg markers. Treg Donor 1 is shown as an example and an identical strategy was used for Treg Donor 1 and Donor 3. Live cell and CD4 gating were based on Fluorescence Minus One (FMO) FMO control determination of the cut-off point between background fluorescence and positive cell populations. Forkhead box P3 (FoxP3), Human Leukocyte Antigen, DR isotype (HLA-DR), chemokine motif (C-C motif) receptor 8 (CCR8), and OX-40 gating was based on the CD4 subset of IgG control-stained sample from the same donor. For FoxP3, the gate was set at approximately 0.2%. For OX-40, HLA-DR and CCR8, the gate was set at approximately 2%.

FIGS. 24A-24B demonstrate multivalent anti-TNFR2 V-body fusion constructs increased expression of the Treg suppression marker HLA-DR and CCR8. Histograms displaying expression of Treg suppression marker HLA-DR for specific 37C7 binders compared to control formats (FIG. 24A). Density plots displaying expression of Treg suppression marker HLA-DR and CCR8 for specific 37C7 binders compared to control formats (FIG. 24B). Fluorescein isothiocyanate, FITC; Phycoerythrin, PE.

FIGS. 25A-25B demonstrates tetravalent anti-TNFR2 V-body fusion constructs strongly increased expression of Treg suppression marker HLA-DR on FoxP3⁺ Tregs. The bar graphs show HLA-DR mean fluorescent intensity (MFI) measured for each of the tetravalent Fc, Vb-Fc-Vb, and rigid bivalent no Fc V-body fusion formats relative to control formats.

FIG. 26 shows dose-response curves based on HLA-DR MFI values of CD4⁺ FoxP3⁺ Tregs for construct 37C7 and control 10 for Donor 2.

FIG. 27 shows dose-dependent induction of Treg suppression marker HLA-DR expression across various concentrations of tetravalent anti-TNFR2 V-body Fc fusion construct 10 for Donor 1 (top panel) and Donor 2 (bottom panel).

FIG. 28 shows dose-dependent induction of Treg suppression marker HLA-DR expression across various concentrations of rigid bivalent anti-TNFR2 V-body fusion construct 2 for Donor 1 (top panel) and Donor 2 (bottom panel).

FIG. 29 shows dose-dependent induction of Treg suppression marker HLA-DR expression across various concentrations of tetravalent anti-TNFR2 V-body Vb-Fc-Vb fusion construct 12 for Donor 1 (top panel) and Donor 2 (bottom panel).

FIG. 30 shows exemplary design of multivalent anti-TNFR2 V-body fusion constructs, Anti-TNFR2 V-bodies are shown as ovals, linkers are shown with flexible (e.g., GS linkers) as curved lines, rigid linkers (e.g., proline linker) as straight lines, and Fc domains as dimeric bars. Figure discloses SEQ ID NO: 4718.

FIG. 31 shows assessment of tetravalent-Fc VHH activity on naïve CD4+CD25+CD45RA+ human Treg. HLA-DR and CCR8 expression on CD4+FOXP3+ and expansion after 5 day stimulation with anti-CD3/IL-2 plus VHH or MR2-1 are shown.

FIG. 32A shows the ability of TNFR2 VHH to stabilize Treg. Naive CD4+CD25+CD45RA+ human Treg from healthy donors were stimulated with IL-2 and anti-CD3 in the presence of TNFR2 agonist VHH or TNFR2 monoclonal agonist MR2-1 for 5 days.

FIG. 32B shows the effect of TNFR2 VHH on early markers of Treg stability. Naive CD4+CD25+CD45RA+ human Treg from healthy donors were stimulated with IL-2 and anti-CD3 in the presence of TNFR2 agonist VHH or IL-2 mutein for 5 days.

FIGS. 33A-33B show additional results of in vitro treatment of human Treg (CD4+ FOXP3+) with VHH WIL_33D4_2×VHH-Fc or IL-2 mutein in the presence of anti-CD3 and IL-2. WIL 33D4_2×VHH-Fc induced and expanded a Treg population with high levels of FOXP3, EZH2 (a marker of stability), CCR8, and HLA-DR (biomarkers of tissue homing and Treg immunosuppressive functionality). T-test: *p<0.05; **p<0.01; n=3.

FIGS. 34A-34C show the effect of TNFR2 VHH on Treg stability under inflammatory conditions. Human Treg were expanded with IL-2 mutein or TNFR2 VHH in the presence of anti-CD3 and IL-2 for 5 days and then cultured with proinflammatory cytokines (IL-1b, IL-21, and IL-23+/−TGFb) for 11 to 12 days; IL-17A or IFNγ production after PMA/ionomycin stimulation was assessed together with FOXP3 by flow cytometry. The conversion of human Treg in vitro to cells that produce Th1/17 cytokines (IFNγ/IL-17A) triggered by the inflammatory cytokines shown was prevented by co-stimulation with TNFR2 VHH but not with IL-2 mutein. P-values indicate results of a paired T-test; *=p<0.05; **=p<0.01; Fc=human IgG4 mutant Fc.

FIG. 35 shows assessment of Treg function upon TNFR2 agonism. Naive Treg were stimulated for 7 days with anti-CD3/IL-2 plus TNFR2 VHH (WIL_33D4_2×VHH-Fc), control VHH, MR2-1, control IgG, or IL-2 mutein; after 7 days, stimuli were removed and cells were incubated with cell tracer-labeled autologous responder cells (naïve CD4+ T cells); bar graph shows effector CD4 cell proliferation measured as % dividing CD4+FOXP3-cells; FACS histograms show dilution of the cell tracer at different Treg:CD4 (responder) ratios of one of four donors. WIL_33D4_2×VHH-Fc induces Treg that are better able to restrain effector CD4 cell proliferation than IL-2 mutein.

FIG. 36 shows the effect of TNFR2 agonist VHH on Treg population size in mice. An exemplary design of the experimental procedure is shown. CD4+FOXP3+ Treg expansion in the spleen of mice 5 days after a single injection of 2.5 mg/kg control VHH or TNFR2-specific VHH WIL_33D4_2×VHH-Fc is shown.

FIG. 37 shows that TNFR2 agonist VHH activates Treg in vivo. CCR8 is a chemokine receptor expressed on highly suppressive Treg and involved in cell migration (Whiteside et al., Immunol 2021; 163:512). ICAM-1 surface adhesion molecule is required for Treg function (Gottrand et al., Immunol 2015; 146 (4): 657). ICOS costimulatory molecule is upregulated upon Treg activation and maintains FOXP3 expression (Landuyt et al., J Immunol 2019; 202 (4): 1039). Proportion of Treg (CD4+CD25+ FOXP3+) in spleen expressing activation markers (CCR8, ICAM-1, or ICOS) 5 days after a single injection of control VHH or TNFR2-specific VHH WIL_33D4_2×VHH-Fc is shown. 1-way ANOVA was performed for control VHH vs. WIL_33D4_2×VHH-Fc; only significant differences are shown; ****=p<0.0001.

FIG. 38 shows that TNFR2 agonist VHH selectively expands Treg in the spleen. Cell subsets as percentage of CD45+ cells in the spleen 5 days after single injection of TNFR2-specific VHH WIL_33D4_2×VHH-Fc or control VHH are shown.

FIG. 39 shows that TNFR2 agonist VHH increases serum level of IL-10. IL-10 is a key anti-inflammatory cytokine (Saraiva et al., J Exp Med 2020; 217 (1):e20190418). Serum cytokine concentration 5 days after single injection of TNFR2-specific VHH WIL_33D4_2×VHH-Fc or control VHH is shown. 1-way ANOVA performed for control VHH vs. WIL_33D4_2×VHH-Fc; only significant differences are shown; ****=p<0.0001.

FIG. 40A-40B show frequency of Treg (CD4+ FOXP3+) among total immune cells (CD45+) in the spleen, blood, colon, and lung in human TNFR2 knock-in mice 5 days after single injection of ODY-520. T-test: ***p<0.001; ****p<0.0001; n=4.

FIGS. 41A-41E show ODY-520 selectively increases the Treg population in the spleen 5 days after a single administration compared to IL-2 N88D, a mutein that is active in mice. WIL_33D4_2×VHH-Fc is more selective for Treg and induces a higher level of FOXP3 and surface markers (FOXP3, ICAM-1, OX-40, ICOS, and CCR8), consistent with superior function and stability. One-way ANOVA test: ****p<0.0001; n=4.

FIGS. 42A-42B show that Treg expansion in the spleen as well as increased expression of FOXP3, linked to Treg stability and function, and Treg activation shown by up-regulation of ICAM-1 and ICOS.

FIGS. 43A-43C show that reduction of arthritis, measured by paw volume and arthritis score, in a model of collagen-antibody induced arthritis upon treatment with TNFR2 agonists ODY-520 and ODY-781.

FIGS. 44A-44B show Treg expansion by TNFR2 agonist without inducing proinflammatory cytokines when compared to CD28 agonist.

### DETAILED DESCRIPTION OF THE INVENTION

Regulatory T cells (Treg) are a population of lymphocytes with immunosuppressive function. Activation and expansion of Treg is an attractive therapeutic approach for autoimmune diseases currently being evaluated clinically. In addition to inducing Treg activation and expansion, an effective Treg-directed therapy needs to generate cells with a stable immunosuppressive phenotype that is resistant to conversion to T effector function under inflammatory conditions. Nevertheless, current clinical approaches to stimulate Treg to treat autoimmunity expand Treg by increasing homeostatic proliferation (e.g., via IL-2 muteins) without improving Treg stability. Clinical trials testing IL-2-based approaches to treat autoimmune disease by enhancing Treg function have demonstrated safety and the ability to expand Treg across diseases; however, Treg specificity, Treg stability, and therapeutic efficacy are not optimal (PNAS 2010; 107 (45): 19402; clinicaltrials.gov/study/NCT03943550; clinicaltrials.gov/study/NCT04433585). Optimal Treg therapy needs to (1) expand Treg population size with cells that (2) migrate to tissues where disease occurs and (3) exert immunosuppressive effects, while (4) resisting conversion to inflammatory Th1/17 cells. Therefore, improved therapeutic approaches to promote and stabilize Treg immunosuppressive activity are needed.

TNFR2 agonism is an alternative approach to enhance Treg function that is projected to address all objectives required for an optimal therapy, including the generation of stable Treg cells that resist conversion to Th1/17 (Front Immunol 2022; 13:888274; Sci Rep 2023; 13 (1): 13762; PNAS 2019; 116 (43): 21666; J Immunol 2013; 190:1076; Arthritis Rheumatol 2020; 72 (4): 576). In one aspect, the present invention provides as described herein, TNFR2 agonists to enhance Treg immunosuppressive activity using a single-domain antibody (e.g., VHH) platform.

### Definitions

Unless defined otherwise, all technical and scientific terms used herein have the same meaning as commonly understood by one of ordinary skill in the art to which this disclosure belongs. For purposes of interpreting this specification, the following description of terms will apply and whenever appropriate, terms used in the singular will also include the plural and vice versa. All patents, applications, published applications and other publications are incorporated by reference in their entirety. In the event that any description of terms set forth conflicts with any document incorporated herein by reference, the description of term set forth below shall control.

As used herein, the term “about,” when used in reference to a particular recited numerical value, means that the value may vary from the recited value by no more than 5%. For example, as used herein, the expression “about 100” includes 95 and 105 and all values in between (e.g., 96, 97, 98, 99, etc.).

The term “antigen” encompasses any agent (e.g., protein, peptide, polysaccharide, glycoprotein, glycolipid, nucleotide, portions thereof, or combinations thereof) that may be specifically bound by the products of specific humoral or cellular immunity, such as an antibody molecule or T-cell receptor. In various embodiments of the present disclosure, the antigen described herein is TNFR2, including human, cynomolgus, and/or mouse TNFR2.

The term “epitope” can refer to an antigenic determinant on the surface of an antigen to which an antibody molecule binds. A single antigen may have more than one epitope. Thus, different antibodies may bind to different areas on an antigen and may have different biological effects (e.g., agnostic, or antagonistic effects). Epitopes may be either conformational or linear. A conformational epitope is formed by spatially juxtaposed amino acids from different segments of the linear polypeptide chain. A linear epitope is formed by adjacent amino acid residues in a polypeptide chain. In some cases, an epitope may include non-peptidic moieties on the antigen, such as saccharides, phosphoryl groups, or sulfonyl groups.

The term “antigen-binding protein” refers in its broadest sense to a protein that specifically binds an antigen (e.g., TNFR2). In certain embodiments, an antigen-binding protein is an antibody or an antigen-binding fragment of an antibody, such as a human antibody, a humanized antibody; a camelid antibody; a chimeric antibody; a recombinant antibody; a heavy chain antibody; a single-domain antibody (e.g., VHH); a single chain antibody (e.g., single chain fragment variable (scFv)); a diabody; a triabody; a tetrabody; a Fab fragment; a F(ab′)2 fragment; an IgD antibody; an IgE antibody; an IgM antibody; an IgG1 antibody; an IgG2 antibody; an IgG3 antibody; or an IgG4 antibody, and fragments thereof. The term “antigen-binding protein” also encompasses, for example, an alternative protein scaffold or artificial scaffold with grafted CDRs or CDR derivatives. Such scaffolds include, but are not limited to, antibody-derived scaffolds comprising mutations introduced to, for example, stabilize the three-dimensional structure of the antigen-binding protein as well as wholly synthetic scaffolds comprising, for example, a biocompatible polymer. In addition, peptide antibody mimetics can be used, as well as scaffolds based on antibody mimetics utilizing fibronectin components (e.g., fibronectin type III domain (FN3) as a scaffold.

The term “antibody” and “immunoglobulin” or “lg” are used interchangeably herein, and is used in the broadest sense and encompasses, for example, individual monoclonal antibodies (including agonist, antagonist, neutralizing antibodies, full length or intact monoclonal antibodies), antibody compositions with polyepitopic or monoepitopic specificity, polyclonal antibodies, monovalent antibodies, multivalent antibodies, multispecific antibodies (e.g., bispecific antibodies), single-domain antibodies (e.g., VHH), single chain antibodies, intrabodies, anti-idiotypic (anti-Id) antibodies, and antigen-binding fragments of antibodies, as described below. An antibody can be human, humanized, camelized, recombinantly produced, chimeric, synthetic, affinity de-matured and/or affinity matured as well as an antibody from other species, for example mouse, camel, llama, rabbit, etc. In specific embodiments, the specific target antigen that can be bound by an antibody provided herein includes a TNFR2 polypeptide, TNFR2 fragment or TNFR2 epitope. An “antigen-binding fragment” generally refers a portion of an antibody heavy and/or light chain polypeptide that retains some or all of the binding activity of the antibody from which the fragment was derived. Non-limiting examples of antigen-binding fragments include single-domain antibody (e.g., VHH), single-chain Fvs (scFv), Fab fragments, F(ab′) fragments, F(ab)2 fragments, F(ab′)2 fragments, disulfide-linked Fvs (sdFv), Fd fragments, Fv fragments, diabody, triabody, tetrabody and minibody, or a chemically modified derivative thereof. In particular, antibodies provided herein include immunoglobulin molecules and molecules that contain immunologically active portion(s) of an immunoglobulin molecule, for example, one or more complementarity determining regions (CDRs) of an antibody that binds to TNFR2. Such antibody fragments can be found described in, for example, Harlow and Lane, Antibodies: A Laboratory Manual, Cold Spring Harbor Laboratory, New York (1989); Myers (ed.), Molec. Biology and Biotechnology: Å Comprehensive Desk Reference, New York: VCH Publisher, Inc.; Huston et al., Cell Biophysics, 22:189-224 (1993); Plückthun and Skerra, Meth. Enzymol., 178:497-515 (1989) and in Day, E.D., Advanced Immunochemistry, Second Ed., Wiley-Liss, Inc., New York, N.Y. (1990). The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule.

The term “single-domain antibody” or “sdAb” as used herein, refers to an antibody or antibody fragment containing a single antibody variable domain that is able to bind to a specific antigen alone, without the requirement of another antibody variable domain. The complementary determining regions (CDRs) of a single-domain antibody are part of a single antibody variable domain. Examples of single-domain antibodies include, but are not limited to, heavy chain antibodies, antibodies naturally devoid of light chains, single domain antibodies derived from conventional four-chain antibodies, engineered antibodies, variable domains derived from the aforementioned antibodies, and single domain scaffolds other than those derived from antibodies. Single domain antibodies may be derived from any species including, but not limited to mouse, human, camel, llama, shark, goat, rabbit, and/or bovine. In some embodiments, a single domain antibody as used herein is a naturally occurring single domain antibody known as heavy chain antibody devoid of light chains. For clarity reasons, the variable domain derived from a heavy chain antibody naturally devoid of light chain is known herein as a VHH to distinguish it from the conventional VH of four-chain immunoglobulins. Such a VHH molecule can be derived from antibodies raised in Camelidae species, e.g., camel, llama, dromedary, alpaca, and guanaco. Other species besides Camelidae may produce heavy chain antibodies naturally devoid of light chain, which are also within the scope of the invention. For example, cartilaginous fishes such as sharks can produce immunoglobulin-like structures known as VNAR. In some embodiments, a single-domain antibody may be obtained from a Camelidae VH domain. In some embodiments, a single-domain antibody may be obtained from human VH by camelization. See Saerens et al., Current Opinion in Pharmacology, 2008, 8:600-608, the disclosure of which being incorporated by reference, for review of single-domain antibodies.

The term “specifically binds” as used herein means that an antigen-binding protein forms a complex with a target antigen that is relatively stable under physiologic conditions. Specific binding can be characterized by a dissociation constant (K_{D}) of about 1×10⁻⁶ M or less (e.g., less than 10⁻⁶ M, less than 5×10⁻⁷M, less than 10⁻⁷ M, less than 5×10⁻⁸ M, less than 10⁻⁸ M, less than 5×10⁻⁹ M, less than 10⁻⁹M, or less than 10⁻¹⁰ M). Methods for determining the binding affinity of an antigen-binding protein, e.g., an antibody or an antibody fragment, to a target antigen are well known in the art and include, e.g., surface plasmon resonance (e.g., BIACORE® assays), bio-layer interferometry, ligand binding assays (e.g., enzyme-linked immunosorbent assay (ELISA)), equilibrium dialysis, fluorescent-activated cell sorting (FACS), or flow cytometry-based binding assays and the like. Specific binding to a particular target antigen from a certain species does not exclude that the antigen-binding protein can also specifically bind to the analogous target from a different species. For example, specific binding to human TNFR2 does not exclude that the antigen-binding protein can also specifically bind to TNFR2 from cynomolgus monkeys (“cyno”).

The term “isolated” when used in the context of antigen-binding proteins (e.g., antibodies, such as single-domain antibodies), polypeptides, polynucleotides, and vectors, means the antigen-binding proteins (e.g., antibodies, such as single-domain antibodies), polypeptides, polynucleotides and vectors are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor, or insignificant amounts of impurity may remain) or to an absence of water, buffers, or salts or to components of a pharmaceutical formulation that includes the antigen-binding proteins (e.g., antibodies, such as single-domain antibodies).

The term “operably linked” as used herein can refer to a functional relationship between two or more regions of a polypeptide chain in which the two or more regions are linked so as to produce a functional polypeptide.

As used herein, the term “variant”, “derivative” or “derived from” in the context of proteins or polypeptides (e.g., antigen-binding proteins or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a variant or derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a variant or derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a variant or derivative of; (d) a polypeptide encoded by nucleic acids can hybridize under high, moderate or typical stringency hybridization conditions to nucleic acids encoding the polypeptide it is a variant or derivative of; (e) a polypeptide encoded by a nucleotide sequence that can hybridize under high, moderate or typical stringency hybridization conditions to a nucleotide sequence encoding a fragment of the polypeptide, it is a variant or derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a variant or derivative of. The terms also encompass a fusion protein or polypeptide comprising the polypeptide it is a variant or derivative of.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs GAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24:307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions, and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

The terms “enhance” or “promote,” or “increase,” or “expand,” or “improve” refer generally to the ability of a composition contemplated herein to produce, elicit, or cause a greater physiological response (i.e., downstream effects) compared to the response caused by either vehicle or a control molecule/composition. A measurable physiological response may include an increase in immune cell expansion, activation, effector function, persistence, and/or an increase in tumor cell death killing ability, among others apparent from the understanding in the art and the description herein. In certain embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

The terms “decrease” or “lower,” or “lessen,” or “reduce,” or “abate” refer generally to the ability of composition contemplated herein to produce, elicit, or cause a lesser physiological response (I.e., downstream effects) compared to the response caused by either vehicle or a control molecule/composition. In certain embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle or a control composition.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The terms “effective amount” or “therapeutically effective amount” refer to a quantity and/or concentration of a composition containing an active ingredient (e.g., anti-TNFR2 antigen-binding protein) that when administered into a patient either alone (i.e., as a monotherapy) or in combination with additional therapeutic agents, yields a significant decrease in disease progression as, for example, by ameliorating or eliminating symptoms and/or the cause of the disease. An effective amount may be an amount that relieves, lessens, or alleviates at least one symptom or biological response or effect associated with a disease or disorder, prevents progression of the disease or disorder, or improves physical functioning of the patient. A therapeutically effective amount of a composition containing an active agent may vary according to factors such as the disease state, age, sex, and weight of the individual, and the ability of the active agent to elicit a desired response in the individual. A therapeutically effective amount is also one in which any toxic or detrimental effects of the active agent are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “individual”, “subject” and “patient” are used interchangeably herein to refer to an animal; for example a mammal. The terms include human and veterinary subjects. In some embodiments, methods of treating mammals, Including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some embodiments, a subject can be a subject in need of treatment for a disease or disorder. In particular embodiments, the subject is a human.

### Anti-TNFR2 Antigen-Binding Proteins

The present disclosure provides antigen-binding proteins (e.g., antibodies, such as single-domain antibodies) that bind to tumor necrosis factor receptor 2 (TNFR2).

TNFR2 is a single pass type-1 membrane protein belonging to the TNFR superfamily. It consists of an extracellular domain with four cysteine rich domains (CRD) and an intracellular domain that is involved in signaling. The cysteine rich domains contain a total of 10 disulfide bonds stabilizing the elongated structure of the protein. Unlike TNFR1 which is widely expressed, the expression of TNFR2 is restricted on immune cells including Tregs, myeloid cells, CD8 and NK cells but also glial cells, endothelial cells, and fibroblasts (Medler and Wajant, 2019).

In some embodiments, the human TNFR2 protein is encoded by the human TNF receptor superfamily member 1B (TNFRSF1B) gene (NCBI Gene ID: 7133) and has the amino acid sequence of

(SEQ ID NO: 4028)
MAPVAVWAALAVGLELWAAAHALPAQVAFTPYAPEPGSTCRLREYYDQTAQMCCSKCSPGQHAKVFCTKTSDTVCD
SCEDSTYTQLWNWVPECLSCGSRCSSDQVETQACTREQNRICTCRPGWYCALSKQEGCRLCAPLRKCRPGFGVARPG
TETSDVVCKPCAPGTFSNTTSSTDICRPHQICNVVAIPGNASMDAVCTSTSPTRSMAPGAVHLPQPVSTRSQHTQPTPE
PSTAPSTSFLLPMGPSPPAEGSTGDFALPVGLIVGVTALGLLIIGVVNCVIMTQVKKKPLCLQREAKVPHLPADKARGTQ
GPEQQHLLITAPSSSSSSLESSASALDRRAPTRNQPQAPGVEASGAGEARASTGSSDSSPGGHGTQVNVTCIVNVCSSS
DHSSQCSSQASSTMGDTDSSPSESPKDEQVPFSKEECAFRSQLETPETLLGSTEEKPLPLGVPDAGMKPS (UniProtKB
Accession No. P20333)

In some embodiments, the cyno TNFR2 protein is encoded by the Cyno TNF receptor superfamily member 1B (TNFRSF1B) gene (Gene ID: 102144224) and has the amino acid sequence of

(SEQ ID NO: 4029)
MVTRRGGDDRRRLKGHRVLGVTLEVLARRCWGGRVGGPAEAGEGRGGGVSKAGWPRPAPPRCLASGPLQRGLSLS
VAAGWRAQRSLGRRRCAARARGREGRGNRIPPAPMAPAAVWAALAVGLELWAAGHALPÅQVAFTPYAPEPGGTCR
LREYYDQTAQMCCSKCPPGQHAKVFCTKTSDTVCDSCEDSTYTQLWNWVPECLSCGSRCSSDQVETQACTREQNRIC
TCRPGWYCALSKQEGCRLCAQLRKCRPGFGVARPGTETSDVVCKPCAPGTFSNTTSSTDICRPHQICHVVAIPGNASM
DAVCTSTSPTRSMAPGAVHLPQPVSTRSQHTQPTPAPSTAPGTSFLLPVGPSPPAEGSTGDIVLPVGLIVGVTALGLLIIG
VVNCVIMTQVKKKPLCLORETKVPHLPADKARGAQGPEQQHLLTTVPSSSSSSLESSASALDRRAPTRNQPQAPGAEK
ASGAGEARASTGSSDSSPGGHGTQVNVTCIVNVCSSSDHSSQCSSQASSTMGDTDASPSGSPKDEQVPFSKEECAFRS
QLETPETLLGSTEEKPLPLGVPDAGMKPS (UniProtKB Accession No. A0A2KSVET2)

In some embodiments, the mouse TNFR2 protein is encoded by the mouse TNF receptor superfamily member 1B (Tnfrsf1b) gene (Gene ID: 21938) and has the amino acid sequence of

(SEQ ID NO: 4030)
MAPAALWVALVFELQLWATGHTVPAQVVLTPYKPEPGYECQISQEYYDRKAQMCCAKCPPGQYVKHFCNKTSDTVC
ADCEASMYTQVWNQFRTCLSCSSSCTTDQVEIRACTKQQNRVCACEAGRYCALKTHSGSCRQCMRLSKCGPGFGVAS
SRAPNGNVLCKACAPGTFSDTTSSTDVCRPHRICSILAIPGNASTDAVCAPESPTLSAIPRTLYVSQPEPTRSQPLDQEPG
PSQTPSILTSLGSTPIIEQSTKGGISLPIGLIVGVTSLGLLMLGLVNCIILVQRKKKPSCLQRDAKVPHVPDEKSQDAVGLEQ
QHLLTTAPSSSSSSLESSASAGDRRAPPGGHPQARVMAEAQGFQEARASSRISDSSHGSHGTHVNVTCIVNVCSSSDHS
SQCSSQASATVGDPDAKPSASPKDEQVPFSQEECPSQSPCETTETLQSHEKPLPLGVPDMGMKPSQAGWFDQIAVKV
A (UniProtKB Accession No. P25119)

In various embodiments, antigen-binding proteins of the present disclosure have an agonist effect upon binding to TNFR2. While not wishing to be bound by theory, an agonistic TNFR2 binder can promote or increase activation of TNFR2 and/or potentiate one or more signal transduction pathways mediated by TNFR2. For example, agonistic TNFR2 binders may promote or increase the proliferation of a population of Treg cells. Agonistic TNFR2 binders may promote or increase TNFR2 activation by binding TNFR2, e.g., to induce a conformational change that renders the receptor biologically active. For example, agonistic TNFR2 binders may nucleate the trimerization of TNFR2 in a manner similar to the interaction between TNFR2 and its cognate ligand, tumor necrosis factor (TNF), thus inducing TNFR2-mediated signaling. In some embodiments, agonistic TNFR2 binding proteins of the present disclosure may be capable of inducing the proliferation of Treg cells (e.g., CD4+, CD25+, FOXP3+ Treg cells). Agonistic TNFR2 binding proteins of the present disclosure may also be capable of suppressing the proliferation of cytotoxic T lymphocytes (e.g., CD8+ T-cells), e.g., through activation of immunomodulatory Treg cells or by directly binding TNFR2 on the surface of an autoreactive cytotoxic T-cell and inducing apoptosis.

In some embodiments, antigen-binding proteins of the present disclosure upon binding to TNFR2 do not impair the binding of its cognate ligand, tumor necrosis factor (TNF), to TNFR2. In some embodiments, antigen-binding proteins of the present disclosure do not have overlapping epitopes with TNF. In some embodiments, antigen-binding proteins of the present disclosure have overlapping epitopes with TNF. In some embodiments, antigen-binding proteins of the present disclosure upon binding to TNFR2 promote or facilitate TNFR2 oligomerization (in the presence or absence of TNF, respectively). In some embodiments, antigen-binding proteins of the present disclosure upon binding to TNFR2 multimerize (e.g., dimerize) the TNFR2 trimers to induce intracellular signaling.

In some embodiments, antigen-binding proteins of the present disclosure bind to human TNFR2. In some embodiments, antigen-binding proteins (e.g., antibodies such as single-domain antibodies) of the present disclosure may bind to human TNFR2 with a K_{D} of less than about 1×10⁻⁶ M, for example, less than about 5×10⁻⁷ M, less than about 3×10⁻⁷ M, less than about 1×10⁻⁷ M, less than about 8×10⁻⁸ M, less than about 5×10⁻⁸ M, less than about 3×10⁻⁸ M, less than about 1×10⁻⁸ M, less than about 8×10⁻⁹ M, less than about 5×10⁻⁹ M, less than about 3×10⁻⁹ M, or less than about 1×10⁻⁹ M, or about 1×10⁻¹⁰ to 1×10⁻⁹ M, 1×10⁻¹⁰ to 5×10⁻⁹ M, about 1×10⁻¹⁰ to 1×10⁻⁸ M, about 1×10⁻¹⁰ to 5×10⁻⁸ M, about 1×10⁻⁹ to 1×10⁻⁸ M, about 1×10⁻³ to 5×10⁻⁸ M, about 1×10⁻⁹ to 1×10⁻⁷ M, or about 1×10⁻⁸ to 1×10⁻⁷ M.

In some embodiments, antigen-binding proteins of the present disclosure bind to cynomolgus monkey (“cyno”) TNFR2. In some embodiments, antigen-binding proteins (e.g., antibodies such as single-domain antibodies) of the present disclosure may bind to cyno TNFR2 with a K_{D} of less than about 1×10⁻⁶ M, for example, less than about 5×10⁻⁷ M, less than about 3×10⁻⁷ M, less than about 1×10⁻⁷M, less than about 8×10⁻⁸ M, less than about 5×10⁻⁸ M, less than about 3×10⁻⁸ M, less than about 1×10⁻⁸ M, less than about 8×10⁻⁹ M, less than about 5×10⁻⁹ M, less than about 3×10⁻⁹ M, or less than about 1×10⁻⁹ M, or about 1×10⁻¹⁰ to 1×10⁻⁹ M, 1×10⁻¹⁰ to 5×10⁻⁹ M, about 1×10⁻¹⁰ to 1×10⁻⁸ M, about 1×10⁻¹⁰ to 5×10⁻⁸ M, about 1×10⁻⁹ to 1×10⁻⁸ M, about 1×10⁻⁹ to 5×10⁻⁸ M, about 1×10⁻⁹ to 1×10⁻⁷ M, about 1×10⁻⁹ to 2×10⁻⁷ M, about 1×10⁻⁹ to 5×10⁻⁷ M, about 1×10⁻⁸ to 1×10⁻⁷ M, about 1×10⁻⁸ to 2×10⁻⁷ M, about 1×10⁻⁸ to 5×10⁻⁷ M, or about 1×10⁻⁸ to 1×10⁻⁶ M.

In some embodiments, antigen-binding proteins of the present disclosure bind to mouse TNFR2. In some embodiments, antigen-binding proteins of the present disclosure may bind to mouse TNFR2 with a K_{D} of less than about 1×10⁻⁶ M, for example, less than about 5×10⁻⁷ M, less than about 3×10⁻⁷ M, less than about 1×10⁻⁷ M, less than about 8×10⁻⁸ M, less than about 5×10⁻³ M, less than about 3×10⁻⁸ M, less than about 1×10⁻⁸ M, less than about 8×10⁻⁹ M, less than about 5×10⁻⁹ M, less than about 3×10⁻⁹ M, or less than about 1×10⁻³ M, or about 1×10⁻¹⁰ to 1×10⁻⁹ M, 1×10⁻¹⁰ to 5×10⁻⁹ M, about 1×10⁻¹⁰ to 1×10⁻⁸ M, about 1×10⁻¹⁰ to 5×10⁻⁸ M, about 1×10⁻⁹ to 1×10⁻¹⁰ M, about 1×10⁻⁹ to 5×10⁻⁸ M, about 1×10⁻⁹ to 1×10⁻⁷ M, about 1×10⁻⁹ to 2×10⁻⁷ M, about 1×10⁻⁹ to 5×10⁻⁷ M, about 1×10⁻⁸ to 1×10⁻⁷ M, about 1×10⁻⁸ to 2×10⁻⁷ M, about 1×10⁻⁸ to 5×10⁻⁷ M, or about 1×10⁻⁸ to 1×10⁻⁶ M. In some embodiments, antigen-binding proteins of the present disclosure do not bind to mouse TNFR2.

In some embodiments, anti-TNFR2 antigen-binding proteins of the present disclosure may specifically bind TNFR2 without exhibiting specific binding for another receptor of the tumor necrosis factor receptor (TNFR) superfamily.

Binding affinity of a molecular interaction between two molecules can be measured via various techniques, such as surface plasmon resonance (SPR), bio-layer interferometry (BLI), enzyme-linked immunosorbent assay (ELISA), equilibrium dialysis, fluorescent-activated cell sorting (FACS), or flow cytometry binding assays and the like. Surface plasmon resonance is a biosensor technique that allows for the analysis of real-time biospecific interactions by detection of alterations in protein concentrations within a biosensor matrix, where one molecule is immobilized on the biosensor chip and the other molecule is passed over the immobilized molecule under flow conditions (see e.g., Ober et al. 2001, Intern. Immunology 13:1551-1559). SPR can for example be performed using the BIACORE® system or Carterra LSA system. Another biosensor technique that can be used to determine affinities of biomolecular interactions is bio-layer interferometry (BLI) (see e.g., Abdiche et al. 2008, Anal. Biochem. 377:209-217). Bio-layer interferometry is a label-free optical technique that analyzes the interference pattern of light reflected from two surfaces: an internal reference layer (reference beam) and a layer of immobilized protein on the biosensor tip (signal beam). A change in the number of molecules bound to the tip of the biosensor causes a shift in the interference pattern, reported as a wavelength shift (nm), the magnitude of which is a direct measure of the number of molecules bound to the biosensor tip surface. Since the interactions can be measured in real-time, association and dissociation rates and affinities can be determined. BUI can for example be performed using the Octet® Systems. Alternatively, affinities can be measured in Kinetic Exclusion Assay (KinExA) (see e.g., Drake et al. 2004, Anal. Biochem., 328:35-43), which is a solution-based method to measure true equilibrium binding affinity and kinetics of unmodified molecules. Equilibrated solutions of an antibody/antigen complex are passed over a column with beads precoated with antigen (or antibody), allowing the free antibody (or antigen) to bind to the coated molecule. Detection of the antibody (or antigen) thus captured is accomplished with a fluorescently labeled protein binding the antibody (or antigen).

Antigen-binding proteins of the present disclosure can include an antibody or an antigen-binding fragment of an antibody, such as a human antibody, a humanized antibody; a camelid antibody; a chimeric antibody; a recombinant antibody; a heavy chain antibody; a single-domain antibody (e.g., VHH); a single chain antibody (e.g., single chain fragment variable (scFv)); a diabody; a triabody; a tetrabody; a Fab fragment; a F(ab′)2 fragment; an IgD antibody; an IgE antibody; an IgM antibody; an IgG1 antibody; an IgG2 antibody; an IgG3 antibody; or an IgG4 antibody, and fragments thereof.

In some embodiments, an antigen-binding protein that binds to TNFR2 is a single-domain antibody (also termed as “sdAb”). The single-domain antibodies of the present disclosure can be derived from numerous sources, including but not limited to VHHs, VNARs, or VH domains (naturally occurring or engineered VH domains). VHHs can be generated from camelid heavy chain only antibodies and libraries thereof. VNARs can be generated from cartilaginous fish heavy chain only antibodies and libraries thereof. Various methods have been implemented to generate monomeric sdAbs from conventionally heterodimeric VH and VL domains, including interface engineering and selection of specific germline families. In some embodiments, the sdAb of the present invention are human or humanized.

In some embodiments, a single-domain antibody described herein is a VHH fragment (also known as a nanobody). VHH fragments are also referred to as “V-bodies” in the present disclosure. In some embodiments, the VHH is a camelid VHH, a humanized VHH or a camelized VH. In some embodiments, a single-domain antibody described herein is a VH domain. In some embodiments, a single-domain antibody described herein is a naturally occurring VH domain or engineered VH domain.

The variable domain of an antigen-binding protein (e.g., antibody such as a single-domain antibody) of the present disclosure comprises at least three complementarity determining regions (CDRs) which determine its binding specificity. Preferably, in a variable domain, the CDRs are distributed between framework regions (FRs). The variable domain typically contains 4 framework regions interspaced by 3 CDR regions, resulting in the following typical antibody variable domain structure: FR1-CDR1-FR2-CDR2-FR3-CDR3-FR4. CDRs and/or FRs of the single domain antibody of the invention may be fragments or derivatives from a naturally occurring antibody variable domain or may be synthetic.

Sequence identifiers corresponding to exemplary anti-TNFR2 VHH antibodies provided herein are listed in Table 1-1. Table 1-1 sets forth the sequence identifiers of amino acid sequences of the complementarity determining regions (CDR1, CDR2 and CDR3), amino acid and DNA sequences of the full-length camelid VHH antibodies, as well as amino acid sequences of corresponding humanized VHH antibodies. Amino acid sequences of additional exemplary anti-TNFR2 VHH antibodies and corresponding humanized VHH antibodies are provided in Table 1-2.

TABLE 1-1
Sequence identifiers for exemplary anti-TNFR2 VHH antibodies

| | | CDR1 | CDR2 | CDR3 | Non-humanized VHH | | Humanized |
|---|---|---|---|---|---|---|---|
| | | Amino | Amino | Amino | Amino | | VHH |
| | | acid | acid | acid | acid | DNA | Amino acid |
| Antibody ID | Group | sequence | sequence | sequence | sequence | sequence | sequence |
| ODY-31G3 | A | 1 | 2 | 3 | 4 | 48 | 81 |
| ODY-31D6 | A | 5 | 2 | 6 | 7 | 49 | 82 |
| ODY-37C7 | B | 8 | 9 | 10 | 11 | 50 | 83 |
| ODY-N2166hu1 | B | 8 | 9 | 4524 | — | — | 4526 |
| ODY-31G11 | C | 12 | 13 | 14 | 15 | 51 | 84 |
| ODY-33D4 | D | 16 | 17 | 18 | 19 | 52 | 85 |
| ODY-N2170hu1 | D | 16 | 4527 | 18 | — | — | 4529 |
| ODY-N2170hu1.A3 | D | 16 | 4527 | 4530 | — | — | 4532 |
| ODY-N1876Hu1 | D | 4719 | 4723 | 4727 | — | — | 4731 |
| ODY-N1876Hu1.NAR | D | 4720 | 4724 | 4728 | — | — | 4732 |
| ODY-N1879Hu1 | D | 4721 | 4725 | 4729 | — | — | 4733 |
| ODY-N1879Hu1.NAR | D | 4722 | 4726 | 4730 | — | — | 4734 |
| ODY-N1277 | G | 20 | 21 | 22 | 23 | 53 or 3765 | 86 |
| ODY-N1364 | J | 24 | 25 | 26 | 27 | 54 or 3800 | 87 |
| ODY-N1365 | J | 28 | 29 | 30 | 31 | 55 or 3768 | 88 |
| ODY-35A10 | K | 32 | 33 | 34 | 35 | 56 | 89 |
| ODY-N1402 | K | 36 | 37 | 38 | 39 | 57 or 3839 | 90 |
| ODY-N1400 | K | 4069 | 4070 | 4071 | 4072 | 4075 | 4078 |
| ODY-N1425 | L | 40 | 41 | 42 | 43 | 58 or 3844 | 91 |
| ODY-N1409 | M | 44 | 45 | 46 | 47 | 59 or 3845 | 92 |
| ODY-N1943 | M | 4520 | 45 | 46 | 4521 | 4522 | 4523 |
| ODY-N1323 | N | 4061 | 4062 | 4063 | 4064 | 4073 | 4076 |
| ODY-33B1 | O | 4065 | 4066 | 4067 | 4068 | 4074 | 4077 |

TABLE 1-2
Sequence identifiers for additional exemplary
VHH antibodies and humanized VHH antibodies

| | | Non-humanized | Humanized |
|---|---|---|---|
| | | VHH Amino | VHH Amino |
| | | Acid | Acid |
| Cluster | Group | Sequence | Sequence |
| ODY-31G3 - 31D6 | A | 93 | 641 |
| ODY-31G3 - 31D6 | A | 94 | 642 |
| ODY-31G3 - 31D6 | A | 95 | 643 |
| ODY-31G3 - 31D6 | A | 4 | 81 |
| ODY-31G3 - 31D6 | A | 96 | 82 |
| ODY-31G3 - 31D6 | A | 7 | 82 |
| ODY-31G3 - 31D6 | A | 97 | 82 |
| ODY-31G3 - 31D6 | A | 98 | 644 |
| ODY-31G3 - 31D6 | A | 99 | 645 |
| ODY-31G3 - 31D6 | A | 100 | 646 |
| ODY-31G3 - 31D6 | A | 101 | 647 |
| ODY-31G3 - 31D6 | A | 102 | 648 |
| ODY-31G3 - 31D6 | A | 103 | 649 |
| ODY-31G3 - 31D6 | A | 104 | 650 |
| ODY-31G3 - 31D6 | A | 105 | 651 |
| ODY-31G3 - 31D6 | A | 106 | 652 |
| ODY-31G3 - 31D6 | A | 107 | 653 |
| ODY-31G3 - 31D6 | A | 108 | 654 |
| ODY-31G3 - 31D6 | A | 109 | 655 |
| ODY-31G3 - 31D6 | A | 110 | 656 |
| ODY-31G3 - 31D6 | A | 111 | 657 |
| ODY-31G3 - 31D6 | A | 112 | 658 |
| ODY-31G3 - 31D6 | A | 113 | 659 |
| ODY-31G3 - 31D6 | A | 114 | 660 |
| ODY-31G3 - 31D6 | A | 115 | 661 |
| ODY-31G3 - 31D6 | A | 116 | 662 |
| ODY-31G3 - 31D6 | A | 117 | 663 |
| ODY-31G3 - 31D6 | A | 118 | 664 |
| ODY-31G3 - 31D6 | A | 119 | 665 |
| ODY-31G3 - 31D6 | A | 120 | 666 |
| ODY-31G3 - 31D6 | A | 121 | 667 |
| ODY-31G3 - 31D6 | A | 122 | 668 |
| ODY-31G3 - 31D6 | A | 123 | 669 |
| ODY-31G3 - 31D6 | A | 124 | 670 |
| ODY-31G3 - 31D6 | A | 125 | 671 |
| ODY-31G3 - 31D6 | A | 126 | 672 |
| ODY-31G3 - 31D6 | A | 127 | 673 |
| ODY-31G3 - 31D6 | A | 128 | 674 |
| ODY-31G3 - 31D6 | A | 129 | 647 |
| ODY-31G3 - 31D6 | A | 130 | 675 |
| ODY-31G3 - 31D6 | A | 131 | 676 |
| ODY-31G3 - 31D6 | A | 132 | 677 |
| ODY-31G3 - 31D6 | A | 133 | 655 |
| ODY-31G3 - 31D6 | A | 134 | 678 |
| ODY-31G3 - 31D6 | A | 135 | 679 |
| ODY-31G3 - 31D6 | A | 136 | 680 |
| ODY-31G3 - 31D6 | A | 137 | 681 |
| ODY-31G3 - 31D6 | A | 138 | 665 |
| ODY-31G3 - 31D6 | A | 139 | 682 |
| ODY-31G3 - 31D6 | A | 140 | 683 |
| ODY-31G3 - 31D6 | A | 141 | 684 |
| ODY-31G3 - 31D6 | A | 142 | 685 |
| ODY-31G3 - 31D6 | A | 143 | 686 |
| ODY-31G3 - 31D6 | A | 144 | 687 |
| ODY-31G3 - 31D6 | A | 145 | 688 |
| ODY-31G3 - 31D6 | A | 146 | 689 |
| ODY-31G3 - 31D6 | A | 147 | 690 |
| ODY-31G3 - 31D6 | A | 148 | 691 |
| ODY-31G3 - 31D6 | A | 149 | 692 |
| ODY-31G3 - 31D6 | A | 150 | 693 |
| ODY-31G3 - 31D6 | A | 151 | 694 |
| ODY-31G3 - 31D6 | A | 152 | 695 |
| ODY-31G3 - 31D6 | A | 153 | 696 |
| ODY-31G3 - 31D6 | A | 154 | 697 |
| ODY-31G3 - 31D6 | A | 155 | 698 |
| ODY-31G3 - 31D6 | A | 156 | 647 |
| ODY-31G3 - 31D6 | A | 157 | 699 |
| ODY-31G3 - 31D6 | A | 158 | 648 |
| ODY-31G3 - 31D6 | A | 159 | 700 |
| ODY-31G3 - 31D6 | A | 160 | 701 |
| ODY-31G3 - 31D6 | A | 161 | 702 |
| ODY-31G3 - 31D6 | A | 162 | 703 |
| ODY-31G3 - 31D6 | A | 163 | 704 |
| ODY-31G3 - 31D6 | A | 164 | 705 |
| ODY-31G3 - 31D6 | A | 165 | 706 |
| ODY-31G3 - 31D6 | A | 166 | 707 |
| ODY-31G3 - 31D6 | A | 167 | 708 |
| ODY-31G3 - 31D6 | A | 168 | 709 |
| ODY-31G3 - 31D6 | A | 169 | 82 |
| ODY-31G3 - 31D6 | A | 170 | 710 |
| ODY-31G3 - 31D6 | A | 171 | 645 |
| ODY-31G3 - 31D6 | A | 172 | 711 |
| ODY-31G3 - 31D6 | A | 173 | 712 |
| ODY-31G3 - 31D6 | A | 174 | 713 |
| ODY-31G3 - 31D6 | A | 175 | 714 |
| ODY-31G3 - 31D6 | A | 176 | 715 |
| ODY-31G3 - 31D6 | A | 177 | 716 |
| ODY-31G3 - 31D6 | A | 178 | 663 |
| ODY-31G3 - 31D6 | A | 179 | 712 |
| ODY-31G3 - 31D6 | A | 180 | 717 |
| ODY-31G3 - 31D6 | A | 181 | 718 |
| ODY-31G3 - 31D6 | A | 182 | 719 |
| ODY-31G3 - 31D6 | A | 183 | 720 |
| ODY-31G3 - 31D6 | A | 184 | 721 |
| ODY-31G3 - 31D6 | A | 185 | 722 |
| ODY-31G3 - 31D6 | A | 186 | 723 |
| ODY-31G3 - 31D6 | A | 187 | 724 |
| ODY-31G3 - 31D6 | A | 188 | 725 |
| ODY-31G3 - 31D6 | A | 189 | 726 |
| ODY-31G3 - 31D6 | A | 190 | 727 |
| ODY-31G3 - 31D6 | A | 191 | 728 |
| ODY-31G3 - 31D6 | A | 192 | 729 |
| ODY-31G3 - 31D6 | A | 193 | 724 |
| ODY-31G3 - 31D6 | A | 194 | 730 |
| ODY-31G3 - 31D6 | A | 195 | 731 |
| ODY-31G3 - 31D6 | A | 196 | 732 |
| ODY-31G3 - 31D6 | A | 197 | 733 |
| ODY-31G3 - 31D6 | A | 198 | 734 |
| ODY-31G3 - 31D6 | A | 199 | 735 |
| ODY-31G3 - 31D6 | A | 200 | 736 |
| ODY-31G3 - 31D6 | A | 201 | 737 |
| ODY-31G3 - 31D6 | A | 202 | 738 |
| ODY-31G3 - 31D6 | A | 203 | 739 |
| ODY-31G3 - 31D6 | A | 204 | 727 |
| ODY-31G3 - 31D6 | A | 205 | 740 |
| ODY-31G3 - 31D6 | A | 206 | 741 |
| ODY-31G3 - 31D6 | A | 207 | 742 |
| ODY-31G3 - 31D6 | A | 208 | 743 |
| ODY-31G3 - 31D6 | A | 209 | 744 |
| ODY-31G3 - 31D6 | A | 210 | 745 |
| ODY-31G3 - 31D6 | A | 211 | 746 |
| ODY-31G3 - 31D6 | A | 212 | 727 |
| ODY-31G3 - 31D6 | A | 213 | 747 |
| ODY-31G3 - 31D6 | A | 214 | 748 |
| ODY-31G3 - 31D6 | A | 215 | 749 |
| ODY-31G3 - 31D6 | A | 216 | 750 |
| ODY-31G3 - 31D6 | A | 217 | 751 |
| ODY-31G3 - 31D6 | A | 218 | 752 |
| ODY-31G3 - 31D6 | A | 219 | 753 |
| ODY-31G3 - 31D6 | A | 220 | 754 |
| ODY-31G3 - 31D6 | A | 221 | 755 |
| ODY-31G3 - 31D6 | A | 222 | 756 |
| ODY-31G3 - 31D6 | A | 223 | 757 |
| ODY-31G3 - 31D6 | A | 224 | 758 |
| ODY-31G3 - 31D6 | A | 225 | 759 |
| ODY-31G3 - 31D6 | A | 226 | 760 |
| ODY-31G3 - 31D6 | A | 227 | 761 |
| ODY-31G3 - 31D6 | A | 228 | 762 |
| ODY-31G3 - 31D6 | A | 229 | 752 |
| ODY-31G3 - 31D6 | A | 230 | 763 |
| ODY-31G3 - 31D6 | A | 231 | 764 |
| ODY-31G3 - 31D6 | A | 232 | 765 |
| ODY-31G3 - 31D6 | A | 233 | 766 |
| ODY-31G3 - 31D6 | A | 234 | 767 |
| ODY-31G3 - 31D6 | A | 235 | 768 |
| ODY-31G3 - 31D6 | A | 236 | 769 |
| ODY-31G3 - 31D6 | A | 237 | 770 |
| ODY-31G3 - 31D6 | A | 238 | 771 |
| ODY-31G3 - 31D6 | A | 239 | 772 |
| ODY-31G3 - 31D6 | A | 240 | 773 |
| ODY-31G3 - 31D6 | A | 241 | 774 |
| ODY-31G3 - 31D6 | A | 242 | 775 |
| ODY-31G3 - 31D6 | A | 243 | 776 |
| ODY-31G3 - 31D6 | A | 244 | 777 |
| ODY-31G3 - 31D6 | A | 245 | 778 |
| ODY-31G3 - 31D6 | A | 246 | 779 |
| ODY-31G3 - 31D6 | A | 247 | 780 |
| ODY-31G3 - 31D6 | A | 248 | 781 |
| ODY-31G3 - 31D6 | A | 249 | 782 |
| ODY-31G3 - 31D6 | A | 250 | 783 |
| ODY-31G3 - 31D6 | A | 251 | 784 |
| ODY-31G3 - 31D6 | A | 252 | 785 |
| ODY-31G3 - 31D6 | A | 253 | 786 |
| ODY-31G3 - 31D6 | A | 254 | 787 |
| ODY-31G3 - 31D6 | A | 255 | 788 |
| ODY-31G3 - 31D6 | A | 256 | 789 |
| ODY-31G3 - 31D6 | A | 257 | 790 |
| ODY-31G3 - 31D6 | A | 258 | 786 |
| ODY-31G3 - 31D6 | A | 259 | 791 |
| ODY-31G3 - 31D6 | A | 260 | 786 |
| ODY-31G3 - 31D6 | A | 261 | 776 |
| ODY-31G3 - 31D6 | A | 262 | 792 |
| ODY-31G3 - 31D6 | A | 263 | 793 |
| ODY-31G3 - 31D6 | A | 264 | 794 |
| ODY-31G3 - 31D6 | A | 265 | 795 |
| ODY-31G3 - 31D6 | A | 266 | 796 |
| ODY-31G3 - 31D6 | A | 267 | 796 |
| ODY-31G3 - 31D6 | A | 268 | 797 |
| ODY-31G3 - 31D6 | A | 269 | 798 |
| ODY-31G3 - 31D6 | A | 270 | 799 |
| ODY-31G3 - 31D6 | A | 271 | 800 |
| ODY-31G3 - 31D6 | A | 272 | 801 |
| ODY-31G3 - 31D6 | A | 273 | 802 |
| ODY-31G3 - 31D6 | A | 274 | 803 |
| ODY-31G3 - 31D6 | A | 275 | 804 |
| ODY-31G3 - 31D6 | A | 276 | 805 |
| ODY-31G3 - 31D6 | A | 277 | 806 |
| ODY-31G3 - 31D6 | A | 278 | 807 |
| ODY-31G3 - 31D6 | A | 279 | 808 |
| ODY-31G3 - 31D6 | A | 280 | 809 |
| ODY-31G3 - 31D6 | A | 281 | 810 |
| ODY-31G3 - 31D6 | A | 282 | 811 |
| ODY-31G3 - 31D6 | A | 283 | 812 |
| ODY-31G3 - 31D6 | A | 284 | 813 |
| ODY-31G3 - 31D6 | A | 285 | 814 |
| ODY-31G3 - 31D6 | A | 286 | 815 |
| ODY-31G3 - 31D6 | A | 287 | 816 |
| ODY-31G3 - 31D6 | A | 288 | 817 |
| ODY-31G3 - 31D6 | A | 289 | 818 |
| ODY-31G3 - 31D6 | A | 290 | 819 |
| ODY-31G3 - 31D6 | A | 291 | 820 |
| ODY-31G3 - 31D6 | A | 292 | 821 |
| ODY-31G3 - 31D6 | A | 293 | 822 |
| ODY-31G3 - 31D6 | A | 294 | 823 |
| ODY-31G3 - 31D6 | A | 295 | 806 |
| ODY-31G3 - 31D6 | A | 296 | 806 |
| ODY-31G3 - 31D6 | A | 297 | 824 |
| ODY-31G3 - 31D6 | A | 298 | 800 |
| ODY-31G3 - 31D6 | A | 299 | 803 |
| ODY-31G3 - 31D6 | A | 300 | 825 |
| ODY-31G3 - 31D6 | A | 301 | 826 |
| ODY-31G3 - 31D6 | A | 302 | 826 |
| ODY-31G3 - 31D6 | A | 303 | 827 |
| ODY-31G3 - 31D6 | A | 304 | 828 |
| ODY-31G3 - 31D6 | A | 305 | 829 |
| ODY-31G3 - 31D6 | A | 306 | 826 |
| ODY-31G3 - 31D6 | A | 307 | 830 |
| ODY-31G3 - 31D6 | A | 308 | 831 |
| ODY-31G3 - 31D6 | A | 309 | 832 |
| ODY-31G3 - 31D6 | A | 310 | 833 |
| ODY-31G3 - 31D6 | A | 311 | 834 |
| ODY-31G3 - 31D6 | A | 312 | 835 |
| ODY-31G3 - 31D6 | A | 313 | 836 |
| ODY-31G3 - 31D6 | A | 314 | 837 |
| ODY-31G3 - 31D6 | A | 315 | 838 |
| ODY-31G3 - 31D6 | A | 316 | 839 |
| ODY-31G3 - 31D6 | A | 317 | 840 |
| ODY-31G3 - 31D6 | A | 318 | 841 |
| ODY-31G3 - 31D6 | A | 319 | 842 |
| ODY-31G3 - 31D6 | A | 320 | 843 |
| ODY-31G3 - 31D6 | A | 321 | 844 |
| ODY-31G3 - 31D6 | A | 322 | 845 |
| ODY-31G3 - 31D6 | A | 323 | 846 |
| ODY-31G3 - 31D6 | A | 324 | 847 |
| ODY-31G3 - 31D6 | A | 325 | 848 |
| ODY-31G3 - 31D6 | A | 326 | 849 |
| ODY-31G3 - 31D6 | A | 327 | 850 |
| ODY-31G3 - 31D6 | A | 328 | 851 |
| ODY-31G3 - 31D6 | A | 329 | 835 |
| ODY-31G3 - 31D6 | A | 330 | 852 |
| ODY-31G3 - 31D6 | A | 331 | 853 |
| ODY-31G3 - 31D6 | A | 332 | 854 |
| ODY-31G3 - 31D6 | A | 333 | 855 |
| ODY-31G3 - 31D6 | A | 334 | 856 |
| ODY-31G3 - 31D6 | A | 335 | 857 |
| ODY-31G3 - 31D6 | A | 336 | 858 |
| ODY-31G3 - 31D6 | A | 337 | 859 |
| ODY-31G3 - 31D6 | A | 338 | 860 |
| ODY-31G3 - 31D6 | A | 339 | 861 |
| ODY-31G3 - 31D6 | A | 340 | 862 |
| ODY-31G3 - 31D6 | A | 341 | 863 |
| ODY-31G3 - 31D6 | A | 342 | 864 |
| ODY-31G3 - 31D6 | A | 343 | 865 |
| ODY-31G3 - 31D6 | A | 344 | 366 |
| ODY-31G3 - 31D6 | A | 345 | 867 |
| ODY-31G3 - 31D6 | A | 346 | 868 |
| ODY-31G3 - 31D6 | A | 347 | 869 |
| ODY-31G3 - 31D6 | A | 348 | 870 |
| ODY-31G3 - 31D6 | A | 349 | 871 |
| ODY-31G3 - 31D6 | A | 350 | 872 |
| ODY-31G3 - 31D6 | A | 351 | 873 |
| ODY-31G3 - 31D6 | A | 352 | 874 |
| ODY-31G3 - 31D6 | A | 353 | 875 |
| ODY-31G3 - 31D6 | A | 354 | 876 |
| ODY-31G3 - 31D6 | A | 355 | 877 |
| ODY-31G3 - 31D6 | A | 356 | 878 |
| ODY-31G3 - 31D6 | A | 357 | 879 |
| ODY-31G3 - 31D6 | A | 358 | 880 |
| ODY-31G3 - 31D6 | A | 359 | 881 |
| ODY-31G3 - 31D6 | A | 360 | 882 |
| ODY-31G3 - 31D6 | A | 361 | 883 |
| ODY-31G3 - 31D6 | A | 362 | 884 |
| ODY-31G3 - 31D6 | A | 363 | 885 |
| ODY-31G3 - 31D6 | A | 364 | 886 |
| ODY-31G3 - 31D6 | A | 365 | 887 |
| ODY-31G3 - 31D6 | A | 366 | 888 |
| ODY-31G3 - 31D6 | A | 367 | 889 |
| ODY-31G3 - 31D6 | A | 368 | 890 |
| ODY-31G3 - 31D6 | A | 369 | 891 |
| ODY-31G3 - 31D6 | A | 370 | 892 |
| ODY-31G3 - 31D6 | A | 371 | 893 |
| ODY-31G3 - 31D6 | A | 372 | 894 |
| ODY-31G3 - 31D6 | A | 373 | 895 |
| ODY-31G3 - 31D6 | A | 374 | 896 |
| ODY-31G3 - 31D6 | A | 375 | 897 |
| ODY-31G3 - 31D6 | A | 376 | 898 |
| ODY-31G3 - 31D6 | A | 377 | 899 |
| ODY-31G3 - 31D6 | A | 378 | 900 |
| ODY-31G3 - 31D6 | A | 379 | 901 |
| ODY-31G3 - 31D6 | A | 380 | 902 |
| ODY-31G3 - 31D6 | A | 381 | 903 |
| ODY-31G3 - 31D6 | A | 382 | 904 |
| ODY-31G3 - 31D6 | A | 383 | 905 |
| ODY-31G3 - 31D6 | A | 384 | 906 |
| ODY-31G3 - 31D6 | A | 385 | 907 |
| ODY-31G3 - 31D6 | A | 386 | 908 |
| ODY-31G3 - 31D6 | A | 387 | 909 |
| ODY-31G3 - 31D6 | A | 388 | 910 |
| ODY-31G3 - 31D6 | A | 389 | 911 |
| ODY-31G3 - 31D6 | A | 390 | 912 |
| ODY-31G3 - 31D6 | A | 391 | 913 |
| ODY-37C7 | B | 11 | 83 |
| ODY-37C7 | B | 392 | 914 |
| ODY-37C7 | B | 393 | 915 |
| ODY-37C7 | B | 394 | 916 |
| ODY-37C7 | B | 395 | 917 |
| ODY-37C7 | B | 396 | 918 |
| ODY-37C7 | B | 397 | 919 |
| ODY-37C7 | B | 398 | 920 |
| ODY-37C7 | B | 399 | 921 |
| ODY-37C7 | B | 400 | 922 |
| ODY-31G11 | C | 401 | 923 |
| ODY-31G11 | C | 402 | 924 |
| ODY-31G11 | C | 15 | 84 |
| ODY-31G11 | C | 403 | 925 |
| ODY-31G11 | C | 404 | 923 |
| ODY-31G11 | C | 405 | 926 |
| ODY-31G11 | C | 406 | 927 |
| ODY-31G11 | C | 407 | 923 |
| ODY-31G11 | C | 408 | 928 |
| ODY-31G11 | C | 409 | 929 |
| ODY-31G11 | C | 410 | 930 |
| ODY-33D4 | D | 411 | 931 |
| ODY-33D4 | D | 412 | 932 |
| ODY-33D4 | D | 413 | 933 |
| ODY-33D4 | D | 414 | 934 |
| ODY-33D4 | D | 415 | 935 |
| ODY-33D4 | D | 416 | 936 |
| ODY-33D4 | D | 417 | 937 |
| ODY-33D4 | D | 418 | 938 |
| ODY-33D4 | D | 419 | 939 |
| ODY-33D4 | D | 420 | 940 |
| ODY-33D4 | D | 421 | 941 |
| ODY-33D4 | D | 422 | 942 |
| ODY-33D4 | D | 423 | 943 |
| ODY-33D4 | D | 424 | 944 |
| ODY-33D4 | D | 425 | 945 |
| ODY-33D4 | D | 426 | 946 |
| ODY-33D4 | D | 427 | 947 |
| ODY-33D4 | D | 428 | 948 |
| ODY-33D4 | D | 429 | 949 |
| ODY-33D4 | D | 430 | 950 |
| ODY-33D4 | D | 431 | 951 |
| ODY-33D4 | D | 432 | 952 |
| ODY-33D4 | D | 433 | 953 |
| ODY-33D4 | D | 434 | 954 |
| ODY-33D4 | D | 435 | 955 |
| ODY-33D4 | D | 436 | 956 |
| ODY-33D4 | D | 437 | 957 |
| ODY-33D4 | D | 19 | 85 |
| ODY-33D4 | D | 438 | 958 |
| ODY-33D4 | D | 439 | 959 |
| ODY-33D4 | D | 440 | 960 |
| ODY-33D4 | D | 441 | 961 |
| ODY-33D4 | D | 442 | 962 |
| ODY-33D4 | D | 443 | 963 |
| ODY-33D4 | D | 444 | 964 |
| ODY-33D4 | D | 445 | 965 |
| ODY-33D4 | D | 446 | 966 |
| ODY-33D4 | D | 447 | 967 |
| ODY-33D4 | D | 448 | 968 |
| ODY-33D4 | D | 449 | 969 |
| ODY-33D4 | D | 450 | 970 |
| ODY-33D4 | D | 451 | 971 |
| ODY-33D4 | D | 452 | 972 |
| ODY-33D4 | D | 453 | 973 |
| ODY-33D4 | D | 454 | 938 |
| ODY-33D4 | D | 455 | 944 |
| ODY-33D4 | D | 456 | 974 |
| ODY-33D4 | D | 457 | 975 |
| ODY-33D4 | D | 458 | 956 |
| ODY-33D4 | D | 459 | 976 |
| ODY-33D4 | D | 460 | 977 |
| ODY-33D4 | D | 461 | 978 |
| ODY-33D4 | D | 462 | 979 |
| ODY-33D4 | D | 463 | 980 |
| ODY-33D4 | D | 464 | 981 |
| ODY-33D4 | D | 465 | 982 |
| ODY-33D4 | D | 466 | 983 |
| ODY-33D4 | D | 467 | 984 |
| ODY-33D4 | D | 468 | 985 |
| ODY-33D4 | D | 469 | 986 |
| ODY-33D4 | D | 470 | 987 |
| ODY-33D4 | D | 471 | 988 |
| ODY-33D4 | D | 472 | 989 |
| ODY-33D4 | D | 473 | 951 |
| ODY-33D4 | D | 474 | 990 |
| ODY-33D4 | D | 475 | 991 |
| ODY-33D4 | D | 476 | 992 |
| ODY-33D4 | D | 477 | 954 |
| ODY-33D4 | D | 478 | 993 |
| ODY-33D4 | D | 479 | 994 |
| ODY-33D4 | D | 480 | 939 |
| ODY-33D4 | D | 481 | 995 |
| ODY-33D4 | D | 482 | 996 |
| ODY-33D4 | D | 483 | 997 |
| ODY-33D4 | D | 484 | 998 |
| ODY-33D4 | D | 485 | 999 |
| ODY-33D4 | D | 486 | 1000 |
| ODY-33D4 | D | 487 | 1001 |
| ODY-33D4 | D | 488 | 1002 |
| ODY-N1277 | G | 23 | 86 |
| ODY-N1277 | G | 489 | 1003 |
| ODY-N1277 | G | 490 | 1004 |
| ODY-N1364 - N1365 | J | 31 | 88 |
| ODY-N1364 - N1365 | J | 491 | 1005 |
| ODY-N1364 - N1365 | J | 492 | 1006 |
| ODY-N1364 - N1365 | J | 493 | 1007 |
| ODY-N1364 - N1365 | J | 494 | 1008 |
| ODY-N1364 - N1365 | J | 495 | 1009 |
| ODY-N1364 - N1365 | J | 496 | 1010 |
| ODY-N1364 - N1365 | J | 497 | 1011 |
| ODY-N1364 - N1365 | J | 498 | 1012 |
| ODY-N1364 - N1365 | J | 499 | 1013 |
| ODY-N1364 - N1365 | J | 500 | 1014 |
| ODY-N1364 - N1365 | J | 501 | 88 |
| ODY-N1364 - N1365 | J | 502 | 1015 |
| ODY-N1364 - N1365 | J | 503 | 1016 |
| ODY-N1364 - N1365 | J | 504 | 1017 |
| ODY-N1364 - N1365 | J | 505 | 1018 |
| ODY-N1364 - N1365 | J | 506 | 1019 |
| ODY-N1364 - N1365 | J | 507 | 1020 |
| ODY-N1364 - N1365 | J | 508 | 1019 |
| ODY-N1364 - N1365 | J | 509 | 1021 |
| ODY-N1364 - N1365 | J | 510 | 1022 |
| ODY-N1364 - N1365 | J | 511 | 1023 |
| ODY-N1364 - N1365 | J | 512 | 1024 |
| ODY-N1364 - N1365 | J | 513 | 1025 |
| ODY-N1364 - N1365 | J | 514 | 1026 |
| ODY-N1364 - N1365 | J | 515 | 1027 |
| ODY-N1364 - N1365 | J | 516 | 1028 |
| ODY-N1364 - N1365 | J | 517 | 1029 |
| ODY-N1364 - N1365 | J | 518 | 1030 |
| ODY-N1364 - N1365 | J | 519 | 1031 |
| ODY-N1364 - N1365 | J | 520 | 1032 |
| ODY-N1364 - N1365 | J | 521 | 1033 |
| ODY-N1364 - N1365 | J | 27 | 87 |
| ODY-N1364 - N1365 | J | 522 | 1034 |
| ODY-N1364 - N1365 | J | 523 | 1035 |
| ODY-N1364 - N1365 | J | 524 | 1036 |
| ODY-N1364 - N1365 | J | 525 | 1037 |
| ODY-N1364 - N1365 | J | 526 | 1038 |
| ODY-N1364 - N1365 | J | 527 | 1039 |
| ODY-N1364 - N1365 | J | 528 | 1040 |
| ODY-N1364 - N1365 | J | 529 | 1041 |
| ODY-N1364 - N1365 | J | 530 | 1042 |
| ODY-N1364 - N1365 | J | 531 | 1043 |
| ODY-N1364 - N1365 | J | 532 | 1044 |
| ODY-N1364 - N1365 | J | 533 | 1045 |
| ODY-N1364 - N1365 | J | 534 | 1046 |
| ODY-N1364 - N1365 | J | 535 | 1035 |
| ODY-N1364 - N1365 | J | 536 | 1035 |
| ODY-N1364 - N1365 | J | 537 | 1047 |
| ODY-N1364 - N1365 | J | 538 | 1048 |
| ODY-35A10 - N1402 | K | 539 | 1049 |
| ODY-35A10 - N1402 | K | 540 | 1050 |
| ODY-35A10 - N1402 | K | 541 | 1051 |
| ODY-35A10 - N1402 | K | 542 | 1051 |
| ODY-35A10 - N1402 | K | 543 | 1050 |
| ODY-35A10 - N1402 | K | 544 | 1052 |
| ODY-35A10 - N1402 | K | 545 | 1053 |
| ODY-35A10 - N1402 | K | 546 | 1054 |
| ODY-35A10 - N1402 | K | 547 | 1055 |
| ODY-35A10 - N1402 | K | 548 | 1051 |
| ODY-35A10 - N1402 | K | 549 | 1056 |
| ODY-35A10 - N1402 | K | 550 | 1057 |
| ODY-35A10 - N1402 | K | 551 | 1058 |
| ODY-35A10 - N1402 | K | 552 | 1059 |
| ODY-35A10 - N1402 | K | 553 | 1060 |
| ODY-35A10 - N1402 | K | 554 | 1061 |
| ODY-35A10 - N1402 | K | 555 | 1062 |
| ODY-35A10 - N1402 | K | 556 | 1063 |
| ODY-35A10 - N1402 | K | 557 | 1064 |
| ODY-35A10 - N1402 | K | 558 | 1065 |
| ODY-35A10 - N1402 | K | 559 | 1066 |
| ODY-35A10 - N1402 | K | 39 | 90 |
| ODY-35A10 - N1402 | K | 560 | 1067 |
| ODY-35A10 - N1402 | K | 561 | 1068 |
| ODY-35A10 - N1402 | K | 562 | 1069 |
| ODY-35A10 - N1402 | K | 563 | 1070 |
| ODY-35A10 - N1402 - | K | 4101 | 4148 |
| N1400 | | | |
| ODY-35A10 - N1402 - | K | 4102 | 4149 |
| N1400 | | | |
| ODY-35A10 - N1402 - | K | 4103 | 4150 |
| N1400 | | | |
| ODY-35A10 - N1402 - | K | 4104 | 4151 |
| N1400 | | | |
| ODY-35A10 - N1402 - | K | 4105 | 4152 |
| N1400 | | | |
| ODY-35A10 - N1402 - | K | 4106 | 4153 |
| N1400 | | | |
| ODY-35A10 - N1402 - | K | 4107 | 4154 |
| N1400 | | | |
| ODY-35A10 - N1402 - | K | 4108 | 4155 |
| N1400 | | | |
| ODY-35A10 - N1402 - | K | 4109 | 4156 |
| N1400 | | | |
| ODY-35A10 - N1402 - | K | 4110 | 4157 |
| N1400 | | | |
| ODY-35A10 - N1402 - | K | 4111 | 4158 |
| N1400 | | | |
| ODY-35A10 - N1402 - | K | 4112 | 4159 |
| N1400 | | | |
| ODY-35A10 - N1402 - | K | 4113 | 4160 |
| N1400 | | | |
| ODY-35A10 - N1402 - | K | 4114 | 4161 |
| N1400 | | | |
| ODY-35A10 - N1402 - | K | 4115 | 4162 |
| N1400 | | | |
| ODY-35A10 - N1402 - | K | 4116 | 4163 |
| N1400 | | | |
| ODY-35A10 - N1402 - | K | 4117 | 4164 |
| N1400 | | | |
| ODY-35A10 - N1402 - | K | 4118 | 4165 |
| N1400 | | | |
| ODY-35A10 - N1402 - | K | 4119 | 4166 |
| N1400 | | | |
| ODY-35A10 - N1402 - | K | 4120 | 4167 |
| N1400 | | | |
| ODY-35A10 - N1402 - | K | 4121 | 4168 |
| N1400 | | | |
| ODY-35A10 - N1402 - | K | 4122 | 4169 |
| N1400 | | | |
| ODY-35A10 - N1402 - | K | 4123 | 4170 |
| N1400 | | | |
| ODY-35A10 - N1402 - | K | 4124 | 4171 |
| N1400 | | | |
| ODY-35A10 - N1402 - | K | 4125 | 4172 |
| N1400 | | | |
| ODY-N1425 | L | 43 | 91 |
| ODY-N1409 | M | 47 | 92 |
| ODY-N1409 | M | 564 | 1071 |
| ODY-N1409 | M | 565 | 1072 |
| ODY-N1409 | M | 566 | 1072 |
| ODY-N1409 | M | 567 | 1073 |
| ODY-N1409 | M | 568 | 1074 |
| ODY-N1409 | M | 569 | 1074 |
| ODY-N1409 | M | 570 | 1075 |
| ODY-N1409 | M | 571 | 1076 |
| ODY-N1409 | M | 572 | 1077 |
| ODY-N1409 | M | 573 | 1078 |
| ODY-N1409 | M | 574 | 1079 |
| ODY-N1409 | M | 575 | 1073 |
| ODY-N1409 | M | 576 | 1080 |
| ODY-N1409 | M | 577 | 1081 |
| ODY-N1409 | M | 578 | 1082 |
| ODY-N1409 | M | 579 | 1083 |
| ODY-N1409 | M | 580 | 1084 |
| ODY-N1409 | M | 581 | 1074 |
| ODY-N1409 | M | 582 | 1085 |
| ODY-N1409 | M | 583 | 1074 |
| ODY-N1409 | M | 584 | 1078 |
| ODY-N1409 | M | 585 | 1086 |
| ODY-N1409 | M | 586 | 1071 |
| ODY-N1409 | M | 587 | 1087 |
| ODY-N1409 | M | 588 | 1073 |
| ODY-N1409 | M | 589 | 1088 |
| ODY-N1409 | M | 590 | 1073 |
| ODY-N1409 | M | 591 | 1078 |
| ODY-N1409 | M | 592 | 1089 |
| ODY-N1409 | M | 593 | 1077 |
| ODY-N1409 | M | 594 | 1090 |
| ODY-N1409 | M | 595 | 1091 |
| ODY-N1409 | M | 596 | 1092 |
| ODY-N1409 | M | 597 | 1093 |
| ODY-N1409 | M | 598 | 1094 |
| ODY-N1409 | M | 599 | 1095 |
| ODY-N1409 | M | 600 | 1096 |
| ODY-N1409 | M | 601 | 1097 |
| ODY-N1409 | M | 602 | 1098 |
| ODY-N1409 | M | 603 | 1099 |
| ODY-N1409 | M | 604 | 1100 |
| ODY-N1409 | M | 605 | 1101 |
| ODY-N1409 | M | 606 | 1102 |
| ODY-N1409 | M | 607 | 1103 |
| ODY-N1409 | M | 608 | 1103 |
| ODY-N1409 | M | 609 | 1103 |
| ODY-N1409 | M | 610 | 1104 |
| ODY-N1409 | M | 611 | 1104 |
| ODY-N1409 | M | 612 | 1105 |
| ODY-N1409 | M | 613 | 1106 |
| ODY-N1409 | M | 614 | 1107 |
| ODY-N1409 | M | 615 | 1108 |
| ODY-N1409 | M | 616 | 1109 |
| ODY-N1409 | M | 617 | 1110 |
| ODY-N1409 | M | 618 | 1111 |
| ODY-N1409 | M | 619 | 1112 |
| ODY-N1409 | M | 620 | 1113 |
| ODY-N1409 | M | 621 | 1114 |
| ODY-N1409 | M | 622 | 1115 |
| ODY-N1409 | M | 623 | 1116 |
| ODY-N1409 | M | 624 | 1099 |
| ODY-N1409 | M | 625 | 1117 |
| ODY-N1409 | M | 626 | 1118 |
| ODY-N1409 | M | 627 | 1119 |
| ODY-N1409 | M | 628 | 1120 |
| ODY-N1409 | M | 629 | 1101 |
| ODY-N1409 | M | 630 | 1099 |
| ODY-N1409 | M | 631 | 1102 |
| ODY-N1409 | M | 632 | 1102 |
| ODY-N1409 | M | 633 | 1106 |
| ODY-N1409 | M | 634 | 1121 |
| ODY-N1409 | M | 635 | 1122 |
| ODY-N1409 | M | 636 | 1123 |
| ODY-N1409 | M | 637 | 1124 |
| ODY-N1409 | M | 638 | 1125 |
| ODY-N1409 | M | 639 | 1126 |
| ODY-N1409 | M | 640 | 1127 |
| ODY-N1323 | N | 4079 | 4126 |
| ODY-N1323 | N | 4080 | 4127 |
| ODY-N1323 | N | 4081 | 4128 |
| ODY-N1323 | N | 4082 | 4129 |
| ODY-N1323 | N | 4083 | 4130 |
| ODY-N1323 | N | 4084 | 4131 |
| ODY-N1323 | N | 4085 | 4132 |
| ODY-N1323 | N | 4086 | 4133 |
| ODY-N1323 | N | 4087 | 4134 |
| ODY-33B1 | O | 4088 | 4135 |
| ODY-33B1 | O | 4089 | 4136 |
| ODY-33B1 | O | 4090 | 4137 |
| ODY-33B1 | O | 4091 | 4138 |
| ODY-33B1 | O | 4092 | 4139 |
| ODY-33B1 | O | 4093 | 4140 |
| ODY-33B1 | O | 4094 | 4141 |
| ODY-33B1 | O | 4095 | 4142 |
| ODY-33B1 | O | 4096 | 4143 |
| ODY-33B1 | O | 4097 | 4144 |
| ODY-33B1 | O | 4098 | 4145 |
| ODY-33B1 | O | 4099 | 4146 |
| ODY-33B1 | O | 4100 | 4147 |

In some embodiments, an anti-TNFR2 antigen-binding protein (e.g., antibody such as a single-domain antibody) of the present disclosure comprises a complementarity determining region 1 (CDR1) comprising an amino acid sequence selected from (amino acids listed in a pair of brackets represent the possible amino acids at the particular position)

a).
(SEQ ID NO: 68)
GSI(V/F)(R/S)(T/A)(N/D)(S/G/A);
b).
(SEQ ID NO: 69)
GFT(F/L)DD(I/Y)A;
c).
(SEQ ID NO: 70)
GFTFS(S/R/G)YA;
d).
(SEQ ID NO: 16)
GRTFSDYG;
e).
(SEQ ID NO: 71)
G(L/F)TLDYYA;
f).
(SEQ ID NO: 72)
GF(T/N)FSMYS;
g).
(SEQ ID NO: 73)
GRTF(G/R/S)(N/S)(Y/L)(T/F);
h).
(SEQ ID NO: 40)
GASLSRNA;
i).
(SEQ ID NO: 74)
GS(I/T)FRFPP;
j).
(SEQ ID NO: 4061)
GFTLDDYA;
and
k).
(SEQ ID NO: 4519)
G(F/V)(S/T)LD(D/Y)(H/Y)T.

In some embodiments, an anti-TNFR2 antigen-binding protein (e.g., antibody such as a single-domain antibody) of the present disclosure comprises a complementarity determining region 2 (CDR2) comprising an amino acid sequence selected from (amino acids listed in a pair of brackets represent the possible amino acids at the particular position)

a).
(SEQ ID NO: 75)
IRSDGF(T/I);
b).
(SEQ ID NO: 76)
I(Y/F)SY(S/G)(S/P)NT;
c).
(SEQ ID NO: 77)
I(Y/S)(S/D)DGS(E/D)T;
d).
(SEQ ID NO: 4699)
INWSN(G/A)RT;
e).
(SEQ ID NO: 78)
I(S/N)(V/T)(S/G)DGST;
f).
(SEQ ID NO: 79)
IDT(R/G)GST;
g).
(SEQ ID NO: 80)
IR(W/R/Y)(T/P)G(G/L)(S/I)T;
h).
(SEQ ID NO: 41)
IYDDGET;
i).
(SEQ ID NO: 45)
LTSGGST;
j).
(SEQ ID NO: 4062)
IFSYSSNT;
and
k).
(SEQ ID NO: 4518)
I(N/S)SNDG(S/T)(T/V).

In some embodiments, an anti-TNFR2 antigen-binding protein (e.g., antibody such as a single-domain antibody) of the present disclosure comprises a complementarity determining region 3 (CDR3) comprising an amino acid sequence selected from (amino acids listed in a pair of brackets represent the possible amino acids at the particular position)

a).
(SEQ ID NO: 60)
(Y/F)YQ(S/A)LS(T/S)(P/A)N(Y/F)GQ(V/T)F;
b).
(SEQ ID NO: 61)
AADSDL(S/R)TV(V/T)VGPHDY;
c).
(SEQ ID NO: 62)
AKDAG(S/G)WG(T/R)GPFG(Y/F)(E/D)YDY;
d).
(SEQ ID NO: 63)
AA(T/A)PSGKAY(T/S)Y;
e).
(SEQ ID NO: 64)
ATPGPY(T/S/M)YCAPYGSSWSRGYDY;
f).
(SEQ ID NO: 65)
ARV(R/G)G(T/S/A)PY(E/D)Y(N/G)Y;
g).
(SEQ ID NO: 66)
(T/A/V)A(S/A)PTGRAF(T/N/A)Y;
h).
(SEQ ID NO: 42)
AGSAFDF;
i).
(SEQ ID NO: 67)
S(V/M)(V/L)GRDM(M/V)TY;
j).
(SEQ ID NO: 4063)
AVGDFEGELVLKGDY;
k).
(SEQ ID NO: 4517)
AAD(L/V)G(F/V/Y)LY(A/T/V)DYV(P/R)LH(M/T)HHFGS;
l).
(SEQ ID NO: 4771)
A(A/G)(T/A)(P/L)(S/T)GKAY(T/S)Y.

In certain embodiments, an anti-TNFR2 antigen-binding protein (e.g., antibody such as a single-domain antibody) of the present disclosure comprises
i) a CDR1 comprising an amino acid sequence of SEQ ID NO: 68, a CDR2 comprising an amino acid sequence of SEQ ID NO: 75, and a CDR3 comprising an amino acid sequence of SEQ ID NO: 60;
ii) a CDR1 comprising an amino acid sequence of SEQ ID NO: 69, a CDR2 comprising an amino acid sequence of SEQ ID NO: 76, a CDR3 comprising an amino acid sequence of SEQ ID NO: 61;
iii) a CDR1 comprising an amino acid sequence of SEQ ID NO: 70, a CDR2 comprising an amino acid sequence of SEQ ID NO: 77, a CDR3 comprising an amino acid sequence of SEQ ID NO: 62;
iv) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 63;
v) a CDR1 comprising an amino acid sequence of SEQ ID NO: 71, a CDR2 comprising an amino acid sequence of SEQ ID NO: 78, a CDR3 comprising an amino acid sequence of SEQ ID NO: 64;
vi) a CDR1 comprising an amino acid sequence of SEQ ID NO: 72, a CDR2 comprising an amino acid sequence of SEQ ID NO: 79, a CDR3 comprising an amino acid sequence of SEQ ID NO: 65;
vii) a CDR1 comprising an amino acid sequence of SEQ ID NO: 73, a CDR2 comprising an amino acid sequence of SEQ ID NO: 80, a CDR3 comprising an amino acid sequence of SEQ ID NO: 66;
viii) a CDR1 comprising an amino acid sequence of SEQ ID NO: 40, a CDR2 comprising an amino acid sequence of SEQ ID NO: 41, a CDR3 comprising an amino acid sequence of SEQ ID NO: 42; or
ix) a CDR1 comprising an amino acid sequence of SEQ ID NO: 74, a CDR2 comprising an amino acid sequence of SEQ ID NO: 45, a CDR3 comprising an amino acid sequence of SEQ ID NO: 67;
x) a CDR1 comprising an amino acid sequence of SEQ ID NO: 4061, a CDR2 comprising an amino acid sequence of SEQ ID NO: 4062, a CDR3 comprising an amino acid sequence of SEQ ID NO: 4063;
xi). a CDR1 comprising an amino acid sequence of SEQ ID NO: 4519, a CDR2 comprising an amino acid sequence of SEQ ID NO: 4518, a CDR3 comprising an amino acid sequence of SEQ ID NO: 4517; or
xii). a CDR1 comprising an amino acid sequence of SEQ ID NO: 16, a CDR2 comprising an amino acid sequence of SEQ ID NO: 4699, a CDR3 comprising an amino acid sequence of SEQ ID NO: 4771.

In certain embodiments, an anti-TNFR2 antigen-binding protein (e.g., antibody such as a single-domain antibody) of the present disclosure comprises
a) a CDR1 comprising an amino acid sequence of SEQ ID NO: 69, a CDR2 comprising an amino acid sequence of SEQ ID NO: 76, a CDR3 comprising an amino acid sequence of SEQ ID NO: 61;
b) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 63;
c) a CDR1 comprising an amino acid sequence of SEQ ID NO: 73, a CDR2 comprising an amino acid sequence of SEQ ID NO: 80, a CDR3 comprising an amino acid sequence of SEQ ID NO: 66; or
d) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 4771.

In certain embodiments, an anti-TNFR2 antigen-binding protein (e.g., antibody such as a single-domain antibody) of the present disclosure comprises
i) a CDR1 with an amino acid sequence of GSI(V/F)(R/S)(A/T)(N/D)(G/A) (SEQ ID NO: 4700), a CDR2 comprising an amino acid sequence of IRSDGFT (SEQ ID NO: 2), and a CDR3 comprising an amino acid sequence of YYQ(S/A)LSSPNYGQ(V/T)F (SEQ ID NO: 4701);
ii) a CDR1 with an amino acid sequence of GFTFDDIA (SEQ ID NO: 8), a CDR2 comprising an amino acid sequence of IYSYGPNT (SEQ ID NO: 9), and a CDR3 comprising an amino acid sequence of AADSDLSTVV(V/T)GPHDY (SEQ ID NO: 4702);
iii) a CDR1 with an amino acid sequence of GFTFSRYA (SEQ ID NO: 12), a CDR2 comprising an amino acid sequence of ISDDGSDT (SEQ ID NO: 13), and a CDR3 comprising an amino acid sequence of AKDAGSWGTGPFGYEYDY (SEQ ID NO: 14);
iv) a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of AA(T/A)PSGKAYSY (SEQ ID NO: 4703);
v) a CDR1 with an amino acid sequence of GLTLDYYA (SEQ ID NO: 20), a CDR2 comprising an amino acid sequence of ISTSDGST (SEQ ID NO: 21), and a CDR3 comprising an amino acid sequence of ATPGPYTYCAPYGSSWSRGYDY (SEQ ID NO: 22);
vi) a CDR1 with an amino acid sequence of GF(T/N)FSMYS (SEQ ID NO: 72), a CDR2 comprising an amino acid sequence of IDT(R/G)GST (SEQ ID NO: 79), and a CDR3 comprising an amino acid sequence of ARV(G/R)G(T/A)PYEY(N/G)Y (SEQ ID NO: 4704);
vii) a CDR1 with an amino acid sequence of GRTF(G/S)S(Y/L)(T/F) (SEQ ID NO: 4705), a CDR2 comprising an amino acid sequence of IR(W/R/Y)(T/P)G(G/L)(S/I)T (SEQ ID NO: 80), and a CDR3 comprising an amino acid sequence of (A/V)A(A/S)PTGRAF(T/N)Y (SEQ ID NO: 4707);
viii) a CDR1 with an amino acid sequence of GASLSRNA (SEQ ID NO: 40), a CDR2 comprising an amino acid sequence of IYDDGET (SEQ ID NO: 41), and a CDR3 comprising an amino acid sequence of AGSAFDF (SEQ ID NO: 42);
ix) a CDR1 with an amino acid sequence of GS(T/I)FRFPP (SEQ ID NO: 4708), a CDR2 comprising an amino acid sequence of LTSGGST (SEQ ID NO: 45), and a CDR3 comprising an amino acid sequence of SVLGRDM(M/V)TY (SEQ ID NO: 4706);
x) a CDR1 with an amino acid sequence of GFTLDDYA (SEQ ID NO: 4061), a CDR2 comprising an amino acid sequence of IFSYSSNT (SEQ ID NO: 4062), and a CDR3 comprising an amino acid sequence of AVGDFEGELVLKGDY (SEQ ID NO: 4063);
xi) a CDR1 with an amino acid sequence of GFTLDYYT (SEQ ID NO: 4065), a CDR2 comprising an amino acid sequence of ISSNDGSV (SEQ ID NO: 4066), and a CDR3 comprising an amino acid sequence of AADLGYLYVDYVRLHTHHFGS (SEQ ID NO: 4067);
xii). a CDR1 with an amino acid sequence of GRTFSDYG (SEQ ID NO: 4719), a CDR2 comprising an amino acid sequence of INWSNGRT (SEQ ID NO: 4723), and a CDR3 comprising an amino acid sequence of AATPTGKAYTY (SEQ ID NO: 4727);
xiii). a CDR1 with an amino acid sequence of GRTFSDYG (SEQ ID NO: 4720), a CDR2 comprising an amino acid sequence of INWSNGRT (SEQ ID NO: 4724), and a CDR3 comprising an amino acid sequence of AATPTGKAYTY (SEQ ID NO: 4728);
xiv). a CDR1 with an amino acid sequence of GRTFSDYG (SEQ ID NO: 4721), a CDR2 comprising an amino acid sequence of INWSNGRT (SEQ ID NO: 4725), and a CDR3 comprising an amino acid sequence of AGTLSGKAYTY (SEQ ID NO: 4729); or
xv). a CDR1 with an amino acid sequence of GRTFSDYG (SEQ ID NO: 4722), a CDR2 comprising an amino acid sequence of INWSNGRT (SEQ ID NO: 4726), and a CDR3 comprising an amino acid sequence of AGTLSGKAYTY (SEQ ID NO: 4730).

In certain embodiments, an anti-TNFR2 antigen-binding protein (e.g., antibody such as a single-domain antibody) of the present disclosure comprises
a) a CDR1 with an amino acid sequence of GFTFDDIA (SEQ ID NO: 8), a CDR2 comprising an amino acid sequence of IYSYGPNT (SEQ ID NO: 9), and a CDR3 comprising an amino acid sequence of AADSDLSTVV(V/T)GPHDY (SEQ ID NO: 4702);
b) a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of AA(T/A)PSGKAYSY (SEQ ID NO: 4703);
c) a CDR1 with an amino acid sequence of GRTF(G/S)S(Y/L)(T/F) (SEQ ID NO: 4705), a CDR2 comprising an amino acid sequence of IR(W/R/Y)(T/P)G(G/L)(S/I)T (SEQ ID NO: 80), and a CDR3 comprising an amino acid sequence of (A/V)A(A/S)PTGRAF(T/N)Y (SEQ ID NO: 4707); or
d). a CDR1 comprising an amino acid sequence of SEQ ID NO: 16, a CDR2 comprising an amino acid sequence of SEQ ID NO: 4699, a CDR3 comprising an amino acid sequence of SEQ ID NO: 4771.

Provided herein are anti-TNFR2 antigen-binding proteins (e.g., antibodies such as single-domain antibodies) comprising a CDR1 (CDR1) comprising an amino acid sequence selected from any of the CDR1 amino acid sequences listed in Table 1-1 or Table 5, or a similar sequence thereof having at least 70%, at least 80%, at least 90%, or at least 95% sequence identity.

In some embodiments, an anti-TNFR2 antigen-binding protein (e.g., antibody such as single-domain antibody) comprises a CDR1 comprising an amino acid sequence selected from SEQ ID NOs: 1, 5, 8, 12, 16, 20, 24, 28, 32, 36, 40, 44, 4061, 4065, 4069, 4520, 4719-4722, 1128-1686, 4173-4234, and 4533-4556, or a similar sequence thereof having at least 70%, at least 80%, at least 90%, or at least 95% sequence identity.

Provided herein are anti-TNFR2 antigen-binding proteins (e.g., antibodies such as single-domain antibodies) comprising a CDR2 (CDR2) comprising an amino acid sequence selected from any of the CDR2 amino acid sequences listed in Table 1-1 or Table 5, or a similar sequence thereof having at least 70%, at least 80%, at least 90%, or at least 95% sequence identity.

In some embodiments, an anti-TNFR2 antigen-binding protein (e.g., antibody such as single-domain antibody) comprises a CDR2 comprising an amino acid sequence selected from SEQ ID NOs: 2, 9, 13, 17, 21, 25, 29, 33, 37, 41, 45, 4062, 4066, 4070, 4527, 4723-4726, 1687-2245, 4235-4296, and 4557-4580, or a similar sequence thereof having at least 70%, at least 80%, at least 90%, or at least 95% sequence identity.

Provided herein are anti-TNFR2 antigen-binding proteins (e.g., antibodies such as single-domain antibodies) comprising a CDR3 (CDR3) comprising an amino acid sequence selected from any of the CDR3 amino acid sequences listed in Table 1-1 or Table 5, or a similar sequence thereof having at least 70%, at least 80%, at least 90%, or at least 95% sequence identity.

In some embodiments, an anti-TNFR2 antigen-binding protein (e.g., antibody such as single-domain antibody) comprises a CDR3 comprising an amino acid sequence selected from SEQ ID NOs: 3, 6, 10, 14, 18, 22, 26, 30, 34, 38, 42, 46, 4063, 4067, 4071, 4524, 4530, 4727-4730, 2246-2804, 4297-4358, and 4581-4604, or a similar sequence thereof having at least 70%, at least 80%, at least 90%, or at least 95% sequence identity.

Provided herein are anti-TNFR2 antigen-binding proteins (e.g., antibodies such as single-domain antibodies) comprising a set of three CDRs (i.e., CDR1-CDR2-CDR3) contained within any of the exemplary anti-TNFR2 VHH antibodies listed in Tables 1-1, Table 1-2, or Table 5. In certain embodiments, an anti-TNFR2 antigen-binding protein (e.g., antibody such as a single-domain antibody) of the present disclosure comprises
i) a CDR1 comprising an amino acid sequence of SEQ ID NO: 1, a CDR2 comprising an amino acid sequence of SEQ ID NO: 2, and a CDR3 comprising an amino acid sequence of SEQ ID NO: 3;
ii) a CDR1 comprising an amino acid sequence of SEQ ID NO: 5, a CDR2 comprising an amino acid sequence of SEQ ID NO: 2, a CDR3 comprising an amino acid sequence of SEQ ID NO: 6;
iii) a CDR1 comprising an amino acid sequence of SEQ ID NO: 8, a CDR2 comprising an amino acid sequence of SEQ ID NO: 9, a CDR3 comprising an amino acid sequence of SEQ ID NO: 10;
iv) a CDR1 comprising an amino acid sequence of SEQ ID NO: 12, a CDR2 comprising an amino acid sequence of SEQ ID NO: 13, a CDR3 comprising an amino acid sequence of SEQ ID NO: 14;
v) a CDR1 comprising an amino acid sequence of SEQ ID NO: 16, a CDR2 comprising an amino acid sequence of SEQ ID NO: 17, a CDR3 comprising an amino acid sequence of SEQ ID NO: 18;
vi) a CDR1 comprising an amino acid sequence of SEQ ID NO: 20, a CDR2 comprising an amino acid sequence of SEQ ID NO: 21, a CDR3 comprising an amino acid sequence of SEQ ID NO: 22;
vii) a CDR1 comprising an amino acid sequence of SEQ ID NO: 24, a CDR2 comprising an amino acid sequence of SEQ ID NO: 25, a CDR3 comprising an amino acid sequence of SEQ ID NO: 26;
viii) a CDR1 comprising an amino acid sequence of SEQ ID NO: 28, a CDR2 comprising an amino acid sequence of SEQ ID NO: 29, a CDR3 comprising an amino acid sequence of SEQ ID NO: 30;
ix) a CDR1 comprising an amino acid sequence of SEQ ID NO: 32, a CDR2 comprising an amino acid sequence of SEQ ID NO: 33, a CDR3 comprising an amino acid sequence of SEQ ID NO: 34;
x) a CDR1 comprising an amino acid sequence of SEQ ID NO: 36, a CDR2 comprising an amino acid sequence of SEQ ID NO: 37, a CDR3 comprising an amino acid sequence of SEQ ID NO: 38;
xi) a CDR1 comprising an amino acid sequence of SEQ ID NO: 40, a CDR2 comprising an amino acid sequence of SEQ ID NO: 41, a CDR3 comprising an amino acid sequence of SEQ ID NO: 42;
xii) a CDR1 comprising an amino acid sequence of SEQ ID NO: 44, a CDR2 comprising an amino acid sequence of SEQ ID NO: 45, a CDR3 comprising an amino acid sequence of SEQ ID NO: 46;
xiii) a CDR1 comprising an amino acid sequence of SEQ ID NO: 4061, a CDR2 comprising an amino acid sequence of SEQ ID NO: 4062, a CDR3 comprising an amino acid sequence of SEQ ID NO: 4063;
xiv). a CDR1 comprising an amino acid sequence of SEQ ID NO: 4065, a CDR2 comprising an amino acid sequence of SEQ ID NO: 4066, a CDR3 comprising an amino acid sequence of SEQ ID NO: 4067;
xv). a CDR1 comprising an amino acid sequence of SEQ ID NO: 4069, a CDR2 comprising an amino acid sequence of SEQ ID NO: 4070, a CDR3 comprising an amino acid sequence of SEQ ID NO: 4071;
xvi). a CDR1 comprising an amino acid sequence of SEQ ID NO: 4520, a CDR2 comprising an amino acid sequence of SEQ ID NO: 45, a CDR3 comprising an amino acid sequence of SEQ ID NO: 46;
xvii). a CDR1 comprising an amino acid sequence of SEQ ID NO: 8, a CDR2 comprising an amino acid sequence of SEQ ID NO: 9, a CDR3 comprising an amino acid sequence of SEQ ID NO: 4524;
xviii). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 18;
ix). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 4530;
x). a CDR1 comprising an amino acid sequence of SEQ ID NO: 4719, a CDR2 comprising an amino acid sequence of SEQ ID NO: 4723, a CDR3 comprising an amino acid sequence of SEQ ID NO: 4727;
xii). a CDR1 comprising an amino acid sequence of SEQ ID NO: 4720, a CDR2 comprising an amino acid sequence of SEQ ID NO: 4724, a CDR3 comprising an amino acid sequence of SEQ ID NO: 4728;
xiii). a CDR1 comprising an amino acid sequence of SEQ ID NO: 4721, a CDR2 comprising an amino acid sequence of SEQ ID NO: 4725, a CDR3 comprising an amino acid sequence of SEQ ID NO: 4729; or
xiv).). a CDR1 comprising an amino acid sequence of SEQ ID NO: 4722, a CDR2 comprising an amino acid sequence of SEQ ID NO: 4726, a CDR3 comprising an amino acid sequence of SEQ ID NO: 4730.

In some embodiments, an anti-TNFR2 antigen-binding protein (e.g., antibody such as single-domain antibody) comprises
a) a CDR1 comprising an amino acid sequence of SEQ ID NO: 16, a CDR2 comprising an amino acid sequence of SEQ ID NO: 17, a CDR3 comprising an amino acid sequence of SEQ ID NO: 18;
b). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 18;
c). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 4530;
d) a CDR1 comprising an amino acid sequence of SEQ ID NO: 8, a CDR2 comprising an amino acid sequence of SEQ ID NO: 9, a CDR3 comprising an amino acid sequence of SEQ ID NO: 10;
e). a CDR1 comprising an amino acid sequence of SEQ ID NO: 8, a CDR2 comprising an amino acid sequence of SEQ ID NO: 9, a CDR3 comprising an amino acid sequence of SEQ ID NO: 4524;
f). a CDR1 comprising an amino acid sequence of SEQ ID NO: 4069, a CDR2 comprising an amino acid sequence of SEQ ID NO: 4070, a CDR3 comprising an amino acid sequence of SEQ ID NO: 4071;
g). a CDR1 comprising an amino acid sequence of SEQ ID NO: 4719, a CDR2 comprising an amino acid sequence of SEQ ID NO: 4723, a CDR3 comprising an amino acid sequence of SEQ ID NO: 4727;
h). a CDR1 comprising an amino acid sequence of SEQ ID NO: 4720, a CDR2 comprising an amino acid sequence of SEQ ID NO: 4724, a CDR3 comprising an amino acid sequence of SEQ ID NO: 4728;
i). a CDR1 comprising an amino acid sequence of SEQ ID NO: 4721, a CDR2 comprising an amino acid sequence of SEQ ID NO: 4725, a CDR3 comprising an amino acid sequence of SEQ ID NO: 4729; or
j).). a CDR1 comprising an amino acid sequence of SEQ ID NO: 4722, a CDR2 comprising an amino acid sequence of SEQ ID NO: 4726, a CDR3 comprising an amino acid sequence of SEQ ID NO: 4730.

In a related embodiment, provided herein are anti-TNFR2 antigen-binding proteins (e.g., antibodies such as single-domain antibodies) comprising a set of three CDRs (I.e., CDR1-CDR2-CDR3) contained within a VHH amino acid sequence as defined by any of the exemplary anti-TNFR2 VHH antibodies listed in Table 1-1, Table 1-2, or Table 5. For example, provided herein are antibodies, or antigen-binding fragments thereof, comprising the set of CDR1-CDR2-CDR3 amino acid sequences contained within a VHH amino acid sequence selected from SEQ ID NOs: 4, 7, 11, 15, 19, 23, 27, 31, 35, 39, 43, 47, 4064, 4068, 4072, 4521, 81-92, 93-640, 4079-4125, 2805-3363, 4359-4420, 4605-4628, 5426, 4529, 4532, 4078, 4523, 4076, 4077, 4078, and 4731-4734.

In some embodiments, an anti-TNFR2 antigen-binding protein (e.g., antibody such as a single-domain antibody) of the present disclosure can include
a) a variable domain that comprises a CDR1, CDR2, and CDR3 contained within a VHH comprising the amino acid sequence of SEQ ID NO: 4;
b) a variable domain that comprises a CDR1, CDR2, and CDR3 contained within a VHH comprising the amino acid sequence of SEQ ID NO: 7;
c) a variable domain that comprises a CDR1, CDR2, and CDR3 contained within a VHH comprising the amino acid sequence of SEQ ID NO: 11;
d) a variable domain that comprises a CDR1, CDR2, and CDR3 contained within a VHH comprising the amino acid sequence of SEQ ID NO: 15;
e) a variable domain that comprises a CDR1, CDR2, and CDR3 contained within a VHH comprising the amino acid sequence of SEQ ID NO: 19;
f) a variable domain that comprises a CDR1, CDR2, and CDR3 contained within a VHH comprising the amino acid sequence of SEQ ID NO: 23;
g) a variable domain that comprises a CDR1, CDR2, and CDR3 contained within a VHH comprising the amino acid sequence of SEQ ID NO: 27;
h) a variable domain that comprises a CDR1, CDR2, and CDR3 contained within a VHH comprising the amino acid sequence of SEQ ID NO: 31;
i) a variable domain that comprises a CDR1, CDR2, and CDR3 contained within a VHH comprising the amino acid sequence of SEQ ID NO: 35;
j) a variable domain that comprises a CDR1, CDR2, and CDR3 contained within a VHH comprising the amino acid sequence of SEQ ID NO: 39;
k) a variable domain that comprises a CDR1, CDR2, and CDR3 contained within a VHH comprising the amino acid sequence of SEQ ID NO: 43;
l) a variable domain that comprises a CDR1, CDR2, and CDR3 contained within a VHH comprising the amino acid sequence of SEQ ID NO: 47;
m). a variable domain that comprises a CDR1, CDR2, and CDR3 contained within a VHH comprising the amino acid sequence of SEQ ID NO: 4064;
n). a variable domain that comprises a CDR1, CDR2, and CDR3 contained within a VHH comprising the amino acid sequence of SEQ ID NO: 4068;
o). a variable domain that comprises a CDR1, CDR2, and CDR3 contained within a VHH comprising the amino acid sequence of SEQ ID NO: 4072;
p). a variable domain that comprises a CDR1, CDR2, and CDR3 contained within a VHH comprising the amino acid sequence of SEQ ID NO: 4521;
q). a variable domain that comprises a CDR1, CDR2, and CDR3 contained within a VHH comprising the amino acid sequence of SEQ ID NO: 4526;
r) a variable domain that comprises a CDR1, CDR2, and CDR3 contained within a VHH comprising the amino acid sequence of SEQ ID NO: 4529;
s) a variable domain that comprises a CDR1, CDR2, and CDR3 contained within a VHH comprising the amino acid sequence of SEQ ID NO: 4532;
t) a variable domain that comprises a CDR1, CDR2, and CDR3 contained within a VHH comprising the amino acid sequence of SEQ ID NO: 4078;
u) a variable domain that comprises a CDR1, CDR2, and CDR3 contained within a VHH comprising the amino acid sequence of SEQ ID NO: 4731;
v) a variable domain that comprises a CDR1, CDR2, and CDR3 contained within a VHH comprising the amino acid sequence of SEQ ID NO: 4732;
w) a variable domain that comprises a CDR1, CDR2, and CDR3 contained within a VHH comprising the amino acid sequence of SEQ ID NO: 4733;
x) a variable domain that comprises a CDR1, CDR2, and CDR3 contained within a VHH comprising the amino acid sequence of SEQ ID NO: 4734.

In some embodiments, an anti-TNFR2 antigen-binding protein (e.g., antibody such as a single-domain antibody) of the present disclosure can include
a) a variable domain that comprises a CDR1, CDR2, and CDR3 contained within a VHH comprising the amino acid sequence of SEQ ID NO: 19;
b). a variable domain that comprises a CDR1, CDR2, and CDR3 contained within a VHH comprising the amino acid sequence of SEQ ID NO: 4072;
c). a variable domain that comprises a CDR1, CDR2, and CDR3 contained within a VHH comprising the amino acid sequence of SEQ ID NO: 4526;
d) a variable domain that comprises a CDR1, CDR2, and CDR3 contained within a VHH comprising the amino acid sequence of SEQ ID NO: 4529;
e) a variable domain that comprises a CDR1, CDR2, and CDR3 contained within a VHH comprising the amino acid sequence of SEQ ID NO: 4532;
f) a variable domain that comprises a CDR1, CDR2, and CDR3 contained within a VHH comprising the amino acid sequence of SEQ ID NO: 4731;
g) a variable domain that comprises a CDR1, CDR2, and CDR3 contained within a VHH comprising the amino acid sequence of SEQ ID NO: 4732;
h) a variable domain that comprises a CDR1, CDR2, and CDR3 contained within a VHH comprising the amino acid sequence of SEQ ID NO: 4733; or
i) a variable domain that comprises a CDR1, CDR2, and CDR3 contained within a VHH comprising the amino acid sequence of SEQ ID NO: 4734.

In an embodiment provided herein, an anti-TNFR2 antigen-binding protein (e.g., antibody such as a single-domain antibody) of the present disclosure can include a VHH amino acid sequence selected from SEQ ID NOs: 4, 7, 11, 15, 19, 23, 27, 31, 35, 39, 43, 47, 4064, 4068, 4072, 4521, 93-640, 4079-4125, 2805-3363, 4359-4420, 4605-4628, and 4653-4685, or a similar sequence thereof having at least 70%, at least 75%, at least 80%, at least 85%, at least 90%, at least 95%, at least 98%, or at least 99% sequence identity.

In an embodiment provided herein, an anti-TNFR2 antigen-binding protein (e.g., antibody such as a single-domain antibody) of the present disclosure can include a VHH amino acid sequence selected from SEQ ID NOs: 4, 7, 11, 15, 19, 23, 27, 31, 35, 39, 43, 47, 4064, 4068, 4072, 4521, and 4653-4685 or a similar sequence thereof having at least 70%, at least 75%, at least 80%, at least 85%, at least 90%, at least 95%, at least 98%, or at least 99% sequence identity.

In an embodiment provided herein, an anti-TNFR2 antigen-binding protein (e.g., antibody such as a single-domain antibody) of the present disclosure can include a humanized VHH amino acid sequence selected from SEQ ID NOs: 81-92, 4076-4078, 4523, 4526, 4529, 4532, 4731-4734, 641-1127, and 4126-4172, or a similar sequence thereof having at least 70%, at least 75%, at least 80%, at least 85%, at least 90%, at least 95%, at least 98%, or at least 99% sequence identity.

In an embodiment provided herein, an anti-TNFR2 antigen-binding protein (e.g., antibody such as a single-domain antibody) of the present disclosure can include a VHH amino acid sequence selected from SEQ ID NOs: 19, 4072, 4078, 4526, 4529, 4532, and 4653-4685 or a similar sequence thereof having at least 70%, at least 75%, at least 80%, at least 85%, at least 90%, at least 95%, at least 98%, or at least 99% sequence identity.

In some embodiments, the present disclosure also provides an anti-TNFR2 antigen-binding protein (e.g., antibody such as a single-domain antibody) that competes for binding to TNFR2 with any one of the exemplary anti-TNFR2 VHH antibodies listed in Table 1-1, Table 1-2, or Table 5.

In some embodiments, the present disclosure also provides an anti-TNFR2 antigen-binding protein (e.g., antibody such as a single-domain antibody) that binds to the same epitope on TNFR2 as any one of the exemplary anti-TNFR2 VHH antibodies listed in Table 1-1, Table 1-2, or Table 5.

### Single-Domain Antibodies

A single-domain antibody (e.g., VHH) can be obtained by immunization of dromedaries, camels, llamas, alpacas, or sharks with the desired antigen and subsequent isolation of the mRNA coding for heavy-chain antibodies. Antigens can be purified from natural sources, or in the course of recombinant production. Immunization and/or screening for immunoglobulin sequences can be performed using peptide fragments of such antigens. By reverse transcription and polymerase chain reaction (PCR), a gene library of single-domain antibodies containing several million clones can be produced. Screening techniques such as phage display, yeast display, and ribosome display help to identify the clones binding the antigen. Methods generation of heavy-chain antibody fragments are described in e.g., WO 94/04678; Hamers-Casterman et al. 1993; Muyldermans et al. 2001; and Arbabi Ghahroudi, M. et al. (1997). FEBS Letters 414 (3): 521-526, each of which is incorporated herein by reference in its entirety.

A different method may use gene libraries from animals that have not been previously immunized. Such naïve libraries usually contain only antibodies with low affinity to the desired antigen, making it necessary to apply affinity maturation by random mutagenesis as an additional step. See e.g., Saerens, D.; et al. (2008). “Single-domain antibodies as building blocks for novel therapeutics”. Current Opinion in Pharmacology 8 (5): 600-608.

Affinity maturation strategies can be categorized as either targeted/rational approaches or untargeted/random approaches. For targeted approaches information about the VHH of interest is needed, such as hot spots for affinity maturation or structural information on the VHH: antigen complex, whereas for untargeted approaches no prior information is needed. Targeted approaches that may be applied for affinity maturation of VHHs include site-directed in-vitro mutagenesis and in-silico/computational approaches. Common untargeted approaches used for affinity maturation of VHHs include random in-vitro mutagenesis, CDR swapping and autonomous hypermutation yeast surface display, with the latter two being novel, emerging and very time efficient techniques. Most of these strategies have in common, that after applying a certain randomization strategy to generate a mutational library, the resulting library can be screened by employing standard display techniques such as yeast, phage or ribosome display to select for the best binders. The choice of the display system is often guided by the library size to be displayed, with yeast display being able to handle library sizes of ~10⁷-10⁹, phage display ~10⁸-10¹⁰ and ribosome display ~10¹²-10¹³ (Chan and Groves, 2021). Notably, during affinity maturation the number of highly interactive residues such as aromatic amino acids usually increase in the CDR regions. The selected affinity matured clones may be further evaluated by a developability assessment to test for undesired properties, such as unspecific binding to off-targets or VHH instability.

For targeted in vitro mutagenesis, a set of selected residues within the CDRs of a VHH may be mutated (Tiller et al., 2017; Yau et al., 2005). Pre-selection of these residues can be either performed using alanine scanning to identify hot spot residues for mutation or by using structural data of the antigen:VHH complex to identify positions to be mutated. These sites can then be either submitted to saturating mutagenesis to substitute a specific site with all possible amino acids or specific amino acid substitutions yielding several smaller libraries. After mutagenesis binders can be displayed to select the best matured candidate. Usually, several rounds of targeted mutagenesis are performed with separate sub-libraries to obtain combinations of individual mutations that cooperatively result in increased binding affinity.

Computer-aided/in silico methods are often used to guide targeted in vitro mutagenesis. Using homology modeling of the target:VHH complex or docking, hotspots for mutations can be identified that are then submitted to in vitro mutagenesis (Bert Schepens et al., 2021; Cheng et al., 2019; Inoue et al., 2013; Mahajan et al., 2018). Further, in silico methods can search all designed variants in a virtual library (~10₄₀ members) in a rather short amount of time to identify a feasible number of promising candidates to be tested experimentally. These techniques can be especially valuable if structural data on the drug-target interaction are available.

Untargeted/random affinity maturation strategies that can be applied to affinity mature VHHs include random in vitro mutagenesis, CDR shuffling/swapping and in vivo affinity maturation via yeast display. For random in vitro mutagenesis the sequence of either the entire VHH or only the CDRs are mutated randomly (Chen et al., 2021; Ye et al., 2021; Zupancic et al., 2021). The most commonly used technique is error prone PCR employing a DNA polymerase that lacks proof reading activity and PCR conditions that increase the polymerase error rate even further. This technique can be applied without further structural knowledge or information on the importance of residues that contribute to antigen:VHH interaction. The resulting mutational library can then be displayed to select the best matured candidate. This technique may also be combined with NGS sequencing of the display elutions to get an in-depth readout of all obtained candidates, enabling the identification of low abundant but still promising clones (Chen et al., 2021).

In some embodiments, CDR shuffling or swapping is applied for VHH affinity maturation, such as described in Zupancic et al., 2021. For CDR swapping, enriched libraries can be used as input material for a PCR reaction to individually amplify the CDR of the VHHs. The PCR products can then be mixed and reassembled using overlapping PCR to generate the entire plasmid for further rounds of display to select for the best matured binder. One limitation of this approach is that it can only be used for VHHs comprising the same framework as it is the case for synthetic libraries.

In some embodiments, in vivo affinity maturation via yeast display is applied for VHH affinity maturation, such as described in Wellner et al., 2021. The method is based on an autonomous hypermutation yeast surface display (AHEAD), which imitates somatic hypermutation during VHH selection using engineered yeast strains. The yeast's error prone orthogonal DNA replication system can generate new variants during plasmid replication by randomly introducing mutations. The new variants can then be displayed and selected using yeast surface display to identify the best binders. This enables the production of high affinity clones in very little time (about 2 weeks), which is significantly faster than classical affinity maturation procedures. The method can be applied using synthetic or immune libraries using unenriched libraries enriched libraries or a subset of preselected clones.

In case binders with medium affinity are required, as it is the case for the anti-TNFR2 V-bodies and the affinity of the identified candidates need to be decreased, very similar techniques can be applied. For example, mutations that are aiming at lowering the affinity can be introduced using the same targeted or untargeted approaches as described for the affinity maturation. The selection afterwards can be adapted accordingly. If larger libraries are generated that need to be screened via a display technique, the selection strategy can be adapted to enrich medium affinity binders while excluding high affinity candidates. This could, for example be a pre-panning in phage display with low antigen concentration to remove all higher affinity candidates, followed by a selection with high antigen concentration to obtain medium affinity VHHs. For library sizes of up to 1000 candidates a kinetic off-rate characterization can be used to get immediate information about the kinetic behavior of the candidates.

When the most potent clones have been identified, their DNA sequence can be optimized, for example to improve their stability towards enzymes. Another goal is humanization to prevent immunological reactions of the human organism against the antibody. Humanization can be achieved based on the homology between camelid VHH and human VH fragments, which is described in further detail below. Finally, the optimized single-domain antibody can be translated and expressed in suitable organisms such as E. coli or Saccharomyces cerevisiae.

Single-domain antibodies can also be derived from conventional antibodies. In some embodiments, single-domain antibodies can be made from conventional murine or human IgG with four chains. The process is similar, comprising gene libraries from immunized or naïve donors and display techniques for identification of the most specific antigens. However, the binding region of a conventional IgG consists of two domains (VH and VL), which tend to dimerize or aggregate because of their lipophilicity. Monomerization can be accomplished by replacing lipophilic by hydrophilic amino acids. (See e.g., Borrebaeck, C. A. K.; Ohlin, M. (2002). “Antibody evolution beyond Nature”, Nature Biotechnology 20 (12): 1189-90.) If affinity can be retained after monomerization, the single-domain antibodies can likewise be produced in E. coli, S. cerevisiae or other suitable organisms.

A “humanized antibody” refers to a chimeric, genetically engineered, antibody in which the amino acid sequences (typically CDRs) from an antibody (donor antibody), e.g., a camelid antibody, are grafted onto a human antibody (acceptor antibody). Thus, a humanized antibody typically comprises CDRs from a donor antibody and variable region framework and constant regions, when present, from a human antibody. Accordingly, a “humanized VHH” comprises CDRs that corresponds to the CDRs of a naturally occurring VHH domain (e.g., a camelid VHH), but that has been “humanized”. Humanized VHH may be prepared by replacing one or more amino acid residues in the amino acid sequence of the naturally occurring VHH sequence (particularly in the framework sequences) by one or more of the amino acid residues that occur at the corresponding position(s) in a VH domain from a conventional 4-chain human antibody. Such humanized VHHs can be obtained in any suitable manner known to a skilled person in the art and thus not strictly limited to methods described herein.

Humanization of VHHs can achieved using resurfacing or CDR grafting. Resurfacing strategies have been described in e.g., Conrath et al., 2005 J Mol Biol; Kazemi-Lomedasht et al., 2018; Vincke et al., 2009 J Biol Chem, and CDR grafting strategies have been described in e.g., ben Abderrazek et al., 2011; van Faassen et al., 2020 FASEB; Li et al., 2018; Vaneycken et al., 2010; Vincke et al., 2009 J Biol Chem; and Yu et al., 2017, each of which is incorporated herein by reference in its entirety.

To humanize a camelid VHH using a resurfacing approach, a human germline reference that is most similar to the camelid germline sequence of the selected VHH may be identified. Most of the isolated camelid VHHs in literature belong to the camelid IGHV3 subfamily 2 (Nguyen et al., 2000, EMBO J) with DP-47/VH3-23 from the IGHV3 family commonly used as human reference. The framework of the camelid VHH can then be compared to the human reference sequence. Surface exposed residues are substituted to their human counterpart as it is assumed that their contribution to protein stability is rather low. Buried residues however remain of camelid origin, as they likely contribute to the overall VHH stability. Humanization of framework regions 1, 3 and 4 usually does not impact the physicochemical properties of the VHHs, whereas a general humanization of framework 2 would significantly increase local hydrophobicity. Residues H37, H44, H45 and H47 (Chothia numbering) in framework 2, the so called tetrade or hallmark residues, have a rather hydrophobic nature in human VHs (VGLW) as they are partially buried and involved in VH/VL paring, while in camelid VHHs these residues are partially charged (FERG), which significantly increases VHH solubility and inhibits paring of camelid VL (Soler et al., 2021, Biomolecules, Conrath et al., 2005 J Mol Biol). Further, residues H37 and H47 are known to interact with the CDR-H3 loop in many VHHs, stabilizing its conformation and thereby contributing to antigen binding affinity. In addition, a significant number of VHHs use framework 2 residues H44, H45 and H47 for antigen binding (Zavrtanik et al., 2018, J Mol Biol). A full humanization of these residues hence frequently results in reduced solubility or aggregation of the VHHs and a reduced or complete loss of binding affinity for the target antigen (van Faassen et al., 2020, Vincke et al., 2009). In consequence, all or at least some of these hallmark residues in framework 2 remain of camelid origin when humanizing VHHs.

Another approach that may be applied to humanize VHHs is CDR grafting. CDRs of the selected VHHs can be transplanted onto a universal VHH framework that has been partially or fully humanized (Saerens et al., 2009 J Biol Chem, Soler et al., 2021, Vincke et al., 2009 J Biol Chem). CDR grafting has been successfully used in some cases but failed for several others, with VHHs frequently losing their potential to bind to the desired antigen and/or becoming structurally instable with a high tendency to aggregate (van Faassen et al., 2020, FASEB). This is mostly attributed to interactions of CDR3 with specific residues in framework 2 that are important for CDR3 conformation, general VHH stability and overall hydrophobicity, which are impaired by this approach. Sometimes camelid backmutations are introduced into the framework to compensate for these effects (van Faassen et al., 2020, FASEB).

An alternative strategy to mitigate the need of humanizing the selected VHH sequences is to use fully or partially humanized synthetic VHH libraries instead of camelid immune libraries for VHH discovery (Moutel et al. 2016, eLife; McMahon, 2018, NSMB; Zimmermann et al., 2018, eLife). In many of these libraries the hallmark residues are still of camelid origin for reasons discussed above.

Other suitable humanizing substitutions are described in WO 09/138519 and WO 08/020079, as well as Tables A-3 to A-8 from WO 08/020079 (which are lists showing possible humanizing substitutions), each of which is incorporated herein by reference in its entirety. Non-limiting examples of such humanizing substitutions include Q108L and A14P. Such humanizing substitutions may also be suitably combined with one or more other mutations as described herein (such as with one or more mutations that reduce binding by pre-existing antibodies).

In some embodiments, humanized VHH sequences still retain the residues that are relevant for protein A binding. In some embodiments, the engineering activities during humanization may be applied to engineer protein A binding properties into a VHH that did previously not interact with protein A (Graille et al., 2000, PNAS).

Like a “humanized antibody”, a “camelized antibody” refers to an antibody having amino acid sequences (typically CDRs) from a donor antibody, e.g., a human antibody, and variable region framework and constant regions, when present, from a camelid antibody. Accordingly, a “camelized VH” comprises an amino acid sequence that corresponds to the amino acid sequence of a naturally occurring VH domain, but that has been “camelized”. Camelized VH may be prepared by replacing one or more amino acid residues in the amino acid sequence of a naturally occurring VH domain from a conventional 4-chain antibody by one or more of the amino acid residues that occur at the corresponding position(s) in a VHH domain of a heavy chain antibody. This can be performed in a manner, for example as described in WO 2008/020079. Such “camelizing” substitutions are usually inserted at amino acid positions that form and/or are present at the VH-VL interface, and/or at the so-called Camelidae hallmark residues, e.g., F37, E44, R45 and F47 (see for example WO 94/04678 and Davies and Riechmann (1994 and 1996)). In one embodiment, the VH sequence that is used as a starting material or starting point for generating or designing the camelized VH is a VH sequence from a mammal, or the VH sequence of a human antibody. However, such camelized VH can be obtained in any suitable manner known to a skilled person in the art and thus are not strictly limited to polypeptides that have been obtained using a polypeptide that comprises a naturally occurring VH domain as a starting material.

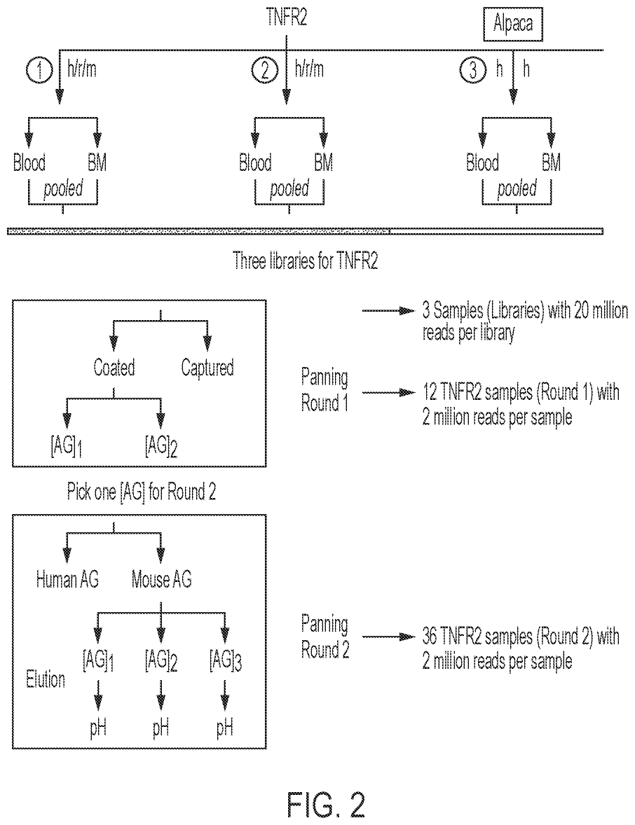

The amino acid residues of a single-domain antibody can be numbered according to the general numbering for VH domains given by Kabat et al. (“Sequence of proteins of immunological interest”, US Public Health Services, NIH Bethesda, Md., Publication No. 91), as applied to VHH domains from Camelids described in Riechmann and Muyldermans, 2000 (J. Immunol. Methods 240 (1-2): 185-195; see for example FIG. 2 of this publication). The total number of amino acid residues in each of the CDRs may vary and may not correspond to the total number of amino acid residues indicated by the Kabat numbering. For example, one or more positions according to the Kabat numbering may not be occupied in the actual sequence, or the actual sequence may contain more amino acid residues than the number allowed for by the Kabat numbering. As a result, the numbering according to Kabat may or may not correspond to the actual numbering of the amino acid residues in the actual sequence. The total number of amino acid residues in a VH domain and a VHH domain is usually in the range of from 110 to 120, often between 112 and 115. However, smaller, and longer sequences may also be suitable for the purposes described herein.

Determination of CDR regions in a single-domain antibody may be accomplished using different methods, including those described by Kabat et al. (1991), “Sequences of Proteins of Immunological Interest,” 5th Ed. Public Health Service, National Institutes of Health, Bethesda, Md. (“Kabat” numbering scheme); Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Martin et al., “Modeling antibody hypervariable loops: a combined algorithm,” PNAS, 1989, 86 (23): 9268-9272, (“AbM” numbering scheme), each reference cited herein is incorporated by reference in its entirety.

The boundaries of a given CDR or framework (FR) may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software.

In some embodiments, CDRs can be defined in accordance with any of the Kabat numbering scheme, the Chothia numbering scheme, a combination of Kabat and Chothia, the AbM numbering scheme, and/or the Contact numbering scheme. A VHH typically comprises three CDRs, designated CDR1, CDR2, and CDR3. Table 1-3, below, lists exemplary position boundaries of CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-H1 located before CDR-H1, FR-H2 located between CDR-H1 and CDR-H2, FR-H3 located between CDR-H2 and CDR-H3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1-3
CDRs definitions according to various numbering schemes

| CDR | Kabat | Chothia | AbM | Contact |
|---|---|---|---|---|
| CDR-H1 | H31-H35B | H26-H32 . . . 34 | H26-H35B | H30-H35B |
| (Kabat | | | | |
| Numbering¹) | | | | |
| CDR-H1 | H31-H35 | H26-H32 | H26-H35 | H30-H35 |
| (Chothia | | | | |
| Numbering²) | | | | |
| CDR-H2 | H50-H65 | H52-H56 | H50-H58 | H47-H58 |
| CDR-H3 | H95-H102 | H95-H102 | H95-H102 | H93-H101 |

¹Kabat et al. (1991), “Sequences of Proteins of Immunological Interest,” 5th Ed. Public Health Service, National Institutes of Health, Bethesda, MD;
²Al-Lazikani et al., (1997) JMB 273, 927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) CDR as defined by any of the above-mentioned schemes. For example, where it is stated that a particular CDR (e.g., a CDR-H3) contains the amino acid sequence of a corresponding CDR in a given VHH amino acid sequence, it is understood that such a CDR has a sequence of the corresponding CDR (e.g., CDR-H3) within the VHH, as defined by any of the above-mentioned schemes. In some embodiments, specific CDR sequences are specified. Exemplary CDR sequences of provided antibodies are described using various numbering schemes (see e.g., Table 1-3), although it is understood that a provided antibody can include CDRs as described according to any of the other above-mentioned numbering schemes or other numbering schemes known to a person of ordinary skill in the art.

In a single-domain antibody sequence of the present disclosure, the framework sequences may be any suitable framework sequences. For example, the framework sequences may be framework sequences derived from a heavy chain variable domain (e.g., a VH sequence or VHH sequence). In some embodiments, the framework sequences are either framework sequences that have been derived from a VHH sequence (in which said framework sequences may optionally have been partially or fully humanized) or are conventional VH sequences (in which said framework sequences may optionally have been partially or fully camelized).

Antigen-binding fragments (or combinations of fragments) of any of single-domain antibodies described herein, such as fragments that contain one or more CDR sequences, suitably flanked by and/or linked via one or more framework sequences, are also encompassed within the present disclosure.

It should be noted, however, that the present disclosure is not limited to the origin of the single-domain antibody (or of the nucleotide sequence used to express it), nor to the way that the single-domain antibody or nucleotide sequence is generated or obtained. Thus, an antigen-binding protein of the present disclosure may comprise naturally occurring sequences (from a suitable species), recombinant sequences, or synthetic or semi-synthetic sequences. Similarly, nucleotide sequences encoding antigen-binding proteins of the present disclosure may comprise naturally occurring nucleotide sequences, recombinant sequences, or synthetic or semi-synthetic sequences (for example, sequences that are prepared by PCR or isolated from a library).

Anti-TNFR2 antigen-binding proteins (e.g., antibodies such single-domain antibodies) of the present disclosure may comprise one or more amino acid substitutions, insertions and/or deletions in the framework and/or CDR regions of the heavy chain variable domains as compared to the exemplary antibody sequences provided herein. Such mutations can be readily ascertained by comparing the amino acid sequences disclosed herein to germline sequences available from, for example, public antibody sequence databases. The antigen-binding molecules of the present disclosure may comprise antigen-binding domains which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another germline sequence, or to a conservative amino acid substitution of the corresponding germline residue(s) (such sequence changes are referred to herein collectively as “germline mutations”). A person of ordinary skill in the art, starting with the heavy chain variable region sequences disclosed herein, can easily produce numerous antibodies and antigen-binding fragments which comprise one or more individual germline mutations or combinations thereof. In certain embodiments, all of the framework and/or CDR residues within the VHH domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antigen-binding domain was originally derived).

Furthermore, the antigen-binding domains may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antigen-binding domains that contain one or more germline mutations can be easily tested for one or more desired property such as, improved binding specificity, increased binding affinity, improved, or enhanced biological properties (e.g., agonistic effect), reduced immunogenicity, etc. Antigen-binding proteins comprising one or more antigen-binding domains obtained in this general manner are encompassed within the present disclosure.

Provided herein are anti-TNFR2 antigen-binding proteins comprising variants of any of the VHH and/or CDR amino acid sequences disclosed herein having one or more amino acid substitutions. For example, the present disclosure includes anti-TNFR2 antigen-binding proteins having VHH and/or CDR amino acid sequences with, e.g., 10 or fewer, 8 or fewer, 6 or fewer, 4 or fewer, 3 or fewer, 2, or 1 amino acid substitutions relative to any of the VHH and/or CDR amino acid sequences set forth in Tables 1-1 and 1-2 herein. Amino acid substitutions may be introduced into an antigen-binding protein of interest and the resultant variants can screened for a desired activity, for example, retained/improved antigen binding, decreased immunogenicity, or reduced ADCC or CDC.

Amino acids may be grouped according to common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. In some embodiments, an amino acid substitution is a conservative substitution, meaning exchanging an amino acid with another amino acid of the same class. In some embodiments, amino acid substitutions may also include a non-conservative substitution, meaning exchanging an amino acid with an amino acid of a different class. Other exemplary amino acid substitutions are shown in Table 1-4.

TABLE 1-4
Exemplary amino acid substitutions

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Årg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

In some embodiments, single-domain antibodies (e.g., VHH) of the present disclosure comprise one or more modifications that reduce binding of the single-domain antibodies (e.g., VHH) by pre-existing antibodies found in human blood or serum. In some embodiments, single-domain antibodies (e.g., VHHs) of the present disclosure are modified by mutation of amino acid position 11, for example Leu11Glu (L11E), Leu11Lys (L11K), or Leu11Val (L11V). In one embodiment, a single-domain antibody (e.g., VHH) of the present disclosure may comprise a valine (V) at amino acid position 11 and a leucine (L) at amino acid position 89 (according to Kabat numbering). As another example, a single-domain antibody (e.g., VHH) of the present disclosure may comprise an extension of 1 to 5 (naturally occurring) amino acids, such as a single alanine (A) extension, at the C-terminus of the single-domain antibody (e.g., VHH). The C-terminus of a VHH is normally VTVSS (SEQ ID NO: 4031). In one embodiment, a single-domain antibody (e.g., VHH) of the present disclosure comprises a lysine (K) or glutamine (Q) at position 110 (according to Kabat numbering). In another embodiment, a single-domain antibody (e.g., VHH) of the present disclosure comprises a lysine (K) or glutamine (Q) at position 112 (according to Kabat numbering). Accordingly, the C-terminus of a single-domain antibody (e.g., VHH) can be any one of VKVSS (SEQ ID NO: 4032), VQVSS (SEQ ID NO: 4033), VTVKS (SEQ ID NO: 4034), VTVQS (SEQ ID NO: 4035), VKVKS (SEQ ID NO: 4036), VKVQS (SEQ ID NO: 4037), VQVKS (SEQ ID NO: 4038), or VQVQS (SEQ ID NO: 4039). In another embodiment, a single-domain antibody (e.g., VHH) of the present disclosure comprises a valine (V) at amino acid position 11 and a leucine (L) at amino acid position 89 (according to Kabat numbering), optionally a lysine (K) or glutamine (Q) at position 110 (according to Kabat numbering) and an extension of 1 to 5 (naturally occurring) amino acids, such as a single alanine (A) extension at the C-terminus of the single-domain antibody (e.g., VHH) (such that the C-terminus of the single-domain antibody (e.g., VHH) for example has the sequence VTVSSA (SEQ ID NO: 4040), VKVSSA (SEQ ID NO: 4041) or VQVSSA (SEQ ID NO: 4042). In further embodiments, single-domain antibodies (e.g., VHH) of the present disclosure are modified by changes in carboxy-terminal region, for example to a terminal sequence having the sequence GQGTLVTVKPGG (SEQ ID NO: 4043) or GQGTLVTVEPGG (SEQ ID NO; 4044) or modification thereof. Additional modification to reduce binding by pre-existing antibodies in human serum can be found in e.g., WO2012/175741, WO2015/173325, WO2016/150845, WO2011/003622, WO2013/024059; U.S. Pat. Nos. 11,426,468, 10,526,397, which are incorporated herein by reference in their entities.

In one embodiment, a single-domain antibody (e.g., VHH) of the present disclosure comprises at the carboxy-terminus starting from position 111 according to Chothia the amino acid sequence VAGG (SEQ ID NO: 4697) or VPAG (SEQ ID NO: 4698). In one embodiment, a single-domain antibody (e.g., VHH) of the present disclosure comprises at the carboxy-terminus starting from position 111 according to Chothia the amino acid sequence VAGG (SEQ ID NO: 4697). In one embodiment, a single-domain antibody (e.g., VHH) of the present disclosure comprises at the carboxy-terminus starting from position 111 according to Chothia the amino acid sequence VPAG (SEQ ID NO: 4698).

In some embodiments, a single-domain antibody (e.g., VHH) of the present disclosure comprises an amino acid sequence selected from any one of SEQ ID NOs: 4, 7, 11, 15, 19, 23, 27, 31, 35, 39, 43, 47, 4064, 4068, 4072, 4521, 93-640, 4079-4125 2805-3363, 4359-4420, and 4605-4628, or a sequence having at least 75% identity thereto, wherein the amino acid sequence at the carboxy-terminus starting from position 111 according to Chothia comprises VAGG (SEQ ID NO: 4697) or VPAG (SEQ ID NO: 4698).

In some embodiments, a single-domain antibody (e.g., VHH) of the present disclosure comprises an amino acid sequence selected from any one of SEQ ID NOs: 4, 7, 11, 15, 19, 23, 27, 31, 35, 39, 43, 47, 4064, 4068, 4072, and 4521, or a sequence having at least 75% identity thereto, wherein the amino acid sequence at the carboxy-terminus starting from position 111 according to Chothia comprises VAGG (SEQ ID NO: 4697) or VPAG (SEQ ID NO: 4698).

In some embodiments, a single-domain antibody (e.g., VHH) of the present disclosure comprises an amino acid sequence selected from any one of SEQ ID NOs: 81-92, 4076-4078, 4523, 4526, 4529, 4532, 4731-4734, 641-1127, and 4126-4172, or a sequence having at least 75% identity thereto, wherein the amino acid sequence at the carboxy-terminus starting from position 111 according to Chothia comprises VAGG (SEQ ID NO: 4697) or VPAG (SEQ ID NO: 4698).

In some embodiments, a single-domain antibody (e.g., VHH) of the present disclosure comprises an amino acid sequence selected from any one of SEQ ID NOs: 81-92, 4076-4078, 4523, 4526, 4529, 4731-4734, and 4532 or a sequence having at least 75% identity thereto, wherein the amino acid sequence at the carboxy-terminus starting from position 111 according to Chothia comprises VAGG (SEQ ID NO: 4697) or VPAG (SEQ ID NO: 4698).

In some embodiments, single-domain antibodies (e.g., VHH) of the present disclosure are modified to enhance binding to staphylococcal protein A (SpA) or streptococcal protein G (SpG). Binding of SpA and SpG to antibodies or antibody fragments can be useful in the manufacturing process of the antibodies or antibody fragments. The high-affinity interaction of the IgG Fc region with SpA and SpG has been extensively exploited and became the gold standard for monoclonal antibody purification (Björck and Kronvall, 1984). Other non-Fc containing antibody fragments, such as VHHs and Fabs do not have the capacity to bind to SpA or SpG via their Fc regions. However, sequence-dependent interaction with SpA has been demonstrated for these non-Fc containing antibody fragments (Graille et al., 2000; Henry et al., 2016). This characteristic circumvents potential use of affinity tags fused to the drug candidate for affinity chromatography that have the disadvantage as being regarded as a sequence liability, as it may impact protein immunogenicity as well as protein structure and stability and could compromise functionality. The interaction of the single-domain antibodies (e.g., VHH) to SpA relies on an alternative binding mode, with a 1-5 μM affinity, which is comparable to the 0.2-3 μM measured for VH-SpA interactions (To et al., JBC, 2005; Henry et al., Plos One, 2016).

In some embodiments, single-domain antibodies (e.g., VHH) of the present disclosure have, or are modified to have a SpA-binding motif. For example, The VHH-SpA interface has been mapped to thirteen residues, which cluster within the framework at the back side of the V-body, distant to the CDRs (Graille et al., 2000, Henry et al., 2016). In the absence of a VHH-SpA co-structure, superposition of a SpA-Fab crystal structure and a VHH allows for visualizing the binding mode. Based on a structural and functional analysis, the thirteen residues of the VHH-SpA interface have been characterized to be intolerant to substitutions (residues Gly15, Arg19, Tyr59, Gly65, and Arg66), tolerant to specific substitutions (residues Thr/Lys/Arg57, Thr68, Gln81, Asn82a, and Ser82b) or generally tolerant to a variety of substitutions (residues Ser17, Lys64, and Ser70) (all residue positions refer to Kabat numbering) (Henry et al., Plos One, 2016). Thus, a SpA-binding motif included in a single-domain antibody (e.g., VHH) of the present disclosure may include one or more, or all of the thirteen residues.

In some embodiments, single-domain antibodies (e.g., VHH) of the present disclosure comprise one or more modifications at N-terminus to prevent formation of a pyroglutamate and product heterogeneity. In one embodiment, the amino acid residue Glu at the first position of the single-domain antibodies (e.g., VHH) is replaced with Asp (E1D).

In some embodiments, a single-domain antibody (e.g., VHH) of the present disclosure comprises an amino acid sequence selected from any one of SEQ ID NOs: 4, 7, 11, 15, 19, 23, 27, 31, 35, 39, 43, 47, 4064, 4068, 4072, 4521, 93-640, 4079-4125 2805-3363, 4359-4420, and 4605-4628, or a sequence having at least 75% identity thereto, wherein the amino acid residue Glu at the first position of the single-domain antibody (e.g., VHH) is replaced with Asp (E1D).

In some embodiments, a single-domain antibody (e.g., VHH) of the present disclosure comprises an amino acid sequence selected from any one of SEQ ID NOs: 4, 7, 11, 15, 19, 23, 27, 31, 35, 39, 43, 47, 4064, 4068, 4072, and 4521, or a sequence having at least 75% identity thereto, wherein the amino acid residue Glu at the first position of the single-domain antibody (e.g., VHH) is replaced with Asp (E1D).

In some embodiments, a single-domain antibody (e.g., VHH) of the present disclosure comprises an amino acid sequence selected from any one of SEQ ID NOs: 81-92, 4076-4078, 4523, 4526, 4529, 4532, 4731-4734, 641-1127, and 4126-4172, or a sequence having at least 75% identity thereto, wherein the amino acid residue Glu at the first position of the single-domain antibody (e.g., VHH) is replaced with Asp.

In some embodiments, a single-domain antibody (e.g., VHH) of the present disclosure comprises an amino acid sequence selected from any one of SEQ ID NOs: 81-92, 4076-4078, 4523, 4526, 4529, 4731-4734, and 4532 or a sequence having at least 75% identity thereto, wherein the amino acid residue Glu at the first position of the single-domain antibody (e.g., VHH) is replaced with Asp (E1D).

### Alternative Protein Scaffolds

In some embodiments, anti-TNFR2 antigen-binding proteins of the present disclosure can adopt an alternative protein scaffold. Such alternative protein scaffold may be a single chain polypeptidic framework, optionally with a reduced size (e.g., less than about 200 amino acids), that contains a highly structured core associated with variable domains of high conformational tolerance allowing insertions, deletions, or other substitutions. Such antigen-binding proteins may be generated by grafting CDRs or variable regions described herein onto a suitable protein scaffold. The structure of alternative scaffolds may vary, but preferably are of human origin for those developed as therapeutics.

Alternative protein scaffolds of the present disclosure can be based either on a conventional immunoglobulin (Ig) backbone, or are derived from a completely unrelated protein. These variable domains can be modified to create novel binding interfaces toward any targeted antigen. In some embodiments, an alternative protein scaffold of the present disclosure can be derived from Protein A, e.g., the Z-domain thereof (affibodies), ImmE7 (immunity proteins), BPTI/APPI (Kunitz domains), Ras-binding protein AF-6 (PDZ-domains), charybdotoxin (Scorpion toxin), CTLA-4, Min-23 (knottins), lipocalins (anticalins), neokarzinostatin, a fibronectin domain (used in “adnectin”), an ankyrin repeat (AR) domain (used in “DARPins”), avidity multimers (also known as “avimers”), or thioredoxin (Skerra, A., Curr. Opin. Biotechnol, 18:295-304 (2005); Hosse et al., Protein Sci. 15:14-27 (2006); Nicaise et al., Protein Sci. 13:1882-1891 (2004); Nygren and Uhlen, Curr. Opin. Struc. Biol. 7:463-469 (1997), all of which are hereby incorporated by reference in their entirety).

Anticalins are a suitable type of non-lg based alternative scaffolds for use in the antigen-binding molecules of the present disclosure. Anticalins are a class of engineered ligand-binding proteins that are based on the lipocalin scaffold. Lipocalins are a family of proteins that transport small hydrophobic molecules such as steroids, bilins, retinoids, and lipids. Lipocalins have limited sequence homology, but share a common tertiary structure architecture based on eight antiparallel β-barrels. Lipocalins contain four exposed loops built on the rigid β-barrel structure. Exemplary anticalin proteins that are commonly used are about a size of about 180 amino acids and a mass of about 20 kDa.

DARPins are another suitable non-Ig based alternative scaffold that can be used in the antigen-binding molecules of the present disclosure. DARPins are genetically engineered antibody mimetic proteins typically exhibiting highly specific and high-affinity target protein binding. They are derived from natural ankyrin repeat (AR) proteins, which usually contain a 33 amino acid protein motif consisting of two α-helices separated by loops, which repeats mediate protein-protein interactions. DARPins can be generated using combinatorial AR libraries constructed based on the 33 amino acid AR motif with seven randomized positions. DARPin libraries can be screened using ribosome display, and library members typically are well produced in Escherichia coli, do not aggregate, and display high thermodynamic stability. Preferably, DARPins contain two to four of these motifs flanked by N- and C-terminal capping motifs to shield hydrophobic regions and allow increased solubility.

The avimer structure can also be used as a protein backbone to generate a suitable non-Ig based alternative scaffold. Avimers typically consist of two or more peptide sequences of 30 to 35 amino acids each, connected by peptide linker. The individual sequences are derived from A-domains of various membrane receptors and have a rigid structure, stabilized by disulfide bridges and calcium. Each A-domain can bind to a certain epitope of the target protein. The combination of domains binding to different epitopes of the same protein increases affinity to this protein, an effect known as avidity.

Proteins derived from fibronectin III (FN3) domains can also be used to generate a suitable non-Ig based alternative scaffold (also known as “monobody”). For example, the tenth fibronectin type III domain (FN10) of human fibronectin corresponds to a β-sandwich with seven β-strands and three connecting loops showing structural homologies to Ig domains without disulfide bridges. In some cases, the connecting loops of FN10, each about 15 to 21 amino acids in length, can be randomized and the domains displayed on both phage and yeast to select for a scaffold with the desirable properties. Adnectins™ is an exemplary scaffold generated using 10^{th} FN3 domains randomized and displayed in this way. Another exemplary scaffold comprising FN3 domains is a Centyrin™. Centryrins™ contain the consensus sequence of FN3 domains of human Tenascin C (TNC), which is found in the extracellular matrix of various tissues. Centyrin™ scaffolds have loops that have structural homology to antibody variable domains (i.e., CDR1, CDR2 and CDR3), and are small (about 10 kDa), simple, and highly stable single domain proteins that do not contain cysteine, disulfides, or glycosylated residues. Centyrin™ possess excellent biophysical properties such as stability to heat, pH, denaturant and organic solvents, reversible unfolding and monodispersity. Another recent exemplary FN3-based scaffold that can be used in the present disclosure is fluctuation-regulated affinity proteins (FLAPs), as described in See et al., 2020. Biotechnology Journal 15 (12): e2000078, which is incorporated herein by reference in its entirety.

### Fusion Proteins and Conjugates

In one aspect, provided herein are fusion proteins and conjugates comprising at least one anti-TNFR2 antigen-binding protein (e.g., antibody such as a single-domain antibody) linked, directly or indirectly, to one or more additional domains or moieties. In some embodiments, the fusion protein or conjugate of the present disclosure comprises a single polypeptide. In other embodiments, the fusion protein or conjugate of the present disclosure comprises more than one polypeptide. In some embodiments, the fusion protein or conjugate of the present disclosure comprises two polypeptides.

In some embodiments, the fusion protein or conjugate of the present disclosure comprises at least one anti-TNFR2 antigen-binding protein (e.g., antibody such as a single-domain antibody) described herein. In some embodiments, the fusion protein or conjugate is multivalent. For example, the fusion protein or conjugate of the present disclosure may be at least bivalent, but can also be e.g., trivalent, tetravalent, pentavalent, hexavalent, etc. The terms “bivalent”, “trivalent”, “tetravalent”, “pentavalent”, or “hexavalent” all fall under the term “multivalent” and indicate the presence of two, three, four, five or six binding units (e.g., VHHs), respectively.

In certain embodiments, the fusion protein or conjugate is multispecific. For example, in some cases, the one or more additional domain or moieties may be one or more additional binding domain that binds to one or more further antigen or protein. The fusion protein or conjugate of the present disclosure may be, for example, bispecific, trispecific, tetraspecific, pentaspecific, etc. The terms “bispecific”, “trispecific”, “tetraspecific”, “pentaspecific”, etc., all fall under the term “multispecific” and refer to binding to two, three, four, five, etc., different target molecules, respectively.

When two or more anti-TNFR2 antigen-binding proteins are included in a fusion protein or conjugate, the two or more anti-TNFR2 antigen-binding proteins may comprise the same sequence or may comprise different sequences. In such embodiments, the two or more anti-TNFR2 antigen-binding proteins may bind to the same epitope on TNFR2 or different epitopes on TNFR2. For example, a fusion protein or conjugate of the present disclosure may be biparatopic, e.g., if two VHHs bind two different epitopes on TNFR2.

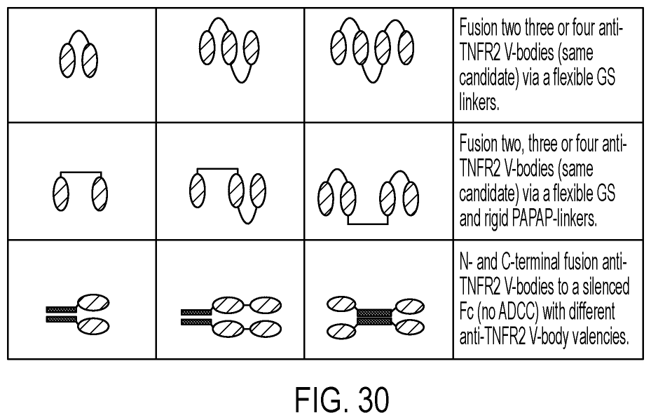

Exemplary designs of multivalent anti-TNFR2 fusion constructs comprising two or more anti-TNFR2 binding units (e.g., VHHs) are shown in FIG. 30.

### Fusion or Conjugation to Fc Regions

In some embodiments, a fusion protein or conjugate of the present disclosure comprises at least one anti-TNFR2 antigen-binding protein (e.g., antibody such as a single-domain antibody) provided herein operably linked to a dimerization domain such as an immunoglobulin Fc region. An immunoglobulin Fc region may be linked indirectly or directly to the at least one anti-TNFR2 antigen-binding protein (e.g., antibody such as a single-domain antibody), In some embodiments, a fusion protein or conjugate of the present disclosure comprises one, two, three, four, five, six or more anti-TNFR2 antigen-binding proteins provided herein operably linked to an Fc region.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge can mediate dimerization between two Fc-containing polypeptides. In various embodiments, an Fc region included in a fusion protein or conjugate of the present disclosure is a human immunoglobulin Fc region, or is derived from a human immunoglobulin Fc region. In some embodiments, the immunoglobulin Fc region is of IgG, IgE, IgM, IgD, IgA or IgY isotype. In some embodiments, the immunoglobulin Fc region is an IgG isotype, such as IgG1, IgG2, IgG3, or IgG4 subclass. The immunoglobulin Fc region may comprise a variant or fragment of a native IgG Fc region.

A native Fc region typically possesses an effector function, including but not limited to, Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

In some embodiments, a fusion protein or conjugate of the present disclosure can comprise a dimer of Fc regions. In some embodiments, an Fc region mediates dimerization of the TNFR2-binding units at physiological conditions, such as when expressed from a cell, such that a dimer is formed that doubles the number of TNFR2 binding units. For example, a fusion polypeptide comprising one VHH domain that binds TNFR2 and an Fc region is monovalent as a monomer, but the Fc region can mediate dimerization; as a result, the fusion protein is bivalent (i.e., having two anti-TNFR2 VHH domains per molecule). Similarly, in some embodiments, two anti-TNFR2 VHH domains (2×) are fused to an IgG Fc region and as a result of dimerization, the fusion protein is tetravalent (I.e., having four anti-TNFR2 VHH domains per molecule). In some embodiments, three anti-TNFR2 VHH domain (3×) are fused to an IgG Fc region and as a result of dimerization, the fusion protein is hexavalent (i.e., having six anti-TNFR2 VHH domains per molecule).

In some embodiments, a fusion protein or conjugate of the present disclosure may comprise two polypeptide chains, each polypeptide chain having the following structure: (anti-TNFR2 VHH) n-Linker-Fc, wherein n can be any integral number (e.g., 1, 2, 3, 4, 5, etc). When n≥2, each anti-TNFR2 VHH may be optionally operably linked to another anti-TNFR2 VHH via a linker.

In some embodiments, a fusion protein or conjugate of the present disclosure may comprise two polypeptide chains, each polypeptide chain having the following structure: (anti-TNFR2 VHH) n-Linker-Fc-(anti-TNFR2 VHH) m, wherein n and m can independently be any integral number (e.g., 1, 2, 3, 4, 5, etc). When n≥2 or m≥2, each anti-TNFR2 VHH may be optionally operably linked to another anti-TNFR2 VHH via a linker.

In some embodiments, a fusion protein or conjugate of the present disclosure is bivalent. In some embodiments, the bivalent fusion protein or conjugate of the disclosure comprises two polypeptide chains, each polypeptide chain having the following structure: (anti-TNFR2 VHH)-Linker-Fc.

In some embodiments, a fusion protein or conjugate of the present disclosure is tetravalent. In some embodiments, the tetravalent fusion protein or conjugate of the disclosure comprises two polypeptide chains, each polypeptide chain having the following structure: (anti-TNFR2 VHH)-Linker-(anti-TNFR2 VHH)-Linker-Fc. In some embodiments, the tetravalent fusion protein or conjugate of the disclosure comprises two polypeptide chains, each polypeptide chain having the following structure: (anti-TNFR2 VHH)-Linker-Fc-Linker-(anti-TNFR2 VHH). The multiple linkers used in the fusion protein are not necessarily the same.

In some embodiments, a fusion protein or conjugate of the disclosure is hexavalent. In some embodiments, the hexavalent fusion protein or conjugate of the disclosure comprises two polypeptide chains, each polypeptide chain having the following structure: (anti-TNFR2 VHH)-Linker-(anti-TNFR2 VHH)-Linker-(anti-TNFR2 VHH)-Linker-Fc. In some embodiments, the hexavalent fusion protein or conjugate of the disclosure comprises two polypeptide chains, each polypeptide chain having the following structure: (anti-TNFR2 VHH)-Linker-(anti-TNFR2 VHH)-Linker-Fc-linker-(anti-TNFR2 VHH). In some embodiments, the hexavalent fusion protein or conjugate of the disclosure comprises two polypeptide chains, each polypeptide chain having the following structure: (anti-TNFR2 VHH)-Linker-Fc-Linker-(anti-TNFR2 VHH)-Linker-(anti-TNFR2 VHH). The multiple linkers used in the fusion protein are not necessarily the same.

In some embodiments, the CH3 domain of the Fc region can be used as homodimerization domain, such that the resulting fusion protein may be formed from two identical polypeptides. In other cases, the CH3 dimer interface region of the Fc region can be mutated to enable heterodimerization. For example, a heterodimerization domain can be incorporated into the fusion protein such that the construct is a heterodimeric fusion protein.

When a dimer of Fc regions is used in a fusion protein or conjugate of the present disclosure, the first and second Fc regions may be of the same IgG isotype such as, e.g., IgG1/IgG1, IgG2/IgG2, IgG4/IgG4. Alternatively, the first and second Fc regions may be of different IgG isotypes such as, e.g., IgG1/IgG2, IgG1/IgG4, IgG2/IgG4, etc.

In some embodiments, the Fc region included in a fusion protein or conjugate of the present disclosure can be mutated or modified. In some embodiments, the mutations include one or more amino acid substitutions to reduce an effector function of the Fc region. Various examples of mutations to Fc regions to alter, such as reduce, effector function are known, including any as described below. In general, the numbering of the residues in an immunoglobulin heavy chain or portion thereof, such as an Fc region, is according to the EU index as in Kabat et al., Sequences of Proteins of Immunological Interest, 5th Ed. Public Health Service, National Institutes of Health, Bethesda, Md. (1991).

In some embodiments, the human IgG Fc region is modified to alter antibody-dependent cellular cytotoxicity (ADCC) and/or complement-dependent cytotoxicity (CDC). Non-limiting examples of amino acid modifications that can alter ADCC and/or CDC are described in Alegre et al, 1992 J Immunol, 148:3461-3468; Idusogie et al., 2001 J Immunol, 166(4): 2571-5; Shields et al., 2001 JBC, 276(9): 6591-6604; Lazar et al., 2006 PNAS, 103(11): 4005-4010; Stavenhagen et al., 2007 Cancer Res, 67(18): 8882-8890; Natsume et al., 2008 Cancer Res, 68(10): 3863-72; Stavenhagen et al., 2008 Advan. Enzyme Regul., 48: 152-164; Moore et al., 2010 mAbs, 2(2): 181-189; and Kaneko and Niwa, 2011 Biodrugs, 25(1):1-11, each of which is incorporated herein by reference in its entirety.

In some embodiments, an Fc region included in a fusion protein or conjugate of the present disclosure exhibits reduced effector functions (such as CDC and ADCC), Various in vitro and/or in vivo cytotoxicity assays can be conducted to confirm the reduction/depletion of CDC and/or ADCC activities. For example, Fc receptor (FcR) binding assays can be conducted to ensure that the fusion protein construct and/or cleaved components thereof lack FcγR binding (hence likely lacking ADCC activity), but retains FcRn binding ability. The primary cells for mediating ADCC are NK cells which express FcγRIII only, whereas monocytes express FcγRI, FcγRII and FcγRIII. Non-limiting examples of in vitro assays to assess ADCC activity of a molecule of interest are described in e.g., U.S. Pat. Nos. 5,500,362; 5,821,337; Hellstrom, et al., Proc. Nat'l Acad. Sci. USA 83:7059-7063 (1986); and Hellstrom et al., Proc. Nat'l Acad. Sci. USA 82:1499-1502 (1985); Bruggemann. et al., J. Exp. Med. 166:1351-1361 (1987). Alternatively, non-radioactive assay methods may be employed, such as ACTI™ non-radioactive cytotoxicity assay for flow cytometry or CytoTox96™ non-radioactive cytotoxicity assay. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the fusion protein construct or cleaved components thereof is unable to bind C1q and hence lacks CDC activity (see, e.g., C1q and C3c binding ELISA in WO 2006/029879 and WO 2005/100402). To assess complement activation, a CDC assay may be performed (see, e.g., Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006).

Examples of mutations that enhance ADCC include modification at Ser239 and Ile332, for example Ser239Asp and Ile332Glu (S239D, 1332E). Examples of mutations that enhance CDC include modifications at Lys326 and Glu333. In some embodiments, the Fc region is modified at one or both of these positions, for example Lys326Ala and/or Glu333Ala (K326A and E333A) using the Kabat numbering system.

In some embodiments, the Fc region of the fusion protein is altered at one or more of the following positions to reduce Fc receptor binding: Leu 234 (L234), Leu235 (L235), Asp265 (D265), Asp270 (D270), Ser298 (S298), Asn297 (N297), Asn325 (N325) or Ala327 (A327) or Pro329 (P329). For example, Leu 234Ala (L234A), Leu235Ala (L235A), Leu235Glu (L235E), Asp265Asn (D265N), Asp265Ala (D265A), Asp270Asn (D270N), Ser298Asn (S298N), Asn297Ala (N297A), Pro329Ala (P329A) or Pro239Gly (P329G), Asn325Glu (N325E) or Ala327Ser (A3275). In some embodiments, modifications within the Fc region reduce binding to Fc-receptor-gamma receptors (FcγRs) while have minimal impact on binding to the neonatal Fc receptor (FcRn).

In some embodiments, the human IgG1 Fc region is modified at amino acid Asn297 (Kabat Numbering) to prevent glycosylation of the fusion protein, e.g., Asn297Ala (N297A) or Asn297Asp (N297D). In some embodiments, the Fc region of the fusion protein is modified at amino acid Leu235 (Kabat Numbering) to alter Fc receptor interactions, e.g., Leu235Glu (L235E) or Leu235Ala (L235A). In some embodiments, the Fc region of the fusion protein is modified at amino acid Leu234 (Kabat Numbering) to alter Fc receptor interactions, e.g., Leu234Ala (L234A). In some embodiments, the Fc region of the fusion protein is modified at amino acid Leu234 (Kabat Numbering) to alter Fc receptor interactions, e.g., Leu235Glu (L235E). In some embodiments, the Fc region of the fusion protein is altered at both amino acids 234 and 235, e.g., Leu234Ala and Leu235Ala (L234A/L235A) or Leu234Val and Leu235Ala (L234V/L235A). In some embodiments, the Fc region of the fusion protein is altered at amino acids at 234, 235, and 297, e.g., Leu234Ala, Leu235Ala, Asn297Ala (L234A/L235A/N297A). In some embodiments, the Fc region of the fusion protein is altered at amino acids at 234, 235, and 329, e.g., Leu234Ala, Leu235Ala, Pro239Ala (L234A/L235A/P329A). In some embodiments, the Fc region of the fusion protein is modified at amino acid Asp265 (Kabat Numbering) to alter Fc receptor interactions, e.g Asp265Ala (D265A). In some embodiments, the Fc region of the fusion protein is modified at amino acid Pro329 (Kabat Numbering) to alter Fc receptor interactions, e.g., Pro329Ala (P329A) or Pro329Gly (P329G). In some embodiments, the Fc region of the fusion protein is altered at both amino acids 265 and 329, e.g., Asp265Ala and Pro329Ala (D265A/P329A) or Asp265Ala and Pro329Gly (D265A/P329G). In some embodiments, the Fc region of the fusion protein is altered at amino acids at 234, 235, and 265, e.g., Leu234Ala, Leu235Ala, Asp265Ala (L234A/L235A/D265A). In some embodiments, the Fc region of the fusion protein is altered at amino acids at 234, 235, and 329, e.g., Leu234Ala, Leu235Ala, Pro329Gly (L234A/L235A/P329G). In some embodiments, the Fc region of the fusion protein is altered at amino acids at 234, 235, 265 and 329, e.g., Leu234Ala, Leu235Ala, Asp265Ala, Pro329Gly (L234A/L235A/D265A/P329G). In some embodiments, the Fc region of the fusion protein is altered at Gly235 to reduce Fc receptor binding. For example, wherein Gly235 is deleted from the fusion protein. In some embodiments, the human IgG1 Fc region is modified at amino acid Gly236 to enhance the interaction with CD32A, e.g., Gly236Ala (G236A). In some embodiments, the human IgG1 Fc region lacks Lys447 (EU index of Kabat et al 1991 Sequences of Proteins of Immunological Interest).

In some embodiments, the Fc region of the fusion protein is altered at amino acids at 234, 235, and 236, e.g., Leu234Gly, Leu235Ser, Gly236Arg (L234G/L235S/G236R). In some embodiments, the Fc region of the fusion protein is altered at amino acids at 234, 235, and 236, e.g., Leu234Ser, Leu235Thr, Gly236Arg (L234S/L235T/G236R). In some embodiments, the Fc region of the fusion protein is altered at amino acids at 234, 235, and 236, e.g., Leu234Ser, Leu235Val, Gly236Arg (L234S/L235V/G236R). In some embodiments, the Fc region of the fusion protein is altered at amino acids at 234, 235, and 236, e.g., Leu234Thr, Leu235Gln, Gly236Arg (L234T/L235Q/G236R). In some embodiments, the Fc region of the fusion protein is altered at amino acids at 234, 235, and 236, e.g., Leu234Thr, Leu235Thr, Gly236Arg (L234T/L235T/G236R). In some embodiments, the Fc region of the fusion protein fusion protein is altered at amino acids at 234, 235, and 329, e.g., Leu234Thr, Leu235Thr, Pro329Gly (L234A/L235A/P329G). In some embodiments, the Fc region of the fusion protein is altered at amino acids at 252, 254, and 256, e.g., Met252Tyr, Ser254Thr, Thr256Glu (M252Y/S254T/T256E).

In some embodiments, the Fc region of the fusion protein is lacking an amino acid at one or more of the following positions to reduce Fc receptor binding: Glu233 (E233), Leu234 (L234), or Leu235 (L235). In some embodiments, the Fc region of the fusion protein is lacking an amino acid at one or more of the following positions Glu233 (E233), Leu234 (L234), or Leu235 (L235) and is modified at one or more of the Asp265 (D265), Asn297 (N297), or Pro329 (P329) to reduce Fc receptor binding. For example, an Fc region included in a TNFR2 binding polypeptide is derived from a human Fc domain, and comprises a three amino acid deletion in the lower hinge corresponding to IgG1 E233, L234, and L235. In some embodiments, such Fc polypeptides do not engage FcγRs and thus are referred to as “effector silent” or “effector null.” For example, Fc deletion of these three amino acids reduces the complement protein C1q binding. In some embodiments, a polypeptide with an Fc region with Fc deletion of these three amino acids retains binding to FcRn and therefore has extended half-life and transcytosis associated with FcRn mediated recycling.

In one embodiment, the immunoglobulin Fc region of the fusion protein is a variant of human IgG1 Fc region, having an amino acid sequence:

IgG1 L234A, L235A (also known as ″LALA″ variant) (mutations bolded in the sequence below)
(SEQ ID NO: 4045)
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPG

In one embodiment, the immunoglobulin Fc region of the fusion protein is a variant of human IgG1 Fc region, having an amino acid sequence:

IgG1 L234A, L235A, and P329A (also known as ″LALAPA″ variant) (mutations bolded in the
sequence below)
(SEQ ID NO: 4046)
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALAAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPG

In one embodiment, the immunoglobulin Fc region of the fusion protein is a variant of human IgG1 Fc region, having an amino acid sequence:

IgG1 D265A, N297A and P329A (also known as ″DANAPA″ variant) (mutations bolded in the
sequence below)
(SEQ ID NO: 4047)
DKTHTCPPCPAPELLGGPSVFLFPPKPKDTLMISRTPEVTCVVVAVSHEDPEVKFNWYVDGVEVHNAKTKPREEQYAST
YRVVSVLTVLHQDWLNGKEYKCKVSNKALAAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYTQKSLSLSPG

In one embodiment, the immunoglobulin Fc region of the fusion protein is a variant of human IgG1 Fc region, having an amino acid sequence:

IgG1 L234A, L235A, and G237A (also known as ″LALAGA″ variant) (mutations bolded in
the sequence below)
(SEQ ID NO: 4048)
DKTHTCPPCPAPEAAGAPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPG

In one embodiment, the immunoglobulin Fc region of the multispecific antigen-binding protein is a variant of human IgG1 Fc region, having an amino acid sequence:

IgG1 L234G/L235S/G236R (mutations bolded in the sequence below)
(SEQ ID NO: 4054)
DKTHTCPPCPAPEGSRGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPG

In one embodiment, the immunoglobulin Fc region of the multispecific antigen-binding protein is a variant of human IgG1 Fc region, having an amino acid sequence:

IgG1 L234S/L235T/G236R (mutations bolded in the sequence below)
(SEQ ID NO: 4055)
DKTHTCPPCPAPESTRGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYTQKSLSLSPG

In one embodiment, the immunoglobulin Fc region of the multispecific antigen-binding protein is a variant of human IgG1 Fc region, having an amino acid sequence:

IgG1 L234S/L235V/G236R (mutations bolded in the sequence below)
(SEQ ID NO: 4056)
DKTHTCPPCPAPESVRGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPG

In one embodiment, the immunoglobulin Fc region of the multispecific antigen-binding protein is a variant of human IgG1 Fc region, having an amino acid sequence:

IgG1 L234T/L235Q/G236R (mutations bolded in the sequence below)
(SEQ ID NO: 4057)
DKTHTCPPCPAPETQRGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPG

In one embodiment, the immunoglobulin Fc region of the multispecific antigen-binding protein is a variant of human IgG1 Fc region, having an amino acid sequence:

IgG1 L234T/L235T/G236R (mutations bolded in the sequence below)
(SEQ ID NO: 4058)
DKTHTCPPCPAPETTRGPSVFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYTQKSLSLSPG

In one embodiment, the immunoglobulin Fc region of the multispecific antigen-binding protein is a variant of human IgG1 Fc region, having an amino acid sequence:

IgG1 L234A/L235A/P329G (mutations bolded in the sequence below)
(SEQ ID NO: 4059)
DKTHTCPPCPAPEAAGGPSVFLFPPKPKDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVMHEALHNHYTQKSLSLSPG

In one embodiment, the immunoglobulin Fc region of the multispecific antigen-binding protein is a variant of human IgG1 Fc region, having an amino acid sequence:

IgG1 M252Y/S254T/T256E (mutations bolded in the sequence below)
(SEQ ID NO: 4060)
DKTHTCPPCPAPELLGGPSVFLFPPKPKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYTQKSLSLSPG

In some embodiments, the human IgG Fc region is modified to enhance FcRn binding. Examples of Fc mutations that enhance binding to FcRn are Met252Tyr, Ser254Thr, Thr256Glu (M252Y, S254T, T256E, respectively) (Kabat numbering, Dall'Acqua et al 2006, J. Biol Chem Vol. 281 (33) 23514-23524), Met428Leu and Asn434Ser (M428L, N434S) (Zalevsky et al 2010 Nature Biotech, Vol. 28 (2) 157-159), or Met252Ile, Thr256Asp, Met428Leu (M252I, T256D, M428L, respectively) (EU index of Kabat et al 1991 Sequences of Proteins of Immunological Interest).

In some embodiments, the Fc region lacks or has reduced fucose attached to the N-linked glycan-chain at N297. There are numerous ways to prevent fucosylation, including but not limited to production in a FUT8 deficient cell line; addition inhibitors to the mammalian cell culture media, for example Castanospermine; and metabolic engineering of the production cell line.

In some embodiments, the Fc domain included in a fusion protein or conjugate of the present disclosure is derived from a human Fc domain and comprises mutations M252Y and M428V. In some embodiments, the mutated or modified Fc polypeptide includes the following mutations: M252Y and M428L using the Kabat numbering system. In some embodiments, such mutations enhance binding to FcRn at the acidic pH of the endosome (near 6.5), while losing detectable binding at neutral pH (about 7.2), allowing for enhanced FcRn mediated recycling and extended half-life.

In some embodiments, the Fc domain included in a fusion protein or conjugate is derived from a human Fc domain and comprises mutations to induce heterodimerization. In some embodiments, such mutations include those referred to as “knob” and “hole” mutations. For example, having an amino acid modification within the CH3 domain at Thr366, which when replaced with a bulkier amino acid, e.g., Try (T366W), is able to preferentially pair with a second CH3 domain having amino acid modifications to less bulky amino acids at positions Thr366, Leu368, and Tyr407, e.g., Ser, Ala, and Val, respectively (T366S/L368A/Y407V). In some embodiments, the “knob” Fc domain comprises the mutation T366W. In some embodiments, the “hole” Fc domain comprises mutations T366S, L368A, and Y407V. Heterodimerization via CH3 modifications can be further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248:7-15). In some embodiments, Fc domains used for heterodimerization comprise additional mutations, such as the mutation $354C on a first member of a heterodimeric Fc pair that forms an asymmetric disulfide with a corresponding mutation Y349C on the second member of a heterodimeric Fc pair. In some embodiments, one member of a heterodimeric Fc pair comprises the modification H435R or H435K to prevent protein A binding while maintaining FcRn binding. In some embodiments, one member of a heterodimeric Fc pair comprises the modification H435R or H435K, while the second member of the heterodimeric Fc pair is not modified at H435. In various embodiments, the hole Fc domain comprises the modification H435R or H435K (referred to as “hole-R” in some instances when the modification is H435R), while the knob Fc domain does not. In some instances, the hole-R mutation improves purification of the heterodimer over homodimeric hole Fc domains that may be present.

In some embodiments, the human IgG Fc region is modified to prevent dimerization. In these embodiments, the fusion proteins of the present disclosure are monomeric. For example, modification at residue Thr366 to a charged residue, e.g. Thr366Lys, Thr366Arg, Thr366Asp, or Thr366Glu (T366K, T366R, T366D, or T366E, respectively), prevents CH3-CH3 dimerization.

In some embodiments, the immunoglobulin Fc region of the fusion protein is of human IgG3 isotype, or a variant thereof. In one embodiment, the IgG3 Fc region is modified at amino acid Asn297 (Kabat Numbering) to prevent to glycosylation of the antibody, e.g., Asn297Ala (N297A) or Asn297Asp (N297D). In some embodiments, the human IgG3 Fc region is modified at amino acid 435 to extend the half-life, e.g., Arg435His (R435H). In some embodiments, the human IgG3 Fc region lacks Lys447 (EU index of Kabat et al 1991).

In some embodiments, the immunoglobulin Fc region of the fusion protein is of human IgG4 isotype, or a variant thereof. In one embodiment, the human IgG4 Fc region is modified at amino acid 235 to alter Fc receptor interactions, e.g., Leu235Glu (L235E). In some embodiments, the human IgG4 Fc region is modified at amino acid Asn297 (Kabat Numbering) to prevent to glycosylation of the antibody, e.g., Asn297Ala (N297A) or Asn297Asp (N297D). In some embodiments, the human IgG4 Fc region is lacks Lys447 (EU index of Kabat et al 1991).

In some embodiments, the IgG4 Fc region of the fusion protein is altered at amino acids at 228 and 235, e.g., Ser228Pro, Leu235Glu or Leu235Ala (S228P/L235E or S228P/L235A). In some embodiments, the IgG4 Fc region of the fusion protein is altered at amino acids at 228, 234 and 235, e.g., Ser228Pro, Phe234Ala, Leu235Glu or Leu235Ala (S228P/F234A/L235E or S228P/F234A/L235A). In some embodiments, the IgG4 Fc region of the fusion protein is altered at amino acids at 228, 235, and 329, e.g., Ser228Pro, Leu235Glu and P329G (S228P/L235E/P329G).

In one embodiment, the immunoglobulin Fc region of the fusion protein is a variant of human IgG4 Fc region, having an amino acid sequence:

IgG4 S228P, L235E (mutations bolded in the sequence below)
(SEQ ID NO: 4049)
ESKYGPPCPPCPAPEFEGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK

In one embodiment, the immunoglobulin Fc region of the fusion protein is a variant of human IgG4 Fc region, having an amino acid sequence:

IgG4 S228P, L235A (mutations bolded in the sequence below)
(SEQ ID NO: 4050)
ESKYGPPCPPCPAPEFAGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK

In one embodiment, the immunoglobulin Fc region of the fusion protein is a variant of human IgG4 Fc region, having an amino acid sequence:

IgG4 S228P, F234A, L235E (mutations bolded in the sequence below)
(SEQ ID NO: 4051)
ESKYGPPCPPCPAPEAEGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK

In one embodiment, the immunoglobulin Fc region of the fusion protein is a variant of human IgG4 Fc region, having an amino acid sequence:

IgG4 S228P, F234A, L235A (mutations bolded in the sequence below)
(SEQ ID NO: 4052)
ESKYGPPCPPCPAPEAAGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMTKNQVSLTCLVKGFYPSD
IAVEWESNGQPENNYKTTPPVLDSDGSFFLYSRLTVDKSRWQEGNVFSCSVMHEALHNHYTQKSLSLSLGK

In one embodiment, the immunoglobulin Fc region of the fusion protein is a variant of human IgG4 Fc region, having an amino acid sequence:

IgG4 P329G, S228P, L235E (mutations bolded in the sequence below)
(SEQ ID NO: 4053)
ESKYGPPCPPCPAPEFEGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLGSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSCSVMHEALHNHYTQKSLSLSLGK

Additional IgG4 heavy chain modifications suitable for use in the fusion proteins or conjugates of the present disclosure include those described in Tables 1 and 2 of Dumet et al., mAbs, 11:8, 1341-1350, which is incorporated herein by reference in its entirety.

In some embodiments, the fusion protein or conjugate contains an immunoglobulin hinge region. In some embodiments, the hinge region serves as a linker to connect one or more TNFR2 binding units (e.g., VHHs) to the Fc region. In other embodiments, the fusion protein can comprise a linker in addition to the hinge region to connect the one or more TNFR2 binding units (e.g., VHHs) to the Fc region. The hinge region can be selected from any of the human IgG subclasses. For example, the fusion protein may contain a modified IgG1 hinge having the sequence of EPKSSDKTHTCPPC (SEQ ID NO: 3923), wherein the Cys220 that typically forms a disulfide bond with the C-terminal cysteine of the light chain is mutated to serine, e.g., Cys220Ser (C220S). In other embodiments, the fusion protein contains a truncated hinge having a sequence DKTHTCPPC (SEQ ID NO: 3924).

In some embodiments, the fusion protein or conjugate has a modified hinge from IgG4, which is modified to prevent or reduce strand exchange, e.g., Ser228Pro (S228P), having the sequence ESKYGPPCPPC (SEQ ID NO: 3925).

In alternative embodiments, a fusion protein or conjugate of the present disclosure may comprise sequences other than an Fc region to achieve multimerization (e.g., dimerization). For example, an amino acid sequence containing at least one cysteine residue may be included to facilitate dimerization of two polypeptides by formation of a disulfide bond between the two polypeptides. In some embodiments, such multimerizing domain may comprise one or more cysteine residues, or a short cysteine-containing peptide. Other multimerizing domains include peptides or polypeptides comprising or consisting of a leucine zipper, a helix-loop motif, or a coiled-coil motif.

Fc mutations suitable for use in the fusion proteins disclosed herein are also discussed in, e.g., Wilkinson et al., Fc-engineered antibodies with immune effector functions completely abolished. PLoS One. 2021; WO2021234402A2; U.S. Pat. No. 8,969,526; EP3692065B1; and U.S. Pat. No. 7,083,784, each of which is incorporated herein by reference.

### Fusion or Conjugation to Half-Life Extension Moieties

In some embodiments, a fusion protein or conjugate of the present disclosure may comprise one or more other moieties which provide the fusion protein or conjugate with increased (in vivo) half-life. In vivo half-life extension means, that the fusion protein or conjugate has an increased half-life in a mammal, such as a human subject, after administration.

Non-limiting examples of half-life extension moieties suitable for use in the present disclosure include polyethylene glycol (PEG) molecules, serum proteins or fragments thereof, binding units that can bind to serum proteins, an Fc portion, and small proteins or peptides that can bind to serum proteins.

In some embodiments, a fusion protein or conjugate of the present disclosure may comprise a binding moiety that can bind to serum albumin, such as human serum albumin, or a serum immunoglobulin, such as IgG. In one embodiment, a fusion protein or conjugate of the present disclosure may comprise a binding moiety that can bind to human serum albumin. In one embodiment, the binding moiety is a single-domain antibody (e.g., VHH).

For example and without limitation, albumin binders that are described in, e.g., WO 04/041865, WO 06/122787, WO2012/175400, WO 2012/175741, WO2015/173325, WO2017/080850, WO2017/085172, WO2018/104444, WO2018/134235, WO2018/134234, each of which is incorporated herein by reference is its entirety, can be used in the fusion protein or conjugate of the present disclosure.

### Fusion or Conjugation to Cytokines

In some embodiments, a fusion protein or conjugate of the present disclosure may comprise one or more cytokine molecules. Non-limiting exemplary cytokine molecules that may be conjugated with include interleukin-2 (IL-2), transforming growth factor beta (TGF-β), thymic stromal lymphopoietin (TSLP), or a variant or combination thereof.

The cytokine IL-2 plays a major role in the activation and function of Tregs. Incorporating IL-2 into the anti-TNFR2 fusion protein or conjugate of the present disclosure may enhance the ability of the anti-TNFR2 antigen-binding protein to promote Tregs expansion and stabilization.

In some embodiments, a fusion protein or conjugate of the present disclosure may comprise two polypeptide chains, each polypeptide chain having the following structure: (anti-TNFR2 VHH) n-Linker-Fc-(IL-2)m, where n and m is independently any integral number (e.g., 1, 2, 3, 4, 5, etc). When n≥2, each anti-TNFR2 VHH may be optionally operably linked to another anti-TNFR2 VHH via a linker. When m≥2, each IL-2 may be optionally operably linked to another IL-2 via a linker.

In one embodiment, a fusion protein or conjugate of the present disclosure comprises two polypeptide chains, each polypeptide chain having the following structure: (anti-TNFR2 VHH)-Linker-Fc-(IL-2).

IL-2 fusion proteins may be prepared as described in e.g., U.S. Pat. No. 10,174,091, WO2014/023752, WO2019/246404, each of which is incorporated by reference in its entirety.

In one embodiment, the IL-2 molecule used in the fusion proteins or conjugates of the present disclosure is a wild-type IL-2 having the amino acid sequence: APTSSSTKKTQLQLEHLLLDLQMILNGINNYKNPKLTRMLTFKFYMPKKATELKHLQCLEEELKPLEEVLNLAQSKNFHLR PRDLISNINVIVLELKGSETTFMCEYADETATIVEFLNRWITFCQSIISTLT (SEQ ID NO: 3926)

In some embodiments, the IL-2 molecule used in the fusion proteins or conjugates of the present disclosure is a variant of IL-2 having an amino acid sequence that is at least 90%, 91%, 92%, 93%, 94%, 95%, 96%, 97%, 98%, or 99% identity to SEQ ID NO: 3926.

In one embodiment, the IL-2 molecule used in the fusion proteins or conjugates of the present disclosure is a variant of IL-2 with a N88D mutation (bolded in the sequence below), having the amino acid sequence: APTSSSTKKTQLQLEHLLLDLQMILNGINNYKNPKLTRMLTFKFYMPKKATELKHLQCLEEELKPLEEVLNLAQSKNFHLR PRDLISDINVIVLELKGSETTFMCEYADETATIVEFLNRWITFCQSIISTLT (SEQ ID NO: 3927)

Other suitable IL-2 variants that can be used in the fusion proteins or conjugates of the present disclosure include those described in U.S. Pat. Nos. 10,174,091, 10,174,092, 11,091,526, 11,091,527, WO2016/164937, U.S. Pat. Nos. 9,580,486, 7,105,653, 9,616,105, 9,428,567, US2017/0051029, US2014/0286898A1, WO2014/153111, WO2010/085495, WO2016/014428, WO2016/025385, and US2006/0269515, each of which are incorporated by reference in its entirety.

### Fusion or Conjugation to Other Moieties

Anti-TNFR2 antigen-binding proteins (e.g., antibodies such as single-domain antibodies) provided herein may be operably linked, directly or indirectly, to a second moiety, such as but not limited to, a detectable label, a drug, a toxin, a radionuclide, an enzyme, an immunomodulatory agent, a cytokine, a cytotoxic agent, a small molecule drug, a chemotherapeutic agent, a therapeutic agent, a diagnostic agent, or a combination thereof.

In some embodiments, a conjugate of the present disclosure comprises a label, which can generate a detectable signal. Such conjugates can be used for research or diagnostic purposes, such as for the in vivo detection of cancer. Preferably, the label is capable of producing, either directly or indirectly, a detectable signal. For example, the label may be radio-opaque or a radioisotope (such as 3H, 14C, 32P, 35S, 123I, 125I, 131I); a fluorescent (fluorophore) or chemiluminescent (chromophore) compound (such as fluorescein isothiocyanate, rhodamine or luciferin); an enzyme (such as β-galactosidase, alkaline phosphatase, or horseradish peroxidase); an imaging agent; or a metal ion. In some embodiments, the label is a radioactive atom for scintigraphic studies, for example 99Tc or 123I, or a spin label for nuclear magnetic resonance (NMR) imaging, such as zirconium-89, iodine-123, iodine-131, indium-111, fluorine-19, carbon-13, nitrogen-15, oxygen-17, gadolinium, manganese, or iron. Zirconium-89 may also be complexed to various metal chelating agents and conjugated to antibodies, e.g., for PET imaging (WO 2011/056983).

Anti-TNFR2 antigen-binding proteins (e.g., antibodies such as single-domain antibodies) of the present disclosure may be conjugated to another moiety, such as an epitope tag, e.g., for the purpose of purification or detection. Examples of such molecules that are useful in protein purification include those that present structural epitopes capable of being recognized by a second molecule. This is commonly employed in protein purification by affinity chromatography, in which a molecule is immobilized on a solid support and exposed to a heterogeneous mixture containing a target protein conjugated to a molecule capable of binding the immobilized compound. Non-limiting examples of epitope tag molecules that can be conjugated to anti-TNFR2 antigen-binding proteins (e.g., antibodies such as single-domain antibodies) of the present disclosure, e.g., for the purposes of molecular recognition include a poly-histidine tag (His-tag), a myc-tag, human influenza hemagglutinin (HA) tag, a FLAG-tag, maltose-binding protein, glutathione-S-transferase, biotin, and streptavidin. Conjugates containing the epitopes presented by these molecules are capable of being recognized by complementary molecules such as maltose, glutathione, a nickel-containing complex, an anti-FLAG antibody, an anti-myc antibody, an anti-HA antibody, streptavidin, or biotin, respectively. For example, one can purify an anti-TNFR2 antigen-binding protein of the present disclosure that has been conjugated to an epitope tag from a complex mixture of other proteins and biomolecules (e.g., DNA, RNA, carbohydrates, phospholipids, etc) by treating the mixture with a solid phase resin containing a complementary molecule that can selectively recognize and bind the epitope tag of the TNFR2 antibody or fragment thereof. Examples of solid phase resins include agarose beads, which are compatible with purifications in aqueous solution.

In some embodiments, a conjugate of the present disclosure may comprise one or more anti-TNFR2 VHH domains described herein conjugated to a therapeutic agent, which can be cytotoxic, cytostatic, or otherwise provides some therapeutic benefit. In some embodiments, the cytotoxic agent is a drug, a chemotherapeutic agent, a growth inhibitory agent, a toxin (e.g., an enzymatically active toxin of bacterial, fungal, plant, or animal origin, or fragments thereof), or a radioactive isotope (e.g., a radioconjugate), Such conjugates may be applicable to, e.g., the treatment or prevention of a disease associated with autoreactive cytotoxic T-cell activity. In some embodiments, antibody drug conjugates described herein may allow targeted delivery of a drug moiety to a target tissue (e.g., tumors).

In some embodiments, a conjugate of the present disclosure comprises a toxin. In some embodiments, the toxin includes, for example, bacterial toxins such as diphtheria toxin, plant toxins such as ricin, small molecule toxins such as geldanamycin (Mandler et al., J. Nat. Cancer Inst. 92 (19): 1573-1581 (2000); Mandler et al., Bioorganic & Med. Chem. Letters 10:1025-1028 (2000); Mandler et al., Bioconjugate Chem. 13:786-791 (2002)), maytansinoids (EP 1391213; Liu et al., Proc. Natl. Acad. Sci. USA 93:8618-8623 (1996)), and calicheamicin (Lode et al., Cancer Res. 58:2928 (1998); Hinman et al., Cancer Res. 53:3336-3342 (1993)). The toxins may exert their cytotoxic and cytostatic effects by mechanisms including tubulin binding, DNA binding, or topoisomerase inhibition. Examples of other therapeutic agents that can be conjugated to an anti-TNFR2 antigen-binding protein of the present disclosure are described herein (see “Treatment Methods and Other Uses” section).

In some embodiments, anti-TNFR2 antigen-binding proteins (e.g., antibodies such as single-domain antibodies) of the present disclosure may be fused or conjugated to one or more moieties that facilitate delivery to the central nervous system (CNS)/brain. The moiety that can facilitate delivery of an anti-TNFR2 antigen-binding protein to the central nervous system (CNS)/brain can be for example, a peptide, a polypeptide, small molecule, a lipid, or a synthetic polymer. Various approaches to deliver single-domain antibodies into the brain are described in Pothin et al., Pharmaceutics 2020, 12 (10), 937, which is incorporated herein by reference in its entirety.

As a non-limiting example, an anti-TNFR2 antigen-binding protein (e.g., antibody such as single-domain antibody) of the present disclosure may be fused or conjugated to a moiety (e.g., an antibody) that binds to the transferrin receptor (TfR) or insulin receptor. The transferrin receptor (TfR) is highly expressed by brain capillary endothelial cells (BCECs) forming the blood-brain barrier (BBB) and has been utilized as a target for brain drug delivery. Monoclonal antibodies binding to the TfR, such as clone Ri7, have been shown to internalize into BCECs in vivo. As another example, an anti-TNFR2 antigen-binding protein (e.g., antibody such as single-domain antibody) of the present disclosure may be conjugated to hydrophobic fatty acid moieties, such as C18 fatty acid (stearic acid), C16 fatty acid (palmitic acid) or C8 fatty acid (octanoic acid) moieties; or amphiphilic block copolymer moieties, such as polyethylene oxide)-poly(propylene oxide)-poly(ethylene oxide) (pluronics or poloxamers) or poly(2-oxasolines). Various fatty acid moieties and block copolymer moieties that can be utilized for brain delivery of proteins are described in, e.g., Yi and Kabanov, J Drug Target. 2013; 21 (10): 940-955, which is incorporated herein by reference in its entirety.

Example methods for attaching a moiety, such as a label, to a binding protein include those described in Hunter, et al., Nature 144:945 (1962); David, et al., Biochemistry 13:1014 (1974); Pain, et al., J. Immunol. Meth. 40:219 (1981); Nygren, J. Histochem, and Cytochem. 30:407 (1982); Wensel and Meares, Elsevier, N.Y. (1983); and Colcher et al., Meth. Enzymol., 121:802-16 (1986). Additional suitable methods for preparing the conjugates of the present disclosure include those described in, e.g., WO 2009/067800, WO 2011/133886, and US2014322129, incorporated by reference herein in their entirety.

In some embodiments, the attachment between an anti-TNFR2 antigen-binding protein and a second moiety can be covalent or non-covalent, e.g., via a biotin-streptavidin non-covalent interaction. In some embodiments, a second moiety can be attached to an anti-TNFR2 antigen-binding protein using any of various molecular biological or chemical conjugation and linkage methods known in the art and described below. In some embodiments, linkers such as peptide linkers, cleavable linkers, non-cleavable linkers, or linkers that aid in the conjugation reaction, can be used to link, or conjugate a second moiety to an anti-TNFR2 antigen-binding protein described herein.

In some embodiments, an anti-TNFR2 antigen-binding protein (e.g., antibody such as single-domain antibody) is conjugated to one or more second moieties, e.g., about 1 to about 20 moieties per molecule, optionally via a linker. In some embodiments, the one or more second moieties can be the same or different. The linker may be composed of one or more linker components. For covalent attachment of an antibody and the second moiety, the linker typically has two reactive functional groups, i.e., bivalency in a reactive sense. Bivalent linker reagents which are useful to attach two or more functional or biologically active moieties, such as peptides, nucleic acids, drugs, toxins, antibodies, haptens, and reporter groups have been described in, e.g., Hermanson, G. T. (1996) Bioconjugate Techniques; Academic Press: New York, p 234-242.

In some embodiments, a linker used in a conjugate of the present disclosure may include 6-maleimidocaproyl (“MC”), maleimidopropanoyl (“MP”), valine-citrulline (“val-cit”), a alanine-phenylalanine (“ala-phe”), p-aminobenzyloxycarbonyl (“PAB”), N-Succinimidyl 4-(2-pyridylthio) pentanoate (“SPP”), N-Succinimidyl 4-(N-maleimidomethyl)cyclohexane-I carboxylate (“SMCC”), or N-Succinimidyl(4-iodo-acetyl)aminobenzoate (“STAB”), or a combination thereof.

In some embodiments, a linker used in a conjugate of the present disclosure may comprise amino acid residues. Exemplary amino acid linker components include a dipeptide, a tripeptide, a tetrapeptide or a pentapeptide. Exemplary dipeptides include valine-citrulline (vc or val-cit), alanine-phenylalanine (af or ala-phe). Exemplary tripeptides include glycine-valine-citrulline (gly-val-cit) and glycine-glycine-glycine (gly-gly-gly), Amino acid residues used in an amino acid linker component may include naturally occurring amino acids, as well as minor amino acids and non-naturally occurring amino acid analogs, such as citrulline. Amino acid linker components can be designed and optimized in their selectivity for enzymatic cleavage by particular enzymes, for example, a tumor-associated protease, cathepsin B, C and D, or a plasmin protease.

Conjugates of an anti-TNFR2 antigen-binding protein (e.g., antibody such as single-domain antibody) and second moiety (e.g., cytotoxic agent) can be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl substrate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Conjugates of the present disclosure can be prepared by a variety of methods. For example, the conjugation method may include: (1) reaction of a nucleophilic group of a VHH domain with a bivalent linker reagent, to form VHH-Linker, via a covalent bond, followed by reaction with a drug moiety; or (2) reaction of a nucleophilic group of a drug moiety with a bivalent linker reagent, to form drug-linker, via a covalent bond, followed by reaction with the nucleophilic group of a VHH domain.

Nucleophilic groups on proteins including antibodies (e.g., VHH domains), include, but are not limited to: (i) N-terminal amine groups, (ii) side chain amine groups (e.g., lysine), (iii) side chain thiol groups (e.g., cysteine), and (iv) sugar hydroxyl or amino groups where the antibody is glycosylated. Amine, thiol, and hydroxyl groups are nucleophilic and capable of reacting to form covalent bonds with electrophilic groups on linker moieties and linker reagents including: (i) active esters such as NHS esters, HOBt esters, haloformates, and acid halides; (ii) alkyl and benzyl halides such as haloacetamides; (iii) aldehydes, ketones, carboxyl, and maleimide groups. Additional nucleophilic groups can be introduced into proteins (e.g., antibodies such as VHH domains) through the reaction of lysines with 2-iminothiolane (Traut's reagent) resulting in conversion of an amine into a thiol. Reactive thiol groups may be introduced into a protein (e.g., antibody such as a VHH domain) by introducing one, two, three, four, or more cysteine residues.

Conjugates, such as antibody drug conjugates, may also be produced by modification of an antibody, such as a VHH domain, to introduce electrophilic moieties, which can react with nucleophilic substituents on the linker reagent or drug. The sugars of glycosylated antibodies may be oxidized, e.g., with periodate oxidizing reagents, to form aldehyde or ketone groups which may lead with the amine group of linker reagents or drug moieties. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the protein that can react with appropriate groups on the drug (Hermanson, Bioconjugate Techniques). In another embodiment, proteins containing N-terminal serine or threonine residues can react with sodium meta-periodate, resulting in production of an aldehyde in place of the first amino acid. Such aldehyde can be reacted with a drug moiety or linker nucleophile.

Likewise, nucleophilic groups on a drug moiety include, but are not limited to: amine, thiol, hydroxyl, hydrazide, oxime, hydrazine, thiosemicarbazone, hydrazine carboxylate, and arylhydrazide groups capable of reacting to form covalent bonds with electrophilic groups on linker moieties and linker reagents including: (i) active esters such as NHS esters, HOBi esters, haloformates, and acid halides; (ii) alkyl and benzyl halides such as haloacetamides; (iii) aldehydes, ketones, carboxyl, and maleimide groups.

Alternatively, a fusion protein containing a VHH domain and cytotoxic agent may be made, e.g., by recombinant DNA techniques or peptide synthesis. A DNA sequence may be engineered to comprise respective regions encoding the two portions of the fusion protein either adjacent to one another or separated by a region encoding a linker peptide which does not impair the desired properties of the fusion protein. The DNA sequence can be then transfected into a host cell that expresses the fusion protein. The fusion protein can be recovered from the cell culture and purified using techniques known in the art.

### Linkers

In some embodiments, the one or more polypeptides of the fusion proteins of the present disclosure are operably linked via peptide linkers. A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In some embodiments, a peptide linker, e.g., a peptide linker separating two VHH domains or an VHH domain and a heavy chain constant region, is at least 5 amino acids, at least 6 amino acids or at least 7 amino acids in length and optionally is up to 30 amino acids, up to 40 amino acids, up to 50 amino acids or up to 60 amino acids in length.

In some embodiments, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, charged (e.g., charged hydrophilic linkers) and/or flexible linkers are used. Examples of flexible linkers that can be used in the fusion proteins of the disclosure include those disclosed by Chen et al, 2013, Adv Drug Deliv Rev. 65 (10): 1357-1369 and Klein et al., 2014, Protein Engineering, Design & Selection 27 (10): 325-330. Particularly useful flexible linkers are or comprise repeats of glycines and serines (termed “GS-linker” herein), e.g., a monomer or multimer of GₙS (SEQ ID NO: 4013) or SGₙ (SEQ ID NO: 4014), where n is an integer from 1 to 10, e.g., 1 2, 3, 4, 5, 6, or 7, 8, 9 or 10. In one embodiment, the linker is or comprises a monomer or multimer of repeat of G₄S (SEQ ID NO: 3969), e.g., (GGGGS)ₙ (SEQ ID NO: 4015).

Polyglycine linkers can suitably be used in the fusion proteins of the disclosure. In some embodiments, a peptide linker used herein comprises two consecutive glycines (2Gly), three consecutive glycines (3Gly), four consecutive glycines (4Gly) (SEQ ID NO: 4016), five consecutive glycines (5Gly) (SEQ ID NO: 4017), six consecutive glycines (6Gly) (SEQ ID NO: 4018), seven consecutive glycines (7Gly) (SEQ ID NO: 4019), eight consecutive glycines (8Gly) (SEQ ID NO: 4020), or nine consecutive glycines (9Gly) (SEQ ID NO: 4021).

In some embodiments, a GS-linker used herein comprises an amino acid sequence selected from GGSGGS, i.e., (GGS)₂ (SEQ ID NO: 4022); GGSGGSGGS, i.e., (GGS)₃ (SEQ ID NO: 4023); GGSGGSGGSGGS, I.e., (GGS)₄ (SEQ ID NO: 4024); and GGSGGSGGSGGSGGS, i.e., (GGS)₅ (SEQ ID NO: 4025). In some embodiments, the fusion proteins can include a combination of a GS-linker and a glycine linker.

In one embodiment, two or more VHHs are linked via a GGGGSGGGGSGGGGS (SEQ ID NO: 3970) linker. In one embodiment, two or more VHHs are linked via a GGGGSGGGGS (SEQ ID NO: 4026) linker. In one embodiment, a VHH and an Fc region are linked via a GGGGSESKYGPPCPSCP (SEQ ID NO: 4008) linker. In one embodiment, a VHH and an Fc region are linked via a GGGGS (SEQ ID NO: 3969) linker.

In some embodiments, the one or more polypeptides of the fusion proteins of the present disclosure are operably linked via a “rigid” peptide linker. Such peptidic linker may comprise a proline-rich peptide. In one embodiment, a rigid peptide linker comprises PAPAPAPAPAPAPAPAP (SEQ ID NO: 4009). In one embodiment, a rigid peptide linker comprises GGGGSPAPAPAPAPAPAPAPAPGGGGS (SEQ ID NO: 4012). In one embodiment, a rigid peptide linker comprises A(EAAAK)nA (SEQ ID NO: 4027), where n is any integer, e.g., 12, 3, 4, 5, 6, or 7, 8, 9 or 10.

Other exemplary peptide linkers that can be used in the fusion proteins described herein are shown in Table 2.

TABLE 2
Exemplary Peptide Linker Sequences

| Linker | Amino acid sequence | SEQ ID NO |
|---|---|---|
| G4S | GGGGS | 3969 |
| (G₄S)₃ | GGGGSGGGGSGGGGS | 3970 |
| Linker 3 | GGSEGKSSGSGSESKSTGGS | 3971 |
| Linker 4 | GGGSGGGS | 3972 |
| Linker 3 | GGGSGGGSGGGS | 3973 |
| Linker 4 | GGGSGGGSGGGSGGGS | 3974 |
| Linker 5 | GGGSGGGSGGGSGGGSGGGS | 3975 |
| Linker 6 | GGGGSGGGGSGGGGSGGGGS | 3976 |
| Linker 7 | GGGGSGGGGSGGGGSGGGGSGGGGS | 3977 |
| Linker 8 | IRPRAIGGSKPRVA | 3978 |
| Linker 9 | GKGGSGKGGSGKGGS | 3979 |
| Linker 10 | GGKGSGGKGSGGKGS | 3980 |
| Linker 11 | GGGKSGGGKSGGGKS | 3981 |
| Linker 12 | GKGKSGKGKSGKGKS | 3982 |
| Linker 13 | GGGKSGGKGSGKGGS | 3983 |
| Linker 14 | GKPGSGKPGSGKPGS | 3984 |
| Linker 15 | GKPGSGKPGSGKPGSGKPGS | 3985 |
| Linker 16 | GKGKSGKGKSGKGKSGKGKS | 3986 |
| Linker 17 | STAGDTHLGGEDFD | 3987 |
| Linker 18 | GEGGSGEGGSGEGGS | 3988 |
| Linker 19 | GGEGSGGEGSGGEGS | 3989 |
| Linker 20 | GEGESGEGESGEGES | 3990 |
| Linker 21 | GGGESGGEGSGEGGS | 3991 |
| Linker 22 | GEGESGEGESGEGESGEGES | 3992 |
| Linker 23 | GSTSGSGKPGSGEGSTKG | 3993 |
| Linker 24 | PRGASKSGSASQTGSAPGS | 3994 |
| Linker 25 | GTAAAGAGAAGGAAAGAAG | 3995 |
| Linker 26 | GTSGSSGSGSGGSGSGGGG | 3996 |
| Linker 27 | GSGS | 3997 |
| Linker 28 | APAPAPAPAP | 3998 |
| Linker 29 | APAPAPAPAPAPAPAPAPAP | 3999 |
| Linker 30 | AEAAAKEAAAKEAAAAKEAAAAKEAAAAKAAA | 4000 |
| (GGGGS)6 linker | GGGGSGGGGSGGGGSGGGGSGGGGSGGGGS | 4001 |
| (GGGGS)7 linker | GGGGSGGGGSGGGGSGGGGSGGGGSGGGGSGGGGS | 4002 |
| G1 hinge | EPKSCDKTHTCPPCP | 4003 |
| 9GS-G1 hinge | GGGGSGGGSEPKSCDKTHTCPPCP | 4004 |
| Llama upper long hinge | EPKTPKPQPAAA | 4005 |
| region | | |
| G3 hinge | ELKTPLGDTTHTCPRCPEPKSCDTPPPCPRCPEPKSCDTPPPC | 4006 |
| | PRCPEPKSCDTPPPCPRCP | |
| G4 hinge | ESKYGPPCPSCP | 4007 |
| G4S-G4 hinge | GGGGSESKYGPPCPSCP | 4008 |
| Proline-linker 1 | PAPAPAPAPAPAPAPAP | 4009 |
| Proline-linker 2 | GGGGSPÅPAPAPAPAPAPAPAP | 4010 |
| Proline-linker 3 | PÅPAPAPAPAPAPAPAPGGGGS | 4011 |
| Proline-linker 4 | GGGGSPAPAPAPAPAPAPAPAPGGGGS | 4012 |

### Exemplary Fusion Proteins of the Present Disclosure

Non-limiting examples of fusion proteins (e.g., bivalent, or tetravalent constructs with/without Fc regions, IL-2 fusion constructs) are disclosed in the “Examples” and “List of Sequences” sections below.

In various embodiments, a fusion protein of the present disclosure comprises any one of SEQ ID NOs: 3933-3964, 4483-4513, 4686-4696, 4709-4716, and 4735-4770, or a similar sequence thereof having at least 70%, at least 75%, at least 80%, at least 85%, at least 90%, at least 95%, at least 98%, or at least 99% sequence identity.

In some embodiments, a fusion protein of the present disclosure comprises any one of SEQ ID NOs: 4483-4513, 4686-4696, 4709-4716, and 4735-4770, or a similar sequence thereof having at least 70%, at least 75%, at least 80%, at least 85%, at least 90%, at least 95%, at least 98%, or at least 99% sequence identity.

In some embodiments, a fusion protein of the present disclosure comprises SEQ ID NO: 4483 or a similar sequence thereof having at least 70%, at least 75%, at least 80%, at least 85%, at least 90%, at least 95%, at least 98%, or at least 99% sequence identity.

In some embodiments, a fusion protein of the present disclosure comprises SEQ ID NO: 4489 or a similar sequence thereof having at least 70%, at least 75%, at least 80%, at least 85%, at least 90%, at least 95%, at least 98%, or at least 99% sequence identity.

In some embodiments, a fusion protein of the present disclosure comprises any one of SEQ ID NO: 4709-4716 or a similar sequence thereof having at least 70%, at least 75%, at least 80%, at least 85%, at least 90%, at least 95%, at least 98%, or at least 99% sequence identity.

In some embodiments, a fusion protein of the present disclosure comprises any one of SEQ ID NO: 4735-4770 or a similar sequence thereof having at least 70%, at least 75%, at least 80%, at least 85%, at least 90%, at least 95%, at least 98%, or at least 99% sequence identity.

In some embodiments, provided herein is a fusion protein that specifically binds TNFR2, comprising two polypeptides, wherein each polypeptide comprises two anti-TNFR2 antigen-binding proteins as described herein which are operably linked to each other, wherein one of the antigen-binding proteins is further operably linked to a dimerization domain (e.g., an immunoglobulin Fc region). The two polypeptides dimerize in the presence of the dimerization domain to form a tetravalent molecule (i.e., having four anti-TNFR2 antigen-binding proteins per molecule).

In some embodiments, the two antigen-binding proteins are operably linked to each other via a peptide linker. In one embodiment, the peptide linker is a (G4S)n (SEQ ID NO: 4015) linker. In one embodiment, the peptide linker is a GGGGSGGGGSGGGGS linker (SEQ ID NO: 3970).

In some embodiments, one of the two antigen-binding proteins is further operably linked to an immunoglobulin Fc region via a peptide linker. In one embodiment, the peptide linker is a (G4S)n (SEQ ID NO: 4015) linker. In one embodiment, the peptide linker is a GGGGS linker (SEQ ID NO: 3969).

In some embodiments, the fusion protein described herein further comprises an immunoglobulin Fc region. In some embodiments, the immunoglobulin Fc region is an Fc region of a human immunoglobulin. In some embodiments, the immunoglobulin Fc region is an Fc region of human IgG1, IgG2, IgG3 or IgG4, or a variant thereof.

In some embodiments, the immunoglobulin Fc region is an Fc region of human IgG1, or a variant thereof. In some embodiments, the Fc region of human IgG1 comprises one or more mutations selected from L234A, L235A, G237A, D265A, N297A, and/or P329A according to EU numbering. In some embodiments, the Fc region of human IgG1 comprises a set of mutations selected from
1). L234A and L235A;
2). L234A, L235A, and P329A;
3). D265A, N297A and P329A; and
4). L234A, L235A, and G237A.

In some embodiments, the immunoglobulin Fc region is an Fc region of human IgG1 comprising L234A, L235A, and P329A.

In some embodiments, the immunoglobulin Fc region is an Fc region of human IgG4, or a variant thereof. In some embodiments, the Fc region of human IgG4 comprises one or more mutations selected from S228P, L235E, L235A, and/or F234A according to EU numbering. In some embodiments, the Fc region of human IgG4 comprises a set of mutations selected from
1). S228P and L235E;
2). S228P and L235A;
3). S228P, F234A, and L235E; and
4). S228P, F234A, and L235A.

In some embodiments, the immunoglobulin Fc region is an Fc region of human IgG4 comprising S228P and L235E.

It is to be understood that although the exemplary fusion proteins described herein contain non-humanized VHH amino acid sequences, such non-humanized VHH amino acid sequences can be replaced with any of the humanized VHH amino acid sequences described herein (e.g., in Tables 1-1 and 1-2).

In some embodiments, the fusion protein described herein may further comprise a signal sequence at its N-terminus. Signal sequences may be present in the precursor molecule of the fusion protein and may be removed after the protein is secreted from the host cell during production. In some embodiments, the signal sequence is MAVMAPRTLVLLLSGALALTQTWA (SEQ ID NO: 3928) or a fragment or variant thereof. In some embodiments, the signal sequence is MYRMQLLSCIALSLALVTNS (SEQ ID NO: 3929), or a fragment or variant thereof.

### Polynucleotide Molecules

In another aspect, provided herein are polynucleotide molecules encoding the anti-TNFR2 antigen-binding proteins (e.g., antibodies including single-domain antibodies) or fusion proteins described herein. Polynucleotide molecules encoding polypeptide portion(s) of a conjugate of the present disclosure are also encompassed within the present disclosure.

In some embodiments, a polynucleotide molecule of the present disclosure encodes an anti-TNFR2 VHH amino acid sequence selected from SEQ ID NOs: 4, 7, 11, 15, 19, 23, 27, 31, 35, 39, 43, 47, 4064, 4068, 4072, 4521, 93-640, 4079-4125 2805-3363, 4359-4420, and 4605-4628, or a similar sequence thereof having at least 70%, at least 75%, at least 80%, at least 85%, at least 90%, at least 95%, at least 98%, or at least 99% sequence identity.

In some embodiments, a polynucleotide molecule of the present disclosure encoding an anti-TNFR2 VHH comprises the nucleotide sequence of any one of SEQ ID NOs: 48-59, 4073-4075, 4522, 4525, 4528, 4531, 3364-3922, 4421-4482, and 4629-4652, or a similar sequence thereof having at least 70%, at least 75%, at least 80%, at least 85%, at least 90%, at least 95%, at least 98%, or at least 99% sequence identity.

In an embodiment provided herein, a polynucleotide molecule of the present disclosure encodes a humanized VHH amino acid sequence selected from SEQ ID NOs: 81-92, 4076, 4078, 4523, 4526, 4529, 4532, 4731-4734, 641-1127, and 4126-4172, or a similar sequence thereof having at least 70%, at least 75%, at least 80%, at least 85%, at least 90%, at least 95%, at least 98%, or at least 99% sequence identity.

In an embodiment provided herein, a polynucleotide molecule of the present disclosure encodes a humanized VHH amino acid sequence of SEQ ID NO: 4526, or a similar sequence thereof having at least 70%, at least 75%, at least 80%, at least 85%, at least 90%, at least 95%, at least 98%, or at least 99% sequence identity. In an embodiment provided herein, the polynucleotide molecule encoding a humanized VHH amino acid sequence of SEQ ID NO: 4526 comprises the nucleotide sequence of SEQ ID NO: 4525, or a similar sequence thereof having at least 70%, at least 75%, at least 80%, at least 85%, at least 90%, at least 95%, at least 98%, or at least 99% sequence identity.

In an embodiment provided herein, a polynucleotide molecule of the present disclosure encodes a humanized VHH amino acid sequence of SEQ ID NO: 4529, or a similar sequence thereof having at least 70%, at least 75%, at least 80%, at least 85%, at least 90%, at least 95%, at least 98%, or at least 99% sequence identity. In an embodiment provided herein, the polynucleotide molecule encoding a humanized VHH amino acid sequence of SEQ ID NO: 4529 comprises the nucleotide sequence of SEQ ID NO: 4528, or a similar sequence thereof having at least 70%, at least 75%, at least 80%, at least 85%, at least 90%, at least 95%, at least 98%, or at least 99% sequence identity.

In an embodiment provided herein, a polynucleotide molecule of the present disclosure encodes a humanized VHH amino acid sequence of SEQ ID NO: 4532, or a similar sequence thereof having at least 70%, at least 75%, at least 80%, at least 85%, at least 90%, at least 95%, at least 98%, or at least 99% sequence identity. In an embodiment provided herein, the polynucleotide molecule encoding a humanized VHH amino acid sequence of SEQ ID NO: 4532 comprises the nucleotide sequence of SEQ ID NO: 4531, or a similar sequence thereof having at least 70%, at least 75%, at least 80%, at least 85%, at least 90%, at least 95%, at least 98%, or at least 99% sequence identity.

In an embodiment provided herein, a polynucleotide molecule of the present disclosure encodes a fusion protein comprising an amino acid sequence selected from SEQ ID NOs: 3933-3964, and 4483-4513, 4686-4696, 4709-4716, and 4735-4770, or a similar sequence thereof having at least 70%, at least 75%, at least 80%, at least 85%, at least 90%, at least 95%, at least 98%, or at least 99% sequence identity.

A polynucleotide molecule may be used to transform/transfect a host cell or host organism, e.g., for expression and/or production of a polypeptide. Suitable hosts or host cells for production of an anti-TNFR2 polypeptides described herein include any suitable fungal, prokaryotic, or eukaryotic cell or cell line or any suitable fungal, prokaryotic, or eukaryotic organism. A host or host cell comprising a polynucleotide molecule encoding an anti-TNFR2 antigen-binding protein polypeptide or fusion protein described herein is also encompassed by the present disclosure.

A polynucleotide molecule may be for example DNA, RNA, or a hybrid thereof, and may also comprise (e.g., chemically) modified nucleotides, like locked nucleic acids (LNA) or peptide nucleic acids (PNA). In some embodiments, the polynucleotide is single-stranded. In some embodiments, the polynucleotide is double-stranded. In one embodiment, the polynucleotide is in the form of double-stranded DNA (e.g., plasmid). In some embodiments, the polynucleotide is in the form of a single-stranded RNA (e.g., mRNA).

Techniques for generating polynucleotides may include, for example but not limited to, automated DNA synthesis; site-directed mutagenesis; combining two or more naturally occurring and/or synthetic sequences (or two or more parts thereof), introduction of mutations that lead to the expression of a truncated expression product; introduction of one or more restriction sites (e.g. to create cassettes and/or regions that may easily be digested and/or ligated using suitable restriction enzymes), and/or the introduction of mutations by means of a PCR reaction using one or more “mismatched” primers. Alternatively, polynucleotides of the present disclosure may be isolated from a suitable natural source. Polynucleotide sequences encoding naturally occurring (poly) peptides can for example be subjected to site-directed mutagenesis, to generate a polynucleotide molecule encoding polypeptide with sequence variation.

### Vectors

Also provided herein are vectors comprising the polynucleotide molecules encoding the anti-TNFR2 antigen-binding proteins (e.g., antibodies including single-domain antibodies), fusion proteins, or other relevant polypeptides of the present disclosure. A “vector” as used herein is a vehicle suitable for carrying genetic material into a host cell. A vector can include a nucleic acid vector, such as a plasmid or mRNA, or nucleic acids embedded into a bigger structure, such as a liposome or viral vector.

A vector can include one or more of the following elements: an origin of replication, one or more regulatory sequences (e.g., promoters, enhancers, terminators) that regulate the expression of a polypeptide of interest, and/or one or more selectable marker genes (such as, for example, antibiotic resistance genes and genes that can be used in colorimetric assays, for example, β-galactosidase). For DNA-based vectors, this usually includes the presence of elements for transcription (e.g., a promoter and a polyA signal) and translation (e.g., Kozak sequence). In some embodiments, the vector is an expression vector, i.e. a vector suitable for expressing an encoded polypeptide or construct under suitable conditions in a host cell.

To express an anti-TNFR2 antigen-binding protein or fusion protein (or fragments thereof) of the present disclosure, polynucleotides encoding partial or full-length polypeptide chains, e.g., obtained as described above (e.g., VHH, VHH-Fc), can be inserted into expression vectors such that the genes are operatively linked to one or more transcriptional and translational control sequences. The expression vector and expression control sequences are chosen to be compatible with the expression host cell used. Polynucleotides encoding the two or more polypeptide chains (when present and differ from one another) of an anti-TNFR2 antigen-binding protein or fusion protein of the present disclosure can be inserted into separate vectors, or, optionally, incorporated into the same expression vector.

In addition to polynucleotides encoding the polypeptide chain(s) of an anti-TNFR2 antigen-binding protein or fusion protein, the recombinant expression vectors of the invention may include regulatory sequences that control the expression of genes encoding the polypeptide chain(s) in a host cell. The design of the expression vector, including the selection of regulatory sequences, may depend on the choice of the host cell to be transformed and/or the desired level of protein expression. For example, suitable regulatory sequences for mammalian host cell expression include viral elements that direct high levels of protein expression in mammalian cells, such as promoters and/or enhancers derived from cytomegalovirus (CMV), Simian Virus 40 (SV40), adenovirus, (e.g., the adenovirus major late promoter (AdMLP)) and polyoma. Additional examples of viral regulatory elements, and sequences thereof, include those described in, e.g., U.S. Pat. Nos. 5,168,062; 4,510,245; and 4,968,615; the disclosures of each of which are incorporated herein by reference.

Recombinant expression vectors of the present disclosure may carry additional sequences, such as sequences that regulate replication of the vector in host cells (e.g., origins of replication) and selectable marker genes. A selectable marker gene facilitates selection of host cells into which the vector has been introduced (see e.g., U.S. Pat. Nos. 4,399,216; 4,634,665; and 5,179,017; the disclosure of each of which is incorporated herein by reference in its entirety). For example, typically the selectable marker gene confers resistance to antibiotics, such as ampicillin, chloramphenicol, kanamycin, or nourseothricin, or cytotoxic drugs, such as G418, puromycin, blasticidin, hygromycin or methotrexate, to a host cell into which the vector has been introduced. Suitable selectable marker genes can include the dihydrofolate reductase (DHFR) gene (for use in DHFR deficient host cells with methotrexate selection/amplification) and the neo gene (for G418 selection).

Vectors of the present disclosure may further include sequence elements that enhance the rate of translation of these genes or improve the stability or nuclear export of the mRNA that results from gene transcription. These sequence elements include, e.g., 5′ and 3′ untranslated regions, an internal ribosomal entry site (IRES), and polyadenylation signal site in order to direct efficient transcription of the gene carried on the expression vector.

Viral vectors can be used for the efficient delivery of exogenous genes into the genome of a cell (e.g., a eukaryotic or prokaryotic cell). Viral vectors are particularly useful for gene delivery because the polynucleotides contained within such genomes are typically incorporated into the genome of a target cell by generalized or specialized transduction. These processes occur as part of the natural viral replication cycle, and do not require added proteins or reagents to induce gene integration. Examples of suitable viral vectors include a retrovirus, adenovirus (e.g., Ad5, Ad26, Ad34, Ad35, and Ad48), parvovirus (e.g., adeno-associated viruses (AAV) such as AAV2, AAV8, AAV9), negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double-stranded DNA viruses including adenovirus, herpes virus (e.g., Herpes Simplex virus types 1 and 2, Epstein-Barr virus, cytomegalovirus), baculovirus, coronavirus, and poxvirus (e.g., vaccinia, modified vaccinia Ankara (MVA), fowlpox and canarypox). Other viruses useful for delivering polynucleotides encoding polypeptides of the present disclosure include, for example Norwalk virus, togavirus, flavivirus, reoviruses, papovavirus, hepadnavirus, and hepatitis virus. Examples of retroviruses include, but are not limited to, avian leukosis-sarcoma, mammalian C-type, B-type viruses, D-type viruses, HTLV-BLV group, lentivirus, spumavirus (Coffin, J. M.1996. Fundamental Virology, DMKDN Fields, P M Howley, ed. (Philadelphia, Lippincott-Raven Publishers): 763-843., the disclosure of which is incorporated herein by reference). Other examples of viral genomes useful in the compositions and methods of the present disclosure include murine leukemia viruses, murine sarcoma viruses, mouse mammary tumor virus, bovine leukemia virus, feline sarcoma virus, feline leukemia virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, and lentiviruses.

### Host Cells

In one aspect, the present disclosure also provides host cells or host organisms that comprise the polynucleotides or vectors encoding the anti-TNFR2 antigen-binding proteins (e.g., antibodies including single-domain antibodies), fusion proteins, or other relevant polypeptides described herein. Suitable host cells or host organisms can be any suitable fungal, prokaryotic, or eukaryotic cell or cell line or any suitable fungal, prokaryotic, or eukaryotic organism. Host cells include progeny of a single host cell, and the progeny may not necessarily be completely identical (in morphology or in genomic DNA complement) to the original parent cell due to natural, accidental, or deliberate mutation. Host cells can also include cells transfected in vivo with a polynucleotide(s) or vector provided herein.

Exemplary eukaryotic cells include mammalian cells, such as primate or non-primate animal cells; fungal cells, such as yeast (e.g., Saccharomyces cerevisiae or Pichia pastoris); plant cells; and insect cells. Non-limiting exemplary mammalian cells include, but are not limited to, NSO cells, PER.C6® cells (Crucell), COS cells, SP2/0 cells, and 293 and CHO cells, and their derivatives, such as 293-6E, CHO-DG44, CHO-K1, CHO-S, and CHO-DS cells, Exemplary prokaryotic cells include bacterial cells such as Escherichia coli.

### Preparation Methods

The present disclosure also provides methods of producing the anti-TNFR2 antigen-binding proteins (e.g., antibodies including single-domain antibodies), fusion proteins, or conjugates described herein.

In some embodiments, a method may comprise transforming/transfecting a host cell or host organism with a polynucleotide encoding an anti-TNFR2 antigen-binding protein (e.g., antibody such as single-domain antibody), fusion protein, or other relevant polypeptide(s) described herein, expressing the anti-TNFR2 antigen-binding protein (e.g., antibody such as single-domain antibody), fusion protein, or other relevant polypeptide(s) in the host, optionally followed by one or more isolation and/or purification steps.

When recombinant expression vectors encoding one or more polypeptide(s) of an anti-TNFR2 antigen-binding protein (e.g., antibody such as single-domain antibody), fusion protein, or conjugate of the present disclosure are introduced into mammalian host cells, the host cells are cultured for a period of time sufficient to allow for expression of the protein(s) or polypeptide(s) in the host cells or secretion of the protein(s) or polypeptide(s) into the culture medium in which the host cells are grown. Protein(s) or polypeptide(s) can be recovered from the culture medium using standard protein purification methods. Host cells can also be used to produce portions of intact antibodies, such as VHH domains.

Once a protein or polypeptide of the present disclosure has been produced by recombinant expression, it can be purified by any method known in the art for purification of a protein or polypeptide, for example, by chromatography (e.g., ion exchange, affinity, particularly by affinity for TNFR2 after Protein A or Protein G selection, and sizing column chromatography), centrifugation, differential solubility, or by any other standard technique for the purification of proteins. Further, the proteins or polypeptides of the present disclosure can be fused to heterologous polypeptide sequences described herein (e.g., His-tag) or otherwise known in the art to facilitate purification or to produce therapeutic conjugates below). Once isolated, a protein or polypeptide of the present disclosure can, if desired, be further purified, e.g., by high performance liquid chromatography, or by gel filtration chromatography, such as on a Superdex™ column.

### Pharmaceutical Compositions and Formulations

The present disclosure also provides a composition comprising anti-TNFR2 antigen-binding protein (e.g., antibody such as single-domain antibody), fusion protein, or conjugate of the present technology, at least one polynucleotide molecule encoding the same, at least one vector comprising such a polynucleotide molecule, or at least one host cell comprising the polynucleotide molecule or vector. The composition may be a pharmaceutical composition. The composition may further comprise at least one pharmaceutically acceptable carrier, diluent, or excipient and/or adjuvant, and optionally comprise one or more further pharmaceutically active polypeptides and/or compounds.

As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. Supplementary active compounds can also be incorporated into the compositions.

Examples of suitable formulations include, but are not limited to, solutions, suspensions, powders, pastes, ointments, jellies, waxes, oils, lipids, lipid (cationic or anionic) containing vesicles (such as LIPOFECTIN™, Life Technologies, Carlsbad, CA), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Phdomain Sci Technol 52:238-311.

A pharmaceutical composition of the present disclosure may be formulated according to its intended route of administration. Examples of suitable routes of administration include, e.g., Intravenous, subcutaneous, intratumoral, oral (e.g., buccal, sublingual), intranasal, inhalation, intraocular, intramuscular, intradermal, transdermal (i.e., topical), intraperitoneal, transmucosal, vaginal, and rectal administration, or injection to the CNS/brain (e.g., intraspinal, intracerebral, or intrathecal administration). Solutions or suspensions used for parenteral, intradermal, or subcutaneous application can include the following components: a sterile diluent such as water for injection, saline solution, polyethylene glycols, glycerine, propylene glycol or other synthetic solvents; antibacterial agents such as benzyl alcohol or methyl parabens; antioxidants such as ascorbic acid or sodium bisulfite; fixed oils; chelating agents such as ethylenediaminetetraacetic acid (EDTA); buffers such as phosphates, acetates, or citrates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of plastic or glass.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include, for example, physiological saline, bacteriostatic water, Cremophor EL®, or phosphate buffered saline (PBS). The composition is preferably sterile and has a proper fluidity. In most embodiments, the composition is stable under the conditions of manufacture and storage and can be preserved against the contaminating action of microorganisms such as bacteria and fungi. The carrier can be a solvent or dispersion medium containing, e.g., water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the contamination by microorganisms can be achieved by the inclusion of various antibacterial and antifungal agents, for example, parabens, chlorobutanol, phenol, ascorbic acid, thimerosal, and the like. In many cases, it is preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients described above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and/or freeze-drying that yields a powder of the active ingredient plus any additional desired ingredient from a previously sterile-filtered solution thereof.

Oral compositions may include an inert diluent or an edible carrier. They can be enclosed in gelatin capsules or compressed into tablets. For the purpose of oral therapeutic administration, the active compound can be incorporated with excipients and used in the form of tablets, troches, capsules, or liquid forms. Formulation in tablet and liquid forms may be used for protease insensitive VHHs. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation, Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

For brain delivery, compounds of the present disclosure may be formulated to facilitate crossing of the blood-brain barrier. For example, anti-TNFR2 antigen-binding proteins (e.g., antibody such as single-domain antibody), fusion proteins, or conjugates of the present disclosure may be encapsulated into brain targeted liposomes, lipid nanoparticles, lipid microparticles, or lipid microcapsules for brain delivery. Example liposomes delivery systems are described in Pothin et al., Pharmaceutics 2020, 12 (10), 937, which is incorporated herein by reference in its entirety.

In some embodiments, the active compounds are prepared with carriers that can protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Liposomal suspensions can also be used as pharmaceutically acceptable carriers. These can be prepared according to methods known to those skilled in the art, for example, as described in U.S. Pat. No. 4,522,811, which is incorporated herein by reference in its entirety.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure is dependent on the unique characteristics of the active compound and the particular therapeutic effect to be achieved, and the limitations inherent in the art of compounding such an active compound for the treatment of individuals.

The pharmaceutical compositions (or components thereof) can be included in a kit, container, pack, or dispenser together with instructions for administration. These pharmaceutical compositions can be included in diagnostic kits with instructions for use.

Pharmaceutical compositions are administered in an amount effective for treatment or prophylaxis of the specific indication. The therapeutically effective amount is typically dependent on the weight of the subject being treated, the physical or health condition of the subject, the extensiveness of the condition to be treated, or the age of the subject being treated. In some embodiments, the pharmaceutical composition may be administered in an amount in the range of about 50 μg/kg body weight to about 50 mg/kg body weight per dose. In some embodiments, the pharmaceutical composition may be administered in an amount in the range of about 100 μg/kg body weight to about 50 mg/kg body weight per dose. In some embodiments, the pharmaceutical composition may be administered in an amount in the range of about 100 μg/kg body weight to about 20 mg/kg body weight per dose. In some embodiments, the pharmaceutical composition may be administered in an amount in the range of about 0.5 mg/kg body weight to about 20 mg/kg body weight per dose. Depending on the severity of the condition, the frequency and the duration of the treatment can be adjusted. Effective dosages and schedules for administering a pharmaceutical composition of the present disclosure may be determined empirically; for example, patient progress can be monitored by periodic assessment, and the dose adjusted accordingly. Moreover, interspecies scaling of dosages can be performed using well-known methods in the art (e.g., Mordenti et al., 1991, Phdomainaceut. Res. 8:1351).

In some embodiments, the pharmaceutical composition may be administered in an amount in the range of about 10 mg to about 1,000 mg per dose. In some embodiments, the pharmaceutical composition may be administered in an amount in the range of about 20 mg to about 500 mg per dose. In some embodiments, the pharmaceutical composition may be administered in an amount in the range of about 20 mg to about 300 mg per dose. In some embodiments, the pharmaceutical composition may be administered in an amount in the range of about 20 mg to about 200 mg per dose.

In some embodiments wherein the antigen-binding proteins of the present disclosure are administered as a viral vector (e.g., an AAV), dose ranges and frequency of administration of the viral vector described herein can vary depending on the nature of the viral vector, and the medical condition, as well as parameters of a specific patient and the route of administration used. In some embodiments, viral vector compositions can be administered to a subject at a dose ranging from about 1×10⁵ plaque forming units (pfu) to about 1×10¹⁵ pfu, depending on mode of administration, the route of administration, the nature of the disease and condition of the subject. In some cases, the viral vector compositions can be administered at a dose ranging from about 1×10⁸ pfu to about 1×10¹⁵ pfu, or from about 1×10¹⁰ pfu to about 1×10¹⁵ pfu, or from about 1×10⁸ pfu to about 1×10¹² pfu. A more accurate dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, for example, a juvenile human subject can receive from about 1×10⁸ pfu to about 1×10¹⁰ pfu, while an adult human subject can receive a dose from about 1×10¹⁰ pfu to about 1×10¹² pfu.

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, Intravenous, subcutaneous, intranasal, intraocular, epidural, intraspinal, intracerebral, intrathecal, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous, intramuscular, intratumoral, intraperitoneal, intraspinal, intracerebral, and intrathecal injections, drip infusions, etc. In one embodiment, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the antigen-binding proteins described herein may be about 5 to about 500 mg per dosage form in a unit dose; especially in the form of injection, the antigen-binding proteins described herein may be contained in about 5 to about 100 mg and in about 10 to about 250 mg for the other dosage forms.

The pharmaceutical composition may be administered as needed to a subject. In some embodiments, an effective dose of the pharmaceutical composition is administered to a subject one or more times. In various embodiments, an effective dose of the pharmaceutical composition is administered to the subject once a month, less than once a month, such as, for example, every two months, every three months, or every six months. In other embodiments, an effective dose of the pharmaceutical composition is administered more than once a month, such as, for example, every two weeks, every week, twice per week, three times per week, daily, or multiple times per day. An effective dose of the pharmaceutical composition is administered to the subject at least once. In some embodiments, the effective dose of the pharmaceutical composition may be administered multiple times, including for periods of at least a month, at least six months, or at least a year. In some embodiments, the pharmaceutical composition is administered to a subject as needed to alleviate one or more symptoms of a condition.

In some embodiments, a pharmaceutical composition of the present disclosure may be administered to a subject at levels lower than that required to achieve the desired therapeutic effect and the dosage may be gradually increased until the desired effect is achieved. Alternatively, a pharmaceutical composition of the present disclosure may be administered at a high dose and subsequently administered progressively lower doses until a therapeutic effect is achieved. In general, a suitable daily dose of an antigen-binding protein of the invention is an amount of the antibody which is the lowest dose effective to produce a therapeutic effect.

Pharmaceutical compositions of the present disclosure may optionally include more than one active agent. For example, compositions of the present disclosure may contain an anti-TNFR2 antigen-binding protein conjugated to, admixed with, or administered separately from another pharmaceutically active molecule, e.g., Treg cell, or an additional agent that is useful for induction of Treg cell expansion. For instance, an anti-TNFR2 antigen-binding protein may be admixed with one or more additional active agents, such as IL-2 or TNFα, to treat an immunological disease, e.g., a disorder described herein. Alternatively, pharmaceutical compositions of the present disclosure may be formulated for co-administration or sequential administration with one or more additional active agents that can be used to attenuate CD8+ T-cell growth. Examples of additional active agents that can be used to attenuate cytotoxic T-cell proliferation and that can be conjugated to, admixed with, or administered separately from an anti-TNFR2 antigen-binding protein of the present disclosure include cytotoxic agents, e.g., those described herein.

### Treatment Methods and Other Uses

In one aspect, provided herein is a method of using anti-TNFR2 antigen-binding proteins, fusion proteins, or conjugates of the present disclosure to stimulate the proliferation of a population of regulatory T (Treg) cells (e.g., CD4+, CD25+, FOXP3+ Treg cells). This response may also have the effect of reducing populations of cytotoxic T-lymphocytes (e.g., CD8+ T-cells) that are often associated with mounting an inappropriate immune response that can cause an immunological disorder. In addition, anti-TNFR2 antigen-binding proteins, fusion proteins or conjugates of the present disclosure may synergize with existing Treg proliferating agents, such as IL-2 and TNFα.

Also provided herein is a method of using anti-TNFR2 antigen-binding proteins, fusion proteins, or conjugates of the present disclosure to activate and/or enhance suppressive function (e.g. inhibition of effector T/B cell function or proliferation or antigen presenting cell function) of a population of Treg cells.

Further provided herein is a method of using anti-TNFR2 antigen-binding proteins, fusion proteins, or conjugates of the present disclosure to stabilize immunosuppressive phenotype (including stable expression of e.g., FOXP3, HELIOS, CTLA-4) of a population of Treg cells.

In various embodiments of the above methods, the methods may comprise contacting the population of regulatory T cells with an anti-TNFR2 antigen-binding protein, fusion protein or conjugate described herein. The methods may be carried out in vitro or in vivo. When such methods are carried out in vivo, the methods further comprise administering the anti-TNFR2 antigen-binding protein, fusion protein or conjugate described herein into a subject.

Tregs are a subset of T cells that play a crucial role in peripheral self-tolerance and the prevention of autoimmunity. Historically, Tregs have been identified as a CD4 subset that specifically express CD25, the high affinity IL-2 receptor alpha chain (Sakaguchi et al., 1995). Subsequently, FOXP3 transcription factor was identified as CD4 Treg's master regulator (Hori et al., 2003). In fact, FOXP3 deficiency leads to systemic autoimmunity in both mouse and human in which it causes the Immunodysregulation polyendocrinopathy enteropathy X-linked (IPEX) syndrome due to Tregs deficiency and unregulated effector T cell function (Bennett et al., 2001). CD4 Tregs can differentiate during T cell development (thymic “tTregs”) or in the periphery (peripheral “pTregs”) under non-inflammatory T cell receptor stimulation (Wing et al., 2019). Numerous subsets have been described including naïve and memory Tregs (Sakaguchi et al., 2020), Th-like Tregs (Halim et al., 2017) as well as CD8 Tregs (Mishra et al., 2021; Niederlova et al., 2021). CD4 Tregs regulate immune response through various mechanisms including the secretion of regulatory cytokines (e.g., IL-10, IL-35, TGF-β), IL-2 scavenging, adenosine production, direct cytotoxicity, and dendritic cell regulation (Vignali et al., 2008). The terms “regulatory T cells” or “Treg” as used herein are meant to encompass all the above-described subsets of regulatory T cells.

Tregs have enhanced affinity for MHC II-presented self-antigen peptide and have a TCR repertoire that is non-overlapping with effector CD4 T cells (Fazilleau et al., 2007; Hsieh et al., 2006; Pacholczyk et al., 2006). Therefore, self-antigen recognition in the periphery can induce tTregs activation (Moran et al., 2011). Importantly however, once activated, Tregs can suppress effector cells that have different antigen specificity through bystander suppression (Thornton and Shevach, 2000; Yeh et al., 2017; Yu et al., 2005) by regulating antigen presenting cells or soluble factors.

It has been shown that over time, Tregs retain some plasticity and can lose FOXP3 expression. These so called “ex-Tregs” have increased level of FOXP3 promoter methylation and lower FOXP3 expression compared to Tregs and can acquire effector function (Zhou et al., 2009). In Tregs, the demethylation of FOXP3 promoter, particularly in the “Treg-specific demethylated region” (TSDR) (Huehn et al., 2009), stabilizes gene expression. Likewise, human Tregs exposed to IL-2+ inflammatory cytokines have been shown to lose FOXP3 expression while upregulating RORg and IL-17, a feature associated with TH17 cells. Instability of the Treg phenotype in the presence of inflammatory cytokines can be referred to as “Treg fragility” and is of crucial relevance for therapeutic purpose in autoimmune diseases. Indeed, to induce a long-lasting therapeutic benefit, it is important to stabilize the phenotype and function of Tregs and prevent their conversion to pathogenic cells that further contribute to disease.

In some embodiments, anti-TNFR2 antigen-binding proteins, fusion proteins or conjugates of the present disclosure may be capable of stimulating the proliferation of a population of Treg cells by between 1% and 100% relative to untreated cells (e.g., about 1%, 2%, 3%, 4%, 5%, 6%, 7%, 8%, 9%, 10%, 15%, 20%, 30%, 40%, 50%, 60%, 70%, 80%, 90%, or 100%), as measured, e.g., by fluorescence activated cell sorting (FACS) analysis. In certain embodiments, anti-TNFR2 antigen-binding proteins, fusion proteins or conjugates of the present disclosure may be capable of reducing the growth of a population of CD8+ T-cells, e.g., by about 10% to about 200% relative to untreated cells (e.g., 10%, 20%, 30%, 40%, 50%, 75%, 100%, 125%, 150%, 175%, or 200%).

In some embodiments, anti-TNFR2 antigen-binding proteins of the present disclosure can be used to promote the proliferation of a population of Treg cells and thus enhance the immunomodulatory activity of these cells. Anti-TNFR2 antigen-binding proteins of the present disclosure can therefore be used to attenuate an aberrant cell-mediated or humoral immune response associated with a variety of human diseases, such as autoimmune disorders, asthma, allergic reactions, and diseases associated with allograft tolerance. For example, anti-TNFR2 antigen-binding proteins of the present disclosure may be administered to suppress cytotoxic T-cell and B-cell activity, thereby attenuating the response of a subject to a self or benign antigen. Anti-TNFR2 antigen-binding proteins of the present disclosure can be administered to a mammalian subject, such as a human, to attenuate an aberrant immune response, such as a response against a self or non-threatening antigen. Alternatively, anti-TNFR2 antigen-binding proteins of the present disclosure can be used to expand a population of Treg cells ex vivo that have been extracted, e.g., from a patient or an MHC-matched donor. After inducing proliferation of these Treg cells in culture by contacting with an anti-TNFR2 antigen-binding protein of the present disclosure, these cells can subsequently be administered to a subject, e.g., using adoptive cell transfer techniques known in the art or described herein. In this way, anti-TNFR2 antigen-binding proteins of the present disclosure may synergize with existing techniques to suppress humoral and cell-mediated immune responses as a treatment modality for patients suffering from a variety of immunological disorders.

In some embodiments, anti-TNFR2 antigen-binding proteins of the present disclosure are capable of interacting with and promoting signal transduction events mediated by TNFR2, Anti-TNFR2 antigen-binding proteins of the present disclosure may be able to induce conformational changes within TNFR2 that lead to receptor trimerization. This spatial configuration has been shown to render TNFR2 active for MAPK/TRAF 2/3 signal transduction, which subsequently leads to activation of NF-κB-mediated transcription of genes involved in Treg cell growth and escape from apoptosis (Faustman, et al., Nat Rev Drug Discov. 9:482-493 (2010), the disclosure of which is incorporated herein by reference).

In some embodiments, anti-TNFR2 antigen-binding proteins of the present disclosure may be capable of increasing the transcription and/or expression of various genes. For example, anti-TNFR2 antigen-binding proteins of the present disclosure may induce the expression of one or more of Akt, clAP2, Etk, TRAF2, VEGFR2, P13K, genes encoding proteins involved in the angiogenic pathway, IKK complexes, RIP, NIK, MAP3K, genes encoding proteins involved in the NF-κB pathway, NIK, JNK, AP-1, a MEK (e.g., MEK1, MEK7), MKK3, NEMO, IL2R, Foxp3, IL2, TNF, and lymphotoxin (e.g., lymphotoxin a and lymphotoxin β). The increase in expression of these genes can be detected using established molecular biology techniques known in the art, e.g., by detecting an increase in mRNA levels by Northern blot analysis or reverse-transcription PCT (RT-PCR) methods, or by detecting an increase in protein levels by immunoblot analysis or ELISA-based techniques. In some embodiments, anti-TNFR2 antigen-binding proteins of the present disclosure may be capable of promoting the activity of one or more proteins associated with the TNFR2 signaling pathway (or related signaling pathways that are activated as a result of TNFR2 signaling). For instance, anti-TNFR2 antigen-binding proteins of the present disclosure may be capable of promoting an increase in the phosphorylation of one or more proteins, such as Akt, clAP2, Etk, TRAF2, VEGFR2, P13K, proteins involved in the angiogenic pathway, IKK complexes, RIP, NIK, MAP3K, proteins involved in the NF-κB pathway, NIK, JNK, AP-1, a MEK (e.g., MEK1, MEK7), MKK3, NEMO, IL2R, Foxp3, IL2, TNF, and lymphotoxin (e.g., lymphotoxin α and lymphotoxin β). An increase in the phosphorylation of one or more proteins that occurs, e.g., as a result of treatment of a subject or of a sample of cells isolated from a subject can be detected using standard molecular biology techniques known in the art, such as by immunoblot analysis or ELISA-based techniques.

In some embodiments, antigen-binding proteins of the present disclosure increases expression of one or more proteins selected from a protein in the NF-κB pathway, FOXP3, HELIOS, EZH2, HLA-DR, ICAM-1, OX-40, ICOS, and CCR8.

In another aspect, provided herein is a method of inhibiting an immune response mediated by a B cell or a CD8+ T cell in a subject, the methods including the step of administering to the subject an anti-TNFR2 antigen-binding protein (e.g., antibody such as single-domain antibody), a fusion protein, a conjugate, a polynucleotide molecule, a vector, or a host cell described herein.

In another aspect, anti-TNFR2 antigen-binding proteins (e.g., antibody such as single-domain antibody), fusion proteins, conjugates, polynucleotide molecules, vectors, and/or host cells described herein, or pharmaceutical compositions thereof, are useful for the (prophylactic or therapeutic) treatment of a wide array of diseases or disorders. Accordingly, the present technology provides an anti-TNFR2 antigen-binding protein (e.g., antibody such as single-domain antibody), a fusion protein, a conjugate, a polynucleotide molecule, a vector, or a host cell for use as a medicament. Also provided is a (prophylactic and/or therapeutic) method of treating a disease or disorder, wherein said method comprises administering, to a subject in need thereof, a pharmaceutically active amount of an anti-TNFR2 antigen-binding protein (e.g., antibody such as single-domain antibody), a fusion protein, a conjugate, a polynucleotide molecule, a vector, or a host cell described herein.

The diseases or disorder that can be treated with the compositions and methods described herein include, but are not limited to, immunological diseases (e.g., autoimmune diseases), inflammatory diseases, cancers, and cardiovascular diseases (e.g. atherosclerosis, heart failure, left heart failure with reduced ejection fraction, left heart failure with preserved ejection fraction, right ventricular failure, congestive heart failure, restrictive cardiomyopathy, dilated cardiomyopathy, hypertrophic cardiomyopathy, ischemic cardiomyopathy, idiopathic cardiomyopathy, hypertension) Infertility and pregnancy-associated diseases (e.g. recurrent pregnancy loss, pre-eclampsia, preterm labor, fetal growth restriction, intrauterine growth restriction).

Examples of immunological diseases that can be treated with the compositions and methods described herein include, but are not limited to, autoimmune diseases, allergies, asthma, neurological diseases, metabolic diseases (e.g., diabetes), macular diseases (e.g., macular degeneration), muscular atrophy, diseases related to miscarriage, vascular diseases (e.g., atherosclerosis), diseases related to bone loss (e.g., bone loss as a result of menopause or osteoporosis), blood disorders (e.g., hemophilia), musculoskeletal disorders, diseases related to growth receptor expression or activity, obesity, graft-versus-host disease (GVHD), or allograft rejections.

In some embodiments, the compositions and methods described herein are used to treat an autoimmune disease. In some embodiments, the autoimmune disease is selected from lupus, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, essential mixed cryoglobulinemia, fibromyalgia-fibromyositis, Goodpastures disease, Graves' disease, Guillain-Barré, Hashimoto's thyroiditis, hypothyroidism, idiopathic pulmonary fibrosis, idiopathic thrombocytopenia purpura (ITP), IgA nephropathy, juvenile arthritis, lichen planus, lichen sclerosis, IgG4-related disease, Meniere's disease, mixed connective tissue disease, multiple sclerosis, myasthenia gravis, neuromyelitis optica spectrum disease, pemphigus vulgaris or related blistering skin disease, pernicious anemia, polyarteritis nodosa, polychondritis, polyglandular syndromes, polymyalgia rheumatica, polymyositis and dermatomyositis, premature ovarian failure, primary agammaglobulinemia, primary biliary cirrhosis, psoriasis, primary ovarian insufficiency, Raynaud's phenomenon, Reiter's syndrome, rheumatic fever, rheumatoid arthritis, sarcoidosis, scleroderma, Sjögren's syndrome, spondyloarthritis, stiff-man syndrome, type I diabetes, Takayasu arteritis, temporal arteritis/giant cell arteritis, ulcerative colitis, uveitis, vasculitis, vitiligo, and Wegener's granulomatosis (Granulomatosis with polyangiitis) or other immune vasculitis.

In some embodiments, the compositions and methods described herein are used to treat lupus. In some embodiments, the lupus is systemic lupus erythematosus (SLE), cutaneous lupus (including acute cutaneous lupus, chronic cutaneous lupus erythematosus, or discoid lupus erythematosus (DLE) and subacute cutaneous lupus erythematosus), lupus nephritis, neonatal lupus, or drug-induced lupus.

In some embodiments, the compositions and methods described herein are used to treat allergy. In some embodiments, the allergy is an allergic conjunctivitis, chemical allergy, cosmetic allergy, drug allergy, dust allergy, food allergy, hay fever, hives, mold allergy, pet allergy, poison ivy allergy oak allergy, or seasonal allergy.

In some embodiments, the compositions and methods described herein are used to treat a neurological condition. In some embodiments, the neurological condition is a brain tumor, a brain metastasis, a spinal cord injury, schizophrenia, epilepsy, Amyotrophic lateral sclerosis (ALS), Alzheimer's disease, Huntington's disease, Parkinson's disease, or stroke.

In some embodiments, the compositions and methods described herein are used to treat a graft rejection. Without wishing to be bound by theory, anti-TNFR2 antigen-binding proteins of the present disclosure may treat graft rejections, e.g., by binding TNFR2 receptors on the surface of autoreactive CD8+ T-cells that bind antigens presented on the surface of the graft and inducing apoptosis in these CD8+ T-cells, or by inducing the expansion of Treg cells that may subsequently eliminate autoreactive CD8+ T-cells. Examples of graft rejections that can be treated with the compositions and methods described herein include, without limitation, skin graft rejection, bone graft rejection, vascular tissue graft rejection, ligament graft rejection (e.g., anterior cruciate ligament graft rejection, anterior sacroiliac ligament graft rejection, caudal cruciate ligament graft rejection, cranial cruciate ligament graft rejection, cricothyroid ligament graft rejection, dorsal radiocarpal ligament graft rejection, inferior pubic ligament graft rejection, lateral collateral ligament graft rejection, medial collateral ligament graft rejection, palmar radiocarpal ligament graft rejection, patellar ligament graft rejection, periodontal ligament graft rejection, posterior cruciate ligament graft rejection, posterior sacroiliac ligament graft rejection, radial collateral ligament graft rejection, sacrospinous ligament graft rejection, sacrotuberous ligament graft rejection, superior pubic ligament graft rejection, suspensory ligament of the breast graft rejection, suspensory ligament of the lens graft rejection, ulnar collateral ligament graft rejection) and organ graft rejection (e.g., heart, lung, kidney, liver, pancreas, intestine, and thymus graft rejection).

In some embodiments, the compositions and methods described herein are used to treat a graft-versus-host disease. In some embodiments, the graft-versus-host disease arises from a bone marrow transplant or one or more blood cells such as B-cells, T-cells, basophils, common myeloid progenitor cells, common lymphoid progenitor cells, dendritic cells, eosinophils, hematopoietic stem cells, neutrophils, natural killer cells, megakaryocytes, monocytes, or macrophages.

In some embodiments, the compositions and methods described herein are used to treat an inflammatory disease. The inflammatory disease may be acute or chronic inflammation. In some embodiments, the inflammatory disease is selected from osteoarthritis, atopic dermatitis, endometriosis, polycystic ovarian syndrome, inflammatory bowel disease, fibrotic lung disease, and cardiac inflammation.

In some embodiments, the compositions and methods described herein are used to treat a cancer. In some embodiments, the cancer is an adenoid cystic carcinoma, adrenal gland tumor, amyloidosis, anal cancer, appendix cancer, astrocytoma, ataxia-telangiectasia, Beckwith-Wiedemann syndrome, bile duct cancer (cholangiocarcinoma), Birt-Hogg-Dubé syndrome, bladder cancer, bone cancer (sarcoma of bone), brain stem glioma, brain tumor, breast cancer, inflammatory breast cancer, metastatic breast cancer, male breast cancer, Carney complex, central nervous system tumors (brain and spinal cord), cervical cancer, childhood cancer, colorectal cancer, Cowden syndrome, craniopharyngioma, desmoid tumor, desmoplastic infantile ganglioglioma, childhood tumor, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor (GIST), germ cell tumor (including childhood germ cell tumor), gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS-related cancer, juvenile polyposis syndrome, kidney cancer, lacrimal gland tumor, laryngeal and hypopharyngeal cancer, acute lymphoblastic leukemia (ALL), acute myeloid leukemia (AML), B-cell prolymphocytic leukemia and hairy cell leukemia, chronic lymphocytic leukemia (CLL), chronic myeloid leukemia (CML), chronic T-cell lymphocytic leukemia, eosinophilic leukemia, Li-Fraumeni syndrome, liver cancer, lung cancer (e.g., non-small cell lung cancer, small cell lung cancer), hodgkin lymphoma, non-hodgkin lymphoma, lynch syndrome, mastocytosis, medulloblastoma (including childhood medulloblastoma), melanoma, meningioma, mesothelioma, multiple endocrine neoplasia type 1, multiple endocrine neoplasia type 2, multiple myeloma, MUTYH (or MYH)-associated polyposis, myelodysplastic syndromes (MDS), nasal cavity and paranasal sinus cancer, nasopharyngeal cancer, neuroblastoma (including childhood neuroblastoma), neuroendocrine tumor of the gastrointestinal tract, neuroendocrine tumor of the lung, neuroendocrine tumor of the pancreas, neuroendocrine tumors, neurofibromatosis type 1, neurofibromatosis type 2, nevoid basal cell carcinoma syndrome, oral and oropharyngeal cancer, osteosarcoma, ovarian, fallopian tube, and peritoneal cancer, pancreatic cancer, parathyroid cancer, penile cancer, Peutz-Jeghers syndrome, pheochromocytoma and paraganglioma, pituitary gland tumor, pleuropulmonary blastoma, prostate cancer, retinoblastoma, rhabdomyosarcoma, salivary gland cancer, Kaposi sarcoma, soft tissue sarcomas, skin cancer (non-melanoma), small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom macroglobulinemia (lymphoplasmacytic lymphoma), Werner syndrome, Wilms tumor, or xeroderma pigmentosum.

In some embodiments, anti-TNFR2 antigen-binding proteins of the present disclosure can also be used to treat a patient in need of organ repair or regeneration, e.g., by inducing the proliferation of cells within a damaged tissue or organ. While not wishing to be bound by any theory, it is contemplated that agonistic TNFR2 antibodies may stimulate organ repair or regeneration, e.g., by binding TNFR2 on the surface of cells within damaged tissue to induce TRAF2/3- and/or NF-κB-mediated cell proliferation. Examples of tissues and organs that may be induced to regenerate by the use of anti-TNFR2 antigen-binding proteins of the present disclosure include the blood vessels including the aorta, bone, cranial nerves, ear, eye, embryonic structures, heart, heart, hematopoietic system, kidney, small intestine, large intestine, liver, lung, nerves, olfactory gland, pancreas, pituitary gland, peripheral nervous system, central nervous system, spinal cord, salivary gland, structures of the head, testes, thymus, and tongue.

Additional diseases that can be treated with the compositions and methods of the present disclosure include genetic diseases with an immunological phenotype. Exemplary genetic diseases with an immunological phenotype are described in, e.g., Table S2 of Tangye et al., Journal of Clinical Immunology volume 42, pages 1473-1507 (2022), which is incorporated herein by reference in its entirety.

In some embodiments, patients receiving an anti-TNFR2 treatment of the present disclosure can be monitored for their responsiveness to the treatment. For example, a physician may monitor the response of a mammalian subject (e.g., a human) to treatment with anti-TNFR2 antigen-binding proteins of the present disclosure by analyzing the quantity of IFNγ secreted by CD8+ T-cells within a particular patient. For example, a composition of the present disclosure may be capable of reducing IFNγ secretion by between 1% and 100% (e.g., 1%, 5%, 10%, 20%, 30%, 40%, 50%, 60%, 70%, 80%, 90%, 95%, 99%, or 100%). Alternatively, a physician may monitor the responsiveness of a subject (e.g., a human) to treatment with a composition of the present disclosure by analyzing the Treg cell population in the lymph of a particular subject. For example, a physician may withdraw a sample of blood from a mammalian subject (e.g., a human) and determine the quantity or density of a population of Treg cells (e.g., CD4+ CD25+ FOXP3+ Treg cells or CD17+ Treg cells) using established procedures, such as FACS analysis. In such embodiments, high counts of Treg cells can be indicative of efficacious therapy, while lower Treg cell counts may indicate that the patient is to be prescribed or administered higher dosages of the anti-TNFR2 antigen-binding protein of the present disclosure until, e.g., an ideal Treg cell count is achieved. In addition, a physician of skill in the art may monitor the effect of treatment by administration of a composition of the present disclosure to a subject suffering from an immunological disorder, such as an autoimmune disease described herein, by analyzing the quantity of autoreactive CD8+ T-cells within a lymph sample isolated from the patient. Anti-TNFR2 antigen-binding proteins of the invention may attenuate the proliferation of autoreactive T-cells, e.g., by binding TNFR2 at the surface of an autoreactive T-cell and inducing apoptosis, and/or by stimulating the expansion of Treg cells that subsequently eliminate autoreactive T lymphocytes. Treatment with anti-TNFR2 antigen-binding proteins may lead to reduced quantities of autoreactive T-cells within the lymph isolated from a patient receiving treatment, and a rapid decline in the population of autoreactive T-cells in a lymph sample isolated from such a patient may indicate effective treatment. In cases where a lymph sample isolated from a patient exhibits an autoreactive T-cell count that has not declined in response to agonistic TNFR2 antibody therapy, a physician may prescribe the patient higher doses of the antibody or an antigen-binding fragment thereof or may administer the anti-TNFR2 antigen-binding proteins with higher frequency, e.g., multiple times per day, week, or month.

Anti-TNFR2 antigen-binding proteins described herein may be administered as a monotherapy or in combination with one or more additional therapeutic agents.

In some embodiments, anti-TNFR2 antigen-binding proteins of the present disclosure may also be admixed, conjugated, or administered with, or administered separately from, another agent that promotes Treg cell proliferation. Additional agents that can be used to promote Treg cell expansion include, e.g., IL-2 and TNFα, the cognate ligand for TNFR2.

In some embodiments, pharmaceutical compositions of the invention may be formulated for co-administration or sequential administration with one or more additional active agents that can be used to inhibit CD8+ T-cell growth. Examples of additional active agents that can be used to inhibit cytotoxic T-cell proliferation and that can be conjugated to, admixed with, or administered separately from an anti-TNFR2 antigen-binding protein of the present disclosure include cytotoxic agents, e.g., those described herein.

Exemplary cytotoxic agents that can be conjugated to, admixed with, or administered separately from anti-TNFR2 antigen-binding protein of the present disclosure include, but not limited to, 13-cis retinoic acid, 14-hydroxy-retro-retinol, 2-chloro-2′-deoxyadenosine, 2-Chloro-2′-arabino-fluoro-2′-deoxyadenosine, 2-chlorodeoxyadenosine, 2-chlorodeoxyadenosine (2-Cda), 2′-deoxycoformycin, 3-methyl TTNEB, 6-mercaptopurine, 6-thioguanine, 9-aminocamptothecin, 9-cis retinoic acid, aclarubicin, acodazole hydrochloride, acronine, adozelesin, adozelesin, adriamycin, aldesleukin, all-trans retinoic acid, all-trans retinol, altretamine, ambomycin, ametantrone acetate, aminoglutethimide, amsacrine, amsacrine, anastrozole, anisomycin, anthramycin, acivicin, asparaginase, asperlin, azacitidine, azacitidine, azetepa, azotomycin, AZQ, batimastat, benzodepa, bicalutamide, Bis (platinum), bisantrene hydrochloride, bisnafide dimesylate, bizelesin, bleomycin sulfate, brequinar sodium, bropirimine, busulfan, busulfan, cactinomycin, calusterone, camptothecin, caracemide, carbetimer, carboplatin, carboplatin, carmustine, carubicin hydrochloride, carzelesin, cedefingol, CEP-751, chlorambucil, chlorambucil, cirolemycin, cisplatin, cisplatin, cladribine, combretestatin a-4, C1-973, CPT-11, crisnatol mesylate, cyclophosphamide, cyclophosphamide, cytarabine, cytarabine, daca (n-[2-(dimethyl-amino) ethyl] acridine-4-carboxamide), dacarbazine, dactinomycin, Dactinomycin (Actinomycin D), darubicin, daunomycin, Daunomycin, daunorubicin hydrochloride, decitabine, dexormaplatin, dezaguanine, dezaguanine mesylate, diacarbazine (DTIC), diaziquone, docetaxel, dolasatins, doxorubicin, Doxorubicin, doxorubicin hydrochloride, droloxifene, droloxifene citrate, dromostanolone propionate, duazomycin, DWA 2114R, edatrexate, eflornithine hydrochloride, ellipticine, elsamitrucin, enloplatin, enpromate, epipropidine, Epirubicin, epirubicin hydrochloride, erbulozole, esorubicin hydrochloride, estramustine, estramustine phosphate sodium, etanidazole, ethiodized oil i 131, etoposide, etoposide phosphate, etoprine, fadrozole hydrochloride, fazarabine, fenretinide, floxuridine, fludarabine (2-F-ara-AMP), fludarabine phosphate, fluorodeoxyuridylate, fluorouracil, flurocitabine, fosquidone, fostriecin sodium, gemcitabine, gemcitabine, gemcitabine hydrochloride, gold ¹⁹⁸AU, homocamptothecin, hPRL-G129R, hydroxyurea, hypoxanthine, idarubicin hydrochloride, ifosfamide, ifosfamide, ilmofosine, interferon γ-1b, Interferon α-2b, interferon α-n1, interferon α-n3, interferon α-2a, interferon β-1a, iproplatin, irinotecan hydrochloride, JM216, JM335, lanreotide acetate, letrozole, leuprolide acetate, liarozole hydrochloride, linomide, lometrexol sodium, lomustine, losoxantrone, losoxantrone hydrochloride, masoprocol, maytansine, mechlorethamine hydrochloride, megestrol acetate, melengestrol acetate, melphalan, melphalan, menogaril, mercaptopurine, methotrexate, methotrexate sodium, metoprine, meturedepa, mitindomide, mitocarcin, mitocromin, mitogillin, mitomalcin, mitomycin, mitomycin C, mitosper, mitotane, mitoxantrone, mitoxantrone hydrochloride, mitozolomide, mycophenolic acid, N-(2-chloroethyl)-N′ cyclohexyl-N-nitrosourea (CCNU), N-(2-chloroethyl)-N′-(diethyl)ethylphosphonate-N-nitrosourea (fotemustine), N-(2-chloroethyl)-N′-(trans-4-methylcyclohexyl-N-nitrosourea (MeCCNU), N-(4-hydroxyphenyl) retinamide, N,N′-Bis (2-chloroethyl)-N-nitrosourea (BCNU), nitrogen mustard (mechlor ethamine), N-methyl-Nnitrosourea (MNU), nocodazole, nogalamycin; ormaplatin, N-propargyl-5,8-dideazafolic acid, ormaplatin, oxaliplatin, oxisuran, paclitaxel, pegaspargase, peliomycin, pentamustine, peploycinsulfate, perfosfamide, pipobroman, piposulfan, piroxantrone hydrochloride, plicamycin, plomestane, porfimer sodium, porfiromycin, prednimustine, procarbazine hydrochloride, puromycin, puromycin hydrochloride, pyrazofurin, pyrazoloacridine, raltitrexed, rhizoxin, rhizoxin d, riboprine, rogletimide, safingol, safingol hydrochloride, semustine, simtrazene, sparfosate sodium, sparsomycin, spirogermanium hydrochloride, spiromustine, spiroplatin, streptonigrin, streptozocin, streptozotocin, strontium chloride Sr 89, sulfur mustard, sulofenur, talisomycin, taxane, taxoid, tecogalan sodium, tegafur, teloxantrone hydrochloride, temoporfin, temozolomide, teniposide, teniposide 9-amino camptothecin, teroxirone, testolactone, thiamiprine, thioguanine, thiotepa, thiotepa, thymitaq, tiazofurin, tirapazamine, tomudex, tomudex, TOP-53, topotecan, topotecan hydrochloride, toremifene citrate, trestolone acetate, trichostatin A, triciribine phosphate, trimetrexate, trimetrexate glucuronate, triptorelin, tubulozole hydrochloride, uracil mustard, uredepa, vapreotide, verteporfin, vinblastine, vinblastine sulfate, vincristine, vincristine sulfate, vindesine, vindesine sulfate, vinepidine sulfate, vinglycinate sulfate, vinleurosine sulfate, vinorelbine tartrate, vinrosidine sulfate, vinzolidine sulfate, vorozole, zeniplatin, zinostatin, or zorubicin hydrochloride.

Other therapeutic agents that can be conjugated to, admixed with, or administered separately from anti-TNFR2 antigen-binding protein of the present disclosure include, but are not limited to, 2′ deoxycoformycin (DCF), 1,25 dihydroxyvitamin D3, 5-ethynyluracil, 9-dioxamycin, abiraterone, acylfulvene, adecypenol, ALL-TK antagonists, ambamustine, amidox, amifostine, aminolevulinic acid, amrubicin, anagrelide, andrographolide, angiogenesis inhibitors, antagonist D, antagonist G, antarelix, antiandrogen, prostatic carcinoma, anti-dorsalizing morphogenetic protein-1, antiestrogen, antineoplaston, antisense oligonucleotides, aphidicolin glycinate, apoptosis gene modulators, apoptosis regulators, apurinic acid, ara-CDP-DL-PTBA, argininedeaminase, asulacrine, atamestane, atrimustine, axinastatin 1, axinastatin 2, axinastatin 3, azasetron, azatoxin, azatyrosine, baccatin III derivatives, balanol, BCR/ABL antagonists, benzochlorins, benzoylstaurosporine, beta lactam derivatives, beta-alethine, betaclamycin B, betulinic acid, bFGF inhibitor, bisantrene, bisaziridinylspermine, bisnafide, bistratene A, bleomycin A2, bleomycin 82, breflate, budotitane, buthionine sulfoximine, calcipotriol, calphostin C, camptothecin derivatives (e.g., 10-hydroxy-camptothecin), canarypox IL-2, capecitabine, carboxamide-amino-triazole, carboxyamidotriazole, CaRest M3, CARN 700, cartilage derived inhibitor, casein kinase inhibitors (ICOS), castanospermine, cecropin B, cetrorelix, chlorins, chloroquinoxaline sulfonamide, cicaprost, cis-porphyrin, clomifene analogues, clotrimazole, collismycin A, collismycin B, combretastatin A4, combretastatin analogue, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, dehydrodidemnin B, deslorelin, dexifosfamide, dexrazoxane, dexverapamil, didemnin B, didox, diethylnorspermine, dihydro-5-azacytidine, dihydrotaxol, diphenyl spiromustine, discodermolide, docosanol, dolasetron, doxifluridine, dronabinol, duocarmycin SA, ebselen, ecomustine, edelfosine, edrecolomab, eflornithine, elemene, emitefur, epithilones, epothilones (A, R=H; B, R=Me), epristeride, erythrocyte gene therapy, estramustine analogue, estrogen agonists, estrogen antagonists, etoposide 4′-phosphate (etopofos), exemestane, fadrozole, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, glutathione inhibitors, hepsulfam, heregulin, hexamethylene bisacetamide, homoharringtonine (HHT), hypericin, ibandronic acid, idarubicin, idoxifene, idramantone, ifepristone, ilomastat, imidazoacridones, imiquimod, immunostimulant peptides, insulin-like growth factor-1 receptor inhibitor, interferon agonists, interferons, interleukins, iobenguane, iododoxorubicin, ipomeanol, irinotecan, iroplact, irsogladine, isobengazole, isohomohalicondrin B, itasetron, jasplakinolide, kahalalide F, lamellarin-N triacetate, lanreotide, leinamycin, lenograstim, lentinan sulfate, leptolstatin, leukemia inhibiting factor, leukocyte alpha interferon, leuprolide+estrogen+progesterone, leuprorelin, levamisole, liarozole, linear polyamine analogue, lipophilic disaccharide peptide, lipophilic platinum compounds, lissoclinamide 7, lobaplatin, lombricine, lometrexol, lonidamine, lovastatin, loxoribine, lurtotecan, lutetium texaphyrin, lysofylline, lytic peptides, mannostatin A, marimastat, maspin, matrilysin inhibitors, matrix metalloproteinase inhibitors, meterelin, methioninase, metoclopramide, MIF inhibitor, miltefosine, mirimostim, mismatched double stranded RNA, mithracin, mitoguazone, mitolactol, mitomycin analogues, mitonafide, mitotoxin fibroblast growth factor-saporin, mofarotene, molgramostim, monoclonal antibody, human chorionic gonadotrophin, monophosphoryl lipid A+myobacterium cell wall sk, mopidamol, multiple drug resistance gene inhibitor, multiple tumor suppressor 1-based therapy, mustard anticancer agent, mycaperoxide B, mycobacterial cell wall extract, myriaporone, N-acetyldinaline, nafarelin, nagrestip, naloxone+pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, N-substituted benzamides, O6-benzylguanine, octreotide, okicenone, oligonucleotides, onapristone, ondansetron, oracin, oral cytokine inducer, osaterone, oxaliplatin, oxaunomycin, paclitaxel analogues, paclitaxel derivatives, palauamine, palmitoylrhizoxin, pamidronic acid, panaxytriol, panomifene, parabactin, pazelliptine, peldesine, pentosan polysulfate sodium, pentostatin, pentrozole, perflubron, perillyl alcohol, phenazinomycin, phenylacetate, phosphatase inhibitors, picibanil, pilocarpine hydrochloride, pirarubicin, piritrexim, placetin A, placetin B, plasminogen activator inhibitor, platinum complex, platinum compounds, platinum-triamine complex, podophyllotoxin, propyl bis-acridone, prostaglandin J2, proteasome inhibitors, protein A-based immune modulator, protein kinase C inhibitor, protein kinase C inhibitors, microalgal, protein tyrosine phosphatase inhibitors, purine nucleoside phosphorylase inhibitors, purpurins, pyridoxylated hemoglobin polyoxyethylene conjugate, raf antagonists, ramosetron, ras farnesyl protein transferase inhibitors, ras inhibitors, ras-GAP inhibitor, retelliptine demethylated, rhenium Re 186 etidronate, ribozymes, RII retinamide, rnerbarone, rohitukine, romurtide, roquinimex, rubiginone B 1, ruboxyl, saintopin, SarCNU, sarcophytol A, sargramostim, Sdi 1 mimetics, senescence derived inhibitor 1, sense oligonucleotides, signal transduction inhibitors, signal transduction modulators, single chain antigen-binding protein, sizofiran, sobuzoxane, sodium borocaptate, sodium phenylacetate, solverol, somatomedin binding protein, sonermin, sparfosic acid, spicamycin D, splenopentin, spongistatin 1, squalamine, stem cell inhibitor, stem-cell division inhibitors, stipiamide, stromelysin inhibitors, sulfinosine, superactive vasoactive intestinal peptide antagonist, suradista, suramin, swainsonine, synthetic glycosaminoglycans, tallimustine, tamoxifen methiodide, tauromustine, tazarotene, tellurapyrylium, telomerase inhibitors, tetrachlorodecaoxide, tetrazomine, thaliblastine, thalidomide, thiocoraline, thrombopoietin, thrombopoietin mimetic, thymalfasin, thymopoietin receptor agonist, thymotrinan, thyroid stimulating hormone, tin ethyl etiopurpurin, titanocene dichloride, topsentin, toremifene, totipotent stem cell factor, translation inhibitors, tretinoin, triacetyluridine, triciribine, tropisetron, turosteride, tyrosine kinase inhibitors, tyrphostins, UBC inhibitors, ubenimex, urogenital sinus-derived growth inhibitory factor, urokinase receptor antagonists, variolin B, velaresol, veramine, verdins, vinorelbine, vinxaltine, vitaxin, zanoterone, zilascorb, or zinostatin stimalamer.

In some embodiments, anti-TNFR2 antigen-binding proteins of the present disclosure may be admixed, conjugated, or administered with, or administered separately from, an anti-inflammatory agent. Exemplary anti-inflammatory agents useful in conjunction with the compositions and methods of the invention include steroids, colchicine, hydroxychloroquine, sulfasalazine, dapsone, methotrexate, mycophenolate mofetil, azathioprine, cyclosporine, sirolimus, everolimus, azathioprine, leflunomide, mycophenolate, inhibitors of IL-1/IL-2/1L-4/1L5/IL-6/IL-13/IL-17/IL-23/TNF/complement/BAFF/interferon/JAK/CD28/IgE/Integrins/T cell costimulation pathway or B-cell depleting agents.

In some embodiments, anti-TNFR2 antigen-binding proteins of the present disclosure may be admixed, conjugated, or administered with, or administered separately from, an immunotherapy agent. Exemplary immunotherapy agents useful in conjunction with the compositions and methods of the invention include an anti-CTLA-4 agent, an anti-PD-1 agent, an anti-PD-L1 agent, an anti-PD-L2 agent, a TNFα cross-linking agent, a TRAIL cross-linking agent, an anti-CD27 agent, an anti-CD30 agent, an anti-CD40 agent, an anti-4-1BB agent, an anti-GITR agent, an anti-OX40 agent, an anti-TRAILR1 agent, an anti-TRAILR2 agent, an anti-TWEAKR agent, an anti-TL1A agent, an anti-LIGHT agent, an anti-BTLA agent, an anti-LAG3 agent, an anti-Siglecs agent, an anti-ICOS ligand agent, an anti-87-H3 antibody; an anti-B7-H4 agent; an anti-VISTA agent; an anti-TMIGD2 agent; an anti-BTNL2 agent; an anti-CD48 agent; an anti-KIR agent; an anti-LIR agent; an anti-ILT agent; an anti-NKG2D agent; an anti-NKG2A agent; an anti-MICA agent; an anti-MICB agent; an anti-CD244 agent; an anti-CSF1 R agent; an anti-IDO agent; an anti-TGFβ agent; an anti-CD39 agent; an anti-CD73 agent; an anti-CXCR4 agent; an anti-CXCL12 agent; an anti-SIRPA agent; an anti-CD47 agent; an anti-VEGF agent; and an anti-neuropilin agent and, e.g., agents directed toward the immunological targets described in Table 1 of Mahoney et al., Cancer Immunotherapy, 14:561-584 (2015), the disclosure of which is incorporated herein by reference. The immunotherapy agent described herein may be, for example, an antibody, a small molecule, or a chimeric antigen receptor.

In some embodiments, anti-TNFR2 antigen-binding protein of the present disclosure can also be admixed with, co-administered with, or administered separately from Bacillus Calmette-Guérin (BCG), a bacterial strain that has been used to treat a variety of immunological disorders, such as type I diabetes, multiple sclerosis, scleroderma, Sjogren's disease, systemic lupus erythematosus, Grave's disease, hypothyroidism, Crohn's disease, colititis, an autoimmune skin disease, and rheumatoid arthritis, among others. For instance, anti-TNFR2 antigen-binding protein of the present disclosure may be included in a therapeutic regimen in combination with BCG for the treatment of an immunological disorder (e.g., one of those described above, such as type I diabetes or rheumatoid arthritis). The anti-TNFR2 antigen-binding protein may be co-administered with BCG, e.g., by an injection route described herein. Alternatively, the anti-TNFR2 antigen-binding protein may be administered separately from a BCG-containing composition. The use of BCG to treat immunological disorders has been described, e.g., in U.S. Pat. Nos. 6,660,487; and 6,599,710; the disclosures of each of which are incorporated herein by reference in its entirety.

### EXAMPLES

The following examples are provided to further describe some of the embodiments disclosed herein. The examples are intended to illustrate, not to limit, the disclosed embodiments.

### Example 1. Camelid Immunization

Three alpacas were immunized by four subcutaneous injections with recombinant human TNFR2 (10417-H08H, Sino Biologicals) and complete/incomplete Freund's or Gerbu FAMA adjuvant using standard protocols to elicit a humoral immune response that included the generation of antigen-specific conventional and heavy-chain only (VHH) antibodies.

Before the first and after the third injection, serum was prepared from blood samples. Antibody induction was monitored by comparing antigen-specific antibody titers in the sera before and after immunization by enzyme-linked immunosorbent assay (ELISA). Briefly, 96-well Maxisorp plates were coated with human TNFR2 (10417-H08H, Sino Biologicals), blocked, and incubated with diluted serum samples. TNFR2-specific antibodies were bound by alkaline phosphatase-conjugated goat anti-alpaca IgG (H+L) (Jackson ImmunoResearch, Cat. No. 128-055-160) and detected using p-Nitrophenyl Phosphate.

### Example 2. Phage Library Construction

Four to ten days after the fourth injection in accordance with procedures described in Example 1, blood samples were collected, and four to six days after the fourth injection a bone marrow sample was aspirated. Peripheral blood mononuclear cells (PBMCs) were isolated from heparinized blood or bone marrow following density gradient purification with Ficoll-Paque™ Plus. Total RNA was extracted from freshly isolated PBMCs.

To generate VHH immune libraries, total RNA was reverse transcribed to cDNA using random hexamer primers. Conventional and heavy chain IgH cDNA fragments were amplified by polymerase chain reaction (PCR) using primers annealing to the IgH leader sequence region and the CH2 region. The resulting amplicons represented the VHH and VH cDNAs, respectively. The VHH fragment was isolated and used as template for a nested PCR to introduce appropriate endonuclease recognition sites for cloning into the pQ81 phagemid in frame with gene III. Libraries were transformed into electrocompetent E. coli TG1 cells. In total, six libraries were built, with 95.5% to 100% VHH insert frequency and maximum library sizes between 4.2×10⁸ and 2.4×10⁹. Phage for phage display was prepared following standard protocols.

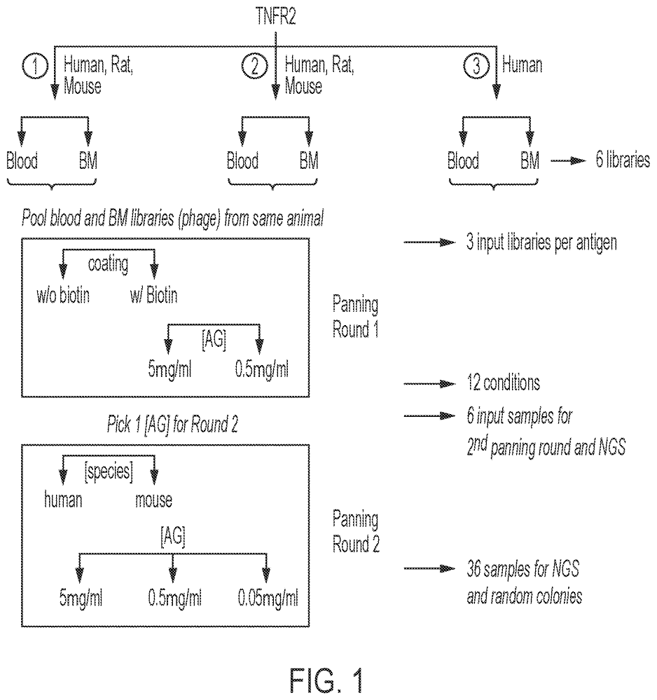

Binders to human and mouse TNFR2 were enriched from VHH immune libraries by two rounds of phage display. The general panning strategy is illustrated in FIG. 1 using the panning substrates listed Table 3. For the Table below: Hu, Human; Ms, mouse; PBS, phosphate buffered saline; Cat., catalog; MW, molecular weight; Calc., Calculated; Seq., Sequence; N-term., N-terminal; aa, amino acid.

TABLE 3
Panning substrates

| | | | | | | | N-term. | |
|---|---|---|---|---|---|---|---|---|
| | | | | | | | Seq., aa | |
| Species | Modification | Buffer | Cat. # | Lot # | Vendor | MW | Seq. | Source |
| Human | His-tag | PBS, | 10417- | MB15AU1103 | Sino | calc. | Leu23- | HEK293 |
| | | pH 7.4 | H08H | | | 26.7 kDa, | Asp257 | |
| | | | | | | apparent | | |
| | | | | | | 42-45 kDa | | |
| Mouse | His-tag | PBS, | 50128- | MB11DE1903 | Sino | calc. | Val23- | HEK293 |
| | | pH 7.4 | M08H | | | 26.8 kDa, | Gly258 | |
| | | | | | | apparent | | |
| | | | | | | 45 kDa | | |

For the first panning round, libraries originating from the first harvested blood sample and the first harvested bone marrow sample of the same animal were pooled in equal parts (at the phage level), resulting in three pooled input libraries per antigen. Each library was panned under four conditions (two antigen concentrations and two ways of antigen immobilization) with human TNFR2, resulting in 12 panning reactions. For the second rounds of panning, six output samples (enriched libraries) from the first round were chosen and served as input libraries for the second round. Preferentially, the enriched libraries from the higher panning substrate concentration were chosen to preserve maximum diversity. Pannings of the second round were performed with three antigen concentrations of human and mouse antigen to result 24 conditions. Three antigen concentrations for TNFR2 were further chosen to recover binders with lower affinity. This panning regimen was implemented to identify binders that cross-reacted with human and mouse TNFR2.

In some cases, binders with medium affinity were preferred since higher valency may drive avidity. Thus, the antigen concentrations in the second panning round were the same, one tenth and one hundredth of the first round.

Phages were produced according to QVQ Holding B.V. (QVQ) standard operating procedures (SOPs) and phage titers were determined to ensure at least 10-fold excess over the maximum diversity of the libraries. Panning substrates were commercially purchased (see Table 1). The panning substrates were immobilized either by direct coating on enzyme-linked immunoassay (ELISA) plates or by binding of biotinylated antigen on neutravidin-coated ELISA plate. Glycerol stocks were prepared from all outputs and are stored at −80° C.

Panning outputs were analyzed by random clone picking/periplasmic extract (PE)-ELISA/Sanger sequencing (QVQ) and next-generation sequencing (NGS; Genewiz/PipeBio).

For random colony picking, rescued outputs of the first and second panning rounds were plated out and 920 random single clones (equal numbers of colonies from each condition) were selected to create masterplates (96-well format). From the masterplates, expression cultures in deep-well plates were inoculated to produce periplasmic extracts containing monoclonal VHH. Periplasmic extracts were used to determine binding of individual VHHs to human, mouse and cynomolgus antigen by ELISA. For conditions where the panning substrate was biotinylated and captured by neutravidin, background binders were identified by ELISA with neutravidin. All masterplates were sequenced by the Sanger method.

For NGS analysis, minipreps from input libraries and outputs after the first and second rounds of panning were prepared, amplified by PCR, and sequenced by NGS.

### Example 3. Next-Generation Sequencing

Following two rounds of panning, phages were eluted and corresponding phagemid DNA was extracted. Identification of initial V-body candidates was performed in a parallelized fashion, employing a random colony picking, as well as a next-generation sequencing (NGS) approach, as orthogonal techniques to yield a particularly diverse set of initial candidates. Prior to NGS techniques, random colony picking was the prevalent method for initial hit identification, which involved transformation of a phagemid pool (from a panning elution) and selection of individual bacterial colonies to isolate single clones. Following this approach, 920 single colonies were randomly picked from the 36 samples of the second panning round (FIG. 2). Then, individual clones were expressed and subject to ELISA screening against the target antigen to select for antigen binding V-bodies, which were further functionally characterized.

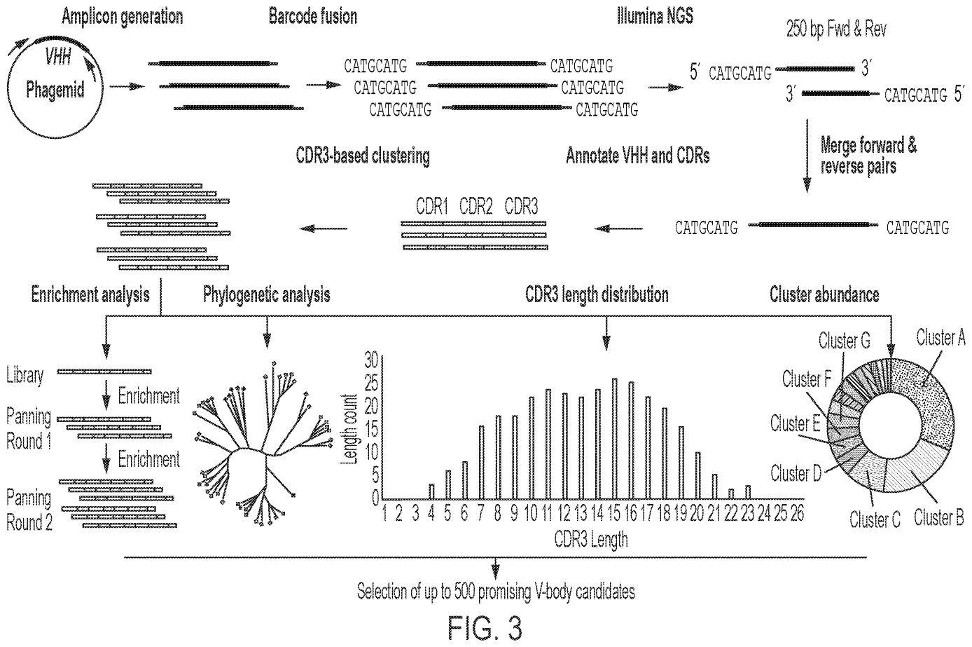

All panning eluates were sequenced using NGS. In brief, the entire VHH region was PCR-amplified from isolated phagemid pools by primers annealing to universal phagemid sequences 5′ and 3′ of the VHH-encoding region. In a second step, the generated amplicons were fused to sequencing-compatible and sample-specific barcodes. By fusing unique barcodes, it was possible to multiplex hundreds of different samples. Following the preparation of 51 samples, an Illumina NovaSeq 6000 with an SP flowcell was employed for sequencing, yielding 250 base pair (bp) reads from each direction and a total of ~600 million reads. To account for differences in the number of expected unique sequences in the library, and both panning rounds, each library was sequenced with a total of 20 million reads, compared to the first and second round of panning with 2 million reads each. This strategy allowed for covering sufficient sequence space in the libraries, as well as in the panning eluates. A spike-in of 30% of a standard PhiX reference genome control into the sequencing reaction helped to provide a technical quality control for assessing sequencing accuracy. The NGS raw data contained multiplexed sequencing reads, which were de-multiplexed based on the sample-specific barcodes. The de-multiplexed data containing unmerged sequencing reads were then processed by employing an NGS analysis platform. In brief, forward and reverse sequence pairs were merged by their overlapping sequence, thereby generating a full VHH sequence from two half sequences (FIG. 3). The framework regions, CDRs, and sequence-specific liabilities were then annotated for the merged V-body sequences.

Based on CDR3 identity, V-body sequences were clustered, allowing for a detailed analysis of V-body enrichment during phage display, sequence diversity, CDR3 length distribution and cluster abundance. Identified V-bodies can be classified into eleven distinct clusters, as follows: 31G3-31D6 (group A), 37C7 (group B), 31G11 (group C), 33D4 (group D), N1277 (group G), N1364-N1365 (group J), 35A10-N1402-N1400 (group K), N1425 (group L), N1409 (group M), N1323 (group N), and 33B1 (group O). The following Table 4-1 to Table 4-33 display the amino acid frequency distribution at each amino acid (AA) position (IMGT) for CDR1, CDR2 and CDR3 for the eleven clusters. Table 5 provides the sequence identifiers of amino acid sequences of the complementarity determining regions (CDR1, CDR2 and CDR3), amino acid and DNA sequences of the full-length VHH domain for the identified V-bodies.

TABLE 4-1
CDR1 amino acid frequency distribution
for cluster 31G3-31D6 (Group A)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 5.3 | 0 | 36 |
| C | 0 | 0 | 0 | 0 | 0 | 0 | 0 | 0 |
| D | 0 | 0 | 0 | 0 | 0 | 0.3 | 4.7 | 0 |
| E | 0 | 0 | 0 | 0 | 0 | 0 | 0 | 0 |
| F | 0 | 0.3 | 0 | 28 | 0 | 7 | 0 | 0 |
| G | 100 | 0.3 | 0 | 0 | 1 | 1.7 | 0 | 44 |
| H | 0 | 0 | 0 | 0 | 0 | 0 | 0 | 0 |
| I | 0 | 0 | 95 | 0 | 0 | 1.3 | 0 | 0 |
| K | 0 | 0 | 0 | 0 | 0 | 0 | 6.3 | 0 |
| L | 0 | 0 | 0 | 0.3 | 0 | 11 | 0 | 0 |
| M | 0 | 0 | 0 | 0 | 0 | 0 | 0 | 0 |
| N | 0 | 1 | 0 | 0.3 | 1.3 | 0 | 83 | 1.3 |
| P | 0 | 0 | 0 | 0 | 0 | 0 | 0 | 0 |
| Q | 0 | 0 | 0 | 0 | 0 | 0 | 0 | 0 |
| R | 0 | 3 | 3.3 | 1 | 4.7 | 0 | 5 | 0 |
| S | 0 | 95 | 0 | 1 | 93 | 4 | 0.7 | 19 |
| T | 0.3 | 0 | 0.7 | 0 | 0 | 69 | 0 | 0 |
| V | 0 | 0.3 | 0.7 | 69 | 0 | 0 | 0 | 0 |
| W | 0 | 0 | 0 | 0 | 0 | 0 | 0 | 0 |
| Y | 0 | 0 | 0 | 0 | 0 | 0 | 0.3 | 0 |

TABLE 4-2
CDR2 amino acid frequency distribution
for cluster 31G3-3106 (Group A)
Position

| AA | 56 | 57 | 58 | 59 | 63 | 64 | 65 |
|---|---|---|---|---|---|---|---|
| A | 0 | 0 | 0 | 0 | 0 | 0 | 10 |
| C | 0 | 0 | 0 | 0 | 0 | 0 | 0 |
| D | 0 | 0 | 0 | 88 | 1.7 | 0 | 0 |
| E | 0 | 0 | 0 | 0 | 0.3 | 0.7 | 1 |
| F | 8 | 0 | 0 | 0 | 0 | 73 | 0 |
| G | 0 | 0 | 0 | 0 | 97 | 0 | 0 |
| H | 0 | 2 | 0 | 0 | 0 | 0 | 0 |
| I | 88 | 0 | 0 | 0 | 0 | 2.7 | 20 |
| K | 0 | 0 | 0 | 0 | 0 | 0 | 0 |
| L | 3.3 | 0 | 0 | 0 | 0 | 4.7 | 0.3 |
| M | 0.7 | 0 | 0 | 0 | 0 | 14 | 0 |
| N | 0 | 1.3 | 0 | 12 | 0 | 0 | 0 |
| P | 0 | 0 | 0 | 0 | 0 | 0 | 0.7 |
| Q | 0 | 0.7 | 0 | 0 | 0 | 0 | 0 |
| R | 0 | 96 | 0.3 | 0 | 0 | 0 | 0 |
| S | 0 | 0 | 99 | 0 | 1 | 0.3 | 0 |
| T | 0 | 0 | 0.7 | 0 | 0 | 0 | 59 |
| V | 0.3 | 0 | 0 | 0 | 0 | 0 | 8.6 |
| W | 0 | 0 | 0 | 0 | 0 | 0 | 0 |
| Y | 0 | 0 | 0 | 0 | 0 | 4.3 | 0 |

TABLE 4-3
CDR3 amino acid frequency distribution for cluster 31G3-3106 (Group A)

| | Position | | | | | | | | | | | | | |
|---|---|---|---|---|---|---|---|---|---|---|---|---|---|---|
| AA | 105 | 106 | 107 | 108 | 109 | 110 | 111 | 112.1 | 112 | 113 | 114 | 115 | 116 | 117 |
| A | 0 | 0 | 0 | 23.9 | 0 | 0 | 2 | 20.6 | 0 | 0 | 0.3 | 0 | 1.7 | 0 |
| C | 0 | 0 | 0 | 0 | 0 | 0 | 0 | 0 | 0 | 0 | 0 | 0 | 0 | 0 |
| D | 0.3 | 5 | 0 | 0 | 0 | 0 | 0 | 0.7 | 1 | 2.3 | 0.3 | 0 | 0 | 0 |
| E | 0 | 0 | 0 | 0 | 0 | 0 | 0 | 0 | 0 | 0 | 0 | 0 | 0 | 0 |
| F | 40.9 | 0 | 0 | 0 | 4 | 0 | 0 | 0 | 0 | 24.3 | 0 | 0 | 0 | 91.7 |
| G | 0 | 0 | 0 | 0 | 0 | 4 | 1.7 | 0 | 0 | 0 | 98.3 | 0 | 0 | 0 |
| H | 0 | 1 | 6.6 | 0 | 1.7 | 0 | 1 | 1.7 | 1 | 0.3 | 0 | 8 | 0 | 0 |
| I | 0.3 | 0 | 0 | 0 | 1 | 0.3 | 0 | 0 | 0.3 | 0 | 0 | 0 | 0 | 3 |
| K | 0 | 0 | 0.3 | 0 | 0 | 0 | 0 | 0 | 5 | 0 | 0 | 1.3 | 0 | 0 |
| L | 0.7 | 0 | 0 | 0 | 90 | 0 | 0 | 0 | 0 | 0 | 0 | 1 | 14.6 | 1 |
| M | 0 | 0 | 0 | 0 | 0 | 0 | 0 | 0 | 0 | 0 | 0 | 0 | 0 | 0 |
| N | 0 | 0 | 0 | 0 | 0 | 0.3 | 0 | 0 | 87 | 0 | 0 | 0 | 0 | 0 |
| P | 0 | 0 | 0 | 2.7 | 0 | 0 | 0 | 75.4 | 0 | 0 | 0 | 0 | 3.3 | 0 |
| Q | 0 | 0 | 93 | 0 | 0 | 0 | 0 | 0 | 0 | 0 | 0 | 86.4 | 0 | 0 |
| R | 0 | 0 | 0 | 0 | 0 | 3.7 | 0 | 0 | 0 | 0 | 0 | 3.3 | 0 | 0 |
| S | 1.7 | 3.7 | 0 | 73.1 | 0 | 85.7 | 42.9 | 0.3 | 2 | 8.6 | 0.7 | 0 | 0 | 1.7 |
| T | 0 | 0 | 0 | 0.3 | 0 | 6 | 52.5 | 0.3 | 3.7 | 0 | 0 | 0 | 36.5 | 0 |
| V | 0 | 0 | 0 | 0 | 0 | 0 | 0 | 1 | 0 | 0.3 | 0.3 | 0 | 43.9 | 0.3 |
| W | 0 | 0 | 0 | 0 | 0 | 0 | 0 | 0 | 0 | 1 | 0 | 0 | 0 | 0 |
| Y | 56.1 | 90.4 | 0 | 0 | 3.3 | 0 | 0 | 0 | 0 | 63.1 | 0 | 0 | 0 | 2.3 |

TABLE 4-4
CDR1 amino acid frequency distribution
for cluster 37C7 (Group B)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 100 |
| C | 0 | 0 | 0 | 0 | 0 | 0 | 0 | 0 |
| D | 0 | 0 | 0 | 0 | 100 | 90 | 0 | 0 |
| E | 0 | 0 | 0 | 0 | 0 | 0 | 0 | 0 |
| F | 0 | 100 | 0 | 80 | 0 | 0 | 0 | 0 |
| G | 100 | 0 | 0 | 0 | 0 | 10 | 0 | 0 |
| H | 0 | 0 | 0 | 0 | 0 | 0 | 0 | 0 |
| I | 0 | 0 | 0 | 0 | 0 | 0 | 80 | 0 |
| K | 0 | 0 | 0 | 0 | 0 | 0 | 0 | 0 |
| L | 0 | 0 | 0 | 20 | 0 | 0 | 0 | 0 |
| M | 0 | 0 | 0 | 0 | 0 | 0 | 0 | 0 |
| N | 0 | 0 | 0 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 0 | 0 | 0 | 0 | 0 | 0 | 0 |
| T | 0 | 0 | 100 | 0 | 0 | 0 | 0 | 0 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 20 | 0 |

TABLE 4-5
CDR2 amino acid frequency distribution
for cluster 37C7 (Group B)

| | Position | | | | | | | |
|---|---|---|---|---|---|---|---|---|
| AA | 56 | 57 | 58 | 59 | 62 | 63 | 64 | 65 |
| A | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 20 | 0 | 0 | 0 | 0 | 0 | 0 |
| G | 0 | 0 | 0 | 0 | 80 | 0 | 0 | 0 |
| H | 0 | 0 | 0 | 0 | 0 | 0 | 0 | 0 |
| I | 100 | 0 | 0 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 0 | 0 | 0 | 0 | 0 | 100 | 0 |
| P | 0 | 0 | 0 | 0 | 0 | 80 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 0 | 100 | 0 | 20 | 20 | 0 | 0 |
| T | 0 | 0 | 0 | 0 | 0 | 0 | 0 | 100 |
| V | 0 | 0 | 0 | 0 | 0 | 0 | 0 | 0 |
| W | 0 | 0 | 0 | 0 | 0 | 0 | 0 | 0 |
| Y | 0 | 80 | 0 | 100 | 0 | 0 | 0 | 0 |

TABLE 4-6
CDR3 amino acid frequency distribution for cluster 37C7 (Group B)

| | Position | | | | | | | | | | | | | | | |
|---|---|---|---|---|---|---|---|---|---|---|---|---|---|---|---|---|
| AA | 105 | 106 | 107 | 108 | 109 | 110 | 111 | 111.1 | 112.2 | 112.1 | 112 | 113 | 114 | 115 | 116 | 117 |
| A | 90 | 100 | 0 | 0 | 0 | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 | 0 | 0 | 0 |
| D | 0 | 0 | 90 | 0 | 100 | 0 | 0 | 0 | 0 | 0 | 10 | 0 | 0 | 0 | 100 | 0 |
| E | 0 | 0 | 10 | 0 | 0 | 0 | 0 | 0 | 0 | 0 | 0 | 0 | 0 | 0 | 0 | 0 |
| F | 0 | 0 | 0 | 0 | 0 | 0 | 0 | 0 | 0 | 0 | 0 | 0 | 0 | 0 | 0 | 0 |
| G | 0 | 0 | 0 | 0 | 0 | 0 | 0 | 0 | 10 | 10 | 0 | 100 | 0 | 0 | 0 | 0 |
| H | 0 | 0 | 0 | 0 | 0 | 0 | 0 | 0 | 0 | 0 | 0 | 0 | 0 | 100 | 0 | 0 |
| I | 0 | 0 | 0 | 0 | 0 | 0 | 0 | 0 | 0 | 0 | 0 | 0 | 0 | 0 | 0 | 0 |
| K | 0 | 0 | 0 | 0 | 0 | 0 | 0 | 0 | 0 | 0 | 0 | 0 | 0 | 0 | 0 | 0 |
| L | 0 | 0 | 0 | 0 | 0 | 100 | 0 | 0 | 0 | 0 | 0 | 0 | 0 | 0 | 0 | 0 |
| M | 0 | 0 | 0 | 0 | 0 | 0 | 0 | 0 | 0 | 0 | 0 | 0 | 0 | 0 | 0 | 0 |
| N | 0 | 0 | 0 | 0 | 0 | 0 | 0 | 0 | 0 | 0 | 0 | 0 | 0 | 0 | 0 | 0 |
| P | 0 | 0 | 0 | 0 | 0 | 0 | 0 | 0 | 0 | 0 | 0 | 0 | 100 | 0 | 0 | 0 |
| Q | 0 | 0 | 0 | 0 | 0 | 0 | 0 | 0 | 0 | 0 | 0 | 0 | 0 | 0 | 0 | 0 |
| R | 0 | 0 | 0 | 0 | 0 | 0 | 40 | 0 | 0 | 0 | 0 | 0 | 0 | 0 | 0 | 0 |
| S | 0 | 0 | 0 | 100 | 0 | 0 | 60 | 0 | 0 | 0 | 0 | 0 | 0 | 0 | 0 | 10 |
| T | 0 | 0 | 0 | 0 | 0 | 0 | 0 | 100 | 0 | 0 | 0 | 0 | 0 | 0 | 0 | 0 |
| V | 10 | 0 | 0 | 0 | 0 | 0 | 0 | 0 | 90 | 90 | 90 | 0 | 0 | 0 | 0 | 0 |
| W | 0 | 0 | 0 | 0 | 0 | 0 | 0 | 0 | 0 | 0 | 0 | 0 | 0 | 0 | 0 | 0 |
| Y | 0 | 0 | 0 | 0 | 0 | 0 | 0 | 0 | 0 | 0 | 0 | 0 | 0 | 0 | 0 | 90 |

TABLE 4-7
CDR1 amino acid frequency distribution
for cluster 31G11 (Group C)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 100 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 100 | 0 | 100 | 0 | 0 | 0 | 0 |
| G | 100 | 0 | 0 | 0 | 0 | 45.5 | 0 | 0 |
| H | 0 | 0 | 0 | 0 | 0 | 0 | 0 | 0 |
| I | 0 | 0 | 0 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 0 | 0 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 18.2 | 0 | 0 |
| S | 0 | 0 | 0 | 0 | 100 | 36.4 | 0 | 0 |
| T | 0 | 0 | 100 | 0 | 0 | 0 | 0 | 0 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 100 | 0 |

TABLE 4-8
CDR2 amino acid frequency distribution
for cluster 31G11 (Group C)

| | Position | | | | | | | |
|---|---|---|---|---|---|---|---|---|
| AA | 56 | 57 | 58 | 59 | 62 | 63 | 64 | 65 |
| A | 0 | 0 | 0 | 0 | 0 | 0 | 0 | 0 |
| C | 0 | 0 | 0 | 0 | 0 | 0 | 0 | 0 |
| D | 0 | 0 | 18.2 | 100 | 0 | 0 | 54.6 | 0 |
| E | 0 | 0 | 0 | 0 | 0 | 0 | 45.5 | 0 |
| F | 0 | 0 | 0 | 0 | 0 | 0 | 0 | 0 |
| G | 0 | 0 | 0 | 0 | 100 | 0 | 0 | 0 |
| H | 0 | 0 | 0 | 0 | 0 | 0 | 0 | 0 |
| I | 100 | 0 | 0 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 0 | 0 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 54.6 | 81.8 | 0 | 0 | 100 | 0 | 0 |
| T | 0 | 0 | 0 | 0 | 0 | 0 | 0 | 100 |
| V | 0 | 0 | 0 | 0 | 0 | 0 | 0 | 0 |
| W | 0 | 0 | 0 | 0 | 0 | 0 | 0 | 0 |
| Y | 0 | 45.5 | 0 | 0 | 0 | 0 | 0 | 0 |

TABLE 4-9
CDR3 amino acid frequency distribution for cluster 31G11 (Group C)

| | Position | | | | | | | | | | | | | | | | | |
|---|---|---|---|---|---|---|---|---|---|---|---|---|---|---|---|---|---|---|
| AA | 105 | 106 | 107 | 108 | 109 | 110 | 111 | 111.1 | 111.2 | 112.3 | 112.2 | 112.1 | 112 | 113 | 114 | 115 | 116 | 117 |
| A | 100 | 0 | 9.1 | 100 | 0 | 0 | 0 | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 | 0 | 0 | 0 | 0 | 0 |
| D | 0 | 0 | 90.9 | 0 | 0 | 0 | 0 | 0 | 0 | 0 | 0 | 0 | 0 | 0 | 45.5 | 0 | 100 | 0 |
| E | 0 | 0 | 0 | 0 | 0 | 0 | 0 | 0 | 0 | 0 | 0 | 0 | 0 | 0 | 54.5 | 0 | 0 | 0 |
| F | 0 | 0 | 0 | 0 | 0 | 0 | 0 | 0 | 0 | 0 | 0 | 100 | 0 | 45.5 | 0 | 0 | 0 | 0 |
| G | 0 | 0 | 0 | 0 | 100 | 18.2 | 0 | 100 | 0 | 100 | 0 | 0 | 100 | 0 | 0 | 0 | 0 | 0 |
| H | 0 | 0 | 0 | 0 | 0 | 0 | 0 | 0 | 0 | 0 | 0 | 0 | 0 | 0 | 0 | 0 | 0 | 0 |
| I | 0 | 0 | 0 | 0 | 0 | 0 | 0 | 0 | 0 | 0 | 0 | 0 | 0 | 0 | 0 | 0 | 0 | 0 |
| K | 0 | 90.9 | 0 | 0 | 0 | 0 | 0 | 0 | 0 | 0 | 0 | 0 | 0 | 0 | 0 | 0 | 0 | 0 |
| L | 0 | 0 | 0 | 0 | 0 | 0 | 0 | 0 | 0 | 0 | 0 | 0 | 0 | 0 | 0 | 0 | 0 | 0 |
| M | 0 | 0 | 0 | 0 | 0 | 0 | 0 | 0 | 0 | 0 | 0 | 0 | 0 | 0 | 0 | 0 | 0 | 0 |
| N | 0 | 9.1 | 0 | 0 | 0 | 0 | 0 | 0 | 0 | 0 | 0 | 0 | 0 | 0 | 0 | 0 | 0 | 0 |
| P | 0 | 0 | 0 | 0 | 0 | 0 | 0 | 0 | 0 | 0 | 100 | 0 | 0 | 0 | 0 | 0 | 0 | 0 |
| Q | 0 | 0 | 0 | 0 | 0 | 0 | 0 | 0 | 0 | 0 | 0 | 0 | 0 | 0 | 0 | 0 | 0 | 0 |
| R | 0 | 0 | 0 | 0 | 0 | 0 | 0 | 0 | 45.5 | 0 | 0 | 0 | 0 | 0 | 0 | 0 | 0 | 0 |
| S | 0 | 0 | 0 | 0 | 0 | 81.8 | 0 | 0 | 0 | 0 | 0 | 0 | 0 | 0 | 0 | 0 | 0 | 0 |
| T | 0 | 0 | 0 | 0 | 0 | 0 | 0 | 0 | 54.5 | 0 | 0 | 0 | 0 | 0 | 0 | 0 | 0 | 0 |
| V | 0 | 0 | 0 | 0 | 0 | 0 | 0 | 0 | 0 | 0 | 0 | 0 | 0 | 0 | 0 | 0 | 0 | 0 |
| W | 0 | 0 | 0 | 0 | 0 | 0 | 100 | 0 | 0 | 0 | 0 | 0 | 0 | 0 | 0 | 0 | 0 | 0 |
| Y | 0 | 0 | 0 | 0 | 0 | 0 | 0 | 0 | 0 | 0 | 0 | 0 | 0 | 54.5 | 0 | 100 | 0 | 100 |

TABLE 4-10
CDR1 amino acid frequency distribution
for cluster 33D4 (Group D)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 97.5 | 0 | 0 |
| E | 0 | 0 | 0 | 0 | 0 | 0 | 0 | 0 |
| F | 0 | 0 | 0 | 100 | 0 | 0 | 0 | 0 |
| G | 100 | 0 | 0 | 0 | 1.3 | 0 | 0 | 97.5 |
| H | 0 | 0 | 0 | 0 | 0 | 0 | 0 | 0 |
| I | 0 | 0 | 0 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 0 | 0 | 0 | 0 | 1.3 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 100 | 0 | 0 | 0 | 0 | 0 | 0 |
| S | 0 | 0 | 0 | 0 | 98.7 | 1.3 | 0 | 0 |
| T | 0 | 0 | 100 | 0 | 0 | 0 | 0 | 1.3 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 100 | 1.3 |

TABLE 4-11
CDR2 amino acid frequency distribution
for cluster 33D4 (Group D)

| | Position | | | | | | | |
|---|---|---|---|---|---|---|---|---|
| AA | 56 | 57 | 58 | 59 | 62 | 63 | 64 | 65 |
| A | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 0 | 0 | 0 | 0 | 0 |
| G | 0 | 0 | 0 | 0 | 2.5 | 98.7 | 0 | 0 |
| H | 0 | 0 | 0 | 0 | 0 | 0 | 0 | 0 |
| I | 100 | 0 | 0 | 0 | 0 | 0 | 0 | 0 |
| K | 0 | 0 | 0 | 0 | 0 | 1.3 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 96 | 0 | 0 | 96.2 | 0 | 0 | 0 |
| P | 0 | 0 | 0 | 0 | 0 | 0 | 0 | 0 |
| Q | 0 | 1.3 | 0 | 0 | 0 | 0 | 0 | 0 |
| R | 0 | 1.3 | 0 | 0 | 0 | 0 | 97.5 | 0 |
| S | 0 | 1.3 | 0 | 99 | 0 | 0 | 2.5 | 0 |
| T | 0 | 0 | 0 | 1.3 | 1.3 | 0 | 0 | 100 |
| V | 0 | 0 | 0 | 0 | 0 | 0 | 0 | 0 |
| W | 0 | 0 | 100 | 0 | 0 | 0 | 0 | 0 |
| Y | 0 | 0 | 0 | 0 | 0 | 0 | 0 | 0 |

TABLE 4-12
CDR3 amino acid frequency distribution for cluster 33D4 (Group D)

| | Position | | | | | | | | | | |
|---|---|---|---|---|---|---|---|---|---|---|---|
| AA | 105 | 106 | 107 | 108 | 109 | 110 | 113 | 114 | 115 | 116 | 117 |
| A | 97.5 | 84.8 | 1.3 | 1.3 | 2.5 | 1.3 | 0 | 88.6 | 0 | 1.3 | 0 |
| C | 0 | 0 | 0 | 0 | 0 | 0 | 0 | 0 | 0 | 0 | 0 |
| D | 0 | 0 | 0 | 0 | 0 | 0 | 0 | 0 | 1.3 | 0 | 0 |
| E | 0 | 0 | 0 | 0 | 0 | 0 | 3.8 | 3.8 | 0 | 0 | 0 |
| F | 0 | 0 | 0 | 0 | 3.8 | 0 | 0 | 0 | 3.8 | 0 | 0 |
| G | 0 | 11.4 | 0 | 0 | 0 | 96.2 | 0 | 1.3 | 0 | 0 | 0 |
| H | 0 | 0 | 0 | 1.3 | 0 | 0 | 0 | 0 | 1.3 | 0 | 1.3 |
| I | 0 | 0 | 1.3 | 0 | 0 | 0 | 0 | 0 | 0 | 0 | 0 |
| K | 0 | 0 | 0 | 0 | 0 | 0 | 78.5 | 0 | 0 | 0 | 0 |
| L | 0 | 0 | 0 | 2.5 | 0 | 0 | 0 | 0 | 0 | 0 | 0 |
| M | 0 | 0 | 0 | 0 | 0 | 0 | 1.3 | 0 | 0 | 0 | 0 |
| N | 0 | 0 | 1.3 | 0 | 0 | 0 | 1.3 | 0 | 1.3 | 0 | 3.8 |
| P | 0 | 0 | 5.1 | 91.1 | 1.3 | 0 | 0 | 1.3 | 0 | 0 | 0 |
| Q | 0 | 0 | 0 | 0 | 0 | 0 | 6.3 | 0 | 0 | 0 | 0 |
| R | 0 | 0 | 0 | 1.3 | 0 | 1.3 | 5.1 | 0 | 0 | 0 | 0 |
| S | 0 | 0 | 1.3 | 1.3 | 86.1 | 0 | 0 | 1.3 | 0 | 19 | 1.3 |
| T | 1.3 | 2.5 | 89.9 | 1.3 | 5.1 | 0 | 3.8 | 2.5 | 0 | 79.7 | 0 |
| V | 1.3 | 1.3 | 0 | 0 | 0 | 1.3 | 0 | 1.3 | 0 | 0 | 0 |
| W | 0 | 0 | 0 | 0 | 0 | 0 | 0 | 0 | 0 | 0 | 0 |
| Y | 0 | 0 | 0 | 0 | 1.3 | 0 | 0 | 0 | 92.4 | 0 | 93.7 |

TABLE 4-13
CDR1 amino acid frequency distribution
for cluster N1277 (Group G)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 100 |
| C | 0 | 0 | 0 | 0 | 0 | 0 | 0 | 0 |
| D | 0 | 0 | 0 | 0 | 100 | 0 | 0 | 0 |
| E | 0 | 0 | 0 | 0 | 0 | 0 | 0 | 0 |
| F | 0 | 66.7 | 0 | 0 | 0 | 0 | 0 | 0 |
| G | 100 | 0 | 0 | 0 | 0 | 0 | 0 | 0 |
| H | 0 | 0 | 0 | 0 | 0 | 0 | 0 | 0 |
| I | 0 | 0 | 0 | 0 | 0 | 0 | 0 | 0 |
| K | 0 | 0 | 0 | 0 | 0 | 0 | 0 | 0 |
| L | 0 | 33.3 | 0 | 100 | 0 | 0 | 0 | 0 |
| M | 0 | 0 | 0 | 0 | 0 | 0 | 0 | 0 |
| N | 0 | 0 | 0 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 0 | 0 | 0 | 0 | 0 | 0 | 0 |
| T | 0 | 0 | 100 | 0 | 0 | 0 | 0 | 0 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 100 | 100 | 0 |

TABLE 4-14
CDR2 amino acid frequency distribution
for cluster N1277 (Group G)

| | Position | | | | | | | |
|---|---|---|---|---|---|---|---|---|
| AA | 56 | 57 | 58 | 59 | 62 | 63 | 64 | 65 |
| A | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 100 | 0 | 0 | 0 |
| E | 0 | 0 | 0 | 0 | 0 | 0 | 0 | 0 |
| F | 0 | 0 | 0 | 0 | 0 | 0 | 0 | 0 |
| G | 0 | 0 | 0 | 33.3 | 0 | 100 | 0 | 0 |
| H | 0 | 0 | 0 | 0 | 0 | 0 | 0 | 0 |
| I | 100 | 0 | 0 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 33.3 | 0 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 66.7 | 0 | 66.7 | 0 | 0 | 100 | 0 |
| T | 0 | 0 | 66.7 | 0 | 0 | 0 | 0 | 100 |
| V | 0 | 0 | 33.3 | 0 | 0 | 0 | 0 | 0 |
| W | 0 | 0 | 0 | 0 | 0 | 0 | 0 | 0 |
| Y | 0 | 0 | 0 | 0 | 0 | 0 | 0 | 0 |

TABLE 4-15
CDR3 amino acid frequency distribution for cluster N1277 (Group G)

| | Position | | | | | | | | | | |
|---|---|---|---|---|---|---|---|---|---|---|---|
| AA | 105 | 106 | 107 | 108 | 109 | 110 | 111 | 111.1 | 111.2 | 111.3 | 111.4 |
| A | 100 | 0 | 0 | 0 | 0 | 0 | 0 | 0 | 0 | 100 | 0 |
| C | 0 | 0 | 0 | 0 | 0 | 0 | 0 | 0 | 100 | 0 | 0 |
| D | 0 | 0 | 0 | 0 | 0 | 0 | 0 | 0 | 0 | 0 | 0 |
| E | 0 | 0 | 0 | 0 | 0 | 0 | 0 | 0 | 0 | 0 | 0 |
| F | 0 | 0 | 0 | 0 | 0 | 0 | 0 | 0 | 0 | 0 | 0 |
| G | 0 | 0 | 0 | 100 | 0 | 0 | 0 | 0 | 0 | 0 | 0 |
| H | 0 | 0 | 0 | 0 | 0 | 0 | 0 | 0 | 0 | 0 | 0 |
| I | 0 | 0 | 0 | 0 | 0 | 0 | 0 | 0 | 0 | 0 | 0 |
| K | 0 | 0 | 0 | 0 | 0 | 0 | 0 | 0 | 0 | 0 | 0 |
| L | 0 | 0 | 0 | 0 | 0 | 0 | 0 | 0 | 0 | 0 | 0 |
| M | 0 | 0 | 0 | 0 | 0 | 0 | 33.3 | 0 | 0 | 0 | 0 |
| N | 0 | 0 | 0 | 0 | 0 | 0 | 0 | 0 | 0 | 0 | 0 |
| P | 0 | 0 | 100 | 0 | 100 | 0 | 0 | 0 | 0 | 0 | 100 |
| Q | 0 | 0 | 0 | 0 | 0 | 0 | 0 | 0 | 0 | 0 | 0 |
| R | 0 | 0 | 0 | 0 | 0 | 0 | 0 | 0 | 0 | 0 | 0 |
| S | 0 | 0 | 0 | 0 | 0 | 0 | 33.3 | 0 | 0 | 0 | 0 |
| T | 0 | 100 | 0 | 0 | 0 | 0 | 33.3 | 0 | 0 | 0 | 0 |
| V | 0 | 0 | 0 | 0 | 0 | 0 | 0 | 0 | 0 | 0 | 0 |
| W | 0 | 0 | 0 | 0 | 0 | 0 | 0 | 0 | 0 | 0 | 0 |
| Y | 0 | 0 | 0 | 0 | 0 | 100 | 0 | 100 | 0 | 0 | 0 |

| | Position | | | | | | | | | | |
|---|---|---|---|---|---|---|---|---|---|---|---|
| AA | 112.5 | 112.4 | 112.3 | 112.2 | 112.1 | 112 | 113 | 114 | 115 | 116 | 117 |
| A | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 |
| D | 0 | 0 | 0 | 0 | 0 | 0 | 0 | 0 | 0 | 100 | 0 |
| E | 0 | 0 | 0 | 0 | 0 | 0 | 0 | 0 | 0 | 0 | 0 |
| F | 0 | 0 | 0 | 0 | 0 | 0 | 0 | 0 | 0 | 0 | 0 |
| G | 0 | 100 | 0 | 0 | 0 | 0 | 0 | 100 | 0 | 0 | 0 |
| H | 0 | 0 | 0 | 0 | 0 | 0 | 0 | 0 | 0 | 0 | 0 |
| I | 0 | 0 | 0 | 0 | 0 | 0 | 0 | 0 | 0 | 0 | 0 |
| K | 0 | 0 | 0 | 0 | 0 | 0 | 0 | 0 | 0 | 0 | 0 |
| L | 0 | 0 | 0 | 0 | 0 | 0 | 0 | 0 | 0 | 0 | 0 |
| M | 0 | 0 | 0 | 0 | 0 | 0 | 0 | 0 | 0 | 0 | 0 |
| N | 0 | 0 | 0 | 0 | 0 | 0 | 0 | 0 | 0 | 0 | 0 |
| P | 0 | 0 | 0 | 0 | 0 | 0 | 0 | 0 | 0 | 0 | 0 |
| Q | 0 | 0 | 0 | 0 | 0 | 0 | 0 | 0 | 0 | 0 | 0 |
| R | 0 | 0 | 0 | 0 | 0 | 0 | 100 | 0 | 0 | 0 | 0 |
| S | 0 | 0 | 100 | 100 | 0 | 100 | 0 | 0 | 0 | 0 | 0 |
| T | 0 | 0 | 0 | 0 | 0 | 0 | 0 | 0 | 0 | 0 | 0 |
| V | 0 | 0 | 0 | 0 | 0 | 0 | 0 | 0 | 0 | 0 | 0 |
| W | 0 | 0 | 0 | 0 | 100 | 0 | 0 | 0 | 0 | 0 | 0 |
| Y | 100 | 0 | 0 | 0 | 0 | 0 | 0 | 0 | 100 | 0 | 100 |

TABLE 4-16
CDR1 amino acid frequency distribution
for cluster N1364-N1365 (Group J)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 2 |
| C | 0 | 0 | 0 | 0 | 0 | 0 | 0 | 0 |
| D | 0 | 0 | 0 | 0 | 2 | 2 | 0 | 0 |
| E | 0 | 0 | 0 | 0 | 0 | 0 | 0 | 0 |
| F | 0 | 100 | 0 | 100 | 0 | 0 | 0 | 0 |
| G | 100 | 0 | 0 | 0 | 0 | 0 | 0 | 0 |
| H | 0 | 0 | 0 | 0 | 0 | 0 | 0 | 0 |
| I | 0 | 0 | 0 | 0 | 0 | 0 | 2 | 0 |
| K | 0 | 0 | 0 | 0 | 0 | 2 | 0 | 0 |
| L | 0 | 0 | 0 | 0 | 0 | 0 | 0 | 0 |
| M | 0 | 0 | 0 | 0 | 0 | 96 | 0 | 0 |
| N | 0 | 0 | 72 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 0 | 0 | 0 | 98 | 0 | 0 | 98 |
| T | 0 | 0 | 28 | 0 | 0 | 0 | 0 | 0 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 98 | 0 |

TABLE 4-17
CDR2 amino acid frequency distribution
for cluster N1364-N1365 (Group J)

Position

| AA | 56 | 57 | 58 | 59 | 62 | 63 | 64 | 65 |
|---|---|---|---|---|---|---|---|---|
| A | 0 | 0 | 0 | 0 | 0 | 0 | 0 | 0 |
| C | 0 | 0 | 0 | 0 | 0 | 0 | 0 | 0 |
| D | 0 | 98 | 0 | 0 | 0 | 2 | 0 | 0 |
| E | 0 | 0 | 0 | 0 | 0 | 0 | 0 | 0 |
| F | 0 | 0 | 0 | 0 | 0 | 0 | 0 | 0 |
| G | 0 | 0 | 0 | 84 | 2 | 96 | 0 | 0 |
| H | 0 | 0 | 0 | 0 | 0 | 0 | 0 | 0 |
| I | 100 | 0 | 0 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 0 | 0 | 0 | 0 | 0 | 4 | 0 |
| P | 0 | 0 | 0 | 0 | 0 | 2 | 0 | 0 |
| Q | 0 | 0 | 0 | 0 | 0 | 0 | 0 | 0 |
| R | 0 | 0 | 0 | 12 | 0 | 0 | 0 | 0 |
| S | 0 | 0 | 2 | 0 | 0 | 0 | 96 | 0 |
| T | 0 | 0 | 98 | 0 | 0 | 0 | 0 | 100 |
| V | 0 | 0 | 0 | 2 | 0 | 0 | 0 | 0 |
| W | 0 | 0 | 0 | 0 | 0 | 0 | 0 | 0 |
| Y | 0 | 2 | 0 | 2 | 0 | 0 | 0 | 0 |

TABLE 4-18
CDR3 amino acid frequency distribution for cluster N1364-N1365 (Group J)

| | Position | | | | | | | | | | | |
|---|---|---|---|---|---|---|---|---|---|---|---|---|
| AA | 105 | 106 | 107 | 108 | 109 | 110 | 112 | 113 | 114 | 115 | 116 | 117 |
| A | 100 | 0 | 0 | 0 | 0 | 18 | 0 | 0 | 2 | 0 | 2 | 0 |
| C | 0 | 0 | 0 | 0 | 0 | 0 | 0 | 0 | 0 | 0 | 0 | 0 |
| D | 0 | 0 | 0 | 0 | 2 | 0 | 0 | 2 | 16 | 0 | 0 | 0 |
| E | 0 | 0 | 0 | 0 | 0 | 0 | 0 | 0 | 70 | 0 | 0 | 0 |
| F | 0 | 0 | 0 | 0 | 0 | 0 | 0 | 2 | 0 | 0 | 0 | 0 |
| G | 0 | 0 | 0 | 16 | 96 | 0 | 0 | 0 | 8 | 0 | 66 | 0 |
| H | 0 | 0 | 0 | 0 | 0 | 0 | 0 | 2 | 0 | 0 | 0 | 0 |
| I | 0 | 0 | 0 | 0 | 0 | 0 | 0 | 0 | 0 | 0 | 0 | 0 |
| K | 0 | 0 | 0 | 0 | 0 | 2 | 0 | 0 | 2 | 0 | 0 | 0 |
| L | 0 | 0 | 0 | 2 | 0 | 0 | 0 | 0 | 0 | 0 | 0 | 0 |
| M | 0 | 0 | 0 | 0 | 0 | 0 | 0 | 0 | 0 | 0 | 0 | 0 |
| N | 0 | 0 | 0 | 0 | 0 | 0 | 0 | 2 | 0 | 0 | 32 | 0 |
| P | 0 | 0 | 0 | 0 | 0 | 2 | 94 | 0 | 0 | 0 | 0 | 0 |
| Q | 0 | 0 | 0 | 2 | 0 | 0 | 2 | 0 | 0 | 0 | 0 | 0 |
| R | 0 | 100 | 0 | 78 | 0 | 0 | 2 | 0 | 0 | 0 | 0 | 0 |
| S | 0 | 0 | 0 | 0 | 0 | 16 | 0 | 2 | 0 | 0 | 0 | 0 |
| T | 0 | 0 | 0 | 0 | 0 | 62 | 2 | 0 | 0 | 0 | 0 | 0 |
| V | 0 | 0 | 100 | 0 | 2 | 0 | 0 | 0 | 2 | 0 | 0 | 0 |
| W | 0 | 0 | 0 | 2 | 0 | 0 | 0 | 0 | 0 | 0 | 0 | 0 |
| Y | 0 | 0 | 0 | 0 | 0 | 0 | 0 | 90 | 0 | 100 | 0 | 100 |

TABLE 4-19
CDR1 amino acid frequency distribution
for cluster 35A10-N1402-N1400 (Group K)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 5.9 | 0 |
| E | 5.9 | 0 | 0 | 0 | 5.9 | 0 | 0 | 0 |
| F | 0 | 0 | 0 | 98.0 | 0 | 0 | 0 | 7.8 |
| G | 94.1 | 0 | 0 | 0 | 56.9 | 0 | 0 | 0 |
| H | 0 | 9.8 | 0 | 0 | 0 | 0 | 2.0 | 0 |
| I | 0 | 0 | 0 | 0 | 0 | 0 | 0 | 0 |
| K | 0 | 0 | 0 | 0 | 0 | 0 | 0 | 0 |
| L | 0 | 0 | 0 | 2.0 | 0 | 0 | 2.0 | 0 |
| M | 0 | 0 | 0 | 0 | 0 | 0 | 0 | 0 |
| N | 0 | 0 | 0 | 0 | 0 | 70.6 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 90.2 | 0 | 0 | 27.5 | 0 | 0 | 0 |
| S | 0 | 0 | 0 | 0 | 9.8 | 29.4 | 0 | 0 |
| T | 0 | 0 | 100 | 0 | 0 | 0 | 0 | 92.2 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 90.2 | 0 |

TABLE 4-20
CDR2 amino acid frequency distribution
for cluster 35A10-N1402-N1400 (Group K)

| | Position | | | | | | | |
|---|---|---|---|---|---|---|---|---|
| AA | 56 | 57 | 58 | 59 | 62 | 63 | 64 | 65 |
| A | 0 | 0 | 0 | 2.0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 0 | 0 | 0 | 0 | 0 |
| G | 0 | 0 | 0 | 0 | 98.0 | 88.2 | 0 | 0 |
| H | 0 | 0 | 0 | 0 | 0 | 0 | 0 | 0 |
| I | 100 | 0 | 0 | 0 | 0 | 0 | 7.8 | 0 |
| K | 0 | 2.0 | 0 | 0 | 0 | 0 | 0 | 0 |
| L | 0 | 0 | 0 | 0 | 0 | 9.8 | 0 | 0 |
| M | 0 | 0 | 0 | 0 | 0 | 0 | 0 | 0 |
| N | 0 | 0 | 0 | 0 | 0 | 0 | 0 | 0 |
| P | 0 | 0 | 0 | 13.7 | 0 | 0 | 0 | 0 |
| Q | 0 | 0 | 0 | 0 | 0 | 0 | 0 | 0 |
| R | 0 | 98.0 | 3.9 | 0 | 0 | 2.0 | 0 | 0 |
| S | 0 | 0 | 0 | 0 | 0 | 0 | 90.2 | 0 |
| T | 0 | 0 | 0 | 84.3 | 2.0 | 0 | 2.0 | 100 |
| V | 0 | 0 | 0 | 0 | 0 | 0 | 0 | 0 |
| W | 0 | 0 | 88.2 | 0 | 0 | 0 | 0 | 0 |
| Y | 0 | 0 | 7.8 | 0 | 0 | 0 | 0 | 0 |

TABLE 4-21
CDR3 amino acid frequency distribution
for cluster 35A10-N1402-N1400 (Group K)

| | Position | | | | | | | | | | |
|---|---|---|---|---|---|---|---|---|---|---|---|
| AA | 105 | 106 | 107 | 108 | 109 | 110 | 113 | 114 | 115 | 116 | 117 |
| A | 21.6 | 100 | 88.2 | 0 | 9.8 | 0 | 0 | 96.1 | 0 | 23.5 | 0 |
| C | 0 | 0 | 0 | 0 | 0 | 0 | 0 | 0 | 0 | 0 | 0 |
| D | 0 | 0 | 0 | 0 | 0 | 0 | 0 | 0 | 0 | 2.0 | 0 |
| E | 0 | 0 | 0 | 0 | 0 | 0 | 0 | 2.0 | 0 | 0 | 0 |
| F | 0 | 0 | 0 | 0 | 0 | 0 | 0 | 0 | 96.1 | 0 | 0 |
| G | 0 | 0 | 0 | 0 | 0 | 86.3 | 0 | 0 | 0 | 0 | 0 |
| H | 0 | 0 | 0 | 2.0 | 0 | 0 | 0 | 0 | 0 | 0 | 2.0 |
| I | 0 | 0 | 0 | 0 | 2.0 | 0 | 0 | 0 | 0 | 0 | 2.0 |
| K | 0 | 0 | 0 | 0 | 0 | 0 | 5.9 | 0 | 0 | 0 | 0 |
| L | 0 | 0 | 0 | 2.0 | 0 | 0 | 0 | 0 | 2.0 | 0 | 0 |
| M | 0 | 0 | 0 | 0 | 0 | 0 | 0 | 0 | 0 | 0 | 0 |
| N | 0 | 0 | 0 | 0 | 0 | 0 | 0 | 0 | 0 | 7.8 | 0 |
| P | 0 | 0 | 0 | 90.2 | 3.9 | 0 | 0 | 0 | 0 | 0 | 0 |
| Q | 0 | 0 | 0 | 0 | 0 | 0 | 3.9 | 0 | 0 | 0 | 0 |
| R | 0 | 0 | 0 | 0 | 0 | 5.9 | 88.2 | 0 | 0 | 0 | 0 |
| S | 0 | 0 | 9.8 | 3.9 | 3.9 | 0 | 2.0 | 2.0 | 0 | 0 | 0 |
| T | 31.4 | 0 | 2.0 | 2.0 | 80.4 | 0 | 0 | 0 | 0 | 66.7 | 0 |
| V | 47.1 | 0 | 0 | 0 | 0 | 5.9 | 0 | 0 | 0 | 0 | 0 |
| W | 0 | 0 | 0 | 0 | 0 | 2.0 | 0 | 0 | 0 | 0 | 0 |
| Y | 0 | 0 | 0 | 0 | 0 | 0 | 0 | 0 | 2.0 | 0 | 96.1 |

TABLE 4-22
CDR1 amino acid frequency distribution
for cluster N1425 (Group L)

| | Position | | | | | | | |
|---|---|---|---|---|---|---|---|---|
| AA | 27 | 28 | 29 | 30 | 35 | 36 | 37 | 38 |
| A | 0 | 100 | 0 | 0 | 0 | 0 | 0 | 100 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 0 | 0 | 0 | 0 | 0 |
| G | 100 | 0 | 0 | 0 | 0 | 0 | 0 | 0 |
| H | 0 | 0 | 0 | 0 | 0 | 0 | 0 | 0 |
| I | 0 | 0 | 0 | 0 | 0 | 0 | 0 | 0 |
| K | 0 | 0 | 0 | 0 | 0 | 0 | 0 | 0 |
| L | 0 | 0 | 0 | 100 | 0 | 0 | 0 | 0 |
| M | 0 | 0 | 0 | 0 | 0 | 0 | 0 | 0 |
| N | 0 | 0 | 0 | 0 | 0 | 0 | 100 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 100 | 0 | 0 |
| S | 0 | 0 | 100 | 0 | 100 | 0 | 0 | 0 |
| T | 0 | 0 | 0 | 0 | 0 | 0 | 0 | 0 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 0 | 0 |

TABLE 4-23
CDR2 amino acid frequency distribution
for cluster N1425 (Group L)

| | Position | | | | | | |
|---|---|---|---|---|---|---|---|
| AA | 56 | 57 | 58 | 59 | 63 | 64 | 65 |
| A | 0 | 0 | 0 | 0 | 0 | 0 | 0 |
| C | 0 | 0 | 0 | 0 | 0 | 0 | 0 |
| D | 0 | 0 | 100 | 100 | 0 | 0 | 0 |
| E | 0 | 0 | 0 | 0 | 0 | 100 | 0 |
| F | 0 | 0 | 0 | 0 | 0 | 0 | 0 |
| G | 0 | 0 | 0 | 0 | 100 | 0 | 0 |
| H | 0 | 0 | 0 | 0 | 0 | 0 | 0 |
| I | 100 | 0 | 0 | 0 | 0 | 0 | 0 |
| K | 0 | 0 | 0 | 0 | 0 | 0 | 0 |
| L | 0 | 0 | 0 | 0 | 0 | 0 | 0 |
| M | 0 | 0 | 0 | 0 | 0 | 0 | 0 |
| N | 0 | 0 | 0 | 0 | 0 | 0 | 0 |
| P | 0 | 0 | 0 | 0 | 0 | 0 | 0 |
| Q | 0 | 0 | 0 | 0 | 0 | 0 | 0 |
| R | 0 | 0 | 0 | 0 | 0 | 0 | 0 |
| S | 0 | 0 | 0 | 0 | 0 | 0 | 0 |
| T | 0 | 0 | 0 | 0 | 0 | 0 | 100 |
| V | 0 | 0 | 0 | 0 | 0 | 0 | 0 |
| W | 0 | 0 | 0 | 0 | 0 | 0 | 0 |
| Y | 0 | 100 | 0 | 0 | 0 | 0 | 0 |

TABLE 4-24
CDR3 amino acid frequency distribution
for cluster N1425 (Group L)

| | Position | | | | | | |
|---|---|---|---|---|---|---|---|
| AA | 105 | 106 | 107 | 108 | 115 | 116 | 117 |
| A | 100 | 0 | 0 | 100 | 0 | 0 | 0 |
| C | 0 | 0 | 0 | 0 | 0 | 0 | 0 |
| D | 0 | 0 | 0 | 0 | 0 | 100 | 0 |
| E | 0 | 0 | 0 | 0 | 0 | 0 | 0 |
| F | 0 | 0 | 0 | 0 | 100 | 0 | 100 |
| G | 0 | 100 | 0 | 0 | 0 | 0 | 0 |
| H | 0 | 0 | 0 | 0 | 0 | 0 | 0 |
| I | 0 | 0 | 0 | 0 | 0 | 0 | 0 |
| K | 0 | 0 | 0 | 0 | 0 | 0 | 0 |
| L | 0 | 0 | 0 | 0 | 0 | 0 | 0 |
| M | 0 | 0 | 0 | 0 | 0 | 0 | 0 |
| N | 0 | 0 | 0 | 0 | 0 | 0 | 0 |
| P | 0 | 0 | 0 | 0 | 0 | 0 | 0 |
| Q | 0 | 0 | 0 | 0 | 0 | 0 | 0 |
| R | 0 | 0 | 0 | 0 | 0 | 0 | 0 |
| S | 0 | 0 | 100 | 0 | 0 | 0 | 0 |
| T | 0 | 0 | 0 | 0 | 0 | 0 | 0 |
| V | 0 | 0 | 0 | 0 | 0 | 0 | 0 |
| W | 0 | 0 | 0 | 0 | 0 | 0 | 0 |
| Y | 0 | 0 | 0 | 0 | 0 | 0 | 0 |

TABLE 4-25
CDR1 amino acid frequency distribution
for cluster N1409 (Group M)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 0 | 0 | 100 | 0 | 100 | 0 | 0 |
| G | 100 | 1.3 | 0 | 0 | 0 | 0 | 0 | 0 |
| H | 0 | 0 | 0 | 0 | 0 | 0 | 0 | 0 |
| I | 0 | 0 | 96 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 2.6 | 0 | 0 | 0 | 0 | 0 | 0 |
| P | 0 | 0 | 0 | 0 | 0 | 0 | 100 | 100 |
| Q | 0 | 0 | 0 | 0 | 0 | 0 | 0 | 0 |
| R | 0 | 0 | 0 | 0 | 100 | 0 | 0 | 0 |
| S | 0 | 96 | 0 | 0 | 0 | 0 | 0 | 0 |
| T | 0 | 0 | 3.9 | 0 | 0 | 0 | 0 | 0 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 0 | 0 |

TABLE 4-26
CDR2 amino acid frequency distribution
for cluster N1409 (Group M)

| | Position | | | | | | |
|---|---|---|---|---|---|---|---|
| AA | 56 | 57 | 58 | 59 | 63 | 64 | 65 |
| A | 0 | 0 | 0 | 0 | 0 | 0 | 1.3 |
| C | 0 | 0 | 0 | 0 | 0 | 0 | 0 |
| D | 0 | 0 | 0 | 0 | 0 | 0 | 0 |
| E | 0 | 0 | 0 | 0 | 0 | 0 | 0 |
| F | 0 | 0 | 0 | 0 | 0 | 0 | 0 |
| G | 0 | 0 | 0 | 100 | 100 | 0 | 0 |
| H | 0 | 0 | 0 | 0 | 0 | 0 | 0 |
| I | 0 | 0 | 0 | 0 | 0 | 0 | 0 |
| K | 0 | 0 | 0 | 0 | 0 | 0 | 0 |
| L | 100 | 0 | 0 | 0 | 0 | 0 | 0 |
| M | 0 | 0 | 0 | 0 | 0 | 0 | 0 |
| N | 0 | 0 | 0 | 0 | 0 | 0 | 0 |
| P | 0 | 0 | 0 | 0 | 0 | 0 | 0 |
| Q | 0 | 0 | 0 | 0 | 0 | 0 | 0 |
| R | 0 | 0 | 0 | 0 | 0 | 0 | 0 |
| S | 0 | 0 | 100 | 0 | 0 | 100 | 0 |
| T | 0 | 100 | 0 | 0 | 0 | 0 | 99 |
| V | 0 | 0 | 0 | 0 | 0 | 0 | 0 |
| W | 0 | 0 | 0 | 0 | 0 | 0 | 0 |
| Y | 0 | 0 | 0 | 0 | 0 | 0 | 0 |

TABLE 4-27
CDR3 amino acid frequency distribution for cluster N1409 (Group M)

| | Position | | | | | | | | | |
|---|---|---|---|---|---|---|---|---|---|---|
| AA | 105 | 106 | 107 | 108 | 109 | 113 | 114 | 115 | 116 | 117 |
| A | 2.6 | 1.3 | 0 | 0 | 0 | 0 | 0 | 0 | 1.3 | 0 |
| C | 0 | 0 | 0 | 0 | 0 | 0 | 0 | 0 | 0 | 0 |
| D | 0 | 0 | 0 | 0 | 0 | 96 | 0 | 0 | 0 | 5.1 |
| E | 0 | 1.3 | 0 | 0 | 0 | 1.3 | 0 | 0 | 0 | 0 |
| F | 1.3 | 0 | 0 | 0 | 0 | 0 | 0 | 0 | 0 | 0 |
| G | 0 | 1.3 | 1.3 | 96 | 3.8 | 0 | 0 | 0 | 0 | 0 |
| H | 0 | 0 | 0 | 0 | 0 | 0 | 0 | 0 | 0 | 1.3 |
| I | 0 | 1.3 | 2.6 | 0 | 1.3 | 0 | 0 | 1.3 | 0 | 0 |
| K | 0 | 0 | 0 | 0 | 0 | 0 | 3.8 | 0 | 1.3 | 0 |
| L | 0 | 2.6 | 64 | 0 | 0 | 0 | 2.6 | 2.6 | 0 | 0 |
| M | 0 | 23 | 0 | 0 | 0 | 0 | 92 | 87 | 0 | 0 |
| N | 0 | 0 | 0 | 0 | 0 | 0 | 0 | 0 | 0 | 0 |
| P | 0 | 0 | 1.3 | 0 | 0 | 0 | 0 | 0 | 1.3 | 0 |
| Q | 0 | 0 | 0 | 0 | 0 | 0 | 0 | 0 | 0 | 0 |
| R | 0 | 1.3 | 1.3 | 1.3 | 90 | 0 | 1.3 | 3.8 | 0 | 0 |
| S | 87 | 0 | 0 | 0 | 5.1 | 0 | 0 | 0 | 1.3 | 1.3 |
| T | 2.6 | 0 | 0 | 0 | 0 | 0 | 0 | 0 | 95 | 0 |
| V | 0 | 68 | 29 | 2.6 | 0 | 1.3 | 0 | 5.1 | 0 | 0 |
| W | 0 | 0 | 0 | 0 | 0 | 0 | 0 | 0 | 0 | 0 |
| Y | 6.4 | 0 | 0 | 0 | 0 | 1.3 | 0 | 0 | 0 | 92 |

TABLE 4-28
CDR1 amino acid frequency distribution
for cluster N1323 (Group N)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 100 |
| C | 0 | 0 | 0 | 0 | 0 | 0 | 0 | 0 |
| D | 0 | 0 | 0 | 0 | 100 | 100 | 0 | 0 |
| E | 0 | 0 | 0 | 0 | 0 | 0 | 0 | 0 |
| F | 0 | 100 | 0 | 11.1 | 0 | 0 | 0 | 0 |
| G | 100 | 0 | 0 | 0 | 0 | 0 | 0 | 0 |
| H | 0 | 0 | 0 | 0 | 0 | 0 | 0 | 0 |
| I | 0 | 0 | 0 | 0 | 0 | 0 | 11.1 | 0 |
| K | 0 | 0 | 0 | 0 | 0 | 0 | 0 | 0 |
| L | 0 | 0 | 0 | 88.9 | 0 | 0 | 0 | 0 |
| M | 0 | 0 | 0 | 0 | 0 | 0 | 0 | 0 |
| N | 0 | 0 | 0 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 0 | 0 | 0 | 0 | 0 | 0 | 0 |
| T | 0 | 0 | 100 | 0 | 0 | 0 | 0 | 0 |
| V | 0 | 0 | 0 | 0 | 0 | 0 | 0 | 0 |
| W | 0 | 0 | 0 | 0 | 0 | 0 | 0 | 0 |
| Y | 0 | 0 | 0 | 0 | 0 | 0 | 88.9 | 0 |

TABLE 4-29
CDR2 amino acid frequency distribution
for cluster N1323 (Group N)

| | Position | | | | | | | |
|---|---|---|---|---|---|---|---|---|
| AA | 56 | 57 | 58 | 59 | 62 | 63 | 64 | 65 |
| A | 0 | 0 | 0 | 0 | 0 | 0 | 0 | 0 |
| C | 0 | 0 | 0 | 0 | 0 | 0 | 0 | 0 |
| D | 0 | 0 | 0 | 0 | 0 | 0 | 0 | 0 |
| E | 0 | 0 | 0 | 0 | 0 | 0 | 0 | 0 |
| F | 0 | 88.9 | 0 | 0 | 0 | 0 | 0 | 0 |
| G | 0 | 0 | 0 | 0 | 11.1 | 0 | 0 | 0 |
| H | 0 | 0 | 0 | 0 | 0 | 0 | 0 | 0 |
| I | 100 | 0 | 0 | 0 | 0 | 0 | 0 | 0 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 0 | 0 | 0 | 0 | 0 | 100 | 0 |
| P | 0 | 0 | 0 | 0 | 0 | 11.1 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 0 | 100 | 0 | 88.9 | 77.8 | 0 | 0 |
| T | 0 | 0 | 0 | 0 | 0 | 11.1 | 0 | 100 |
| V | 0 | 0 | 0 | 0 | 0 | 0 | 0 | 0 |
| W | 0 | 0 | 0 | 0 | 0 | 0 | 0 | 0 |
| Y | 0 | 11.1 | 0 | 100 | 0 | 0 | 0 | 0 |

TABLE 4-30
CDR3 amino acid frequency distribution for cluster N1323 (Group N)

| | Position | | | | | | | | | | | | | | |
|---|---|---|---|---|---|---|---|---|---|---|---|---|---|---|---|
| AA | 105 | 106 | 107 | 108 | 109 | 110 | 111 | 111.1 | 112.1 | 112 | 113 | 114 | 115 | 116 | 117 |
| A | 100 | 0 | 0 | 0 | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 | 0 | 0 | 0 | 0 |
| D | 0 | 0 | 0 | 100 | 0 | 0 | 0 | 0 | 0 | 0 | 0 | 0 | 0 | 100 | 0 |
| E | 0 | 0 | 0 | 0 | 0 | 100 | 0 | 100 | 0 | 0 | 0 | 11.1 | 0 | 0 | 0 |
| F | 0 | 0 | 0 | 0 | 88.9 | 0 | 0 | 0 | 0 | 0 | 0 | 0 | 0 | 0 | 0 |
| G | 0 | 0 | 100 | 0 | 0 | 0 | 100 | 0 | 0 | 0 | 0 | 0 | 100 | 0 | 0 |
| H | 0 | 0 | 0 | 0 | 0 | 0 | 0 | 0 | 0 | 0 | 0 | 0 | 0 | 0 | 0 |
| I | 0 | 0 | 0 | 0 | 0 | 0 | 0 | 0 | 0 | 0 | 0 | 0 | 0 | 0 | 0 |
| K | 0 | 0 | 0 | 0 | 0 | 0 | 0 | 0 | 0 | 0 | 0 | 88.9 | 0 | 0 | 0 |
| L | 0 | 0 | 0 | 0 | 11.1 | 0 | 0 | 0 | 100 | 0 | 100 | 0 | 0 | 0 | 0 |
| M | 0 | 0 | 0 | 0 | 0 | 0 | 0 | 0 | 0 | 0 | 0 | 0 | 0 | 0 | 0 |
| N | 0 | 0 | 0 | 0 | 0 | 0 | 0 | 0 | 0 | 0 | 0 | 0 | 0 | 0 | 0 |
| P | 0 | 0 | 0 | 0 | 0 | 0 | 0 | 0 | 0 | 0 | 0 | 0 | 0 | 0 | 0 |
| Q | 0 | 0 | 0 | 0 | 0 | 0 | 0 | 0 | 0 | 0 | 0 | 0 | 0 | 0 | 0 |
| R | 0 | 0 | 0 | 0 | 0 | 0 | 0 | 0 | 0 | 0 | 0 | 0 | 0 | 0 | 0 |
| S | 0 | 0 | 0 | 0 | 0 | 0 | 0 | 0 | 0 | 0 | 0 | 0 | 0 | 0 | 0 |
| T | 0 | 0 | 0 | 0 | 0 | 0 | 0 | 0 | 0 | 0 | 0 | 0 | 0 | 0 | 0 |
| V | 0 | 100 | 0 | 0 | 0 | 0 | 0 | 0 | 0 | 100 | 0 | 0 | 0 | 0 | 0 |
| W | 0 | 0 | 0 | 0 | 0 | 0 | 0 | 0 | 0 | 0 | 0 | 0 | 0 | 0 | 0 |
| Y | 0 | 0 | 0 | 0 | 0 | 0 | 0 | 0 | 0 | 0 | 0 | 0 | 0 | 0 | 100 |

TABLE 4-31
CDR1 amino acid frequency distribution
for cluster 33B1 (Group O)

| | Position | | | | | | | |
|---|---|---|---|---|---|---|---|---|
| AA | 27 | 28 | 29 | 30 | 35 | 36 | 37 | 38 |
| A | 0 | 0 | 0 | 0 | 0 | 0 | 0 | 7.7 |
| C | 0 | 0 | 0 | 0 | 0 | 0 | 0 | 0 |
| D | 0 | 0 | 0 | 0 | 92.3 | 15.4 | 0 | 0 |
| E | 0 | 0 | 0 | 0 | 0 | 0 | 0 | 0 |
| F | 0 | 38.5 | 0 | 7.7 | 0 | 0 | 0 | 0 |
| G | 100 | 0 | 0 | 0 | 7.7 | 0 | 0 | 0 |
| H | 0 | 0 | 0 | 0 | 0 | 7.7 | 61.5 | 0 |
| I | 0 | 0 | 0 | 0 | 0 | 0 | 0 | 0 |
| K | 0 | 0 | 0 | 0 | 0 | 0 | 0 | 0 |
| L | 0 | 0 | 0 | 92.3 | 0 | 0 | 0 | 0 |
| M | 0 | 0 | 0 | 0 | 0 | 0 | 0 | 0 |
| N | 0 | 0 | 0 | 0 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 0 | 0 | 0 | 0 | 0 | 0 |
| S | 0 | 0 | 69.2 | 0 | 0 | 0 | 0 | 0 |
| T | 0 | 0 | 30.8 | 0 | 0 | 0 | 0 | 92.3 |
| V | 0 | 61.5 | 0 | 0 | 0 | 0 | 0 | 0 |
| W | 0 | 0 | 0 | 0 | 0 | 0 | 0 | 0 |
| Y | 0 | 0 | 0 | 0 | 0 | 76.9 | 38.5 | 0 |

TABLE 4-32
CDR2 amino acid frequency distribution
for cluster 33B1 (Group O)

| | Position | | | | | | | |
|---|---|---|---|---|---|---|---|---|
| AA | 56 | 57 | 58 | 59 | 62 | 63 | 64 | 65 |
| A | 0 | 0 | 0 | 0 | 0 | 0 | 0 | 0 |
| C | 0 | 0 | 0 | 0 | 0 | 0 | 0 | 0 |
| D | 0 | 7.7 | 0 | 7.7 | 100 | 0 | 0 | 0 |
| E | 0 | 0 | 0 | 0 | 0 | 0 | 0 | 0 |
| F | 0 | 0 | 0 | 7.7 | 0 | 0 | 0 | 0 |
| G | 0 | 0 | 0 | 0 | 0 | 100 | 0 | 0 |
| H | 0 | 0 | 7.7 | 7.7 | 0 | 0 | 0 | 0 |
| I | 100 | 0 | 0 | 7.7 | 0 | 0 | 0 | 7.7 |
| K | 0 | 0 | 0 | 0 | 0 | 0 | 0 | 0 |
| L | 0 | 0 | 0 | 0 | 0 | 0 | 0 | 0 |
| M | 0 | 0 | 0 | 0 | 0 | 0 | 0 | 0 |
| N | 0 | 23.1 | 7.7 | 69.2 | 0 | 0 | 0 | 0 |
| P | 0 | 0 | 0 | 0 | 0 | 0 | 0 | 0 |
| Q | 0 | 0 | 0 | 0 | 0 | 0 | 0 | 0 |
| R | 0 | 0 | 7.7 | 0 | 0 | 0 | 0 | 0 |
| S | 0 | 69.2 | 76.9 | 0 | 0 | 0 | 76.9 | 0 |
| T | 0 | 0 | 0 | 0 | 0 | 0 | 23.1 | 61.5 |
| V | 0 | 0 | 0 | 0 | 0 | 0 | 0 | 30.8 |
| W | 0 | 0 | 0 | 0 | 0 | 0 | 0 | 0 |
| Y | 0 | 0 | 0 | 0 | 0 | 0 | 0 | 0 |

TABLE 4-33
CDR3 amino acid frequency distribution for cluster 33B1 (Group O)

| | Position | | | | | | | | | |
|---|---|---|---|---|---|---|---|---|---|---|
| AA | 105 | 106 | 107 | 108 | 109 | 110 | 111 | 111.1 | 111.2 | 111.3 |
| A | 100 | 100 | 0 | 0 | 0 | 0 | 0 | 0 | 46.2 | 0 |
| C | 0 | 0 | 0 | 0 | 0 | 0 | 0 | 0 | 0 | 0 |
| D | 0 | 0 | 100 | 0 | 0 | 0 | 0 | 0 | 0 | 100 |
| E | 0 | 0 | 0 | 0 | 0 | 0 | 0 | 0 | 0 | 0 |
| F | 0 | 0 | 0 | 0 | 0 | 30.8 | 0 | 0 | 0 | 0 |
| G | 0 | 0 | 0 | 0 | 100 | 0 | 0 | 0 | 0 | 0 |
| H | 0 | 0 | 0 | 0 | 0 | 0 | 0 | 0 | 0 | 0 |
| I | 0 | 0 | 0 | 0 | 0 | 0 | 0 | 0 | 0 | 0 |
| K | 0 | 0 | 0 | 0 | 0 | 0 | 0 | 0 | 0 | 0 |
| L | 0 | 0 | 0 | 61.5 | 0 | 0 | 100 | 0 | 0 | 0 |
| M | 0 | 0 | 0 | 0 | 0 | 0 | 0 | 0 | 0 | 0 |
| N | 0 | 0 | 0 | 0 | 0 | 0 | 0 | 0 | 0 | 0 |
| P | 0 | 0 | 0 | 0 | 0 | 0 | 0 | 0 | 0 | 0 |
| Q | 0 | 0 | 0 | 0 | 0 | 0 | 0 | 0 | 0 | 0 |
| R | 0 | 0 | 0 | 0 | 0 | 0 | 0 | 0 | 0 | 0 |
| S | 0 | 0 | 0 | 0 | 0 | 0 | 0 | 0 | 0 | 0 |
| T | 0 | 0 | 0 | 0 | 0 | 0 | 0 | 0 | 23.1 | 0 |
| V | 0 | 0 | 0 | 38.5 | 0 | 61.5 | 0 | 0 | 30.8 | 0 |
| W | 0 | 0 | 0 | 0 | 0 | 0 | 0 | 0 | 0 | 0 |
| Y | 0 | 0 | 0 | 0 | 0 | 7.7 | 0 | 100 | 0 | 0 |

| | Position | | | | | | | | | | |
|---|---|---|---|---|---|---|---|---|---|---|---|
| AA | 111.4 | 112.4 | 112.3 | 112.2 | 112.1 | 112 | 113 | 114 | 115 | 116 | 117 |
| A | 0 | 0 | 0 | 0 | 0 | 0 | 0 | 0 | 0 | 0 | 0 |
| C | 0 | 0 | 0 | 0 | 0 | 0 | 0 | 0 | 0 | 0 | 0 |
| D | 0 | 0 | 0 | 0 | 0 | 0 | 0 | 0 | 0 | 0 | 0 |
| E | 0 | 0 | 0 | 0 | 0 | 0 | 0 | 0 | 0 | 0 | 0 |
| F | 0 | 0 | 0 | 0 | 0 | 0 | 0 | 0 | 100 | 0 | 0 |
| G | 0 | 0 | 0 | 0 | 0 | 0 | 0 | 0 | 0 | 100 | 0 |
| H | 0 | 0 | 0 | 0 | 92.3 | 0 | 100 | 100 | 0 | 0 | 0 |
| I | 0 | 0 | 0 | 0 | 0 | 0 | 0 | 0 | 0 | 0 | 0 |
| K | 0 | 0 | 0 | 0 | 0 | 0 | 0 | 0 | 0 | 0 | 0 |
| L | 0 | 0 | 0 | 100 | 0 | 0 | 0 | 0 | 0 | 0 | 0 |
| M | 0 | 0 | 0 | 0 | 0 | 46.2 | 0 | 0 | 0 | 0 | 0 |
| N | 0 | 0 | 0 | 0 | 0 | 0 | 0 | 0 | 0 | 0 | 0 |
| P | 0 | 0 | 15.4 | 0 | 0 | 0 | 0 | 0 | 0 | 0 | 0 |
| Q | 0 | 0 | 0 | 0 | 0 | 0 | 0 | 0 | 0 | 0 | 0 |
| R | 0 | 0 | 84.6 | 0 | 0 | 0 | 0 | 0 | 0 | 0 | 0 |
| S | 0 | 0 | 0 | 0 | 0 | 0 | 0 | 0 | 0 | 0 | 100 |
| T | 0 | 0 | 0 | 0 | 0 | 53.8 | 0 | 0 | 0 | 0 | 0 |
| V | 0 | 100 | 0 | 0 | 0 | 0 | 0 | 0 | 0 | 0 | 0 |
| W | 0 | 0 | 0 | 0 | 0 | 0 | 0 | 0 | 0 | 0 | 0 |
| Y | 100 | 0 | 0 | 0 | 7.7 | 0 | 0 | 0 | 0 | 0 | 0 |

TABLE 5
Sequence Identifiers of V-bodies identified from panning

| | CDR1 | CDR2 | CDR3 | VHH | |
|---|---|---|---|---|---|
| | Amino | Amino | Amino | Amino | VHH |
| | Acid | Acid | Acid | Acid | DNA |
| Group | Sequence | Sequence | Sequence | Sequence | Sequence |
| A | 1128 | 1687 | 2246 | 2805 | 3364 |
| A | 1129 | 1688 | 2247 | 2806 | 3365 |
| A | 1130 | 1689 | 2248 | 2807 | 3366 |
| A | 1131 | 1690 | 2249 | 2808 | 3367 |
| A | 1132 | 1691 | 2250 | 2809 | 3368 |
| A | 1133 | 1692 | 2251 | 2810 | 3369 |
| A | 1134 | 1693 | 2252 | 2811 | 3370 |
| A | 1135 | 1694 | 2253 | 2812 | 3371 |
| A | 1136 | 1695 | 2254 | 2813 | 3372 |
| A | 1137 | 1696 | 2255 | 2814 | 3373 |
| A | 1138 | 1697 | 2256 | 2815 | 3374 |
| A | 1139 | 1698 | 2257 | 2816 | 3375 |
| A | 1140 | 1699 | 2258 | 2817 | 3376 |
| A | 1141 | 1700 | 2259 | 2818 | 3377 |
| A | 1142 | 1701 | 2260 | 2819 | 3378 |
| A | 1143 | 1702 | 2261 | 2820 | 3379 |
| A | 1144 | 1703 | 2262 | 2821 | 3380 |
| A | 1145 | 1704 | 2263 | 2822 | 3381 |
| A | 1146 | 1705 | 2264 | 2823 | 3382 |
| A | 1147 | 1706 | 2265 | 2824 | 3383 |
| A | 1148 | 1707 | 2266 | 2825 | 3384 |
| A | 1149 | 1708 | 2267 | 2826 | 3385 |
| A | 1150 | 1709 | 2268 | 2827 | 3386 |
| A | 1151 | 1710 | 2269 | 2828 | 3387 |
| A | 1152 | 1711 | 2270 | 2829 | 3388 |
| A | 1153 | 1712 | 2271 | 2830 | 3389 |
| A | 1154 | 1713 | 2272 | 2831 | 3390 |
| A | 1155 | 1714 | 2273 | 2832 | 3391 |
| A | 1156 | 1715 | 2274 | 2833 | 3392 |
| A | 1157 | 1716 | 2275 | 2834 | 3393 |
| A | 1158 | 1717 | 2276 | 2835 | 3394 |
| A | 1159 | 1718 | 2277 | 2836 | 3395 |
| A | 1160 | 1719 | 2278 | 2837 | 3396 |
| A | 1161 | 1720 | 2279 | 2838 | 3397 |
| A | 1162 | 1721 | 2280 | 2839 | 3398 |
| A | 1163 | 1722 | 2281 | 2840 | 3399 |
| A | 1164 | 1723 | 2282 | 2841 | 3400 |
| A | 1165 | 1724 | 2283 | 2842 | 3401 |
| A | 1166 | 1725 | 2284 | 2843 | 3402 |
| A | 1167 | 1726 | 2285 | 2844 | 3403 |
| A | 1168 | 1727 | 2286 | 2845 | 3404 |
| A | 1169 | 1728 | 2287 | 2846 | 3405 |
| A | 1170 | 1729 | 2288 | 2847 | 3406 |
| A | 1171 | 1730 | 2289 | 2848 | 3407 |
| A | 1172 | 1731 | 2290 | 2849 | 3408 |
| A | 1173 | 1732 | 2291 | 2850 | 3409 |
| A | 1174 | 1733 | 2292 | 2851 | 3410 |
| A | 1175 | 1734 | 2293 | 2852 | 3411 |
| A | 1176 | 1735 | 2294 | 2853 | 3412 |
| A | 1177 | 1736 | 2295 | 2854 | 3413 |
| A | 1178 | 1737 | 2296 | 2855 | 3414 |
| A | 1179 | 1738 | 2297 | 2856 | 3415 |
| A | 1180 | 1739 | 2298 | 2857 | 3416 |
| A | 1181 | 1740 | 2299 | 2858 | 3417 |
| A | 1182 | 1741 | 2300 | 2859 | 3418 |
| A | 1183 | 1742 | 2301 | 2860 | 3419 |
| A | 1184 | 1743 | 2302 | 2861 | 3420 |
| A | 1185 | 1744 | 2303 | 2862 | 3421 |
| A | 1186 | 1745 | 2304 | 2863 | 3422 |
| A | 1187 | 1746 | 2305 | 2864 | 3423 |
| A | 1188 | 1747 | 2306 | 2865 | 3424 |
| A | 1189 | 1748 | 2307 | 2866 | 3425 |
| A | 1190 | 1749 | 2308 | 2867 | 3426 |
| A | 1191 | 1750 | 2309 | 2868 | 3427 |
| A | 1192 | 1751 | 2310 | 2869 | 3428 |
| A | 1193 | 1752 | 2311 | 2870 | 3429 |
| A | 1194 | 1753 | 2312 | 2871 | 3430 |
| A | 1195 | 1754 | 2313 | 2872 | 3431 |
| A | 1196 | 1755 | 2314 | 2873 | 3432 |
| A | 1197 | 1756 | 2315 | 2874 | 3433 |
| A | 1198 | 1757 | 2316 | 2875 | 3434 |
| A | 1199 | 1758 | 2317 | 2876 | 3435 |
| A | 1200 | 1759 | 2318 | 2877 | 3436 |
| A | 1201 | 1760 | 2319 | 2878 | 3437 |
| A | 1202 | 1761 | 2320 | 2879 | 3438 |
| A | 1203 | 1762 | 2321 | 2880 | 3439 |
| A | 1204 | 1763 | 2322 | 2881 | 3440 |
| A | 1205 | 1764 | 2323 | 2882 | 3441 |
| A | 1206 | 1765 | 2324 | 2883 | 3442 |
| A | 1207 | 1766 | 2325 | 2884 | 3443 |
| A | 1208 | 1767 | 2326 | 2885 | 3444 |
| A | 1209 | 1768 | 2327 | 2886 | 3445 |
| A | 1210 | 1769 | 2328 | 2887 | 3446 |
| A | 1211 | 1770 | 2329 | 2888 | 3447 |
| A | 1212 | 1771 | 2330 | 2889 | 3448 |
| A | 1213 | 1772 | 2331 | 2890 | 3449 |
| A | 1214 | 1773 | 2332 | 2891 | 3450 |
| A | 1215 | 1774 | 2333 | 2892 | 3451 |
| A | 1216 | 1775 | 2334 | 2893 | 3452 |
| A | 1217 | 1776 | 2335 | 2894 | 3453 |
| A | 1218 | 1777 | 2336 | 2895 | 3454 |
| A | 1219 | 1778 | 2337 | 2896 | 3455 |
| A | 1220 | 1779 | 2338 | 2897 | 3456 |
| A | 1221 | 1780 | 2339 | 2898 | 3457 |
| A | 1222 | 1781 | 2340 | 2899 | 3458 |
| A | 1223 | 1782 | 2341 | 2900 | 3459 |
| A | 1224 | 1783 | 2342 | 2901 | 3460 |
| A | 1225 | 1784 | 2343 | 2902 | 3461 |
| A | 1226 | 1785 | 2344 | 2903 | 3462 |
| A | 1227 | 1786 | 2345 | 2904 | 3463 |
| A | 1228 | 1787 | 2346 | 2905 | 3464 |
| A | 1229 | 1788 | 2347 | 2906 | 3465 |
| A | 1230 | 1789 | 2348 | 2907 | 3466 |
| A | 1231 | 1790 | 2349 | 2908 | 3467 |
| A | 1232 | 1791 | 2350 | 2909 | 3468 |
| A | 1233 | 1792 | 2351 | 2910 | 3469 |
| A | 1234 | 1793 | 2352 | 2911 | 3470 |
| A | 1235 | 1794 | 2353 | 2912 | 3471 |
| A | 1236 | 1795 | 2354 | 2913 | 3472 |
| A | 1237 | 1796 | 2355 | 2914 | 3473 |
| A | 1238 | 1797 | 2356 | 2915 | 3474 |
| A | 1239 | 1798 | 2357 | 2916 | 3475 |
| A | 1240 | 1799 | 2358 | 2917 | 3476 |
| A | 1241 | 1800 | 2359 | 2918 | 3477 |
| A | 1242 | 1801 | 2360 | 2919 | 3478 |
| A | 1243 | 1802 | 2361 | 2920 | 3479 |
| A | 1244 | 1803 | 2362 | 2921 | 3480 |
| A | 1245 | 1804 | 2363 | 2922 | 3481 |
| A | 1246 | 1805 | 2364 | 2923 | 3482 |
| A | 1247 | 1806 | 2365 | 2924 | 3483 |
| A | 1248 | 1807 | 2366 | 2925 | 3484 |
| A | 1249 | 1808 | 2367 | 2926 | 3485 |
| A | 1250 | 1809 | 2368 | 2927 | 3486 |
| A | 1251 | 1810 | 2369 | 2928 | 3487 |
| A | 1252 | 1811 | 2370 | 2929 | 3488 |
| A | 1253 | 1812 | 2371 | 2930 | 3489 |
| A | 1254 | 1813 | 2372 | 2931 | 3490 |
| A | 1255 | 1814 | 2373 | 2932 | 3491 |
| A | 1256 | 1815 | 2374 | 2933 | 3492 |
| A | 1257 | 1816 | 2375 | 2934 | 3493 |
| A | 1258 | 1817 | 2376 | 2935 | 3494 |
| A | 1259 | 1818 | 2377 | 2936 | 3495 |
| A | 1260 | 1819 | 2378 | 2937 | 3496 |
| A | 1261 | 1820 | 2379 | 2938 | 3497 |
| A | 1262 | 1821 | 2380 | 2939 | 3498 |
| A | 1263 | 1822 | 2381 | 2940 | 3499 |
| A | 1264 | 1823 | 2382 | 2941 | 3500 |
| A | 1265 | 1824 | 2383 | 2942 | 3501 |
| A | 1266 | 1825 | 2384 | 2943 | 3502 |
| A | 1267 | 1826 | 2385 | 2944 | 3503 |
| A | 1268 | 1827 | 2386 | 2945 | 3504 |
| A | 1269 | 1828 | 2387 | 2946 | 3505 |
| A | 1270 | 1829 | 2388 | 2947 | 3506 |
| A | 1271 | 1830 | 2389 | 2948 | 3507 |
| A | 1272 | 1831 | 2390 | 2949 | 3508 |
| A | 1273 | 1832 | 2391 | 2950 | 3509 |
| A | 1274 | 1833 | 2392 | 2951 | 3510 |
| A | 1275 | 1834 | 2393 | 2952 | 3511 |
| A | 1276 | 1835 | 2394 | 2953 | 3512 |
| A | 1277 | 1836 | 2395 | 2954 | 3513 |
| A | 1278 | 1837 | 2396 | 2955 | 3514 |
| A | 1279 | 1838 | 2397 | 2956 | 3515 |
| A | 1280 | 1839 | 2398 | 2957 | 3516 |
| A | 1281 | 1840 | 2399 | 2958 | 3517 |
| A | 1282 | 1841 | 2400 | 2959 | 3518 |
| A | 1283 | 1842 | 2401 | 2960 | 3519 |
| A | 1284 | 1843 | 2402 | 2961 | 3520 |
| A | 1285 | 1844 | 2403 | 2962 | 3521 |
| A | 1286 | 1845 | 2404 | 2963 | 3522 |
| A | 1287 | 1846 | 2405 | 2964 | 3523 |
| A | 1288 | 1847 | 2406 | 2965 | 3524 |
| A | 1289 | 1848 | 2407 | 2966 | 3525 |
| A | 1290 | 1849 | 2408 | 2967 | 3526 |
| A | 1291 | 1850 | 2409 | 2968 | 3527 |
| A | 1292 | 1851 | 2410 | 2969 | 3528 |
| A | 1293 | 1852 | 2411 | 2970 | 3529 |
| A | 1294 | 1853 | 2412 | 2971 | 3530 |
| A | 1295 | 1854 | 2413 | 2972 | 3531 |
| A | 1296 | 1855 | 2414 | 2973 | 3532 |
| A | 1297 | 1856 | 2415 | 2974 | 3533 |
| A | 1298 | 1857 | 2416 | 2975 | 3534 |
| A | 1299 | 1858 | 2417 | 2976 | 3535 |
| A | 1300 | 1859 | 2418 | 2977 | 3536 |
| A | 1301 | 1860 | 2419 | 2978 | 3537 |
| A | 1302 | 1861 | 2420 | 2979 | 3538 |
| A | 1303 | 1862 | 2421 | 2980 | 3539 |
| A | 1304 | 1863 | 2422 | 2981 | 3540 |
| A | 1305 | 1864 | 2423 | 2982 | 3541 |
| A | 1306 | 1865 | 2424 | 2983 | 3542 |
| A | 1307 | 1866 | 2425 | 2984 | 3543 |
| A | 1308 | 1867 | 2426 | 2985 | 3544 |
| A | 1309 | 1868 | 2427 | 2986 | 3545 |
| A | 1310 | 1869 | 2428 | 2987 | 3546 |
| A | 1311 | 1870 | 2429 | 2988 | 3547 |
| A | 1312 | 1871 | 2430 | 2989 | 3548 |
| A | 1313 | 1872 | 2431 | 2990 | 3549 |
| A | 1314 | 1873 | 2432 | 2991 | 3550 |
| A | 1315 | 1874 | 2433 | 2992 | 3551 |
| A | 1316 | 1875 | 2434 | 2993 | 3552 |
| A | 1317 | 1876 | 2435 | 2994 | 3553 |
| A | 1318 | 1877 | 2436 | 2995 | 3554 |
| A | 1319 | 1878 | 2437 | 2996 | 3555 |
| A | 1320 | 1879 | 2438 | 2997 | 3556 |
| A | 1321 | 1880 | 2439 | 2998 | 3557 |
| A | 1322 | 1881 | 2440 | 2999 | 3558 |
| A | 1323 | 1882 | 2441 | 3000 | 3559 |
| A | 1324 | 1883 | 2442 | 3001 | 3560 |
| A | 1325 | 1884 | 2443 | 3002 | 3561 |
| A | 1326 | 1885 | 2444 | 3003 | 3562 |
| A | 1327 | 1886 | 2445 | 3004 | 3563 |
| A | 1328 | 1887 | 2446 | 3005 | 3564 |
| A | 1329 | 1888 | 2447 | 3006 | 3565 |
| A | 1330 | 1889 | 2448 | 3007 | 3566 |
| A | 1331 | 1890 | 2449 | 3008 | 3567 |
| A | 1332 | 1891 | 2450 | 3009 | 3568 |
| A | 1333 | 1892 | 2451 | 3010 | 3569 |
| A | 1334 | 1893 | 2452 | 3011 | 3570 |
| A | 1335 | 1894 | 2453 | 3012 | 3571 |
| A | 1336 | 1895 | 2454 | 3013 | 3572 |
| A | 1337 | 1896 | 2455 | 3014 | 3573 |
| A | 1338 | 1897 | 2456 | 3015 | 3574 |
| A | 1339 | 1898 | 2457 | 3016 | 3575 |
| A | 1340 | 1899 | 2458 | 3017 | 3576 |
| A | 1341 | 1900 | 2459 | 3018 | 3577 |
| A | 1342 | 1901 | 2460 | 3019 | 3578 |
| A | 1343 | 1902 | 2461 | 3020 | 3579 |
| A | 1344 | 1903 | 2462 | 3021 | 3580 |
| A | 1345 | 1904 | 2463 | 3022 | 3581 |
| A | 1346 | 1905 | 2464 | 3023 | 3582 |
| A | 1347 | 1906 | 2465 | 3024 | 3583 |
| A | 1348 | 1907 | 2466 | 3025 | 3584 |
| A | 1349 | 1908 | 2467 | 3026 | 3585 |
| A | 1350 | 1909 | 2468 | 3027 | 3586 |
| A | 1351 | 1910 | 2469 | 3028 | 3587 |
| A | 1352 | 1911 | 2470 | 3029 | 3588 |
| A | 1353 | 1912 | 2471 | 3030 | 3589 |
| A | 1354 | 1913 | 2472 | 3031 | 3590 |
| A | 1355 | 1914 | 2473 | 3032 | 3591 |
| A | 1356 | 1915 | 2474 | 3033 | 3592 |
| A | 1357 | 1916 | 2475 | 3034 | 3593 |
| A | 1358 | 1917 | 2476 | 3035 | 3594 |
| A | 1359 | 1918 | 2477 | 3036 | 3595 |
| A | 1360 | 1919 | 2478 | 3037 | 3596 |
| A | 1361 | 1920 | 2479 | 3038 | 3597 |
| A | 1362 | 1921 | 2480 | 3039 | 3598 |
| A | 1363 | 1922 | 2481 | 3040 | 3599 |
| A | 1364 | 1923 | 2482 | 3041 | 3600 |
| A | 1365 | 1924 | 2483 | 3042 | 3601 |
| A | 1366 | 1925 | 2484 | 3043 | 3602 |
| A | 1367 | 1926 | 2485 | 3044 | 3603 |
| A | 1368 | 1927 | 2486 | 3045 | 3604 |
| A | 1369 | 1928 | 2487 | 3046 | 3605 |
| A | 1370 | 1929 | 2488 | 3047 | 3606 |
| A | 1371 | 1930 | 2489 | 3048 | 3607 |
| A | 1372 | 1931 | 2490 | 3049 | 3608 |
| A | 1373 | 1932 | 2491 | 3050 | 3609 |
| A | 1374 | 1933 | 2492 | 3051 | 3610 |
| A | 1375 | 1934 | 2493 | 3052 | 3611 |
| A | 1376 | 1935 | 2494 | 3053 | 3612 |
| A | 1377 | 1936 | 2495 | 3054 | 3613 |
| A | 1378 | 1937 | 2496 | 3055 | 3614 |
| A | 1379 | 1938 | 2497 | 3056 | 3615 |
| A | 1380 | 1939 | 2498 | 3057 | 3616 |
| A | 1381 | 1940 | 2499 | 3058 | 3617 |
| A | 1382 | 1941 | 2500 | 3059 | 3618 |
| A | 1383 | 1942 | 2501 | 3060 | 3619 |
| A | 1384 | 1943 | 2502 | 3061 | 3620 |
| A | 1385 | 1944 | 2503 | 3062 | 3621 |
| A | 1386 | 1945 | 2504 | 3063 | 3622 |
| A | 1387 | 1946 | 2505 | 3064 | 3623 |
| A | 1388 | 1947 | 2506 | 3065 | 3624 |
| A | 1389 | 1948 | 2507 | 3066 | 3625 |
| A | 1390 | 1949 | 2508 | 3067 | 3626 |
| A | 1391 | 1950 | 2509 | 3068 | 3627 |
| A | 1392 | 1951 | 2510 | 3069 | 3628 |
| A | 1393 | 1952 | 2511 | 3070 | 3629 |
| A | 1394 | 1953 | 2512 | 3071 | 3630 |
| A | 1395 | 1954 | 2513 | 3072 | 3631 |
| A | 1396 | 1955 | 2514 | 3073 | 3632 |
| A | 1397 | 1956 | 2515 | 3074 | 3633 |
| A | 1398 | 1957 | 2516 | 3075 | 3634 |
| A | 1399 | 1958 | 2517 | 3076 | 3635 |
| A | 1400 | 1959 | 2518 | 3077 | 3636 |
| A | 1401 | 1960 | 2519 | 3078 | 3637 |
| A | 1402 | 1961 | 2520 | 3079 | 3638 |
| A | 1403 | 1962 | 2521 | 3080 | 3639 |
| A | 1404 | 1963 | 2522 | 3081 | 3640 |
| A | 1405 | 1964 | 2523 | 3082 | 3641 |
| A | 1406 | 1965 | 2524 | 3083 | 3642 |
| A | 1407 | 1966 | 2525 | 3084 | 3643 |
| A | 1408 | 1967 | 2526 | 3085 | 3644 |
| A | 1409 | 1968 | 2527 | 3086 | 3645 |
| A | 1410 | 1969 | 2528 | 3087 | 3646 |
| A | 1411 | 1970 | 2529 | 3088 | 3647 |
| A | 1412 | 1971 | 2530 | 3089 | 3648 |
| A | 1413 | 1972 | 2531 | 3090 | 3649 |
| A | 1414 | 1973 | 2532 | 3091 | 3650 |
| A | 1415 | 1974 | 2533 | 3092 | 3651 |
| A | 1416 | 1975 | 2534 | 3093 | 3652 |
| A | 1417 | 1976 | 2535 | 3094 | 3653 |
| A | 1418 | 1977 | 2536 | 3095 | 3654 |
| A | 1419 | 1978 | 2537 | 3096 | 3655 |
| A | 1420 | 1979 | 2538 | 3097 | 3656 |
| A | 1421 | 1980 | 2539 | 3098 | 3657 |
| A | 1422 | 1981 | 2540 | 3099 | 3658 |
| A | 1423 | 1982 | 2541 | 3100 | 3659 |
| A | 1424 | 1983 | 2542 | 3101 | 3660 |
| A | 1425 | 1984 | 2543 | 3102 | 3661 |
| A | 1426 | 1985 | 2544 | 3103 | 3662 |
| A | 1427 | 1986 | 2545 | 3104 | 3663 |
| A | 1428 | 1987 | 2546 | 3105 | 3664 |
| B | 1429 | 1988 | 2547 | 3106 | 3665 |
| B | 1430 | 1989 | 2548 | 3107 | 3666 |
| B | 1431 | 1990 | 2549 | 3108 | 3667 |
| B | 1432 | 1991 | 2550 | 3109 | 3668 |
| B | 1433 | 1992 | 2551 | 3110 | 3669 |
| B | 1434 | 1993 | 2552 | 3111 | 3670 |
| B | 1435 | 1994 | 2553 | 3112 | 3671 |
| B | 1436 | 1995 | 2554 | 3113 | 3672 |
| B | 1437 | 1996 | 2555 | 3114 | 3673 |
| B | 1438 | 1997 | 2556 | 3115 | 3674 |
| B | 4173 | 4235 | 4297 | 4359 | 4421 |
| B | 4174 | 4236 | 4298 | 4360 | 4422 |
| B | 4533 | 4557 | 4581 | 4605 | 4629 |
| B | 4534 | 4558 | 4582 | 4606 | 4630 |
| B | 4535 | 4559 | 4583 | 4607 | 4631 |
| B | 4536 | 4560 | 4584 | 4608 | 4632 |
| B | 4537 | 4561 | 4585 | 4609 | 4633 |
| B | 4538 | 4562 | 4586 | 4610 | 4634 |
| C | 1439 | 1998 | 2557 | 3116 | 3675 |
| C | 1440 | 1999 | 2558 | 3117 | 3676 |
| C | 1441 | 2000 | 2559 | 3118 | 3677 |
| C | 1442 | 2001 | 2560 | 3119 | 3678 |
| C | 1443 | 2002 | 2561 | 3120 | 3679 |
| C | 1444 | 2003 | 2562 | 3121 | 3680 |
| C | 1445 | 2004 | 2563 | 3122 | 3681 |
| C | 1446 | 2005 | 2564 | 3123 | 3682 |
| C | 1447 | 2006 | 2565 | 3124 | 3683 |
| C | 1448 | 2007 | 2566 | 3125 | 3684 |
| C | 1449 | 2008 | 2567 | 3126 | 3685 |
| D | 1450 | 2009 | 2568 | 3127 | 3686 |
| D | 1451 | 2010 | 2569 | 3128 | 3687 |
| D | 1452 | 2011 | 2570 | 3129 | 3688 |
| D | 1453 | 2012 | 2571 | 3130 | 3689 |
| D | 1454 | 2013 | 2572 | 3131 | 3690 |
| D | 1455 | 2014 | 2573 | 3132 | 3691 |
| D | 1456 | 2015 | 2574 | 3133 | 3692 |
| D | 1457 | 2016 | 2575 | 3134 | 3693 |
| D | 1458 | 2017 | 2576 | 3135 | 3694 |
| D | 1459 | 2018 | 2577 | 3136 | 3695 |
| D | 1460 | 2019 | 2578 | 3137 | 3696 |
| D | 1461 | 2020 | 2579 | 3138 | 3697 |
| D | 1462 | 2021 | 2580 | 3139 | 3698 |
| D | 1463 | 2022 | 2581 | 3140 | 3699 |
| D | 1464 | 2023 | 2582 | 3141 | 3700 |
| D | 1465 | 2024 | 2583 | 3142 | 3701 |
| D | 1466 | 2025 | 2584 | 3143 | 3702 |
| D | 1467 | 2026 | 2585 | 3144 | 3703 |
| D | 1468 | 2027 | 2586 | 3145 | 3704 |
| D | 1469 | 2028 | 2587 | 3146 | 3705 |
| D | 1470 | 2029 | 2588 | 3147 | 3706 |
| D | 1471 | 2030 | 2589 | 3148 | 3707 |
| D | 1472 | 2031 | 2590 | 3149 | 3708 |
| D | 1473 | 2032 | 2591 | 3150 | 3709 |
| D | 1474 | 2033 | 2592 | 3151 | 3710 |
| D | 1475 | 2034 | 2593 | 3152 | 3711 |
| D | 1476 | 2035 | 2594 | 3153 | 3712 |
| D | 1477 | 2036 | 2595 | 3154 | 3713 |
| D | 1478 | 2037 | 2596 | 3155 | 3714 |
| D | 1479 | 2038 | 2597 | 3156 | 3715 |
| D | 1480 | 2039 | 2598 | 3157 | 3716 |
| D | 1481 | 2040 | 2599 | 3158 | 3717 |
| D | 1482 | 2041 | 2600 | 3159 | 3718 |
| D | 1483 | 2042 | 2601 | 3160 | 3719 |
| D | 1484 | 2043 | 2602 | 3161 | 3720 |
| D | 1485 | 2044 | 2603 | 3162 | 3721 |
| D | 1486 | 2045 | 2604 | 3163 | 3722 |
| D | 1487 | 2046 | 2605 | 3164 | 3723 |
| D | 1488 | 2047 | 2606 | 3165 | 3724 |
| D | 1489 | 2048 | 2607 | 3166 | 3725 |
| D | 1490 | 2049 | 2608 | 3167 | 3726 |
| D | 1491 | 2050 | 2609 | 3168 | 3727 |
| D | 1492 | 2051 | 2610 | 3169 | 3728 |
| D | 1493 | 2052 | 2611 | 3170 | 3729 |
| D | 1494 | 2053 | 2612 | 3171 | 3730 |
| D | 1495 | 2054 | 2613 | 3172 | 3731 |
| D | 1496 | 2055 | 2614 | 3173 | 3732 |
| D | 1497 | 2056 | 2615 | 3174 | 3733 |
| D | 1498 | 2057 | 2616 | 3175 | 3734 |
| D | 1499 | 2058 | 2617 | 3176 | 3735 |
| D | 1500 | 2059 | 2618 | 3177 | 3736 |
| D | 1501 | 2060 | 2619 | 3178 | 3737 |
| D | 1502 | 2061 | 2620 | 3179 | 3738 |
| D | 1503 | 2062 | 2621 | 3180 | 3739 |
| D | 1504 | 2063 | 2622 | 3181 | 3740 |
| D | 1505 | 2064 | 2623 | 3182 | 3741 |
| D | 1506 | 2065 | 2624 | 3183 | 3742 |
| D | 1507 | 2066 | 2625 | 3184 | 3743 |
| D | 1508 | 2067 | 2626 | 3185 | 3744 |
| D | 1509 | 2068 | 2627 | 3186 | 3745 |
| D | 1510 | 2069 | 2628 | 3187 | 3746 |
| D | 1511 | 2070 | 2629 | 3188 | 3747 |
| D | 1512 | 2071 | 2630 | 3189 | 3748 |
| D | 1513 | 2072 | 2631 | 3190 | 3749 |
| D | 1514 | 2073 | 2632 | 3191 | 3750 |
| D | 1515 | 2074 | 2633 | 3192 | 3751 |
| D | 1516 | 2075 | 2634 | 3193 | 3752 |
| D | 1517 | 2076 | 2635 | 3194 | 3753 |
| D | 1518 | 2077 | 2636 | 3195 | 3754 |
| D | 1519 | 2078 | 2637 | 3196 | 3755 |
| D | 1520 | 2079 | 2638 | 3197 | 3756 |
| D | 1521 | 2080 | 2639 | 3198 | 3757 |
| D | 1522 | 2081 | 2640 | 3199 | 3758 |
| D | 1523 | 2082 | 2641 | 3200 | 3759 |
| D | 1524 | 2083 | 2642 | 3201 | 3760 |
| D | 1525 | 2084 | 2643 | 3202 | 3761 |
| D | 1526 | 2085 | 2644 | 3203 | 3762 |
| D | 1527 | 2086 | 2645 | 3204 | 3763 |
| D | 1528 | 2087 | 2646 | 3205 | 3764 |
| D | 4175 | 4237 | 4299 | 4361 | 4423 |
| D | 4176 | 4238 | 4300 | 4362 | 4424 |
| D | 4177 | 4239 | 4301 | 4363 | 4425 |
| D | 4178 | 4240 | 4302 | 4364 | 4426 |
| D | 4179 | 4241 | 4303 | 4365 | 4427 |
| D | 4180 | 4242 | 4304 | 4366 | 4428 |
| D | 4181 | 4243 | 4305 | 4367 | 4429 |
| D | 4182 | 4244 | 4306 | 4368 | 4430 |
| D | 4183 | 4245 | 4307 | 4369 | 4431 |
| D | 4184 | 4246 | 4308 | 4370 | 4432 |
| D | 4539 | 4563 | 4587 | 4611 | 4635 |
| D | 4540 | 4564 | 4588 | 4612 | 4636 |
| D | 4541 | 4565 | 4589 | 4613 | 4637 |
| D | 4542 | 4566 | 4590 | 4614 | 4638 |
| D | 4543 | 4567 | 4591 | 4615 | 4639 |
| D | 4544 | 4568 | 4592 | 4616 | 4640 |
| D | 4545 | 4569 | 4593 | 4617 | 4641 |
| D | 4546 | 4570 | 4594 | 4618 | 4642 |
| D | 4547 | 4571 | 4595 | 4619 | 4643 |
| D | 4548 | 4572 | 4596 | 4620 | 4644 |
| D | 4549 | 4573 | 4597 | 4621 | 4645 |
| D | 4550 | 4574 | 4598 | 4622 | 4646 |
| G | 1529 | 2088 | 2647 | 3206 | 3765 |
| G | 1530 | 2089 | 2648 | 3207 | 3766 |
| G | 1531 | 2090 | 2649 | 3208 | 3767 |
| J | 1532 | 2091 | 2650 | 3209 | 3768 |
| J | 1533 | 2092 | 2651 | 3210 | 3769 |
| J | 1534 | 2093 | 2652 | 3211 | 3770 |
| J | 1535 | 2094 | 2653 | 3212 | 3771 |
| J | 1536 | 2095 | 2654 | 3213 | 3772 |
| J | 1537 | 2096 | 2655 | 3214 | 3773 |
| J | 1538 | 2097 | 2656 | 3215 | 3774 |
| J | 1539 | 2098 | 2657 | 3216 | 3775 |
| J | 1540 | 2099 | 2658 | 3217 | 3776 |
| J | 1541 | 2100 | 2659 | 3218 | 3777 |
| J | 1542 | 2101 | 2660 | 3219 | 3778 |
| J | 1543 | 2102 | 2661 | 3220 | 3779 |
| J | 1544 | 2103 | 2662 | 3221 | 3780 |
| J | 1545 | 2104 | 2663 | 3222 | 3781 |
| J | 1546 | 2105 | 2664 | 3223 | 3782 |
| J | 1547 | 2106 | 2665 | 3224 | 3783 |
| J | 1548 | 2107 | 2666 | 3225 | 3784 |
| J | 1549 | 2108 | 2667 | 3226 | 3785 |
| J | 1550 | 2109 | 2668 | 3227 | 3786 |
| J | 1551 | 2110 | 2669 | 3228 | 3787 |
| J | 1552 | 2111 | 2670 | 3229 | 3788 |
| J | 1553 | 2112 | 2671 | 3230 | 3789 |
| J | 1554 | 2113 | 2672 | 3231 | 3790 |
| J | 1555 | 2114 | 2673 | 3232 | 3791 |
| J | 1556 | 2115 | 2674 | 3233 | 3792 |
| J | 1557 | 2116 | 2675 | 3234 | 3793 |
| J | 1558 | 2117 | 2676 | 3235 | 3794 |
| J | 1559 | 2118 | 2677 | 3236 | 3795 |
| J | 1560 | 2119 | 2678 | 3237 | 3796 |
| J | 1561 | 2120 | 2679 | 3238 | 3797 |
| J | 1562 | 2121 | 2680 | 3239 | 3798 |
| J | 1563 | 2122 | 2681 | 3240 | 3799 |
| J | 1564 | 2123 | 2682 | 3241 | 3800 |
| J | 1565 | 2124 | 2683 | 3242 | 3801 |
| J | 1566 | 2125 | 2684 | 3243 | 3802 |
| J | 1567 | 2126 | 2685 | 3244 | 3803 |
| J | 1568 | 2127 | 2686 | 3245 | 3804 |
| J | 1569 | 2128 | 2687 | 3246 | 3805 |
| J | 1570 | 2129 | 2688 | 3247 | 3806 |
| J | 1571 | 2130 | 2689 | 3248 | 3807 |
| J | 1572 | 2131 | 2690 | 3249 | 3808 |
| J | 1573 | 2132 | 2691 | 3250 | 3809 |
| J | 1574 | 2133 | 2692 | 3251 | 3810 |
| J | 1575 | 2134 | 2693 | 3252 | 3811 |
| J | 1576 | 2135 | 2694 | 3253 | 3812 |
| J | 1577 | 2136 | 2695 | 3254 | 3813 |
| J | 1578 | 2137 | 2696 | 3255 | 3814 |
| J | 1579 | 2138 | 2697 | 3256 | 3815 |
| J | 1580 | 2139 | 2698 | 3257 | 3816 |
| J | 1581 | 2140 | 2699 | 3258 | 3817 |
| K | 1582 | 2141 | 2700 | 3259 | 3818 |
| K | 1583 | 2142 | 2701 | 3260 | 3819 |
| K | 1584 | 2143 | 2702 | 3261 | 3820 |
| K | 1585 | 2144 | 2703 | 3262 | 3821 |
| K | 1586 | 2145 | 2704 | 3263 | 3822 |
| K | 1587 | 2146 | 2705 | 3264 | 3823 |
| K | 1588 | 2147 | 2706 | 3265 | 3824 |
| K | 1589 | 2148 | 2707 | 3266 | 3825 |
| K | 1590 | 2149 | 2708 | 3267 | 3826 |
| K | 1591 | 2150 | 2709 | 3268 | 3827 |
| K | 1592 | 2151 | 2710 | 3269 | 3828 |
| K | 1593 | 2152 | 2711 | 3270 | 3829 |
| K | 1594 | 2153 | 2712 | 3271 | 3830 |
| K | 1595 | 2154 | 2713 | 3272 | 3831 |
| K | 1596 | 2155 | 2714 | 3273 | 3832 |
| K | 1597 | 2156 | 2715 | 3274 | 3833 |
| K | 1598 | 2157 | 2716 | 3275 | 3834 |
| K | 1599 | 2158 | 2717 | 3276 | 3835 |
| K | 1600 | 2159 | 2718 | 3277 | 3836 |
| K | 1601 | 2160 | 2719 | 3278 | 3837 |
| K | 1602 | 2161 | 2720 | 3279 | 3838 |
| K | 1603 | 2162 | 2721 | 3280 | 3839 |
| K | 1604 | 2163 | 2722 | 3281 | 3840 |
| K | 1605 | 2164 | 2723 | 3282 | 3841 |
| K | 1606 | 2165 | 2724 | 3283 | 3842 |
| K | 1607 | 2166 | 2725 | 3284 | 3843 |
| K | 4185 | 4247 | 4309 | 4371 | 4433 |
| K | 4186 | 4248 | 4310 | 4372 | 4434 |
| K | 4187 | 4249 | 4311 | 4373 | 4435 |
| K | 4188 | 4250 | 4312 | 4374 | 4436 |
| K | 4189 | 4251 | 4313 | 4375 | 4437 |
| K | 4190 | 4252 | 4314 | 4376 | 4438 |
| K | 4191 | 4253 | 4315 | 4377 | 4439 |
| K | 4192 | 4254 | 4316 | 4378 | 4440 |
| K | 4193 | 4255 | 4317 | 4379 | 4441 |
| K | 4194 | 4256 | 4318 | 4380 | 4442 |
| K | 4195 | 4257 | 4319 | 4381 | 4443 |
| K | 4196 | 4258 | 4320 | 4382 | 4444 |
| K | 4197 | 4259 | 4321 | 4383 | 4445 |
| K | 4198 | 4260 | 4322 | 4384 | 4446 |
| K | 4199 | 4261 | 4323 | 4385 | 4447 |
| K | 4200 | 4262 | 4324 | 4386 | 4448 |
| K | 4201 | 4263 | 4325 | 4387 | 4449 |
| K | 4202 | 4264 | 4326 | 4388 | 4450 |
| K | 4203 | 4265 | 4327 | 4389 | 4451 |
| K | 4204 | 4266 | 4328 | 4390 | 4452 |
| K | 4205 | 4267 | 4329 | 4391 | 4453 |
| K | 4206 | 4268 | 4330 | 4392 | 4454 |
| K | 4207 | 4269 | 4331 | 4393 | 4455 |
| K | 4208 | 4270 | 4332 | 4394 | 4456 |
| K | 4209 | 4271 | 4333 | 4395 | 4457 |
| K | 4551 | 4575 | 4599 | 4623 | 4647 |
| K | 4552 | 4576 | 4600 | 4624 | 4648 |
| K | 4553 | 4577 | 4601 | 4625 | 4649 |
| K | 4554 | 4578 | 4602 | 4626 | 4650 |
| K | 4555 | 4579 | 4603 | 4627 | 4651 |
| K | 4556 | 4580 | 4604 | 4628 | 4652 |
| L | 1608 | 2167 | 2726 | 3285 | 3844 |
| M | 1609 | 2168 | 2727 | 3286 | 3845 |
| M | 1610 | 2169 | 2728 | 3287 | 3846 |
| M | 1611 | 2170 | 2729 | 3288 | 3847 |
| M | 1612 | 2171 | 2730 | 3289 | 3848 |
| M | 1613 | 2172 | 2731 | 3290 | 3849 |
| M | 1614 | 2173 | 2732 | 3291 | 3850 |
| M | 1615 | 2174 | 2733 | 3292 | 3851 |
| M | 1616 | 2175 | 2734 | 3293 | 3852 |
| M | 1617 | 2176 | 2735 | 3294 | 3853 |
| M | 1618 | 2177 | 2736 | 3295 | 3854 |
| M | 1619 | 2178 | 2737 | 3296 | 3855 |
| M | 1620 | 2179 | 2738 | 3297 | 3856 |
| M | 1621 | 2180 | 2739 | 3298 | 3857 |
| M | 1622 | 2181 | 2740 | 3299 | 3858 |
| M | 1623 | 2182 | 2741 | 3300 | 3859 |
| M | 1624 | 2183 | 2742 | 3301 | 3860 |
| M | 1625 | 2184 | 2743 | 3302 | 3861 |
| M | 1626 | 2185 | 2744 | 3303 | 3862 |
| M | 1627 | 2186 | 2745 | 3304 | 3863 |
| M | 1628 | 2187 | 2746 | 3305 | 3864 |
| M | 1629 | 2188 | 2747 | 3306 | 3865 |
| M | 1630 | 2189 | 2748 | 3307 | 3866 |
| M | 1631 | 2190 | 2749 | 3308 | 3867 |
| M | 1632 | 2191 | 2750 | 3309 | 3868 |
| M | 1633 | 2192 | 2751 | 3310 | 3869 |
| M | 1634 | 2193 | 2752 | 3311 | 3870 |
| M | 1635 | 2194 | 2753 | 3312 | 3871 |
| M | 1636 | 2195 | 2754 | 3313 | 3872 |
| M | 1637 | 2196 | 2755 | 3314 | 3873 |
| M | 1638 | 2197 | 2756 | 3315 | 3874 |
| M | 1639 | 2198 | 2757 | 3316 | 3875 |
| M | 1640 | 2199 | 2758 | 3317 | 3876 |
| M | 1641 | 2200 | 2759 | 3318 | 3877 |
| M | 1642 | 2201 | 2760 | 3319 | 3878 |
| M | 1643 | 2202 | 2761 | 3320 | 3879 |
| M | 1644 | 2203 | 2762 | 3321 | 3880 |
| M | 1645 | 2204 | 2763 | 3322 | 3881 |
| M | 1646 | 2205 | 2764 | 3323 | 3882 |
| M | 1647 | 2206 | 2765 | 3324 | 3883 |
| M | 1648 | 2207 | 2766 | 3325 | 3884 |
| M | 1649 | 2208 | 2767 | 3326 | 3885 |
| M | 1650 | 2209 | 2768 | 3327 | 3886 |
| M | 1651 | 2210 | 2769 | 3328 | 3887 |
| M | 1652 | 2211 | 2770 | 3329 | 3888 |
| M | 1653 | 2212 | 2771 | 3330 | 3889 |
| M | 1654 | 2213 | 2772 | 3331 | 3890 |
| M | 1655 | 2214 | 2773 | 3332 | 3891 |
| M | 1656 | 2215 | 2774 | 3333 | 3892 |
| M | 1657 | 2216 | 2775 | 3334 | 3893 |
| M | 1658 | 2217 | 2776 | 3335 | 3894 |
| M | 1659 | 2218 | 2777 | 3336 | 3895 |
| M | 1660 | 2219 | 2778 | 3337 | 3896 |
| M | 1661 | 2220 | 2779 | 3338 | 3897 |
| M | 1662 | 2221 | 2780 | 3339 | 3898 |
| M | 1663 | 2222 | 2781 | 3340 | 3899 |
| M | 1664 | 2223 | 2782 | 3341 | 3900 |
| M | 1665 | 2224 | 2783 | 3342 | 3901 |
| M | 1666 | 2225 | 2784 | 3343 | 3902 |
| M | 1667 | 2226 | 2785 | 3344 | 3903 |
| M | 1668 | 2227 | 2786 | 3345 | 3904 |
| M | 1669 | 2228 | 2787 | 3346 | 3905 |
| M | 1670 | 2229 | 2788 | 3347 | 3906 |
| M | 1671 | 2230 | 2789 | 3348 | 3907 |
| M | 1672 | 2231 | 2790 | 3349 | 3908 |
| M | 1673 | 2232 | 2791 | 3350 | 3909 |
| M | 1674 | 2233 | 2792 | 3351 | 3910 |
| M | 1675 | 2234 | 2793 | 3352 | 3911 |
| M | 1676 | 2235 | 2794 | 3353 | 3912 |
| M | 1677 | 2236 | 2795 | 3354 | 3913 |
| M | 1678 | 2237 | 2796 | 3355 | 3914 |
| M | 1679 | 2238 | 2797 | 3356 | 3915 |
| M | 1680 | 2239 | 2798 | 3357 | 3916 |
| M | 1681 | 2240 | 2799 | 3358 | 3917 |
| M | 1682 | 2241 | 2800 | 3359 | 3918 |
| M | 1683 | 2242 | 2801 | 3360 | 3919 |
| M | 1684 | 2243 | 2802 | 3361 | 3920 |
| M | 1685 | 2244 | 2803 | 3362 | 3921 |
| M | 1686 | 2245 | 2804 | 3363 | 3922 |
| M | 4210 | 4272 | 4334 | 4396 | 4458 |
| M | 4211 | 4273 | 4335 | 4397 | 4459 |
| M | 4212 | 4274 | 4336 | 4398 | 4460 |
| N | 4213 | 4275 | 4337 | 4399 | 4461 |
| N | 4214 | 4276 | 4338 | 4400 | 4462 |
| N | 4215 | 4277 | 4339 | 4401 | 4463 |
| N | 4216 | 4278 | 4340 | 4402 | 4464 |
| N | 4217 | 4279 | 4341 | 4403 | 4465 |
| N | 4218 | 4280 | 4342 | 4404 | 4466 |
| N | 4219 | 4281 | 4343 | 4405 | 4467 |
| N | 4220 | 4282 | 4344 | 4406 | 4468 |
| N | 4221 | 4283 | 4345 | 4407 | 4469 |
| O | 4222 | 4284 | 4346 | 4408 | 4470 |
| O | 4223 | 4285 | 4347 | 4409 | 4471 |
| O | 4224 | 4286 | 4348 | 4410 | 4472 |
| O | 4225 | 4287 | 4349 | 4411 | 4473 |
| O | 4226 | 4288 | 4350 | 4412 | 4474 |
| O | 4227 | 4289 | 4351 | 4413 | 4475 |
| O | 4228 | 4290 | 4352 | 4414 | 4476 |
| O | 4229 | 4291 | 4353 | 4415 | 4477 |
| O | 4230 | 4292 | 4354 | 4416 | 4478 |
| O | 4231 | 4293 | 4355 | 4417 | 4479 |
| O | 4232 | 4294 | 4356 | 4418 | 4480 |
| O | 4233 | 4295 | 4357 | 4419 | 4481 |
| O | 4234 | 4296 | 4358 | 4420 | 4482 |

### Example 4. Flow Cytometry Binding

To measure the binding of V-bodies to cell-displayed TNFR2 from human, cynomolgus or mouse, HEK293 cells were transfected with plasmids encoding for respective antigens. After 48 to 72 hours, binding was measured by incubation of His-tagged V-bodies with cells at various fixed concentrations, followed by washing and detection with Alexa488 fluorophore-labelled anti-His antibodies. To detect the binding of V-bodies to specific cysteine-rich domains (CRDs) of TNFR2, binding to HEK293 transfected with constructs where a single CRD was exchanged for camel CRD was measured. Lack of binding to a particular construct indicated binding to the domain which was exchanged.

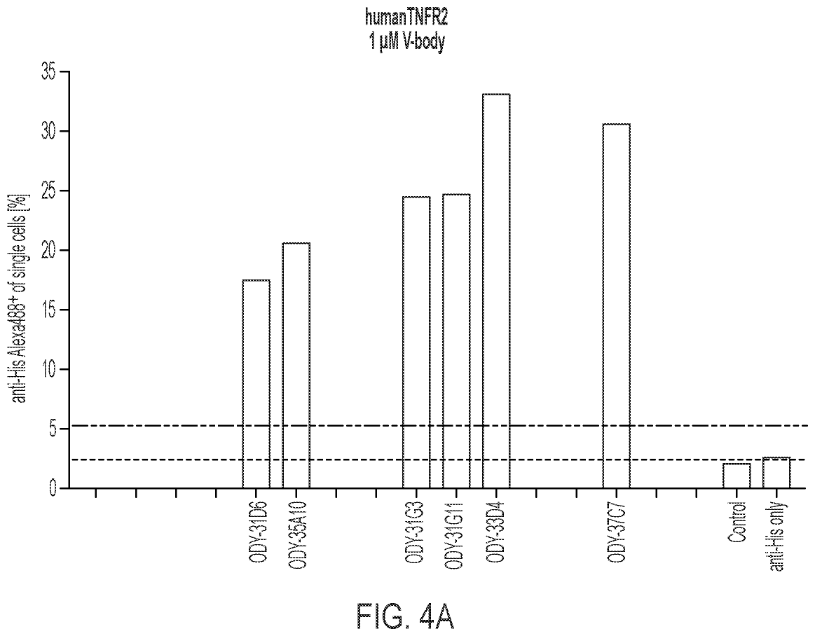

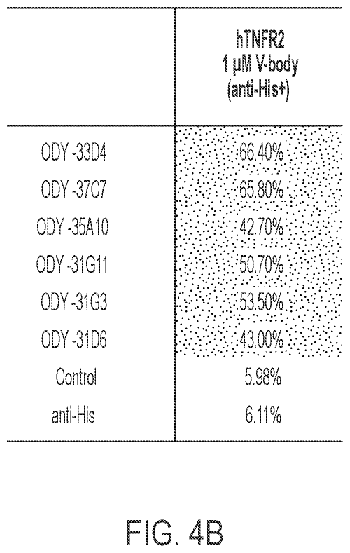

For generation of data depicted in FIGS. 4A-48, HEK293T cells were transiently transfected with a plasmid encoding human TNFR2 (hTNFR2; hTNFR2_pcDNA3.4.dna). After 48 hours, HEK293T cells were harvested and incubated with 1 μM purified His-tagged (myc-his tag) VHHs, including a control VHH against an irrelevant antigen. VHH binding was then detected using an Alexa488-labeled anti-His tag antibody and measured by flow cytometry (iQue).

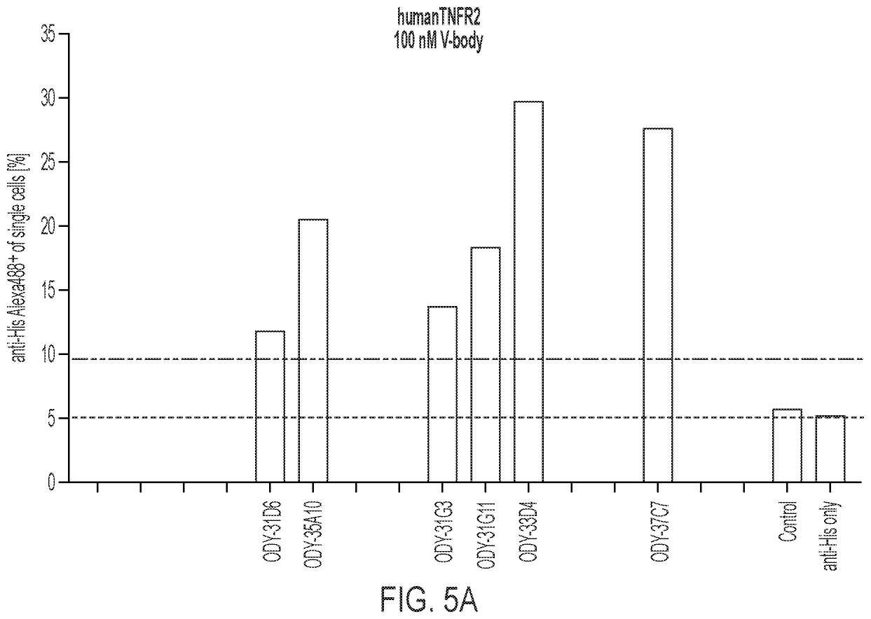

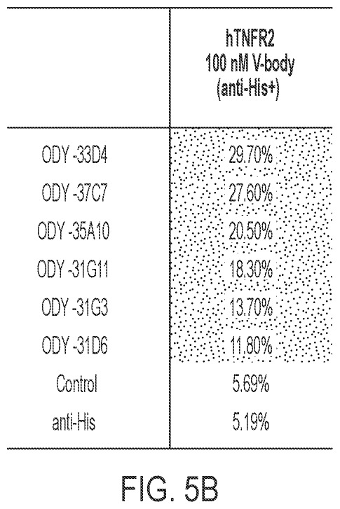

For generation of data depicted in FIGS. 5A-5B HEK293T cells were transiently transfected with a plasmid encoding human TNFR2 (hTNFR2; hTNFR2_pcDNA3.4.dna), After 48 hours, HEK293T cells were harvested and incubated with 100 nM purified His-tagged (myc-his tag) VHHs, including a control VHH against an irrelevant antigen. VHH binding was then detected using an Alexa488-labeled anti-His tag antibody and measured by flow cytometry (iQue).

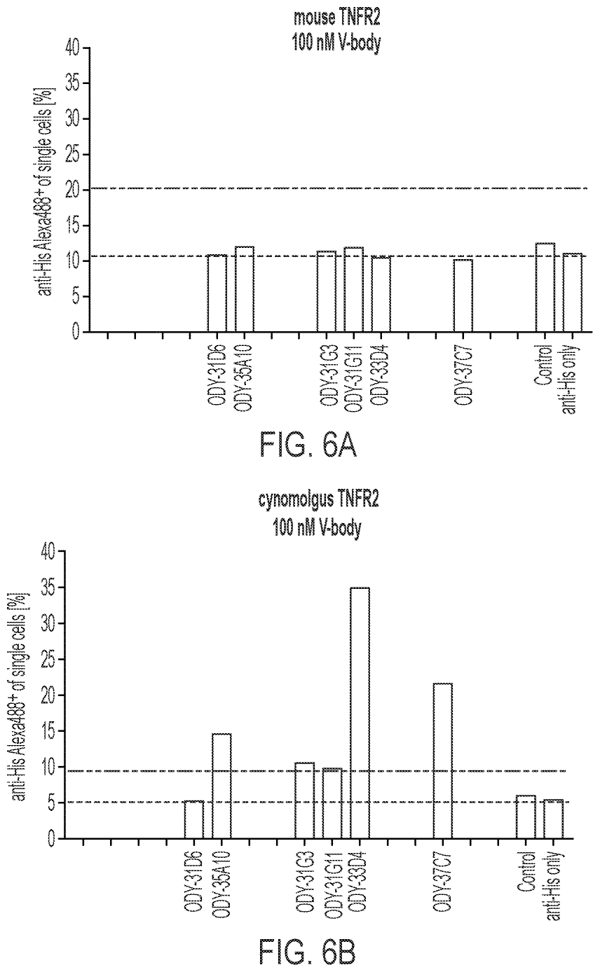

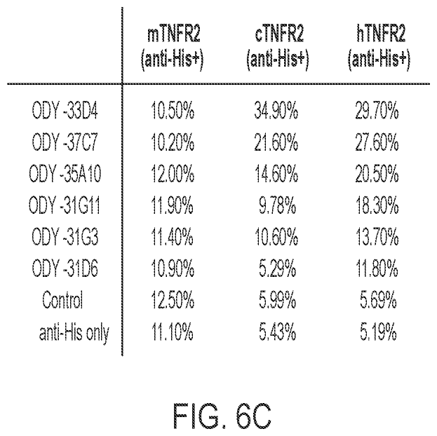

For generation of data depicted in FIGS. 6A-68, HEK293T cells were transiently transfected with a plasmid encoding cynomolgus TNFR2 (cTNFR2; cTNFR2_pcDNA3.4.dna) or mouse TNFR2 (mTNFR2; mTNFR2_pcDNA3.4.dna). After 48 hours, HEK293T cells were harvested and incubated with 100 nM purified His-tagged (myc-his tag) VHHs, including a control VHH against an irrelevant antigen. VHHs binding was then detected using a Alexa488-labeled anti-His tag antibody and measured by flow cytometry (IQue).

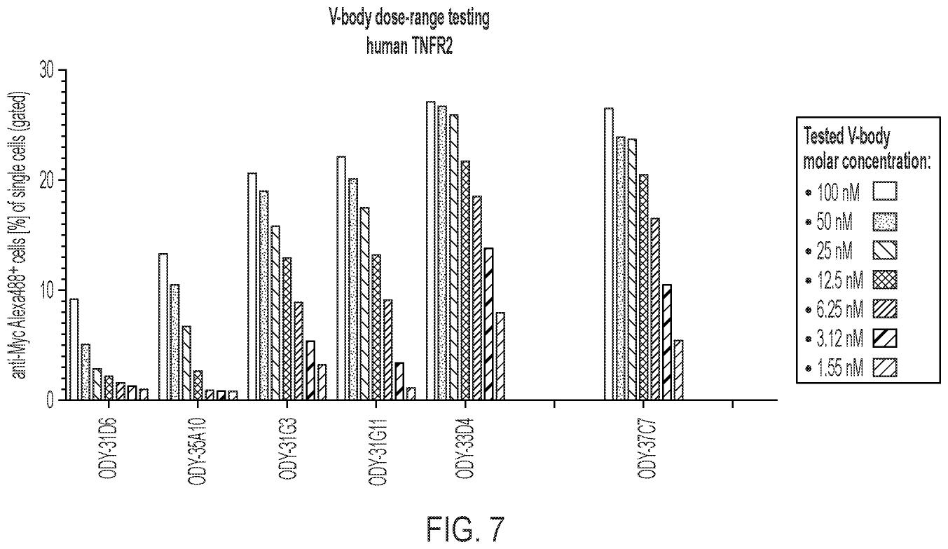

FIG. 7 shows testing of human TNFR2 V-body binding across a range of concentrations for ODY-31D6, ODY-35A10, ODY-31G3, ODY-31G11, ODY-33D4, ODY-37C7, and. V-bodies were tested at the following molar concentrations: 100 nM, 50 nM, 12.5 nM, 6.25 nM, 3.12 nM, and 1.55 nM. For generation of data depicted in FIG. 7, HEK293T cells were transiently transfected with a plasmid encoding human TNFR2 (hTNFR2; hTNFR2_pcDNA3.4.dna). After 48 hours, HEK293T cells were harvested and incubated with increasing molar concentrations of purified His-tagged (myc-his tag) VHHs, including a control VHH against an irrelevant antigen. VHH binding was then detected using an Alexa488-labeled anti-His tag antibody and measured by flow cytometry (iQue). The bar histogram (FIG. 7) shows the percentage of Alexa488 positive cells for ODY-31D6, ODY-35A10, ODY-31G3, ODY-31G11, ODY-33D4, ODY-37C7.

### Example 5. Surface Plasmon Resonance Binding Affinities

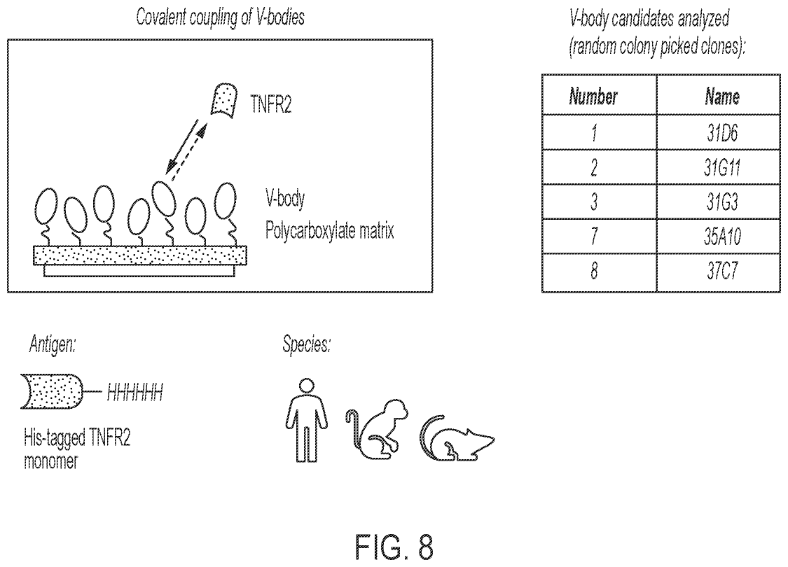

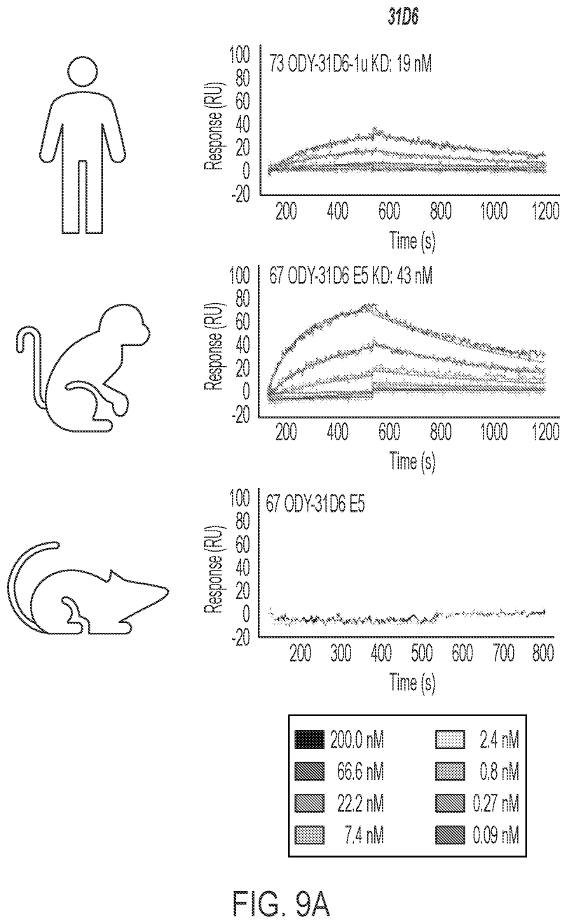

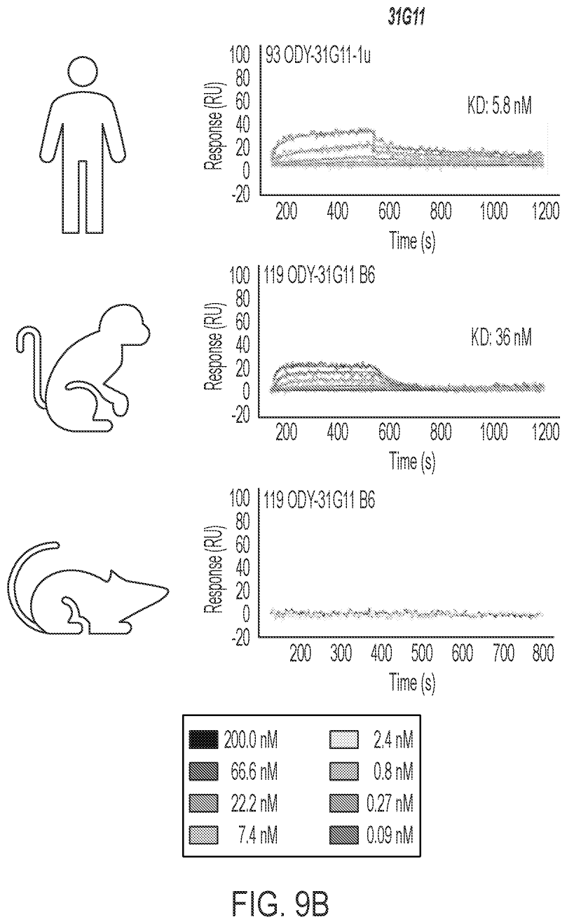

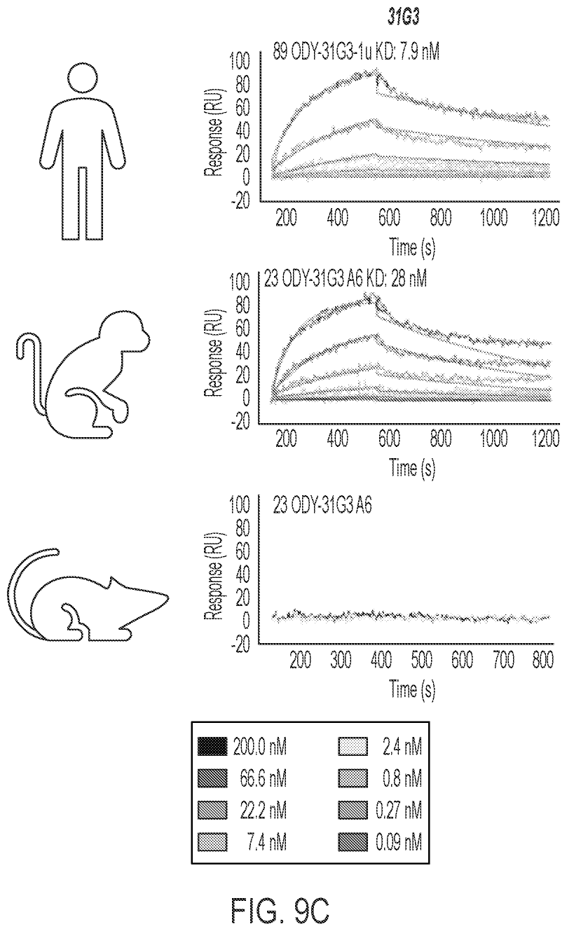

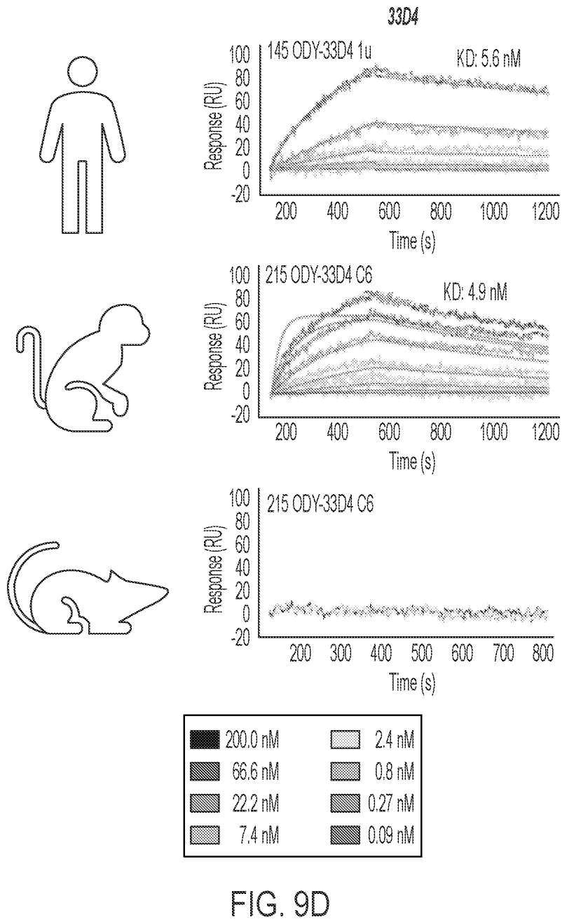

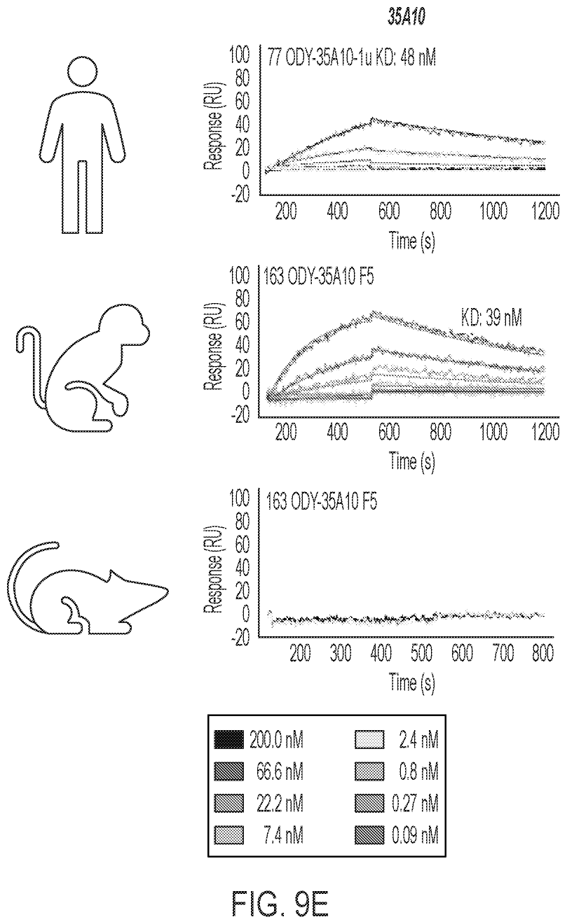

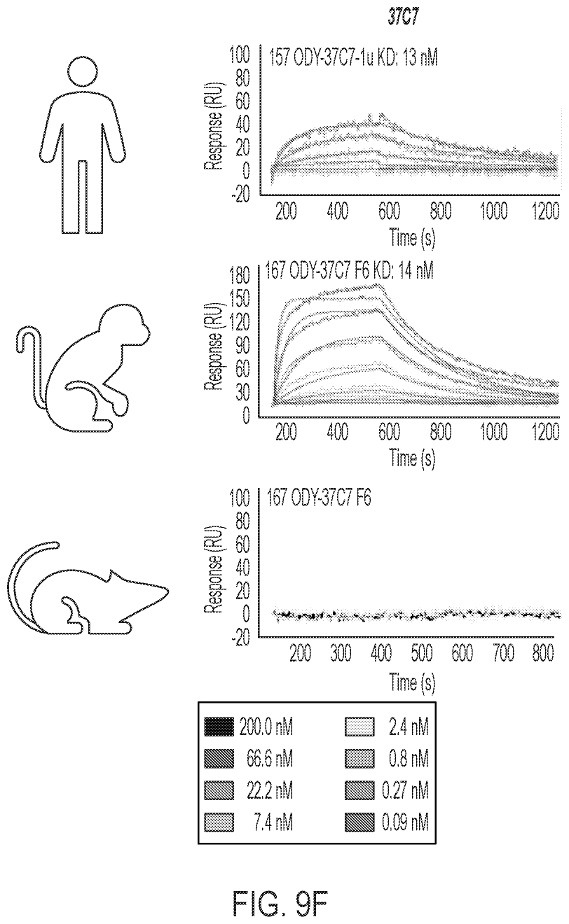

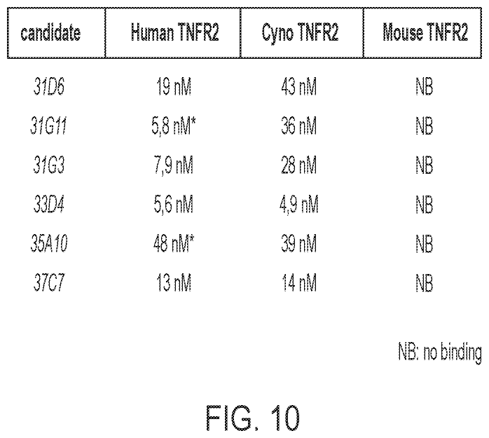

Binding affinities of the V-bodies to their respective target was determined by surface plasmon resonance (SPR) using a Caterra LSA instrument. A schematic diagram depicting the experimental setup of the present Example is shown in FIG. 8. Affinity purified V-bodies were covalently crosslinked onto an LSA HC200M chip using EDC/Sulfo NHS. The interaction with human, cynomolgus, and mouse TNFR2 (extracellular domain) (V-body coupling concentration: human, 1 M; cynomolgus/mouse, 4 μM) was measured under physiological conditions (Running Buffer: HBST-50 mM HEPES pH 7.4, 150 mM NaCl, 0.1% (w/v) BSA, 0.05% (v/v) Tween20, 25° C.) using eight different antigen concentrations (3-fold serial dilutions, start at 200 nM). Resulting sensorgrams (FIGS. 9A-9F) were analyzed and equilibrium-binding affinities (K_{D}s) were calculated using Carterra's data analysis software. FIG. 10 shows a summary of binding affinities of 16 anti-TNFR2 V-bodies: 31D6, 31G11, 31G3, 33D4, 35A10, and 37C7. Seven of the 16 V-body candidates tested (31D6, 31G11, 31G3, 33D4, 35A10, and 37C7) were shown to bind human TNFR2 with 1-2-digit nM affinity. Nine and zero V-bodies demonstrated cross-reactivity to cynomolgus TNFR2 (96% identical to hTNFR2 sequence) and mouse TNFR2 (63% identical to hTNFR2 sequence), respectively.

### Example 6. MR2-1 Competition

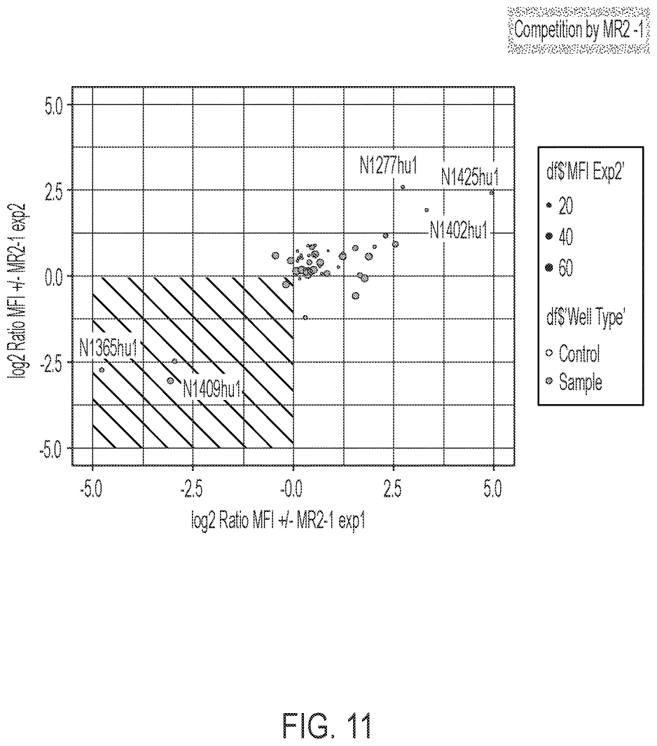

To investigate whether humanized TNFR2 V-bodies targeted the epitope recognized by a MR2-1 bivalent agonist, TNFR2 expressing HEK cells (clone 25) were incubated with or without MR2-1 (5 or 10 μg/ml) prior to (pre-) and/or during (co-) incubation with His-tagged TNFR2-specific V-bodies. After washing, V-body binding was detected by labelled anti-His antibody. Binding inhibition in the presence of MR2-1 indicated binding of TNFR2 V-bodies and MR2-1 bivalent agonist to an overlapping epitope. Specifically, N1365hu1 and N1409hu1 may recognize the same epitope as MR2-1. MR2-1 binding enhanced binding of N1402hu1, N1425hu1 and N1277hu1 to TNFR2 (FIG. 11).

### Example 7. TNFR2 Reporter Assay

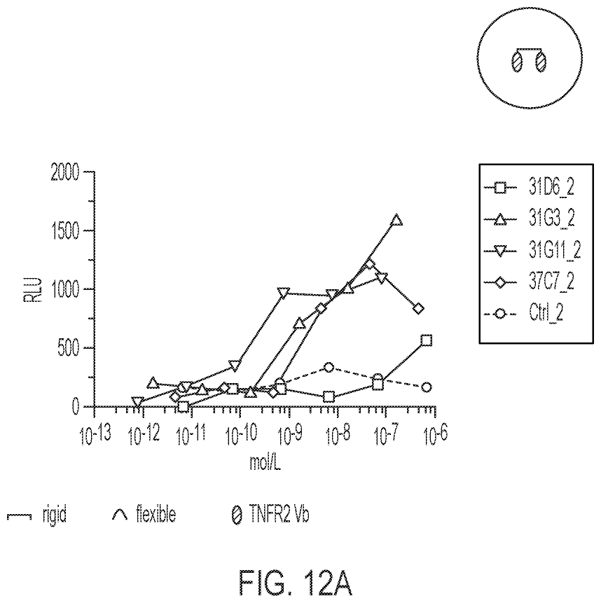

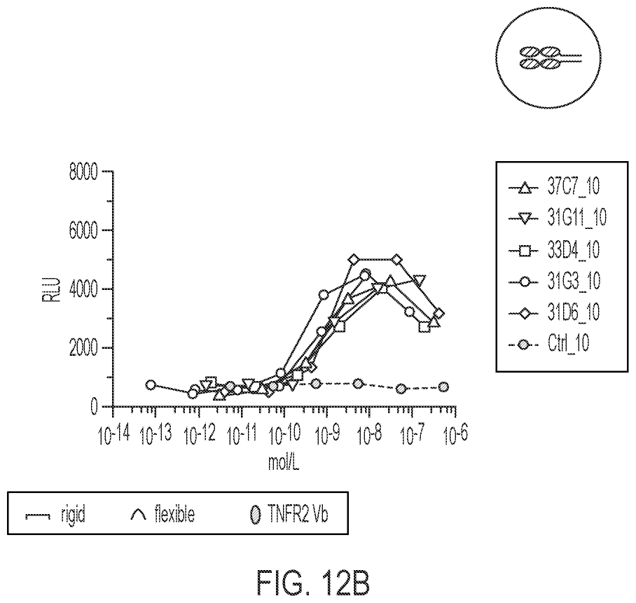

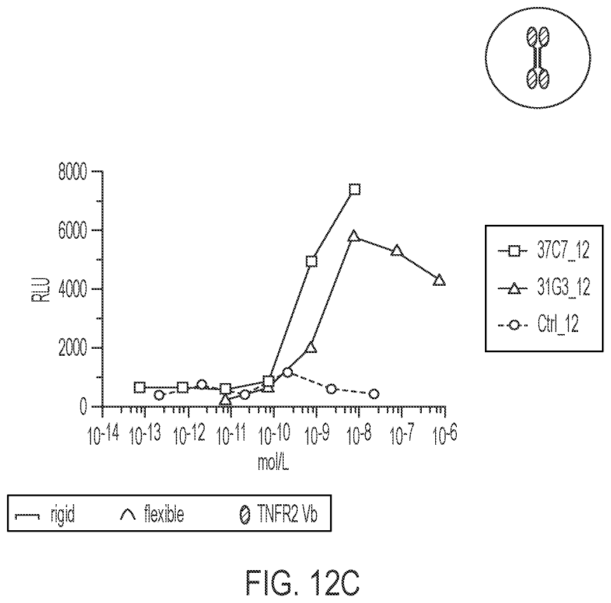

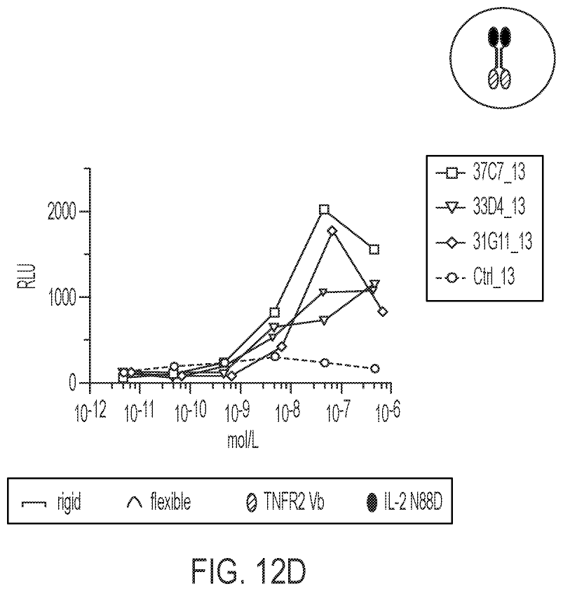

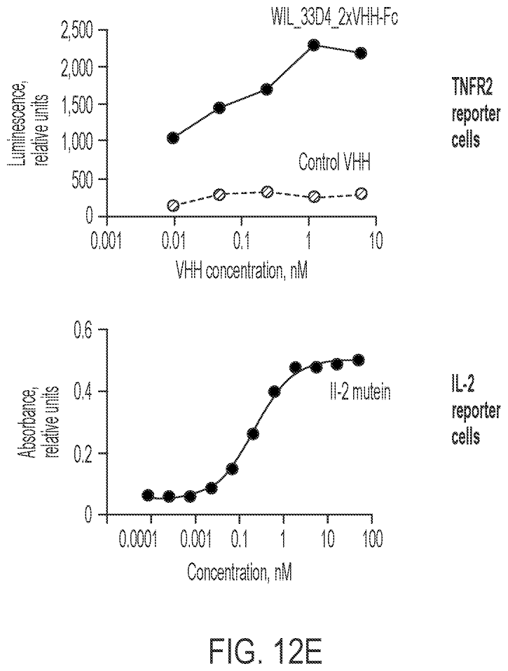

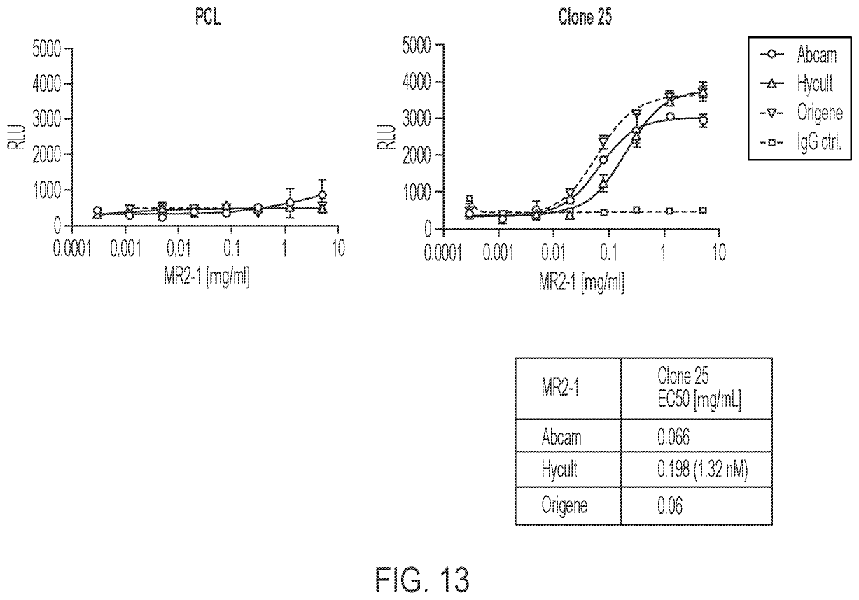

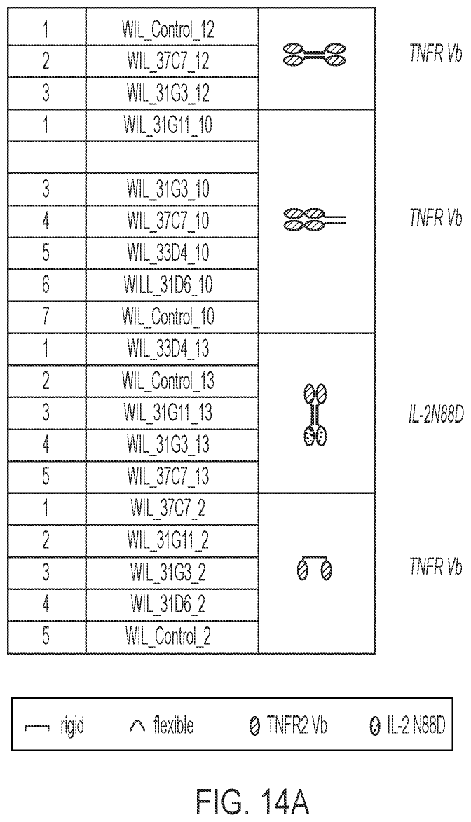

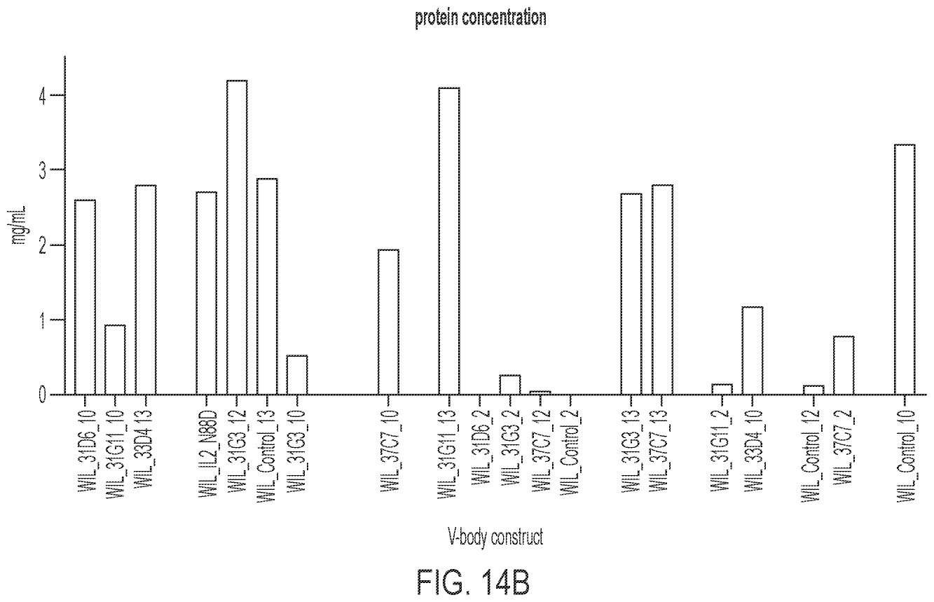

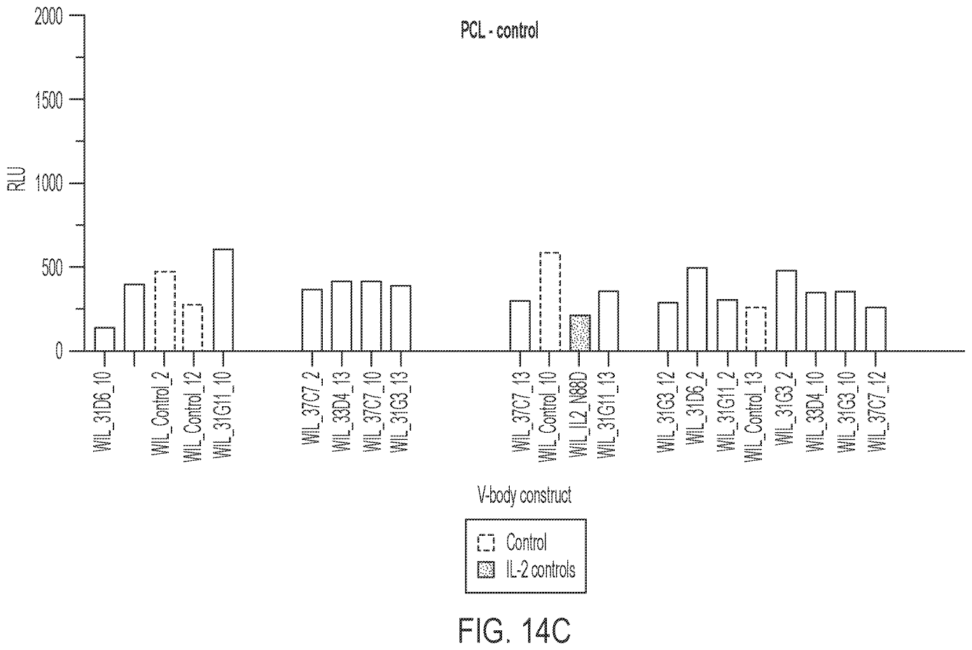

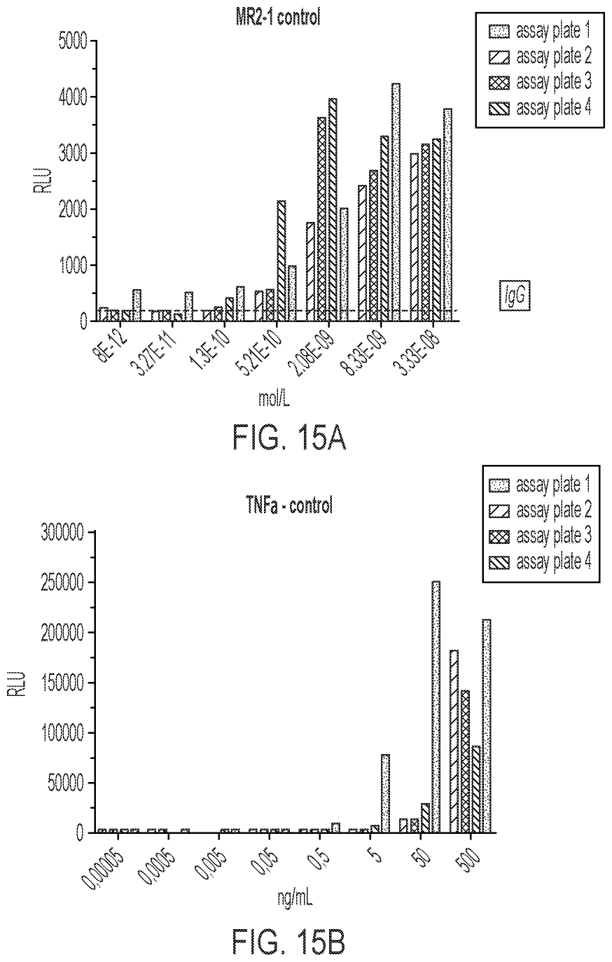

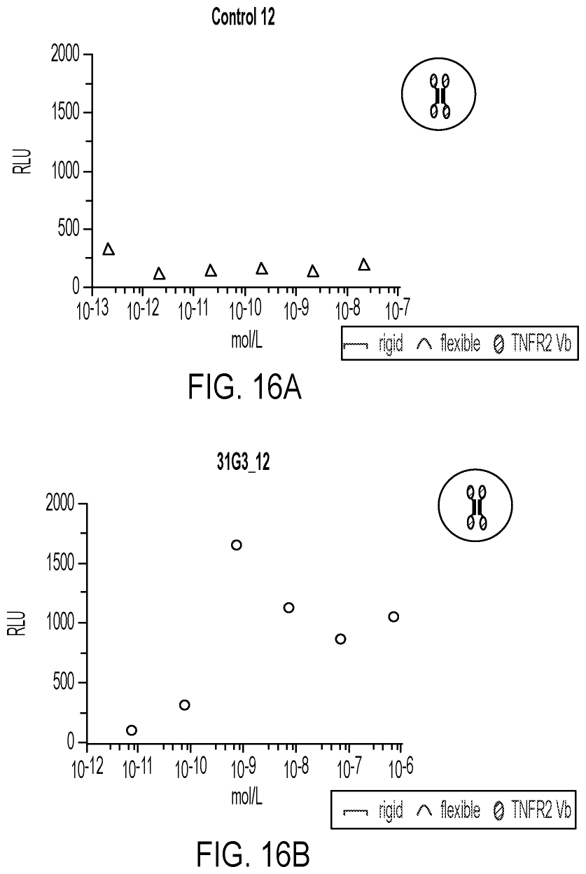

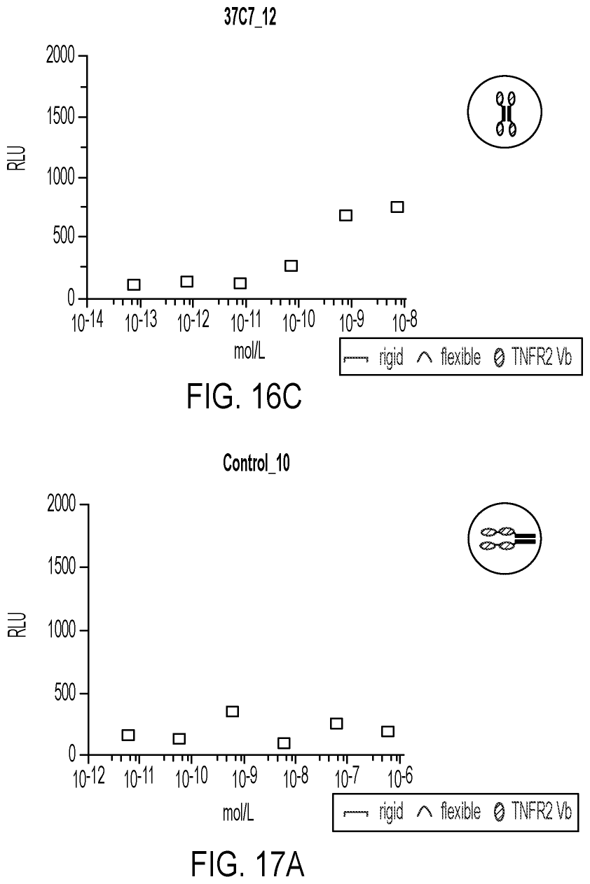

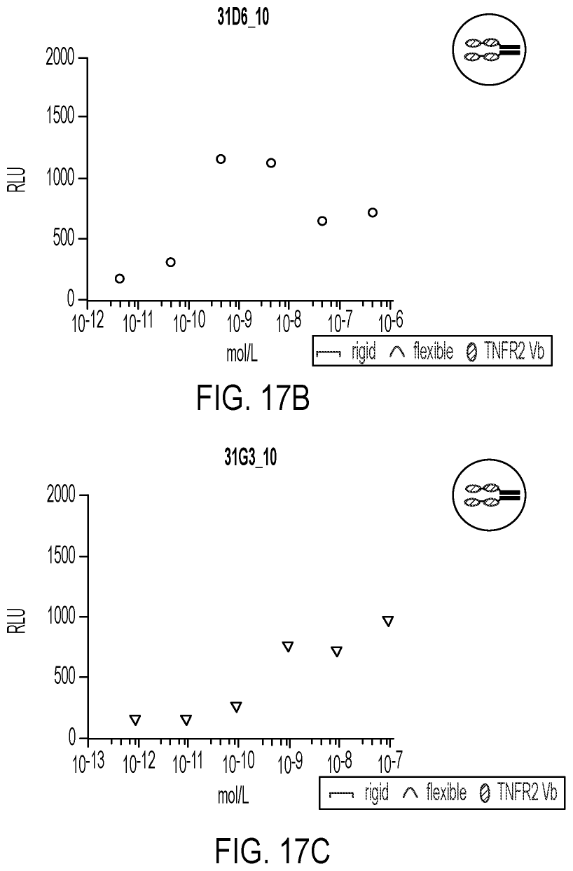

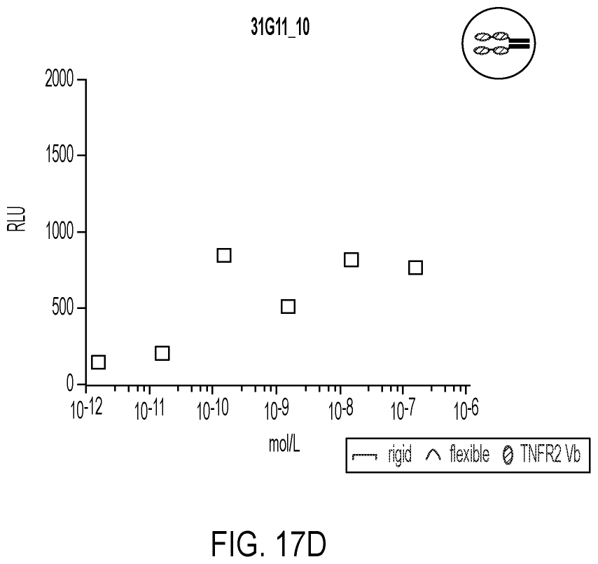

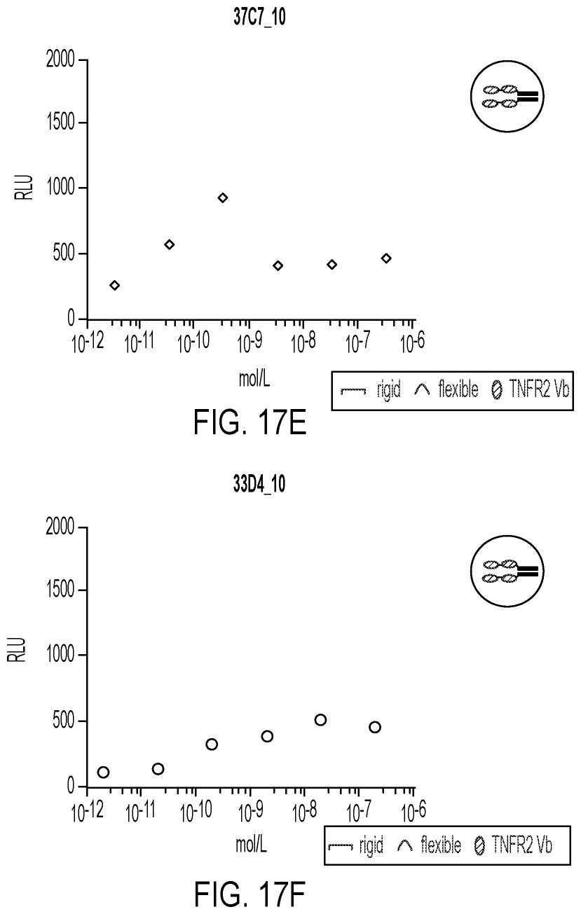

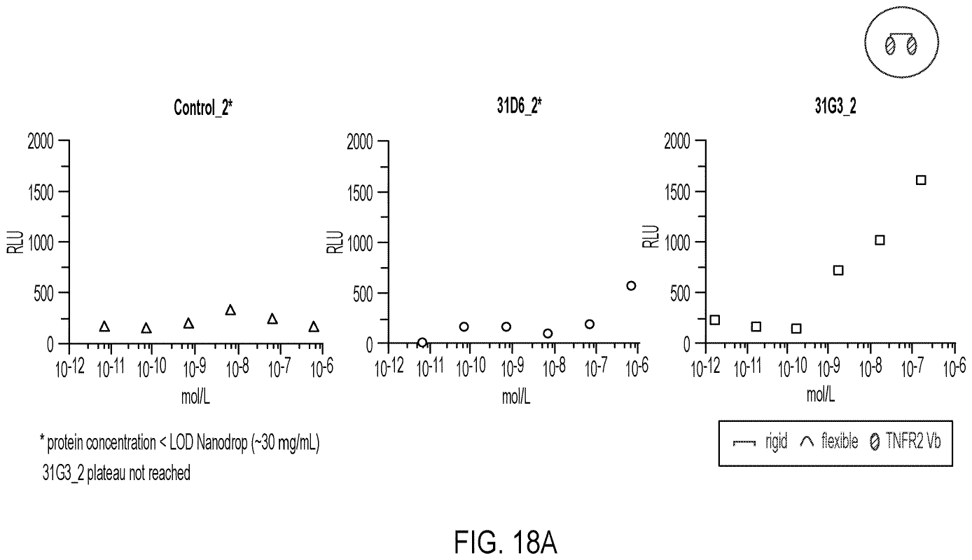

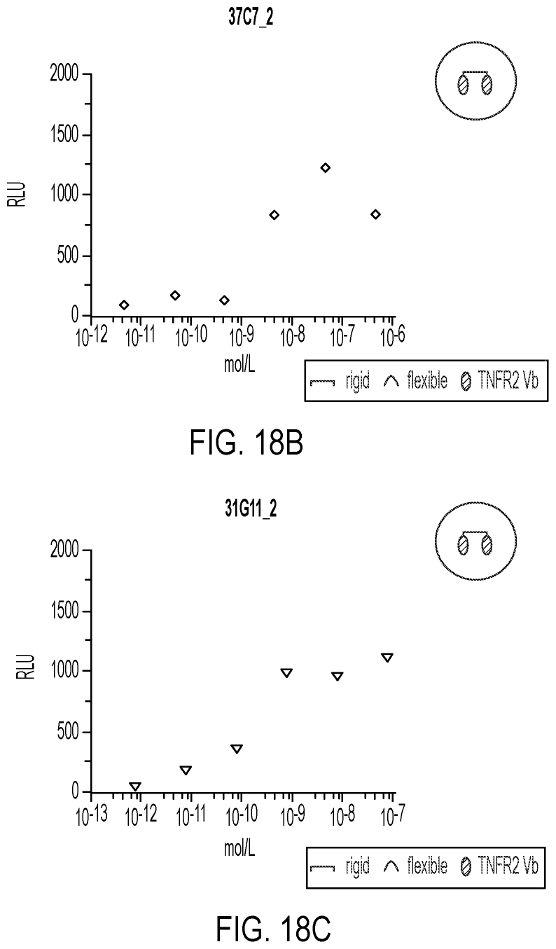

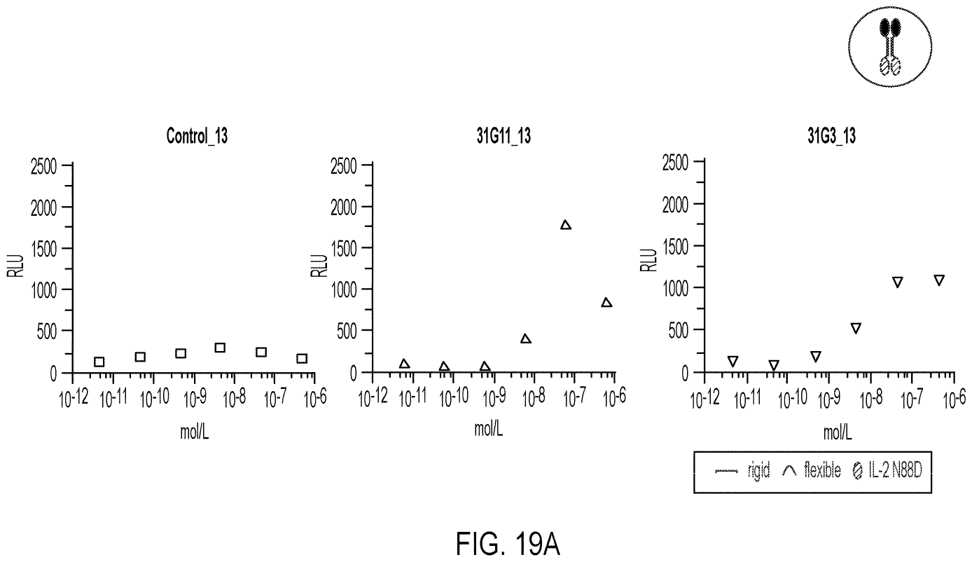

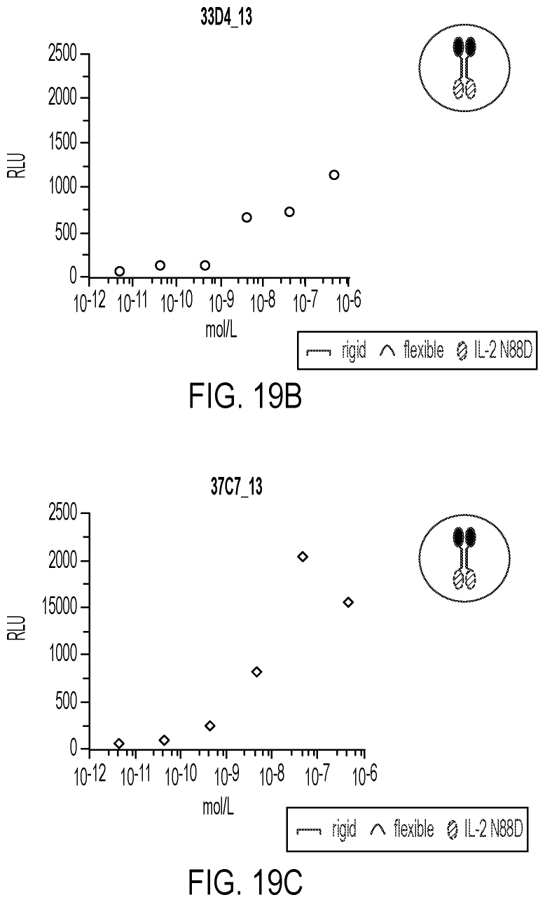

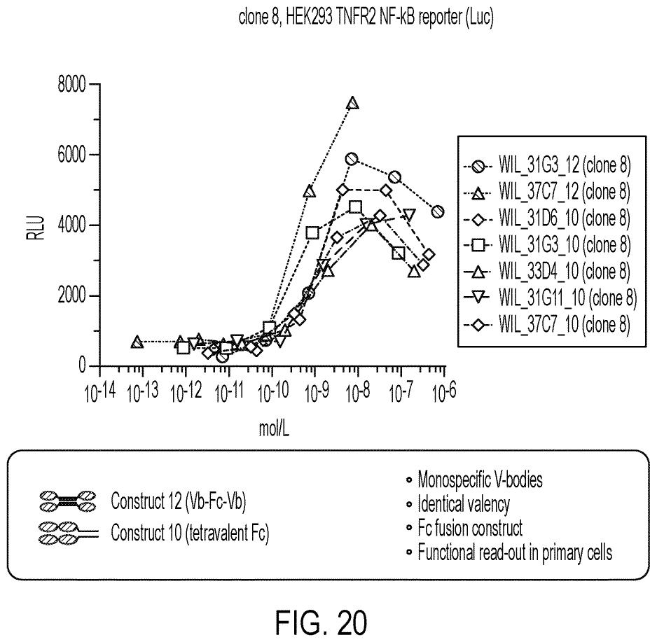

HEK293 nuclear factor kappa B (NF-κB) reporter (Luc) cells stably expressing TNFR2 were established by transfection and antibiotic selection. Several clones were derived from the cell pool. In these cells, TNFR2 signaling was measured by NF-κB-induced expression of firefly luciferase (Luc). Data showing agonism of multivalent constructs characterized on NF-κB reporter HEK293 cells stably expressing TNFR2 are displayed in FIGS. 12A-12D. Typically, luciferase activity was measured 16- to 24-hours after incubation with the V-bodies. For assay control experiments, commercially available human TNFR2 agonist MR2-1 monoclonal antibodies were tested on NF-κB reporter (Luc) HEK293 reporter cell-line stably expressing TNFR2 (clone 25) versus parental cell line (PCL) (FIG. 13). For the sample analysis (n=1), 2 μL of purified sample (43 mM Citrate 148 mM HEPES, pH 6) was diluted using 10-fold serial dilutions (1:10) in assay medium. A bar graph showing protein concentration (mg/ml) for the V-body constructs and respective controls (see FIG. 14A, for sample details) is shown in FIG. 14B. A bar graph showing relative luciferase units (RLUs) for V-body constructs and respective controls (see FIG. 14A, for sample details) tested on a PCL control is shown in FIG. 14C. Concentration range curve data generated using MR2-1 (Hycult) and TNFα controls revealed increasing RLU measured with increasing concentrations of MR2-1 (mol/L) and TNFα (ng/ml) (FIGS. 15A-15B, respectively). FIGS. 16A-16C show dot plots of RLU measured across increasing concentrations (mol/L) of control (control 12) and tetravalent V-body fusion constructs comprising four V-bodies mounted onto an Fc of a IgG4 variant comprising S228P and L235E mutations. FIGS. 17A-17F show dot plots of RLU measured across increasing concentrations (mol/L) of control (control 10) and tetravalent V-body fusion constructs comprising four V-bodies mounted onto an Fc of a IgG4 variant comprising $228P and L235E mutations, FIGS. 18A-18C show dot plots of RLU measured across increasing concentrations (mol/L) of control (control 10) and tetravalent V-body fusion constructs comprising four V-bodies mounted onto an Fc of a IgG4 variant comprising S228P and L235E mutations. FIGS. 19A-19C show exemplary dot plots of RLUs measured across increasing concentrations (mol/L) of control (control 13) and IL-2N88D V-body fusion constructs. Limit of detection, LOD. A comparison of RLU measured across increasing concentrations (mol/L) of monospecific constructs 10 and 12 tested on NF-κB reporter (Luc) HEK293 reporter cell-line stably expressing TNFR2 (clone 8) is shown in FIG. 20.

### Example 8. Testing of TNFR2 Agonism by Multivalent Constructs

The ability of multivalent constructs to agonize TNFR2 on human regulatory T cells (Tregs) was demonstrated by the upregulation of Human Leukocyte Antigen, DR isotype (HLA-DR) and chemokine motif (C-C motif) receptor 8 (CCR8) upon activation of TNFR2, HLA-DR and CCR8 are markers expressed on highly suppressive Tregs, which have been shown to be upregulated by TNFR2 agonist MR2-1. A description of established Treg markers (e.g., forkhead box P3 (FoxP3), HLA-DR, CCR8, and OX-40) useful in, e.g., screening of multivalent constructs in first wave binders on primary cells, is described in Table 6.

TABLE 6
Treg Markers for Screening Multivalent Constructs

| Marker | Exemplary biological function(s) |
|---|---|
| FoxP3 | Treg marker |
| HLA- | Expressed on a functionally distinct population of Tregs |
| DR | associated with high FoxP3 expression and high |
| | suppression of effector T-cells |
| | Has been shown to be upregulated by TNFR2 agonist |
| CCR8 | Chemokine receptor expressed on Treg cells, a subset of Type |
| | 2 T helper (Th-2) cells, monocytic cells and natural killer (NK) |
| | cells |
| | High levels of CCR8 mark cells with high levels of suppressive |
| | function |
| OX-40 | T-cell activity marker expressed on Treg cells |

For testing of multivalent constructs in Tregs, human Tregs were isolated from buffy coats using magnetic beads, and subsequently stimulated with anti-CD3 and anti-CD28 coated stimulation beads with IL-2, in the presence or absence of fixed or increasing concentrations of a control VHH or TNFR2-specific V-bodies.

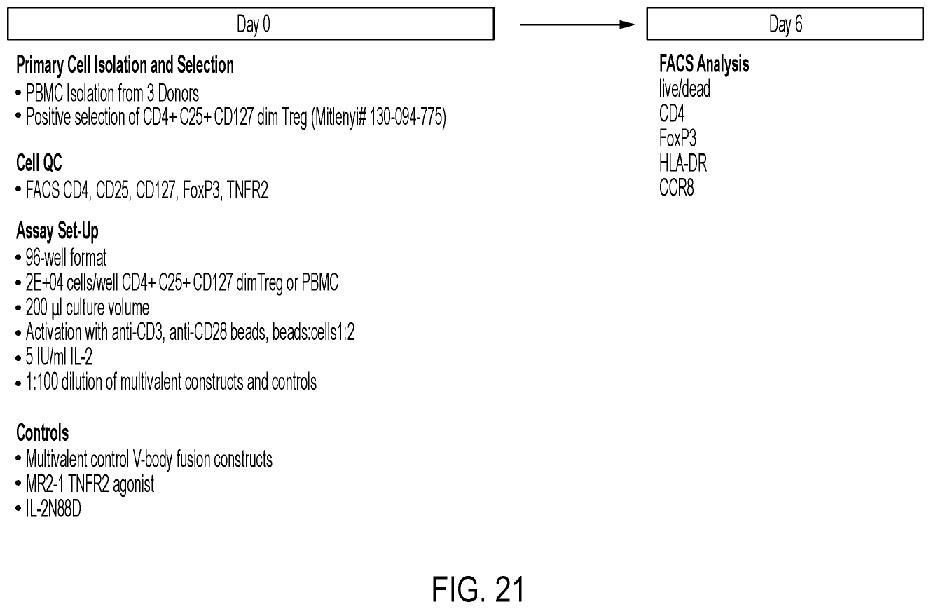

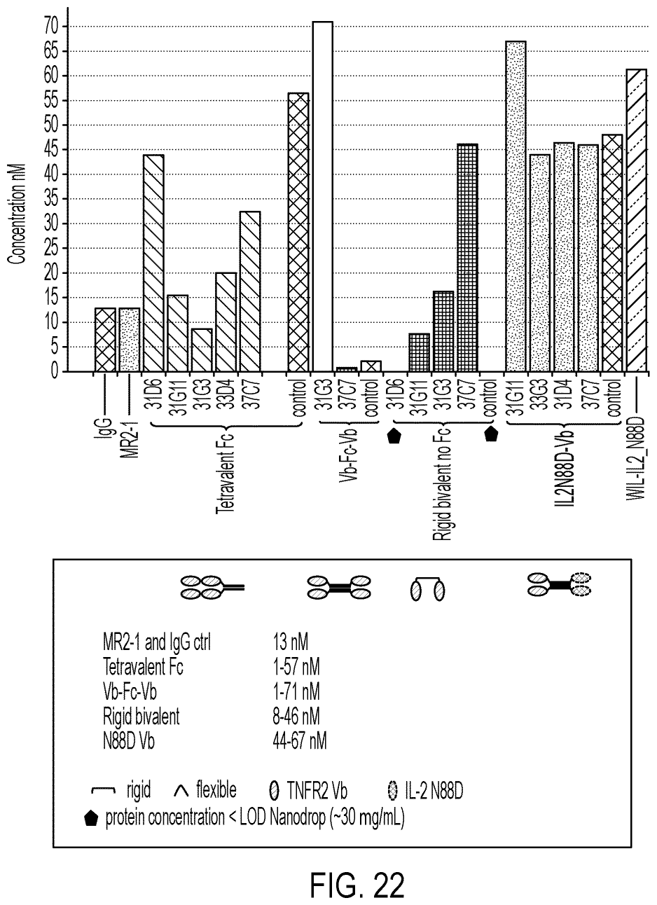

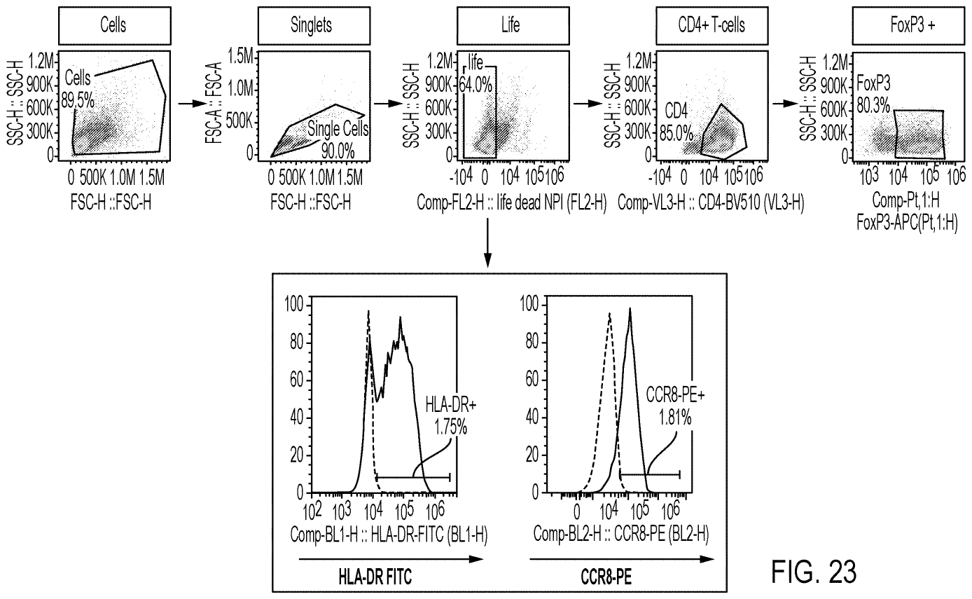

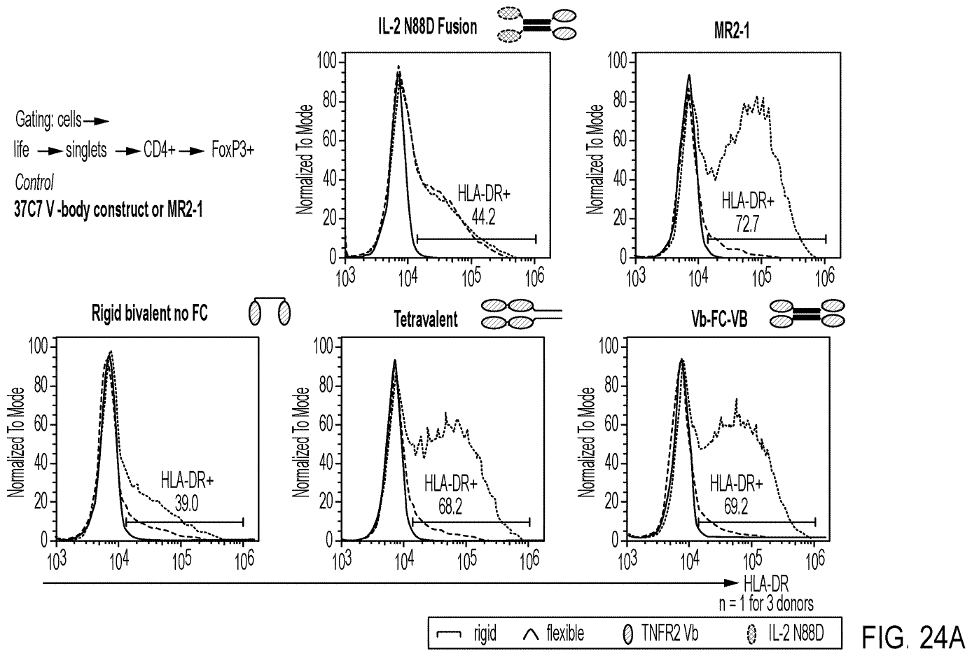

An exemplary experimental timeline of TNFR2 stimulation by multivalent constructs on primary human peripheral blood mononuclear cells (PBMCs) and CD4⁺ CD25⁺ CD127dim Tregs is shown in FIG. 21. Briefly, on Day 0, PBMCs were isolated and selected from three donors (Donors 1-3). Tregs were positively selected for CD4⁺ CD25⁺ CD127dim (Mitlenyi Cat. No. 130-094-775). Fluorescence-activated cell sorting (FACS) analysis was used in cell quality control (QC) experiments to confirm cell populations based on CD4, CD25, CD127, FoxP3, and TNFR2 markers. For the assay set-up, a 96-well plate was seeded with 2×10⁴ cells/well CD4+ C25+ CD127dim Tregs or PBMCs (200 μl total culture volume). Cells were activated with anti-CD3/anti-CD28 coated stimulation beads (using a ratio of 1:2 for beads:cells) and IL-2 (5 IU/mL). Multivalent constructs and controls were added to each well at a 1:100 dilution. Controls were multivalent control V-body fusion constructs, IL-2 N88D fusion constructs, and TNFR2 agonist MR2-1. On Day 6, FACS analysis was performed for live/dead quantification of cells and assessment of cell populations based on expression levels and density of CD4, FoxP3, HLA-DR, and/or CCR8 markers. A bar graph of an overview of in-assay concentrations (nM) of multivalent (e.g., tetravalent Fc, Vb-Fc-Vb, rigid bivalent no Fc) binders is shown in FIG. 22. An exemplary gating strategy for Treg markers useful in the practice of the present Example is shown in FIG. 23. Briefly, Treg Donor 1 is shown as an example and an identical strategy was used for Treg Donor 2 and Donor 3. Live cell and CD4 gating were based on Fluorescence Minus One (FMO) control determination of the cut-off point between background fluorescence and positive cell populations. FOXP3, HLA-DR, CCR8, and OX-40 gating was based on the CD4 subset of IgG control-stained sample from the same donor. For FoxP3, the gate was set at approximately 0.2%. For OX-40, HLA-DR and CCR8, the gate was set at approximately 2%.

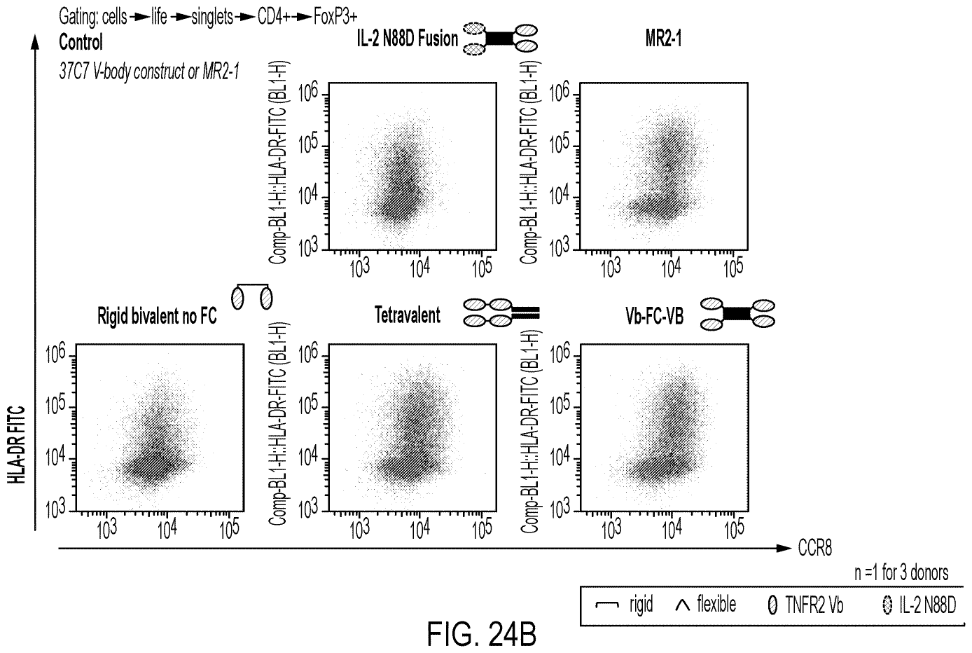

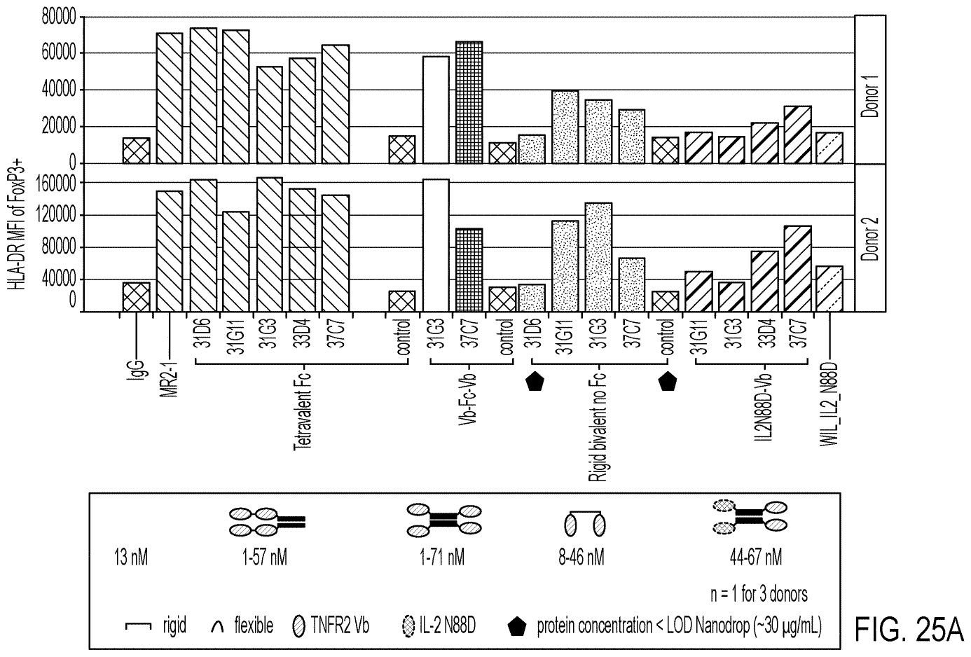

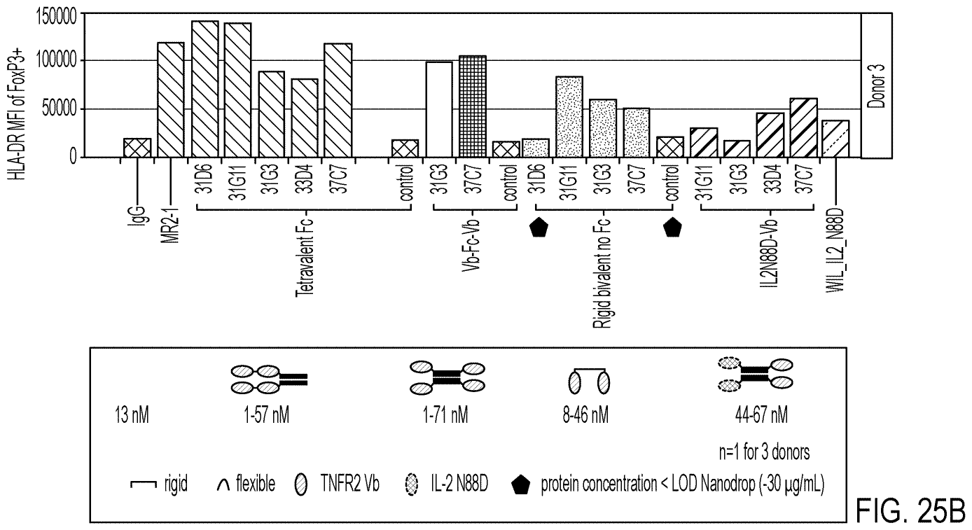

Findings from the present Example showed that multivalent anti-TNFR2 constructs increased expression of the Treg suppression marker HLA-DR (FIGS. 24A-B) and CCR8 (FIG. 248). In particular, each of the multivalent formats tested herein resulted in increased HLA-DR and CCR8 expression for specific 37C7 binder compared to control with formats, with 4 binders showing the strongest increase. IL-2 N88D fusion constructs increased HLA-DR expression however a tetravalent anti-TNFR2 V-body formal was required for an additional increase in HLA-DR expression compared to control. Further, tetravalent anti-TNFR2 V-bodies strongly increased expression of Treg suppression marker HLA-DR on Fox P3⁺ Tregs (FIGS. 25A-25B).

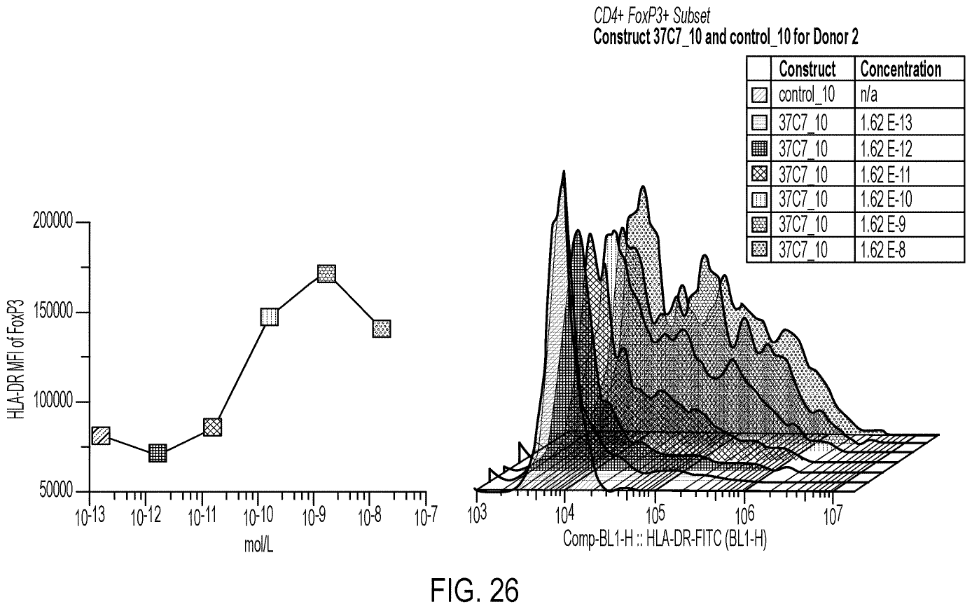

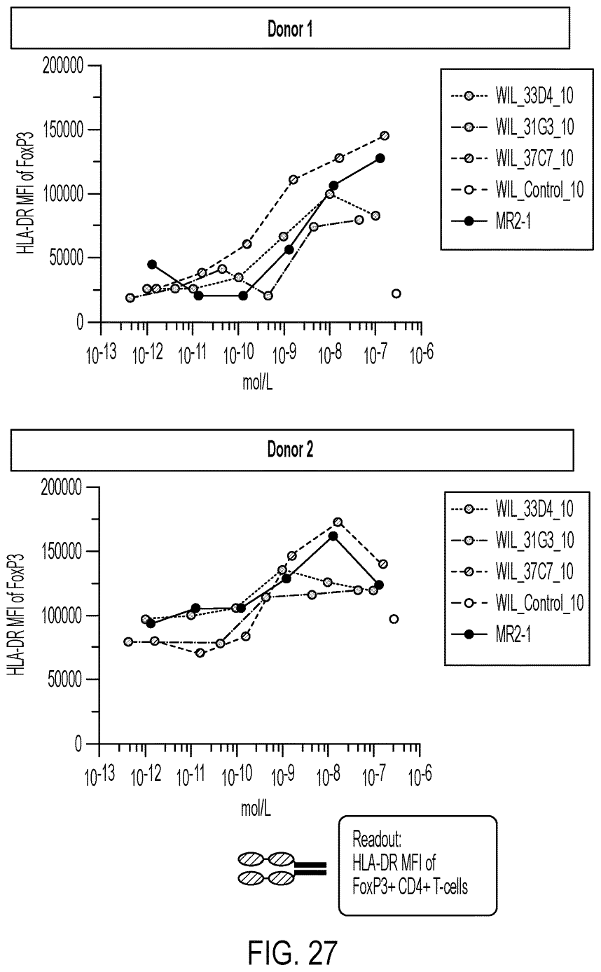

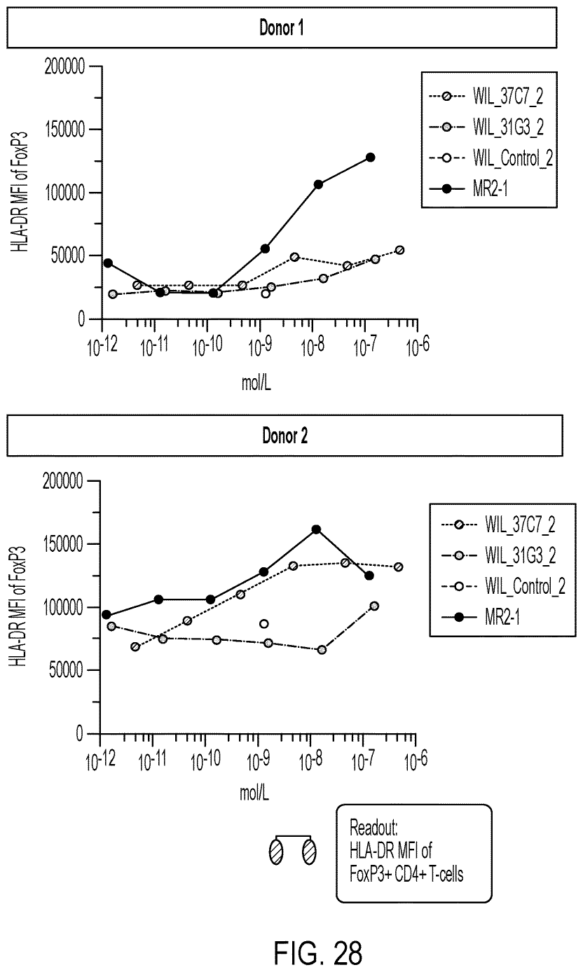

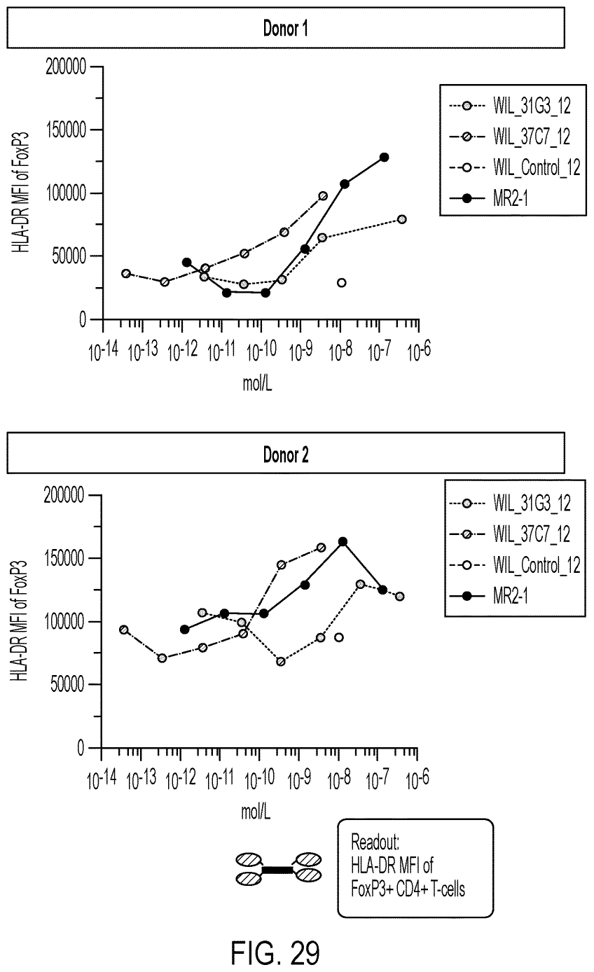

Results additionally revealed anti-TNFR2 V-body fusion construct dose-dependent induction of Treg-suppression marker HLA-DR in FoxP3+ CD4+ Tregs (see, e.g., FIGS. 26-29). Dose-dependent induction of Treg suppression marker HLA-DR expression across various concentrations of tetravalent anti-TNFR2 V-body Fc fusion construct 10 for Donor 1 (top panel) and Donor 2 (bottom panel) are shown in FIG. 27. Tetravalent Fc constructs, irrespective of binder, showed dose-dependent induction of Treg suppression marker HLA-DR expression. Maximal induction of HLA-DR expression was binder dependent. Construct 37C7_10 reached a higher plateau compared to monoclonal TNFR2 agonist MR2-1. Dose-dependent induction of Treg suppression marker HLA-DR expression across various concentrations of rigid bivalent anti-TNFR2 V-body fusion construct 2 for Donor 1 (top panel) and Donor 2 (bottom panel) is shown in FIG. 28. Rigid bivalent constructs at higher concentrations showed modest induction of Treg suppression marker HLA-DR expression with donor dependent differences in maximal expression compared to MR2-1. Dose-dependent induction of Treg suppression marker HLA-DR expression across various concentrations of tetravalent anti-TNFR2 V-body (Vb-Fc-Vb) fusion construct 12 for Donor 1 (top panel) and Donor 2 (bottom panel) is shown in FIG. 29. Vb-Fc-Vb constructs demonstrated dose-dependent induction of Treg suppression marker HLA-DR expression. Maximal induction and potency was dose dependent.

Amino acid sequences of exemplary fusion protein constructs described in Examples 7 and 8 are provided below. In the sequences, VHH sequences are indicated with a straight underline (e.g., VHH), Fc regions are indicated with bold letters (e.g., higG4-P329G SPLE or higG1 LALAGA), IL-2 N88D sequence is indicated with italic letters (e.g., IL2N88D), linker sequences are indicated with a wavy underline (e.g., Linker), and hinge regions are indicated with both a wavy underline and bold letters (e.g., hinge).

WIL_31D6_2xVHH-Fc
(SEQ ID NO: 3933)
EVQLVESGGGLVQAGGSLRLSCAASGSIVSTNGMGWHROVPGKGRELVAGIRSDGFTNYADSVKGRFTISSDNVKNT

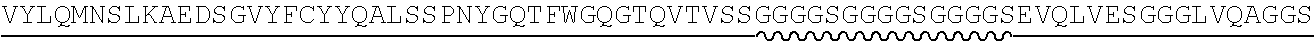

LRLSCAASGSIVSTNGMGWHRQVPGKGRELVAGIRSDGFTNYADSVKGRFTISSDNVKNTVYLQMNSLKAEDSGVYFC

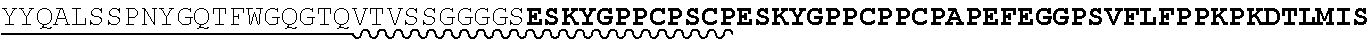

RTPEVTCVVVDVSQEDPEVQFNWYVDGVEVHNAKTKPREEQFNSTYRVVSVLTVLHQDWLNGKEYKCKVSNKGL
GSSIEKTISKAKGQPREPQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
WIL_31D6_VHH-Fc-VHH
(SEQ ID NO: 3935)
EVQLVESGGGLVQAGGSLRLSCÅASGSIVSTNGMGWHROVPGKGRELVAGIRSDGFTNYADSVKGRFTISSDNVKNT

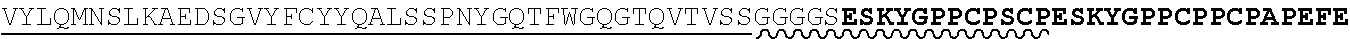

GGPSVFLFPPKPKDTLMISRTPEVTCVVVDVSQEDPEVQFNWYVDGVEVHNAKTKPREEQFNSTYRVVSVLTVLHQ
DWLNGKEYKCKVSNKGLGSSIEKTISKAKGQPREPQVYTLPPSQEEMTKNQVSLTCLVKGFYPSDIAVEWESNGQPE

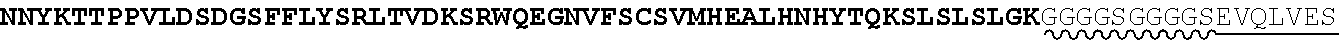

GGGLVQAGGSLRLSCAASGSIVSTNGMGWHRQVPGKGRELVAGIRSDGFTNYADSVKGRFTISSDNVKNTVYLQMNS
LKAEDSGVYFCYYQALSSPNYGQTFWGQGTQVTVSS
WIL_31D6_IL2N88D-Fc-VHH
(SEQ ID NO: 3937)
APTSSSTKKTQLQLEHLLLDLOMILNGINNYKNPKLTRMLTFKFYMPKKATELKHLQCLEEELKPLEEVLNLAQSKNFHLR

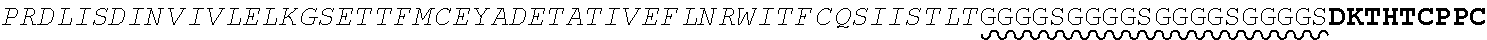

PAPEAAGAPSVFLFPPKPKDTLMISRTPEVTCVVVDVSHEDPEVKFNWYVDGVEVHNAKTKPREEQYNSTYRVVSV
LTVLHQDWINGKEYKCKVSNKALPAPIEKTISKAKGQPREPQVYTLPPSREEMTKNQVSLTCLVKGFYPSDIAVEWES

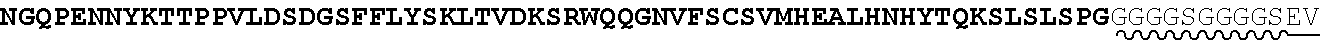

QLVESGGGLVOAGGSLRLSCAASGSIVSTNGMGWHROVPGKGRELVAGIRSDGFTNYADSVKGRFTISSDNVKNTVYL
QMNSLKAEDSGVYECYYQALSSPNYGQTFWGQGTQVTVSS
WIL_31D6_VHH-rigid_linker-VHH
(SEQ ID NO: 3938)
EVQLVESGGGLVQAGGSLRLSCAASGSIVSTNGMGWHRQVPGKGRELVAGIRSDGFTNYADSVKGRFTISSDNVKNT

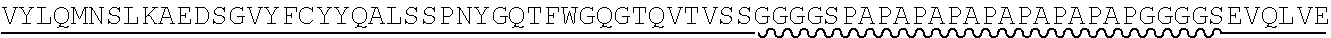

SGGGLVQAGGSLRLSCAASGSIVSTNGMGWHRQVPGKGRELVAGIRSDGFTNYADSVKGRFTISSDNVKNTVYLQMN
SLKAEDSGVYFCYYQALSSPNYGQTFWGQGTQVTVSS
WIL_31G11_2xVHH-Fc
(SEQ ID NO: 3939)
EVQLVESGGGLAQPGGSLRLSCAASGFTFSRYAMSWARQAPGKGLEWVSGISDDGSDTYYADSVKGRFTISRDNAKN

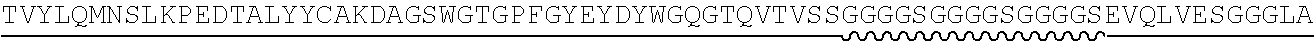

QPGGSLRLSCAASGFTFSRYAMSWARQAPGKGLEWVSGISDDGSDTYYADSVKGRFTISRDNAKNTVYLQMNSLKPE

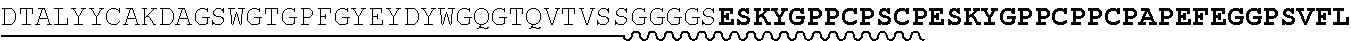

FPPKPKDTLMISRTPEVTCVVVDVSQEDPEVQFNWYVDGVEVHNAKTKPREEQFNSTYRVVSVLTVLHQDWLNGK
EYKCKVSNKGLGSSIEKTISKAKGQPREPQVYTLPPSQEEMTKNQVSLTCLVKGFYPSDIAVEWESNGQPENNYKTTP
PVLDSDGSFFLYSRLTVDKSRWQEGNVFSCSVMHEALHNHYTQKSLSLSLGK
WIL_31G11_VHH-Fc-VHH
(SEQ ID NO: 3941)
EVQLVESGGGLAQPGGSLRISCAASGFTFSRYAMSWARQAPGKGLEWVSGISDDGSDTYYADSVKGRFTISRDNAKN

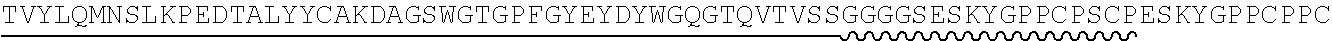

PAPEFEGGPSVFLFPPKPKDTLMISRTPEVTCVVVDVSQEDPEVQFNWYVDGVEVHNAKTKPREEQFNSTYRVVSV
LTVLHQDWINGKEYKCKVSNKGLGSSIEKTISKAKGQPREPQVYTLPPSQEEMTKNQVSLTCLVKGFYPSDIAVEWE

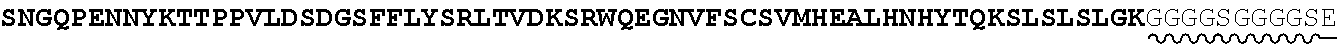

VQLVESGGGLAQPGGSLRLSCAASGFTFSRYAMSWARQAPGKGLEWVSGISDDGSDTYYADSVKGRFTISRDNAKNT
VYLQMNSLKPEDTALYYCAKDAGSWGTGPFGYEYDYWGQGTQVTVSS
WIL_31G11_IL2N88D-Fc-VHH
(SEQ ID NO: 3943)
APTSSSTKKTQLQLEHLLLDLOMILNGINNYKNPKLTRMLTFKFYMPKKATELKHLQCLEEELKPLEEVILNLAQSKNFHLR
PRDLISDINVIVLELKGSETTFMCEYADETATIVEFLNRWITFCQSIISTLTGGGGSGGGGGGGGSGGGGSDKTHTCPPC
PAPEAAGAPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLVKGFYPSDIAVEWES

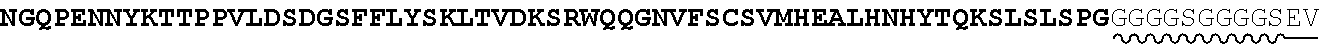

QLVESGGGLAQPGGSLRLSCAASGFTFSRYAMSWARQAPGKGLEWVSGISDDGSDTYYADSVKGRFTISRDNAKNTV
YLQMNSLKPEDTALYCAKDAGSWGTGPFGYEYDYWGQGTQVTVSS
WIL_31G11_VHH-rigid_linker-VHH
(SEQ ID NO: 3944)
EVQLVESGGGLAQPGGSLRLSCAASGFTFSRYAMSWARQAPGKGLEWVSGISDDGSDTYYADSVKGRFTISRDNAKN

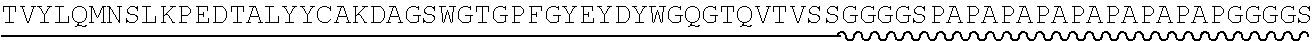

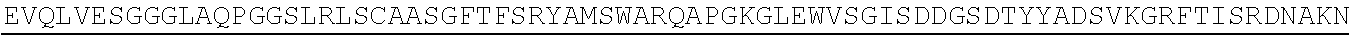

TVYLQMNSLKPEDTALYYCAKDAGSWGTGPFGYEYDYWGQGTQVTVSS
WIL_31G3_2xVHH-Fc
(SEQ ID NO: 3945)
EVQLVESGGGLVOAGGSLRVSCAASGSIFRADAMGWHROVPGKPREFVAGIRSDGFTNYAEAVKGRETISWDTVKNT
VYLQMNSLKPEDTAVYTCYYQSLSSPNYGQVFWGQGTQVTVSSGGGGGGGGGGGGSEVALVESGGGLVQAGGS
LRVSCAASGSIFRADAMGWHRQVPGKPREFVAGIRSDGFTNYAEAVKGRFTISWDTVKNTVYLQMNSLKPEDTAVYT

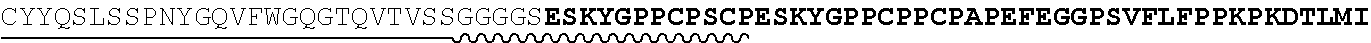

SRTPEVTCVVVDVSQEDPEVQFNWYVDGVEVHNAKTKPREEQFNSTYRVVSVLTVLHQDWLNGKEYKCKVSNKGL
GSSIEKTISKAKGQPREPQVYTLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK
WIL_31G3_VHH-Fc-VHH
(SEQ ID NO: 3947)
EVQLVESGGGLVQAGGSLRVSCAASGSIFRADAMGWHRQVPGKPREFVAGIRSDGFTNYAEAVKGRFTISWDTVKNT

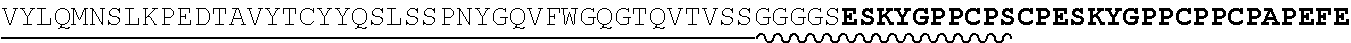

GGPSVFLFPPKPKDTLMISRTPEVTCVVVDVSQEDPEVQFNWYVDGVEVHNAKTKPREEQFNSTYRVVSVLTVLHQ
DWLNGKEYKCKVSNKGLGSSIEKTISKAKGQPREPQVYTLPPSQEEMTKNQVSLTCLVKGFYPSDIAVEWESNGQPE

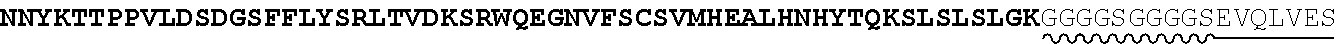

GGGLVQAGGSLRVSCAASGSIFRADAMGWHROVPGKPREFVAGIRSDGFTNYAEAVKGRFTISWDTVKNTVYLQMN
SLKPEDTAVYTCYYQSLSSPNYGQVFWGOGTQVTVSS
WIL_31G3_IL2N88D-Fc-VHH
(SEQ ID NO: 3949)
APTSSSTKKTQLQLEHLLLDLQMILNGINNYKNPKLTRMLTFKFYMPKKATELKHLQCLEEELKPLEEVLNLAQSKNFHLR

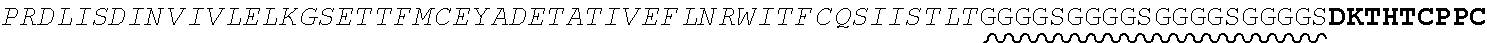

PAPEAAGAPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLVKGFYPSDIAVEWES

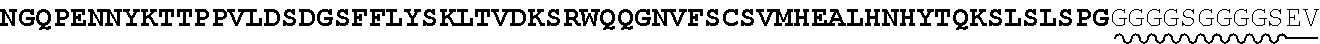

QLVESGGGLVQAGGSLRVSCAASGSIFRADAMGWHRQVPGKPREFVAGIRSDGFTNYAEAVKGRFTISWDTVKNTVY
LQMNSLKPEDTAVYTCYYQSLSSPNYGOVFWGQGTQVTVSS
WIL_31G3_VHH-rigid_linker-VHH
(SEQ ID NO: 3950)
EVQLVESGGGLVQAGGSLRVSCAASGSIFRADAMGWHROVPGKPREFVAGIRSDGETNYAEAVKGRETISWDTVKNT

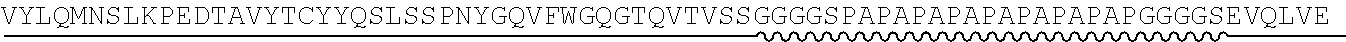

SGGGLVQAGGSLRVSCAASGSIFRADAMGWHRQVPGKPREFVAGIRSDGFTNYAEAVKGRFTISWDTVKNTVYLQM
NSLKPEDTAVYTCYYQSLSSPNYGOVEWGQGTQVTVSS
WIL_33D4_2xVHH-Fc
(SEQ ID NO: 3951)
EVQLVESGGGLVQAGGSLRLSCAASGRTFSDYGMGWFRQAPGKDSEFVAAINWSNGRTNYADSVKGRFTISRDNAK

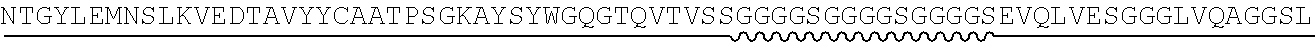

RLSCAASGRTFSDYGMGWFRQAPGKDSEFVAAINWSNGRTNYADSVKGRFTISRDNAKNTGYLEMNSLKVEDTAVYY

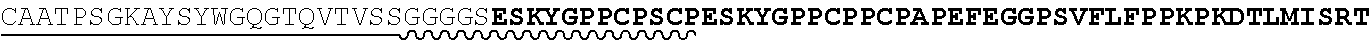

PEVTCVVVDVSQEDPEVQFNWYVDGVEVHNAKTKPREEQFNSTYRVVSVLTVLHQDWLNGKEYKCKVSNKGLGSS
IEKTISKAKGQPREPQVYTLPPSQEEMTKNQVSLTCLVKGFYPSDIAVEWESNGQPENNYKTTPPVLDSDGSFFLYSR
LTVDKSRWQEGNVFSCSVMHEALHNHYTQKSLSLSLGK
WIL_33D4_VHH-Fc-VHH
(SEQ ID NO: 3953)
EVQLVESGGGLVQAGGSLRLSCAASGRTFSDYGMGWFRQAPGKDSEFVAAINWSNGRTNYADSVKGRFTISRDNAK

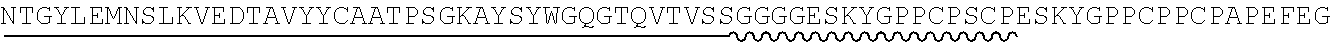

GPSVFLFPPKPKDTLMISRTPEVTCVVVDVSQEDPEVQFNWYVDGVEVHNAKTKPREEQFNSTYRVVSVLTVLHQD
WLNGKEYKCKVSNKGLGSSIEKTISKAKGQPREPQVYTLPPSQEEMTKNQVSLTCLVKGFYPSDIAVEWESNGQPEN

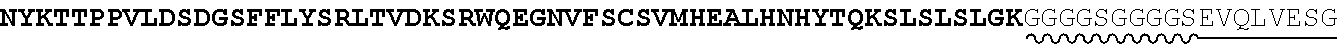

GGLVQAGGSLRLSCAASGRTFSDYGMGWFRQAPGKDSEFVAAINWSNGRTNYADSVKGRFTISRDNAKNTGYLEMN
SLKVEDTAVYYCAATPSGKAYSYWGQGTQVTVSS
WIL_33D4_IL2N88D-Fc-VHH
(SEQ ID NO: 3955)
APTSSSTKKTQLQLEHLLLDLQMILNGINNYKNPKLTRMLTFKFYMPKKATELKHLQCLEEELKPLEEVLNLAQSKNFHLR

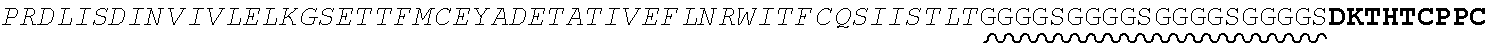

PAPEAAGAPSVFLFPPKPKDTLMISRTPEVTCVVVDVSHEDPEVKFNWYVDGVEVHNAKTKPREEQYNSTYRVVSV
LTVLHQDWINGKEYKCKVSNKALPAPIEKTISKAKGQPREPQVYTLPPSREEMTKNQVSLTCLVKGFYPSDIAVEWES

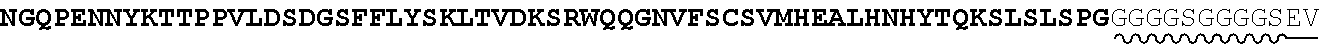

QLVESGGGLVQAGGSLRLSCAASGRTESDYGMGWFRQAPGKDSEFVAAINWSNGRTNYADSVKGRFTISRDNAKNT
GYLEMNSLKVEDTAVYYCAATPSGKAYSYWGQGTQVTVSS
WIL_33D4_VHH-rigid_linker-VHH
(SEQ ID NO: 3956)
EVQLVESGGGLVQAGGSLRLSCAASGRTFSDYGMGWFRQAPGKDSEFVAAINWSNGRTNYADSVKGRFTISRDNAK

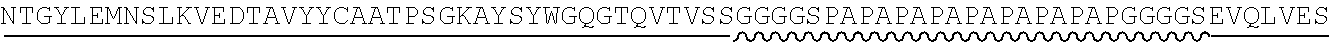

GGGLVOAGGSLRLSCAASGRTFSDYGMGWFRQAPGKDSEFVAAINWSNGRTNYADSVKGRFTISRDNAKNTGYLEM
NSLKVEDTAVYYCAATPSGKAYSYWGOGTQVTVSS
WIL_35A10_VHH-Fc-VHH
(SEQ ID NO: 3957)
EVQLVESGGGLVEAGGSLRLSCAASGRTFGSYTMGWFRQAPGREQEFLASIRRTGGSTSYADSVKGRFTISRDNAKKAV

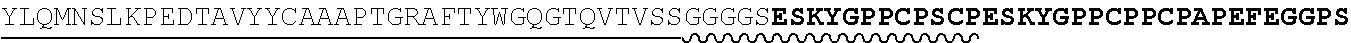

VFLFPPKPKDTLMISRTPEVTCVVVDVSQEDPEVQFNWYVDGVEVHNAKTKPREEQFNSTYRVVSVLTVLHQDWL
NGKEYKCKVSNKGLGSSIEKTISKAKGQPREPQVYTLPPSQEEMTKNQVSLTCLVKGFYPSDIAVEWESNGQPENNY

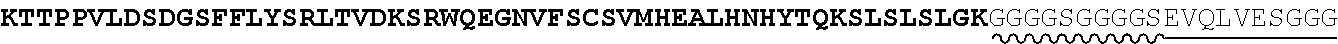

LVEAGGSLRLSCAASGRTFGSYTMGWFRQAPGREQEFLASIRRTGGSTSYADSVKGRFTISRDNAKKAVYLQMNSLKPE
DTAVYYCAAAPTGRAFTYWGQGTQVTVSS
WIL_37C7_2xVHH-Fc
(SEQ ID NO: 3959)
EVQLVESGGGLVQPGGSLRLSCAASGFTFDDIAMTWVRQAPGKGLEWVSSIYSYGPNTYYADSVKGRFTISTDSAKNTL

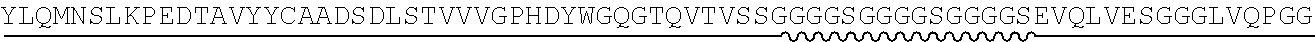

SLRLSCAASGFTFDDIAMTWVRQAPGKGLEWVSSIYSYGPNTYYADSVKGRFTISTDSAKNTLYLQMNSLKPEDTAVYY

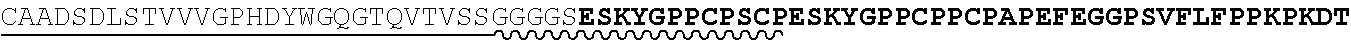

LMISRTPEVTCVVVDVSQEDPEVQFNWYVDGVEVHNAKTKPREEQFNSTYRVVSVLTVLHQDWLNGKEYKCKVSN
KGLGSSIEKTISKAKGQPREPQVYTLPPSQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK
WIL_37C7_VHH-Fc-VHH
(SEQ ID NO: 3961)
EVQLVESGGGLVQPGGSLRLSCAASGFTFDDIAMTWVRQAPGKGLEWVSSIYSYGPNTYYADSVKGRFTISTDSAKNTL

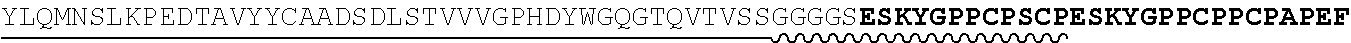

EGGPSVFLFPPKPKDTLMISRTPEVTCVVVDVSQEDPEVQFNWYVDGVEVHNAKTKPREEQFNSTYRVVSVLTVLH
QDWINGKEYKCKVSNKGLGSSIEKTISKAKGQPREPQVYTLPPSQEEMTKNQVSLTCLVKGFYPSDIAVEWESNGQP

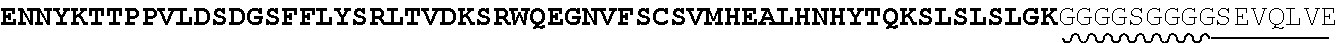

SGGGLVQPGGSLRLSCAASGFTFDDIAMTWVRQAPGKGLEWVSSIYSYGPNTYYADSVKGRFTISTDSAKNTLYLQMN
SLKPEDTAVYYCAADSDLSTVVVGPHDYWGQGTOVTVSS
WIL_37C7_IL2N88D-Fc-VHH
(SEQ ID NO: 3963)
APTSSSTKKTQLQLEHLLLDLQMILNGINNYKNPKLTRMLTFKFYMPKKATELKHLQCLEEELKPLEEVLNLAQSKNFHLR

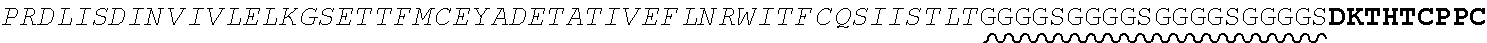

PAPEAAGAPSVFLFPPKPKDTLMISRTPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSREEMTKNQVSLTCLVKGFYPSDIAVEWES

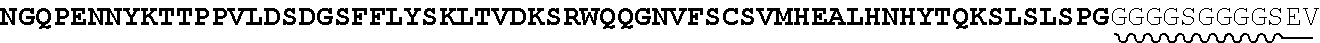

QLVESGGGLVOPGGSLRLSCAASGFTFDDIAMTWVRQAPGKGLEWVSSIYSYGPNTYYADSVKGRFTISTDSAKNTLYL
QMNSLKPEDTAVYYCAADSDLSTVVVGPHDYWGQGTQVTVSS
WIL_37C7_VHH-rigid_linker-VHH
(SEQ ID NO: 3964)
EVQLVESGGGLVQPGGSLRLSCAASGFTEDDIAMTWVRQAPGKGLEWVSSIYSYGPNTYYADSVKGRFTISTDSAKNTL

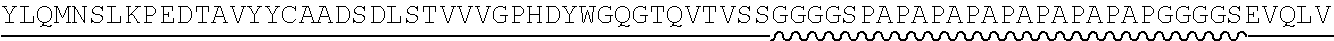

ESGGGLVOPGGSLRLSCAASGETEDDIAMTWVRQAPGKGLEWVSSIYSYGPNTYYADSVKGRETISTDSAKNTLYLQM
NSLKPEDTAVYYCAADSDLSTVVVGPHDYWGQGTQVTVSS

### Example 9. TNFR2 Agonism by Tetravalent VHH-Fc Fusion Increases Suppressive Function and Stabilizes the Phenotype of Treg by Preventing Effector Differentiation In Vitro

TNFR2 agonism induces Treg proliferation and activation. It was hypothesized that TNFR2 stimulation will also increase Treg stability based on the phenotype of TNFR2 knockout mice in which Treg are unstable and convert into effector cells. To test this hypothesis, multivalent anti-TNFR2 VHH proteins with agonist activity on TNFR2 were assessed for their impact on Treg proliferation, activation, immune suppressive function, and stability compared to IL-2 muteins.

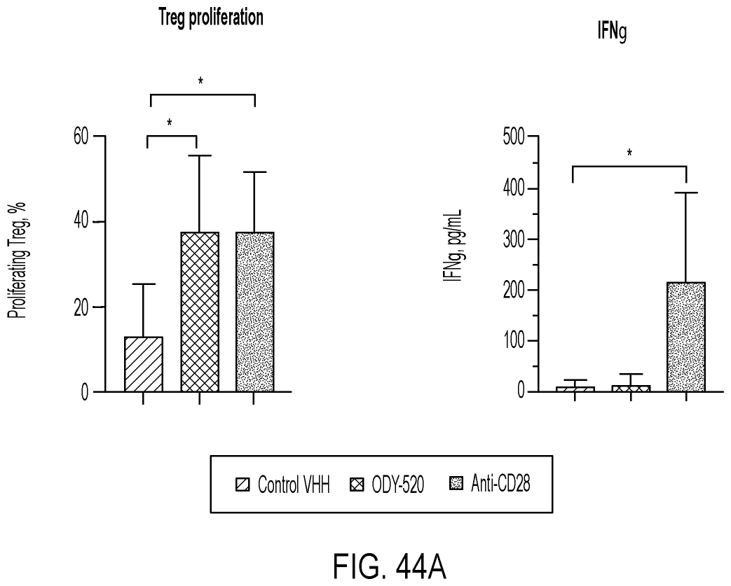

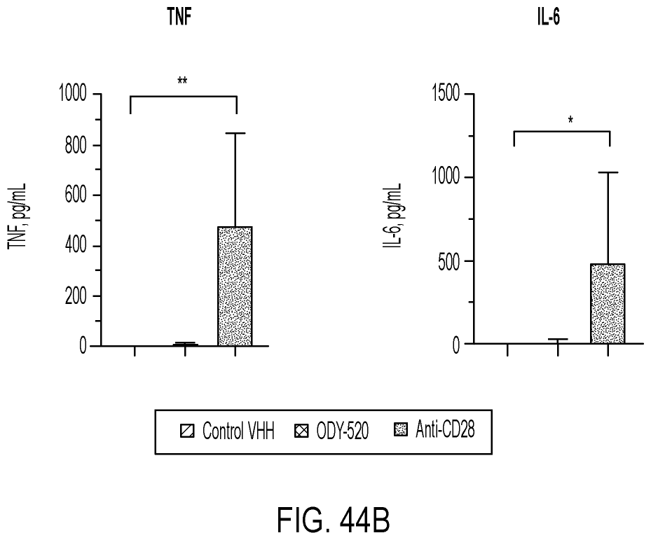

Human naïve Treg treated with anti-TNFR2 VHH or IL-2 mutein were evaluated to detect effects on proliferation, activation, suppressive function, and stability in inflammatory conditions, Specifically, tetravalent-Fc agonists (WIL_33D4_2×VHH-Fc and WIL_37C7_2×VHH-Fc) were characterized in vitro for their ability to expand and activate Treg in the presence of anti-CD3 and IL-2 stimulation. The stability of human naïve Treg (CD4+CD25+CD45RA+) sorted from healthy donor PBMC by magnetic beads was investigated upon stimulation with tetravalent VHH for 5 days followed by 11 to 12 days of inflammatory cytokine challenge (IL-21, IL-23, IL-1b+/−TGFb) and IL-17A or IFNγ intracellular staining after PMA/ionomycin restimulation. Suppressive function was measured by assessing Treg ability to inhibit the proliferation of naïve CD4 responder cells. Similarly, the effect of TNFR2 agonist ODY-520 on proinflammatory cytokine production was evaluated from human PBMC treated with 5 nM TNFR2 V-body or CD28 agonist for 5 days in the presence of anti-CD3. Treg proliferation was measured by assessing percent of proliferating Treg (CD4+FOXP3+) by cell tracer dilution by flow cytometry and cytokine production was measured in the supernatant by homogeneous time resolved fluorescence (HTRF) assay (FIGS. 44A and 44B),

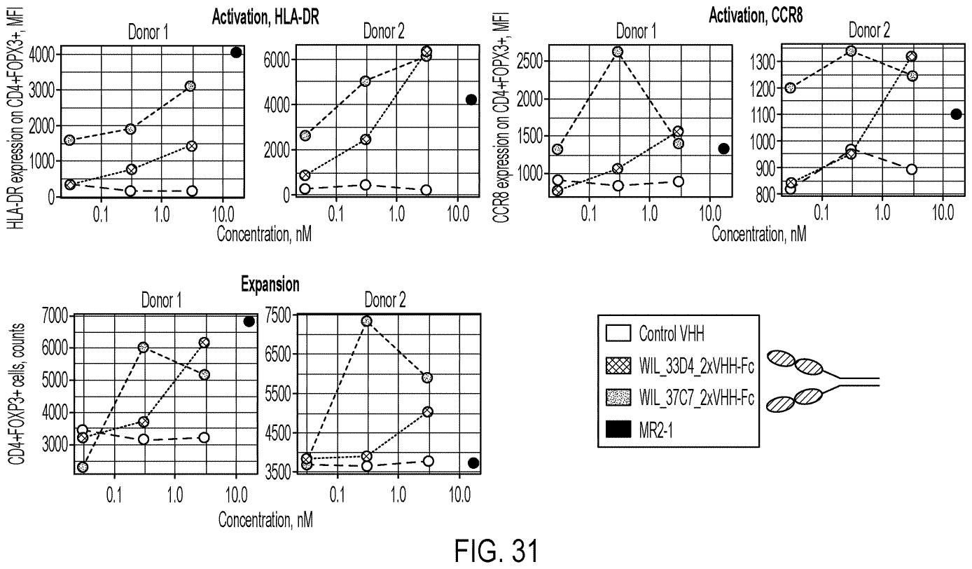

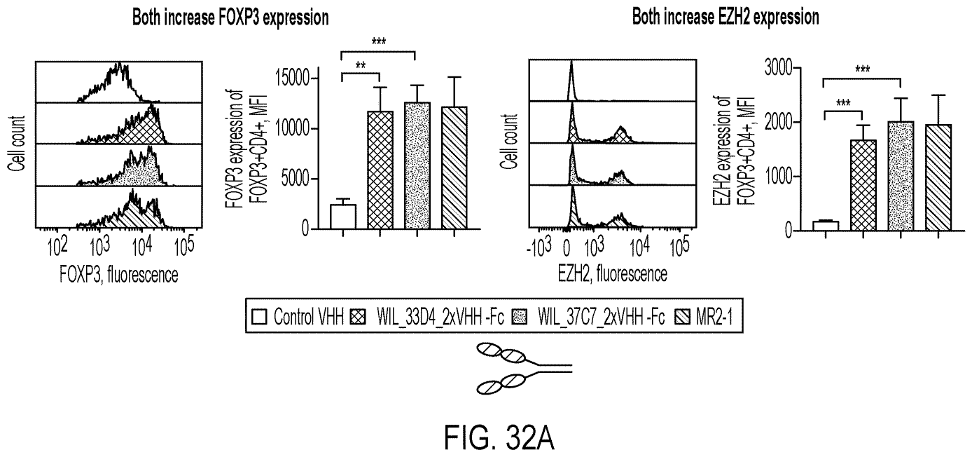

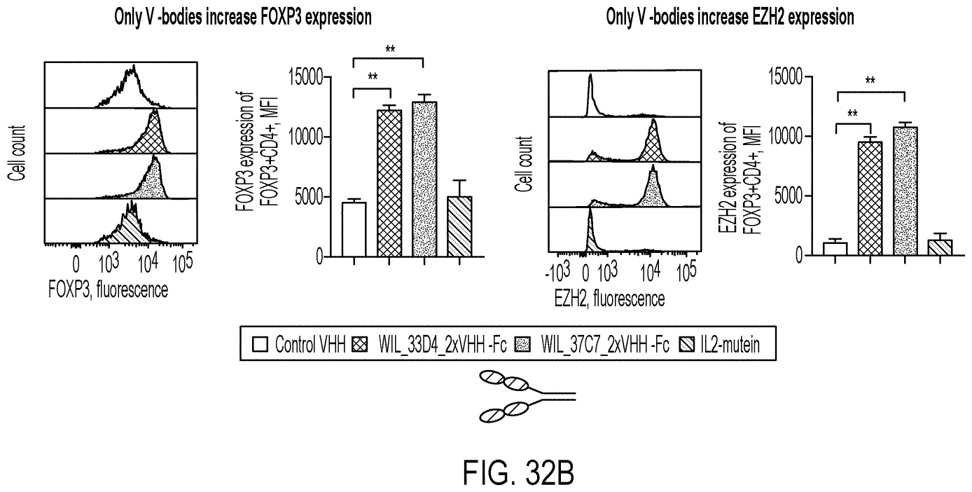

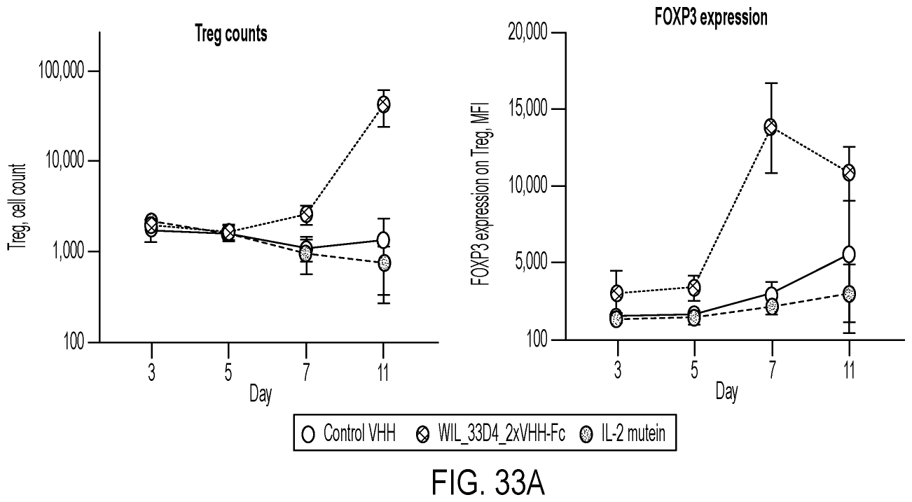

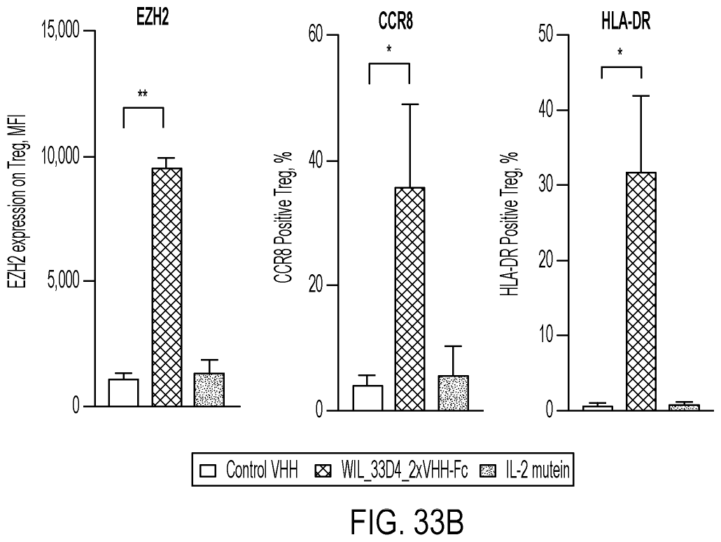

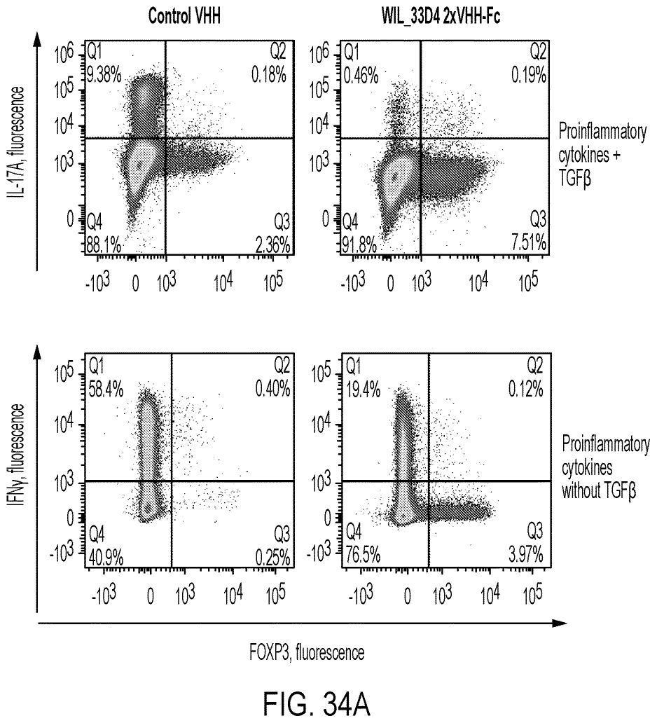

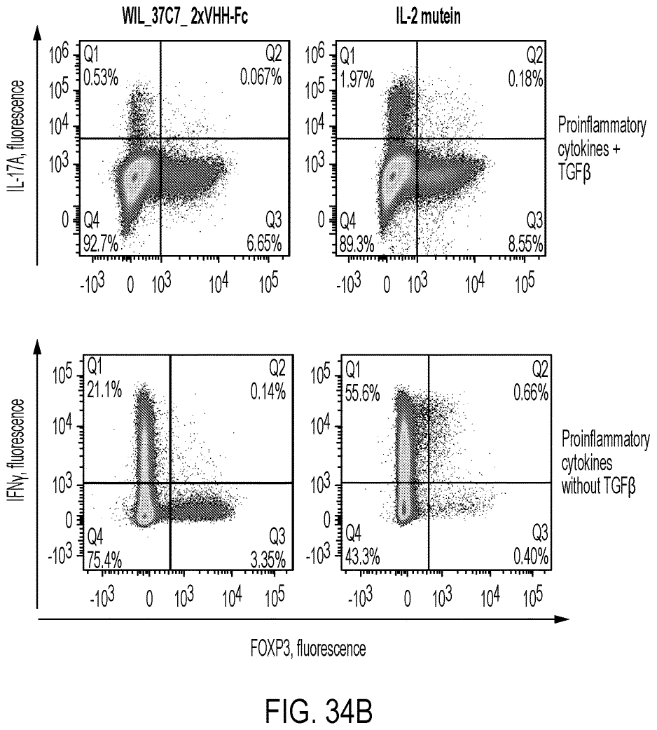

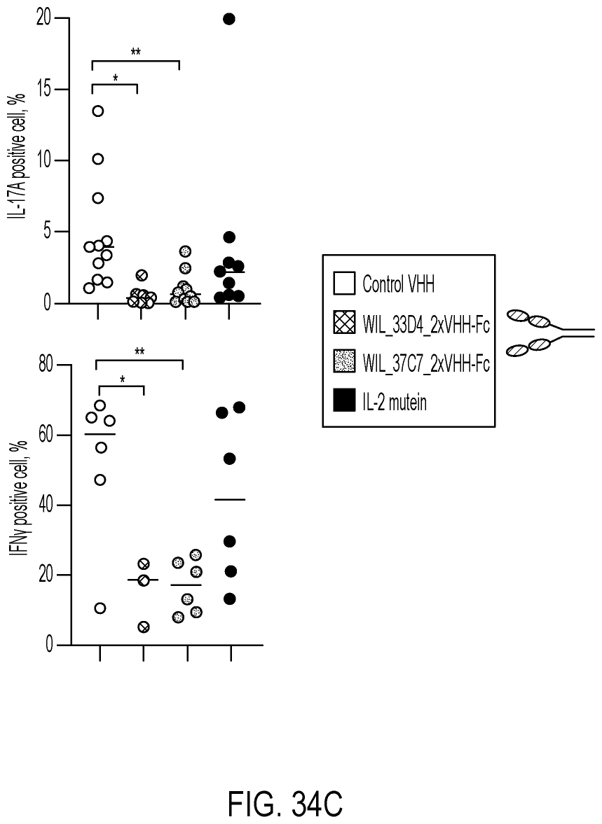

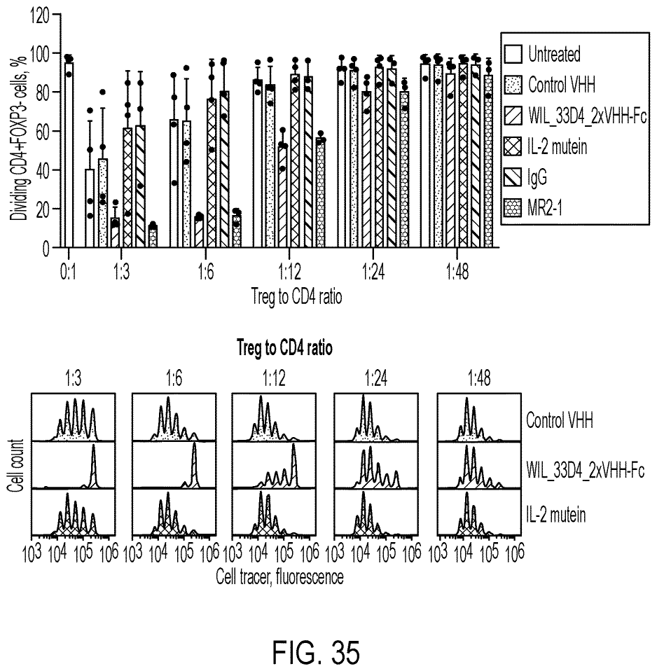

FIGS. 31 and 33A-33B show that tetravalent TNFR2 VHH-Fc antibodies induced the expansion of naive human Treg and up-regulated biomarkers of enhanced immunosuppressive activity such as CCR8 and HLA-DR and stability marker EZH2. In line with these results, activation of TNFR2 increased the ability of naïve human Treg to suppress the proliferation of CD4 responder cells (FIG. 35), Furthermore, stimulation of naïve human Treg with VHH-Fc prevented the loss of FOXP3 and their differentiation into IL-17A- or IFNγ-secreting cells when cultivated in the presence of inflammatory cytokines (FIGS. 32A, 32B and 34A-34C). TNFR2 agonist expanded Treg (CD4+FOXP3+) without inducing proinflammatory cytokines compared to CD28 agonist. (FIG. 44).

Agonistic TNFR2 VHH induced the expansion of naïve human Treg, increased the expression of activation markers and stimulated suppressive effects against CD4+ T effector cells. TNFR2 VHH constructs, but not IL-2 muteins, increased human Treg stability in the presence of proinflammatory cytokines based on sustained presence of FOXP3 bright cells and prevention of conversion of Treg into IL-17A- or IFNγ-secreting cells. These data demonstrate that agonism of TNFR2 by a tetravalent VHH-Fc fusion antibody expands human Treg, increases their suppressive function and stabilize their phenotype under inflammatory conditions in vitro.

### Example 10. TNFR2 Agonism by Tetravalent VHH-Fc Fusion and Expands and Activates Treg Cell In Vivo

The effects of anti-TNFR2 VHH agonists on spleen lymphocyte population in human TNFR2 knock-in mice were assessed by flow cytometry 5 days after a single intravenous injection (2.5 mg/kg) of tetravalent VHH-Fc antibody WIL_33D4_2×VHH-Fc, or control VHH, or mAb into human TNFR2 knock-in mice.

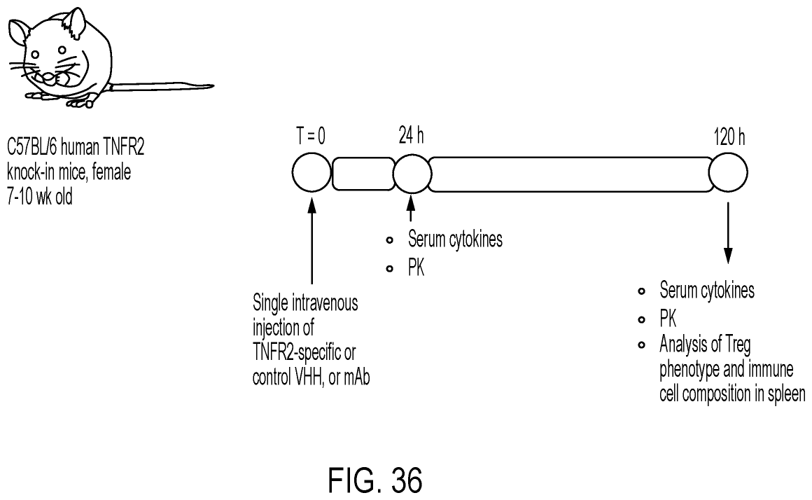

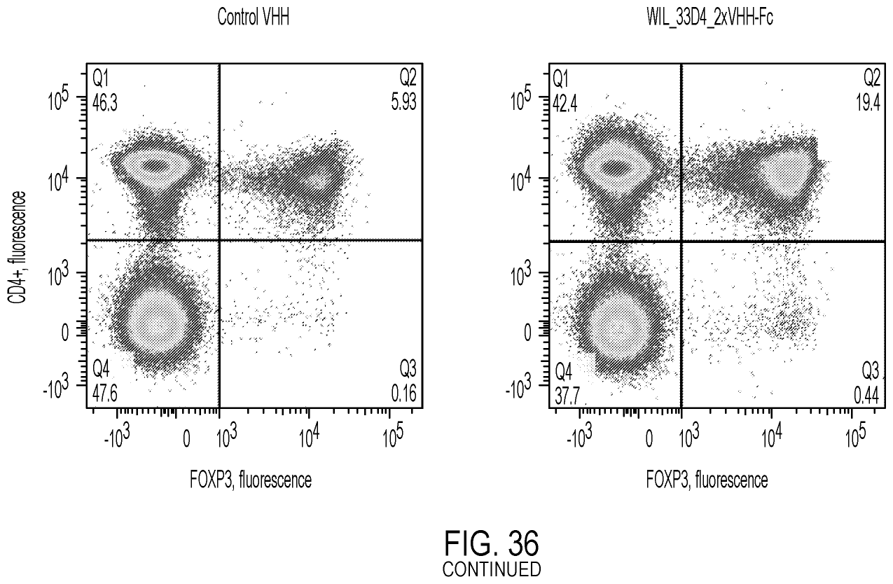

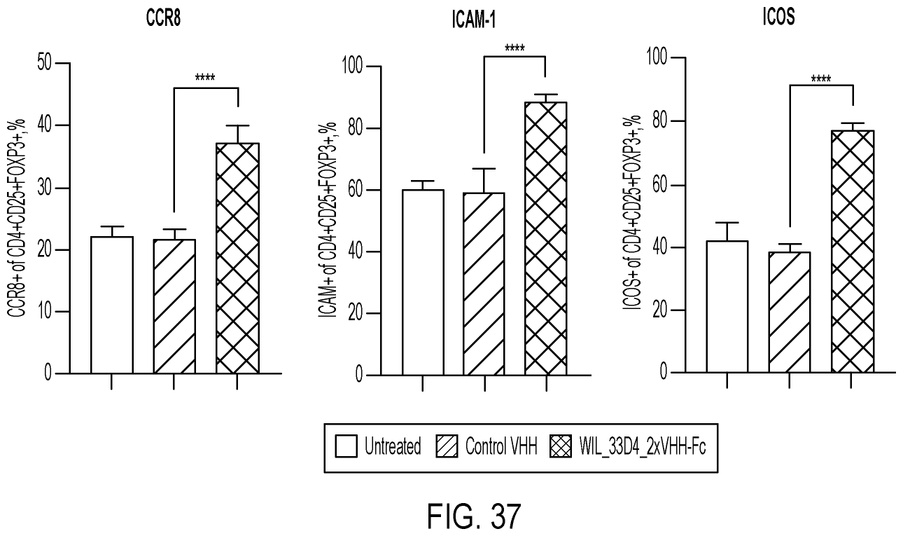

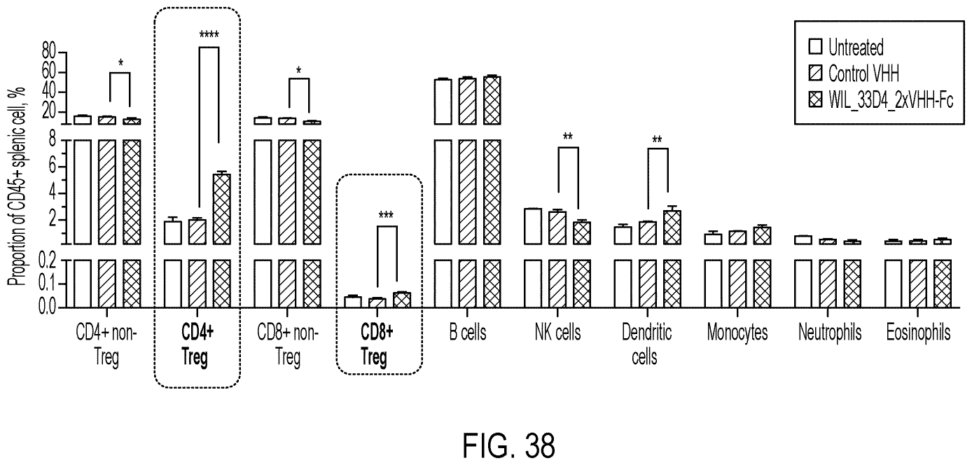

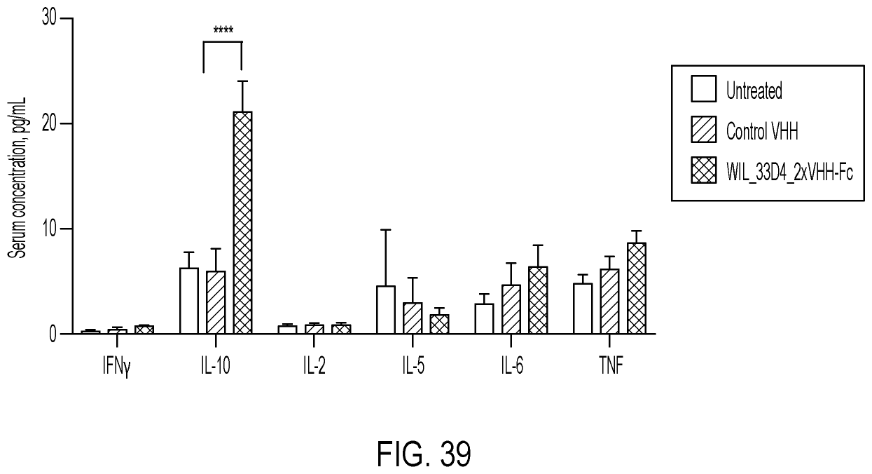

Upon injection in human TNFR2 knock-in mice, tetravalent VHH-Fc antibody WIL 33D4_2×VHH-Fc led to a 3-fold increase in the percentage of FOXP3+ cell among CD4+ cells in the spleen, while no increase of CD4+FOXP3− and CD8+FOXP3− conventional T cells and only a minor impact on overall splenic immune cell composition was observed (FIGS. 36 and 38). Treatment led to Treg activation as shown by the upregulation of ICOS and ICAM-1 (FIG. 37). Importantly, injection was well tolerated and did not induce weight loss or changes in systemic levels of inflammatory cytokines (TNF, IL-6, IFNγ, IL-5) but increased serum level of IL-10, a cytokine with demonstrated immune-regulatory functions (FIG. 39). TNFR2 agonists, but not IL-2 muteins, increased human Treg stability in the presence of proinflammatory cytokines based on sustained high expression of FOXP3 and prevention of conversion of Treg into IL-17A or IFNγ-secreting cells (FIGS. 34A-34C).

These data demonstrated that in vivo, a single injection is safe in mice and leads to Treg expansion and activation. Anti-TNFR2 VHH agonistic proteins are effective at expanding Tregs that have a more stable immunosuppressive capacity and phenotype (preventing pathogenic conversion to Teff) compared to IL-2 muteins and may provide a durable and disease modifying therapy for multiple autoimmune diseases.

### Example 11. Evaluation of Effect of VHH-Fc Fusion on Treg Function In Vivo

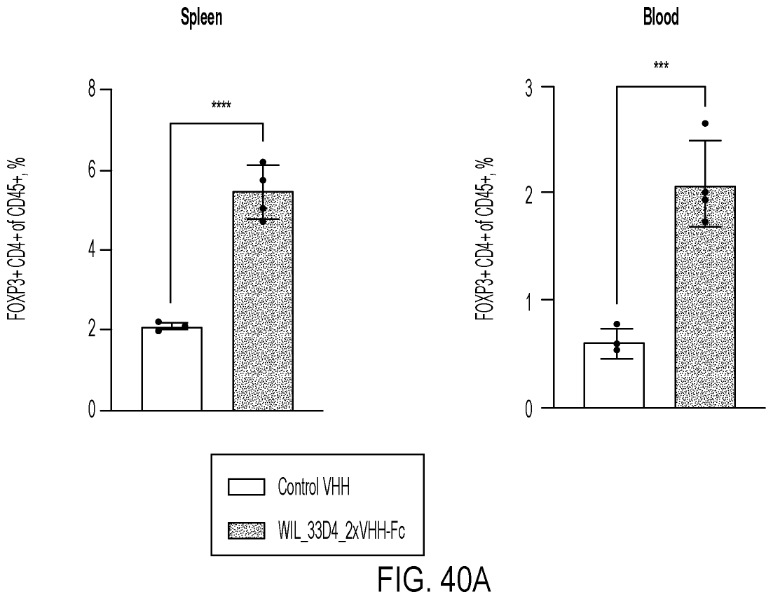

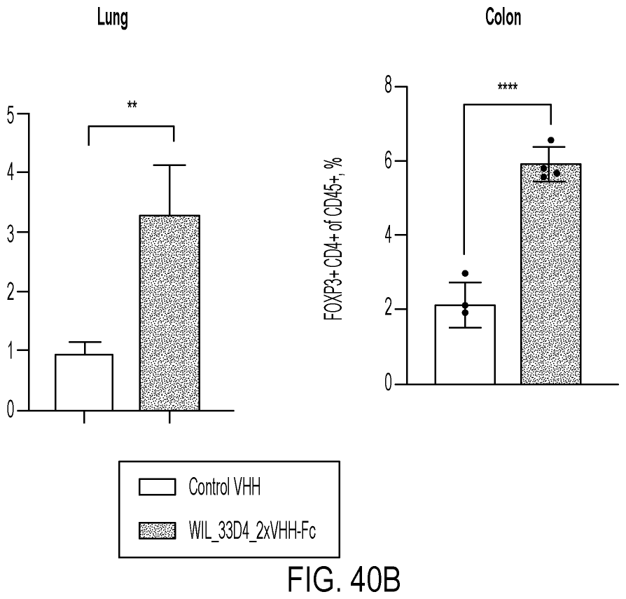

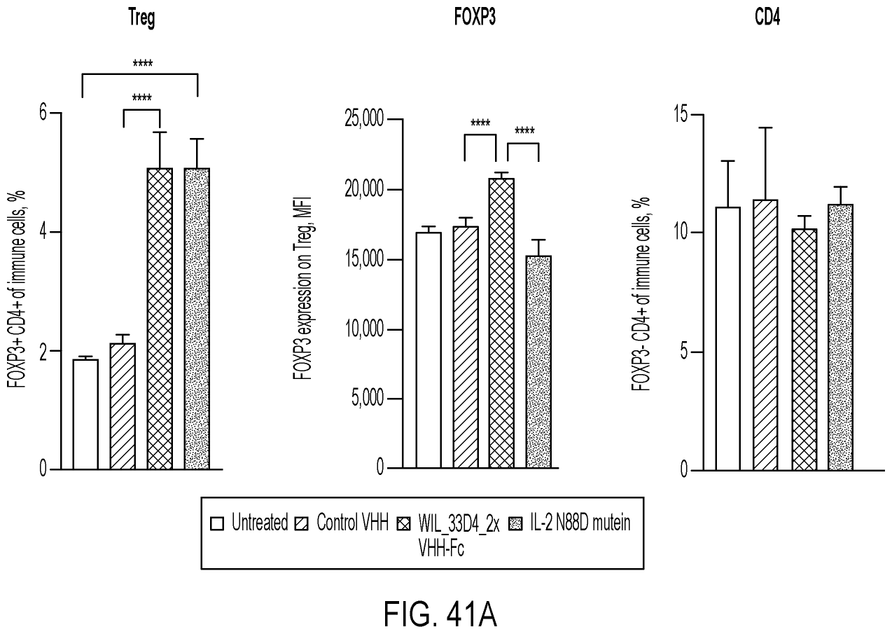

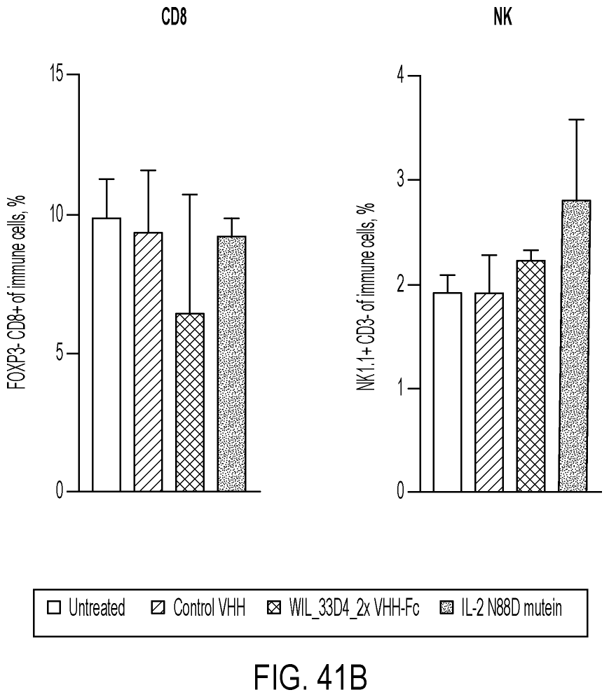

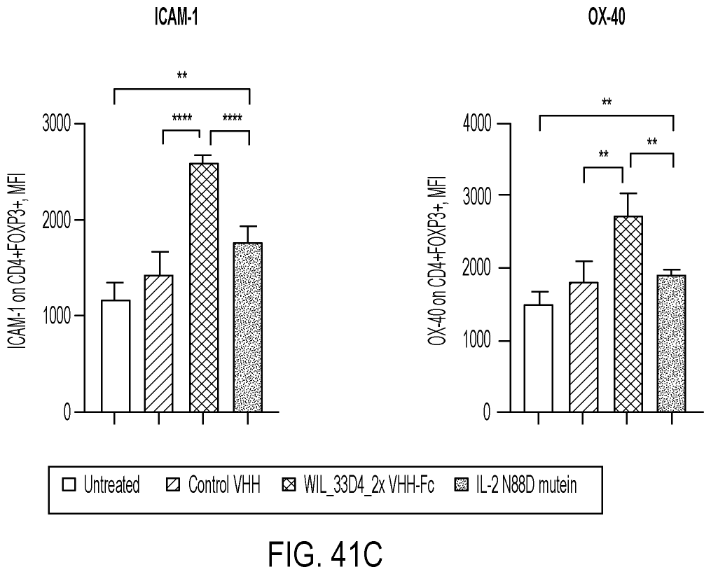

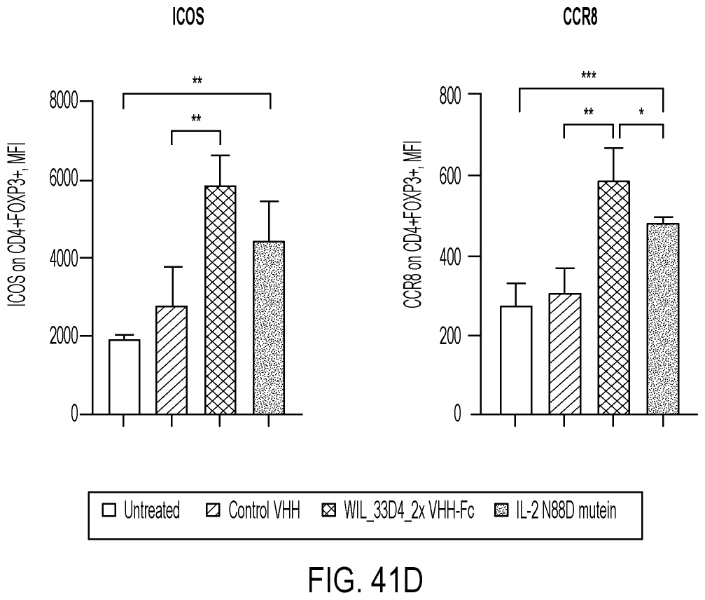

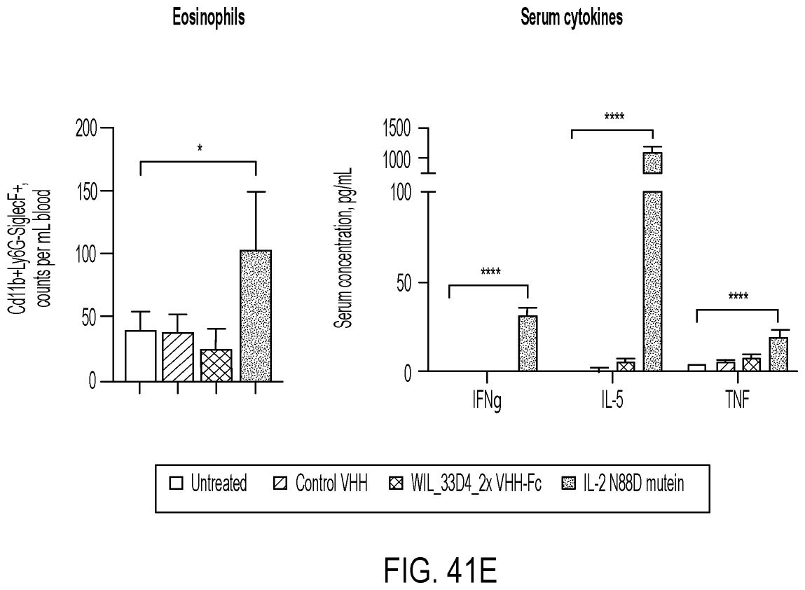

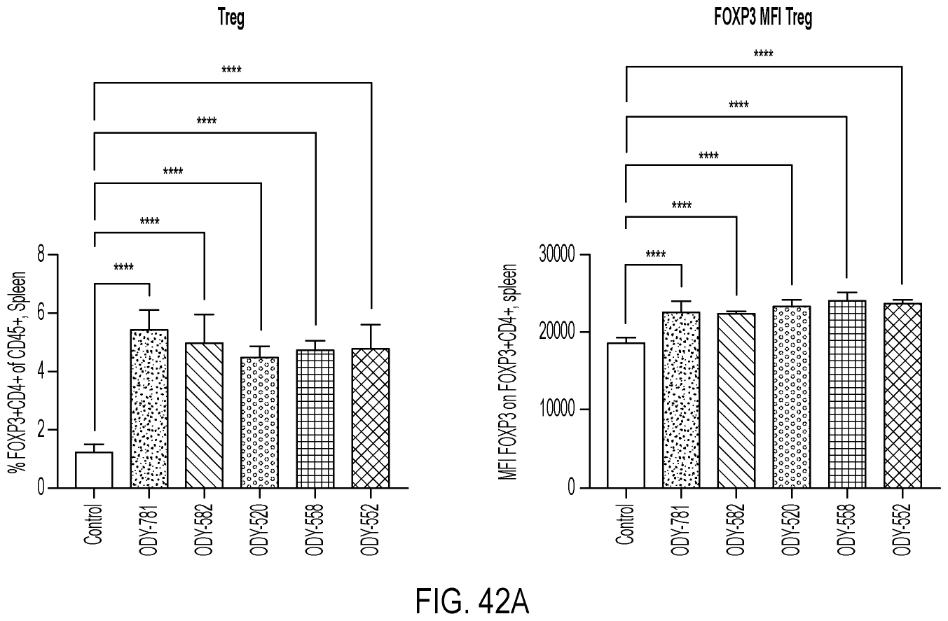

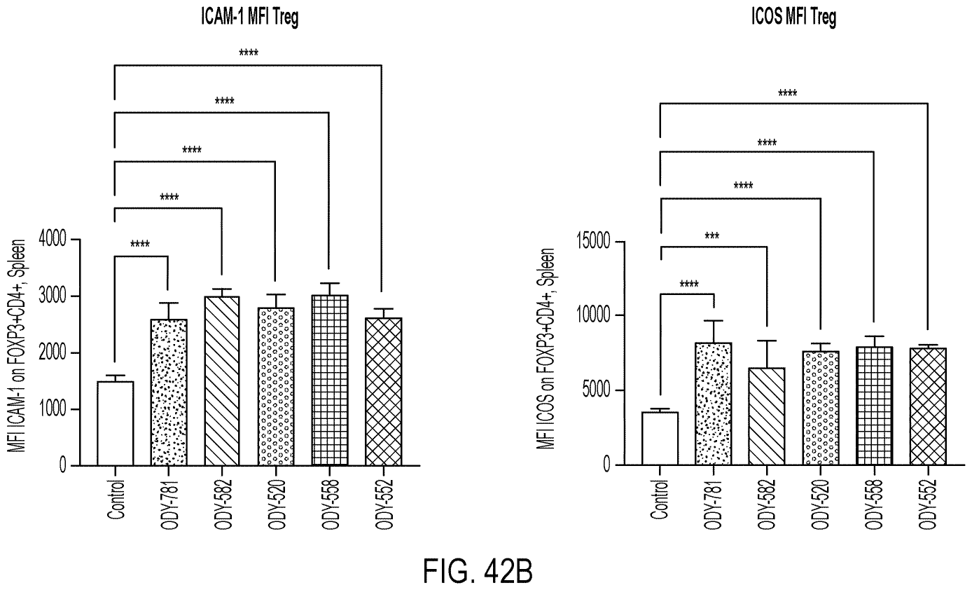

Human TNFR2 knock-in mice were injected with the constructs as indicated in FIGS. 42A-42B at 3 mg/kg intravenously. After 5 days, percentage of Treg (FOXP3+CD4+) among CD45+ cell as well as FOXP3, ICAM-1, ICOS expression (MFI, mean fluorescent intensity) on Treg was measured by flow cytometry. Data as depicted in FIGS. 42A and 428 show Treg expansion in the spleen as well as increased expression of FOXP3, linked to Treg stability and function, and Treg activation shown by up-regulation of ICAM-1 and ICOS. Administration of anti-TNFR2 VHH agonists to mice increased Treg by three-fold in blood and tissues without increasing conventional Teff or NK cells as it induced a higher level of FOXP3 and surface markers (FOXP3, ICAM-1, OX-40, ICOS, and CCR8). The effects of anti-TNFR2 VHH agonists on spleen lymphocyte population or on blood cells (eosinophils) in human TNFR2 knock-in mice were assessed after a single intravenous injection (3 mg/kg) of tetravalent VHH-Fc antibody ODY-520, or control VHH, or IL-2 N88D into human TNFR2 knock-in mice. Similarly, the effect of anti-TNFR2 VHH agonist was assessed on serum cytokines 1 day after the single injection of tetravalent VHH-Fc antibody ODY-520, control VHH or IL-2 N88D. (FIGS. 41A-41E). Further, anti-TNFR2 VHH agonists expand Treg across tissues. (FIGS. 40A-40B).

### Example 12. Evaluation of the Effect of VHH-Fc Fusion on Arthritis In Vivo

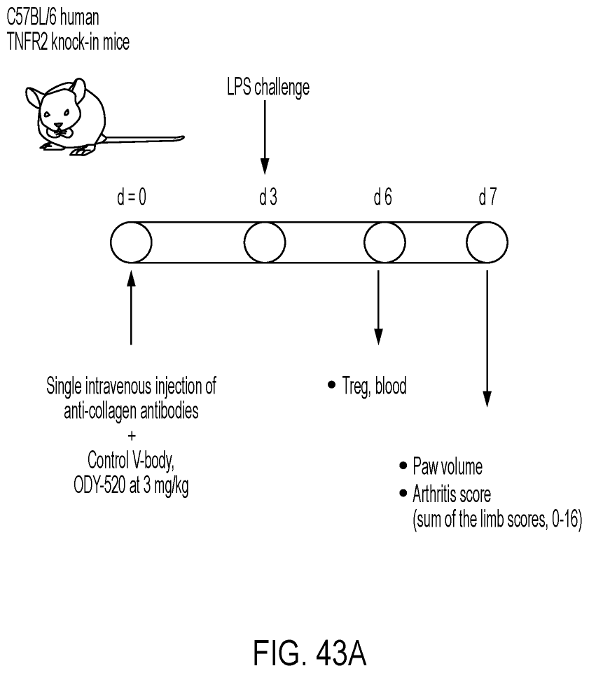

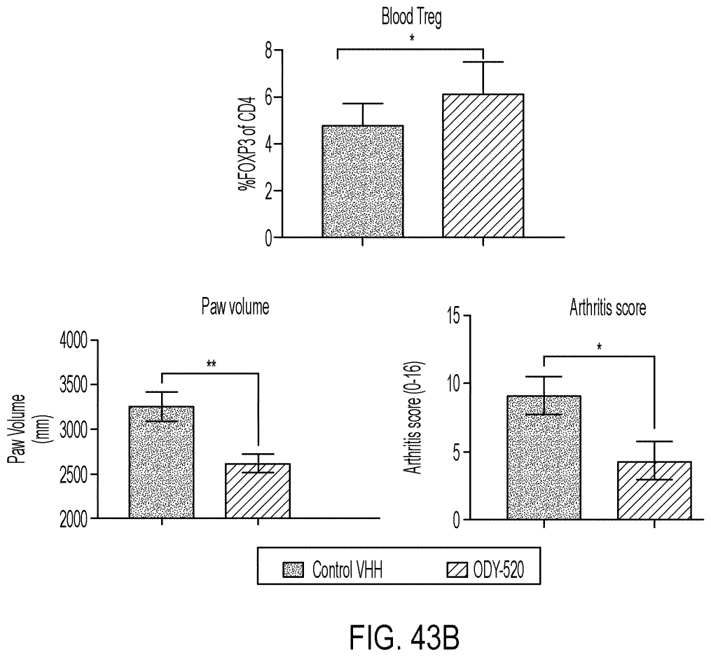

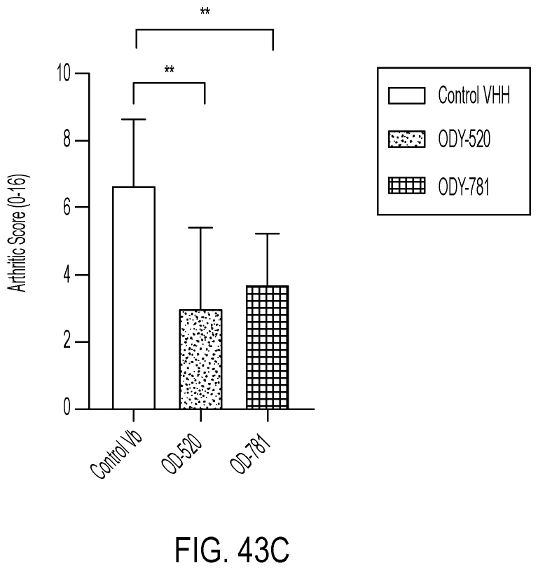

At day 0, human TNFR2 knock-in mice were injected with anti-collagen antibodies as well construct ODY-520 at 3 mg/kg intravenously or control VHH. At day 3, mice were challenged with lipopolysaccharide (LPS). Treg cells were measured at day 6 and at day 7, arthritis score (total number of affected digits) and paw volume were measured. The same experiment was repeated with ODY-781 construct. Data as depicted in FIGS. 43B and 43C show reduction of arthritis, measured by paw volume and arthritis score, in a model of collagen-antibody induced arthritis upon treatment with TNFR2 agonist ODY-520 and ODY-781.

Amino acid sequences of exemplary fusion protein constructs described in Examples 11 and 12 as well as additional engineered constructs are provided below. In the sequences, VHH sequences are indicated with a straight underline (e.g., VHH), Fc regions are indicated with bold letters (e.g., hIgG4-SPLE or hIgG1 LALAPA), N-terminal modification are indicated with both bold and a straight underline (e.g., N-term modification), C-terminal modification are indicated with both italic and a wavy underline (e.g., C-term modification) linker sequences are indicated with a wavy underline (e.g., Linker), and hinge regions are indicated with both a wavy underline and bold letters (e.g., hinge).

ODY-520
(SEQ ID NO: 4483)
EVQLLESGGGLVQPGGSLRLSCAASGRTFSDYGMGWFRQAPGKDSEFVAAINWSNGRTNYADSVKGRFTISRDNAKN

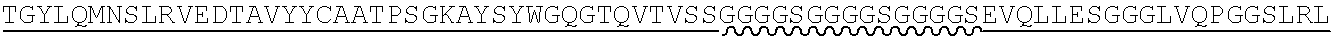

SCAASGRTESDYGMGWFRQAPGKDSEFVAAINWSNGRTNYADSVKGRETISRDNAKNTGYLQMNSLRVEDTAVYYC

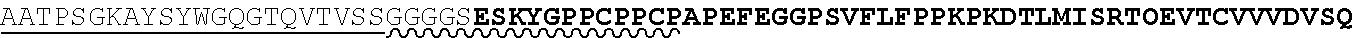

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-552
(SEQ ID NO: 4484)
EVQLLESGGGLVOPGGSLRLSCAASGRTFSDYGMGWFRQAPGKDSEFVAAINWSNGRTNYADSVKGRFTISRDNAKN

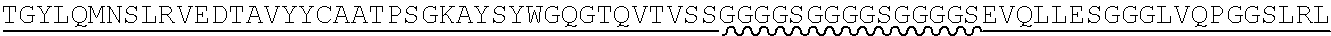

SCAASGRTFSDYGMGWFRQAPGKDSEFVAAINWSNGRTNYADSVKGRFTISRDNAKNTGYLQMNSLRVEDTAVYYC

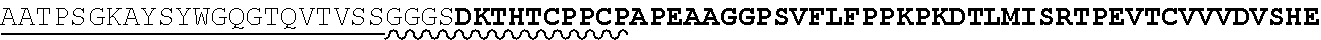

DPEVKFNWYVDGVEVHNAKTKPREEQYNSTYRVVSVLTVLHQDWLNGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPG
ODY-558
(SEQ ID NO: 4485)
EVQLLESGGGLVQPGGSLRLSCAASGRTFSDYGMGWFRQAPGKDSEFVAAINWSNGRTNYADSVKGRFTISRDNAKN

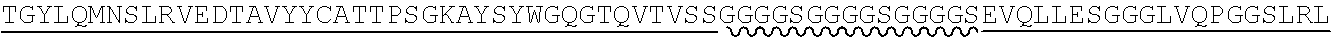

SCAASGRTFSDYGMGWFRQAPGKDSEFVAAINWSNGRTNYADSVKGRFTISRDNAKNTGYLQMNSLRVEDTAVYYC

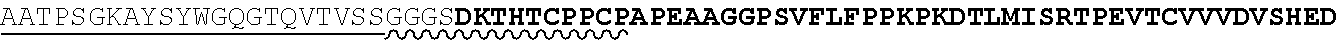

PEVKFNWYVDGVEVHNAKTKPREEQYNSTYRVVSVLTVLHQDWLNGKEYKCKVSNKALAAPIEKTISKAKGQPREP
QVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPG
ODY-582
(SEQ ID NO: 4486)
EVQLLESGGGLVQPGGSLRLSCAASGRTFGSYTMGWFRQAPGKEREFLASIRWTGGSTSYADSVKGRFTISRDDAKKT

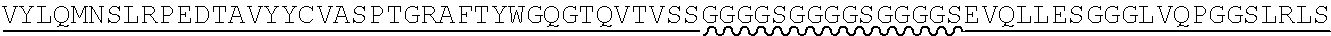

CAASGRTFGSYTMGWFROAPGKEREFLASIRWTGGSTSYADSVKGRFTISRDDAKKTVYLQMNSLRPEDTAVYYCVAS

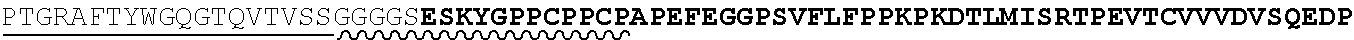

EVQFNWYVDGVEVHNAKTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYSRLTVDKSRWQEGNVFS
CSVMHEALHNHYTQKSLSLSLGK
ODY-583
(SEQ ID NO: 4487)
EVQLLESGGGLVQPGGSLRLSCAASGRTFGSYTMGWFRQAPGKEREFLASIRWTGGSTSYADSVKGRFTISRDDAKKT

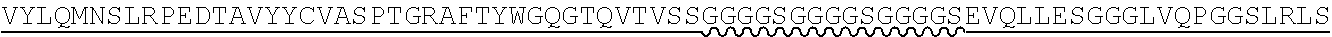

CAASGRTFGSYTMGWFRQAPGKEREFLASIRWTGGSTSYADSVKGRFTISRDDAKKTVYLOMNSLRPEDTAVYYCVAS

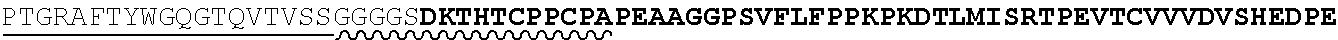

VKFNWYVDGVEVHNAKTKPREEQYNSTYRVVSVLTVLHQDWINGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-780
(SEQ ID NO: 4488)
EVQLLESGGGLVQPGGSLRLSCAASGRTFSDYGMGWFRQAPGKDSEFVAAINWSNGKTNYADSVKGRFTISRDNAKN

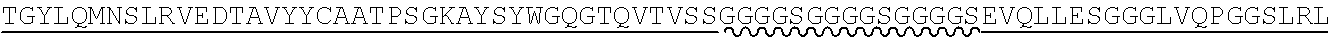

SCAASGRTFSDYGMGWFRQAPGKDSEFVAAINWSNGKTNYADSVKGRFTISRDNAKNTGYLQMNSLRVEDTAVYYC

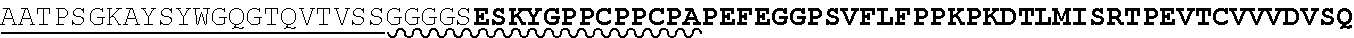

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-781
(SEQ ID NO:4489)
EVQLLESGGGLVQPGGSLRLSCAASGETEDDIAMTWVRQAPGKGLEWVSSIYSYGPNTYYADSVKGRETISTDSAKNTL

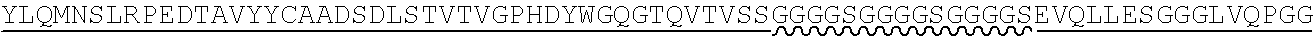

SLRLSCAASGETEDDIAMTWVROAPGKGLEWVSSIYSYGPNTYYADSVKGRETISTDSAKNTLYLOMNSLRPEDTAVYY

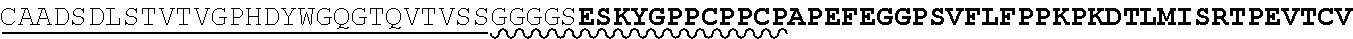

VVDVSQEDPEVQFNWYVDGVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSISLGK
ODY-794
(SEQ ID NO:4490)
EVQLLESGGGLVQPGGSLRLSCAASGRTESDYGMGWFRQAPGKDSEFVAAINWSNARTNYADSVKGRFTISRDNAKN

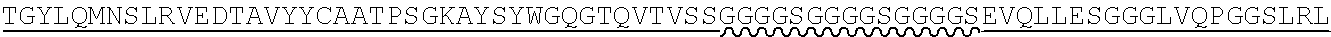

SCAASGRTFSDYGMGWFRQAPGKDSEFVAAINWSNARTNYADSVKGRFTISRDNAKNTGYLQMNSLRVEDTAVYYC

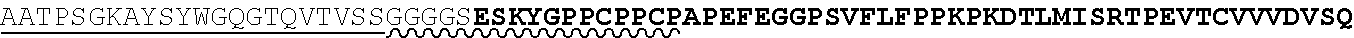

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-840
(SEQ ID NO: 4491)
EVQLLESGGGLVQPGGSLRLSCAASGRTESDYGMGWFRQAPGKDSEFVAAINWSNARTNYADSVKGRFTISRDNAKN

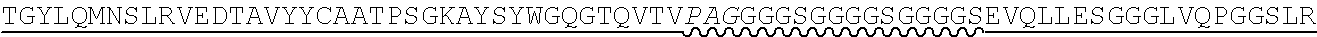

LSCAASGRTESDYGMGWFRQAPGKDSEFVAAINWSNARTNYADSVKGRFTISRDNAKNTGYLQMNSLRVEDTAVYY

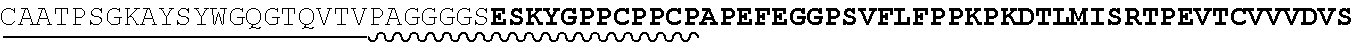

QEDPEVQFNWYVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-906
(SEQ ID NO: 4492)
EVQLLESGGGLVQPGGSLRLSCAASGRTFSDYGMGWFRQAPGKDSEFVAAINWSNARTNYADSVKGRFTISRDNAKN

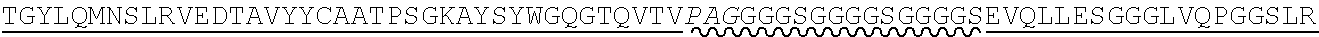

LSCAASGRTFSDYGMGWFRQAPGKDSEFVAAINWSNARTNYADSVKGRFTISRDNAKNTGYLQMNSLRVEDTAVYY

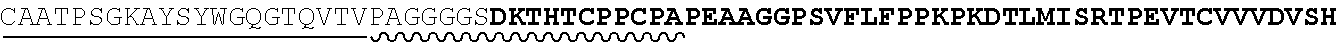

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-910
(SEQ ID NO: 4493)
DVQLLESGGGLVQPGGSLRLSCAASGRTFGSYTMGWFRQAPGKEREFLASIRWTGGSTSYADSVKGRETISRDDAKKT

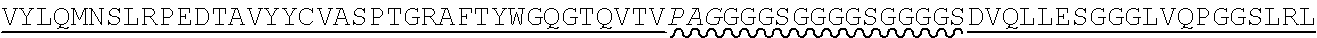

SCAASGRTFGSYTMGWFRQAPGKEREFLASIRWTGGSTSYADSVKGRFTISRDDAKKTVYLQMNSLRPEDTAVYYCVA

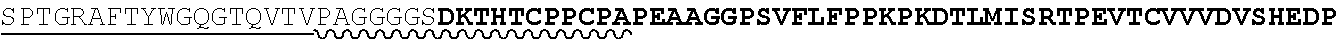

EVKFNWYVDGVEVHNAKTKPREEQYNSTYRVVSVLTVLHQDWLNGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-939
(SEQ ID NO: 4494)
DVQLLESGGGLVQPGGSLRLSCAASGRTFGSYTMGWFRQAPGKEREFLASIRWTGGSTSYADSVKGRETISRDDAKKT

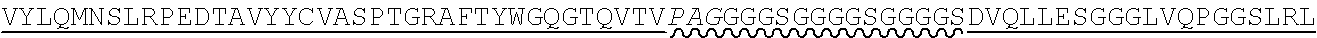

SCAASGRTFGSYTMGWFRQAPGKEREFLASIRWTGGSTSYADSVKGRFTISRDDAKKTVYLOMNSLRPEDTAVYYCVA

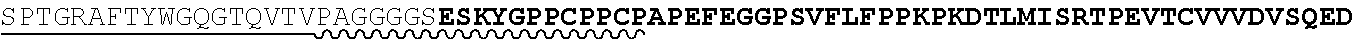

PEVQFNWYVDGVEVHNAKTKPREEQFNSTYRVVSVLTVLHQDWINGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSRWQEGNV
FSCSVMHEALHNHYTQKSLSLSLGK
ODY-941
(SEQ ID NO: 4686)
DVQLVESGGGLVQPGGSLRLSCAASGSIFRFPPMGWYRQAPGKQREQVAQLTSGGSTNYADSVKGRFTISRDNAKNT

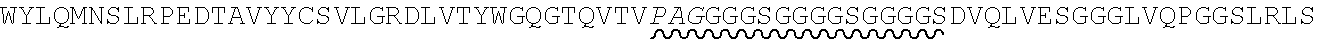

CAASGSIFRFPPMGWYRQAPGKQREQVAQLTSGGSTNYADSVKGRFTISRDNAKNTWYLQMNSLRPEDTAVYYCSVL

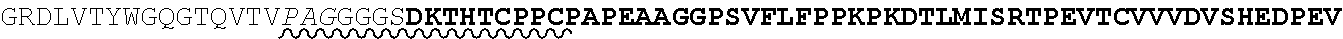

KFNWYVDGVEVHNAKTKPREEQYNSTYRVVSVLTVLHQDWLNGKEYKCKVSNKALAAPIEKTISKAKGQPREPQVY
TLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGK
ODY-942
(SEQ ID NO: 4687)
DVQLVESGGGLVQPGGSLRLSCAASGSIFRFPPMGWYRQAPGKQREQVAQLTSGGSTNYADSVKGRFTISRDNAKNT

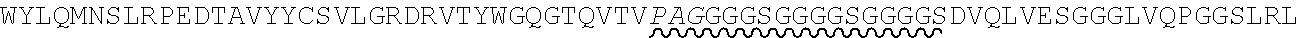

SCAASGSIFRFPPMGWYRQAPGKQREQVAQLTSGGSTNYADSVKGRFTISRDNAKNTWYLQMNSLRPEDTAVYYCSV

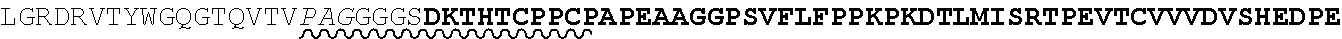

VKFNWYVDGVEVHNAKTKPREEQYNSTYRVVSVLTVLHQDWLNGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-943
(SEQ ID NO: 4688)
DVQLVESGGGLVQPGGSLRLSCAASGSIFRFPPMGWYRQAPGKQREQVAQLTSGGSTNYADSVKGRFTISRDNAKNT

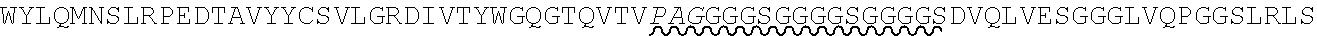

CAASGSIFRFPPMGWYRQAPGKQREQVAQLTSGGSTNYADSVKGRFTISRDNAKNTWYLQMNSLRPEDTAVYYCSVL

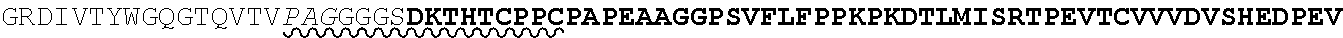

KFNWYVDGVEVHNAKTKPREEQYNSTYRVVSVLTVLHQDWLNGKEYKCKVSNKALAAPIEKTISKAKGQPREPQVY
TLPPSREEMTKNQVSLTCLVKGFYPSDIAVEWESNGQPENNYKTTPPVLDSDGSFFLYSKLTVDKSRWQQGNVFSCS
VMHEALHNHYTQKSLSLSPGK
ODY-944
(SEQ ID NO: 4689)
DVQLLESGGGLVQPGGSLRLSCAASGRTFSDYGMGWFROAPGKDSEFVAAINWSNARTNYADSVKGRETISRDNAKN

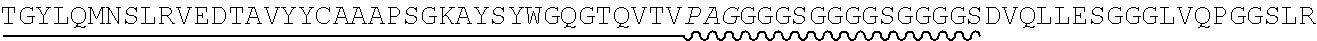

LSCAASGRTESDYGMGWFRQAPGKDSEFVAAINWSNARTNYADSVKGRFTISRDNAKNTGYLOMNSLRVEDTAVYY

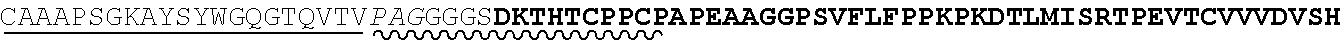

EDPEVKFNWYVDGVEVHNAKTKPREEQYNSTYRVVSVLTVLHQDWLNGKEYKCKVSNKALAAPIEKTISKAKGQPR
EPQVYTIPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-945
(SEQ ID NO: 4690)
DVQLLESGGGLVQPGGSLRLSCAASGRTFSDYGMGWFRQAPGKDSEFVAAINWSNARTNYADSVKGRFTISRDNAKN

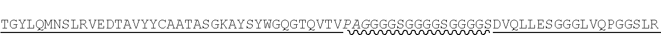

LSCAASGRTFSDYGMGWFRQAPGKDSEFVAAINWSNARTNYADSVKGRFTISRDNAKNTGYLQMNSLRVEDTAVYY

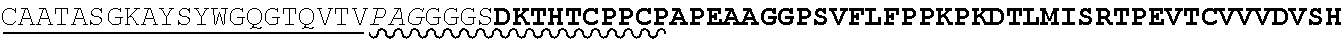

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-946
(SEQ ID NO: 4691)
DVQLLESGGGLVQPGGSLRLSCAASGRTFSDYGMGWFRQAPGKDSEFVAAINWSNARTNYADSVKGRFTISRDNAKN

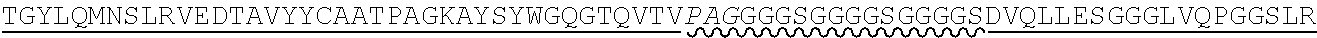

LSCAASGRTFSDYGMGWFRQAPGKDSEFVAAINWSNARTNYADSVKGRETISRDNAKNTGYLQMNSLRVEDTAVYY

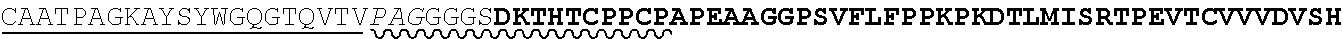

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-947
(SEQ ID NO: 4692)
DVQLLESGGGLVOPGGSLRLSCAASGRTESDYGMGWFROAPGKDSEFVAAINWSNARTNYADSVKGRETISRDNAKN

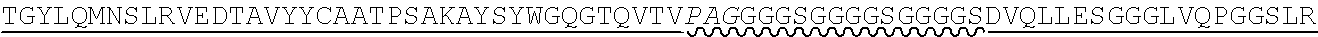

LSCAASGRTFSDYGMGWFRQAPGKDSEFVAAINWSNARTNYADSVKGRFTISRDNAKNTGYLQMNSLRVEDTAVYY

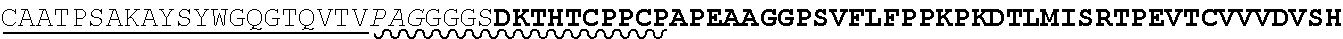

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-948
(SEQ ID NO: 4693)
DVQLLESGGGLVQPGGSLRLSCAASGRTFSDYGMGWFRQAPGKDSEFVAAINWSNARTNYADSVKGRFTISRDNAKN

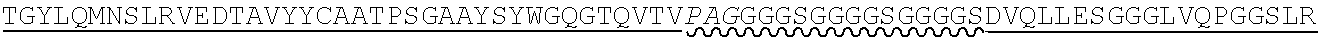

LSCAASGRTFSDYGMGWFRQAPGKDSEFVAAINWSNARTNYADSVKGRFTISRDNAKNTGYLQMNSLRVEDTAVYY

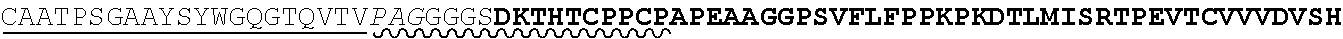

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-949
(SEQ ID NO: 4694)
DVQLLESGGGLVQPGGSLRLSCAASGRTESDYGMGWFRQAPGKDSEFVAAINWSNARTNYADSVKGRETISRDNAKN

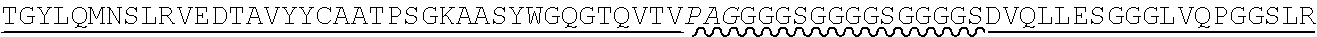

LSCAASGRTFSDYGMGWFRQAPGKDSEFVAAINWSNARTNYADSVKGRFTISRDNAKNTGYLQMNSLRVEDTAVYY

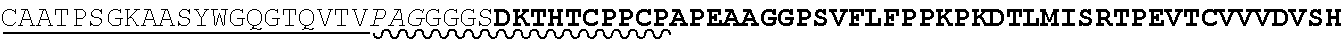

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-950
(SEQ ID NO: 4695)
DVQLLESGGGLVQPGGSLRLSCAASGRTFSDYGMGWFRQAPGKDSEFVAAINWSNARTNYADSVKGRFTISRDNAKN

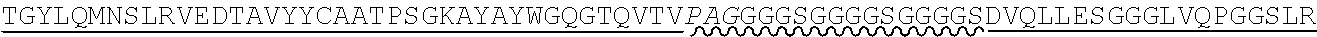

LSCAASGRTFSDYGMGWFRQAPGKDSEFVAAINWSNARTNYADSVKGRFTISRDNAKNTGYLQMNSLRVEDTAVYY

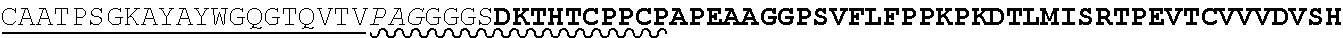

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-951
(SEQ ID NO: 4696)
DVQLLESGGGLVQPGGSLRLSCAASGRTESDYGMGWFROAPGKDSEFVAAINWSNARTNYADSVKGRETISRDNAKN

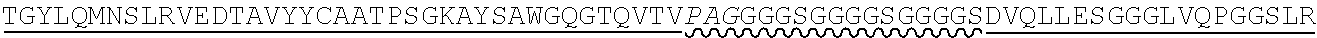

LSCAASGRTESDYGMGWFRQAPGKDSEFVAAINWSNARTNYADSVKGRETISRDNAKNTGYLQMNSLRVEDTAVYY

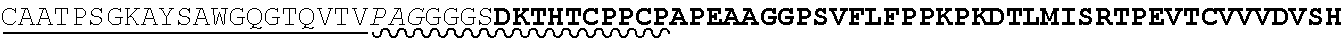

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-958
(SEQ ID NO: 4495)
DVQLLESGGGLVQPGGSLRLSCAASGRTFSDYGMGWFRQAPGKDSEFVAAINWSNARTNYADSVKGRFTISRDNAK

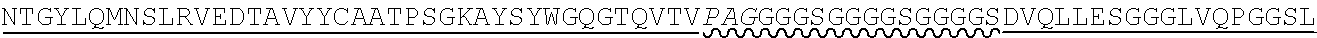

RLSCAASGRTFSDYGMGWFRQAPGKDSEFVAAINWSNARTNYADSVKGRFTISRDNAKNTGYLQMNSLRVEDTAVY

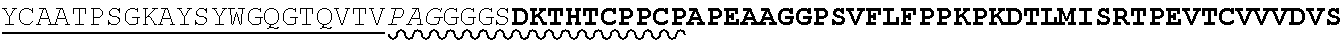

HEDPEVKFNWYVDGVEVHNAKTKPREEQYNSTYRVVSVLTVLHQDWLNGKEYKCKVSNKALAAPIEKTISKAKGQP
REPQVYTLPPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGK
ODY-961
(SEQ ID NO: 4496)
DVQLLESGGGLVQPGGSLRLSCAASGRTFSDYGMGWFRQAPGKDSEFVAAINWSNARTNYADSVKGRFTISRDNAK

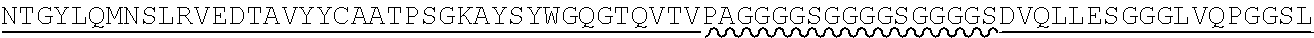

RLSCAASGRTFSDYGMGWFROAPGKDSEFVAAINWSNARTNYADSVKGRFTISRDNAKNTGYLOMNSLRVEDTAVY

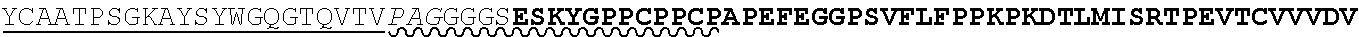

SQEDPEVQFNWYVDGVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSFFLYSRLTVDKSRWQE
GNVFSCSVMHEALHNHYTQKSLSLSLGK
ODY-1094
(SEQ ID NO: 4497)
EVQLLESGGGLVOPGGSLRLSCAASGETEDDIAMTWVRQAPGKGLEWVSSIYSYGPNTYYADSVKGRETISTDSAKNTL

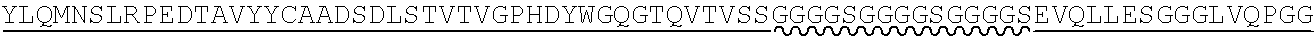

SLRLSCAASGFTFDDIAMTWVRQAPGKGLEWVSSIYSYGPNTYYADSVKGRFTISTDSAKNTLYLQMNSLRPEDTAVYY

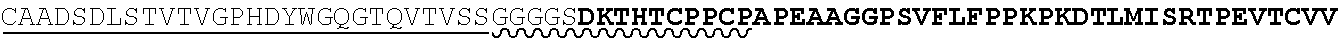

VDVSHEDPEVKFNWYVDGVEVHNAKTKPREEQYNSTYRVVSVLTVLHQDWLNGKEYKCKVSNKALAAPIEKTISKA
KGQPREPQVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
ODY-1095
(SEQ ID NO: 4498)
EVQLLESGGGLVQPGGSLRLSCAASGRTFSDYGMGWFRQAPGKDSEFVAAINWSNARTNYADSVKGRFTISRDNAKN

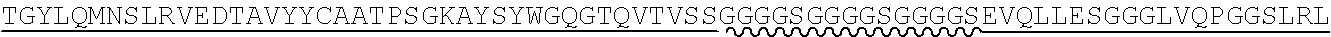

SCAASGRTESDYGMGWFRQAPGKDSEFVAAINWSNARTNYADSVKGRETISRDNAKNTGYLOMNSLRVEDTAVYYC

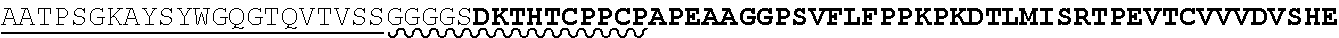

DPEVKFNWYVDGVEVHNAKTKPREEQYNSTYRVVSVLTVLHQDWINGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1096
(SEQ ID NO: 4499)
DVQLLESGGGLVQPGGSLRLSCAASGRTEGSYTMGWFRQAPGKEREFLASIRWTGGSTSYADSVKGRFTISRDDAKKT

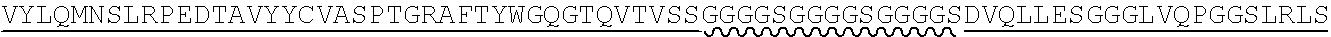

CAASGRTEGSYTMGWFRQAPGKEREFLASIRWTGGSTSYADSVKGRFTISRDDAKKTVYLQMNSLRPEDTAVYYCVAS

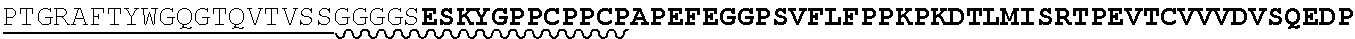

EVQFNWYVDGVEVHNAKTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYSRLTVDKSRWQEGNVFS
CSVMHEALHNHYTQKSLSLSLGK
ODY-1097
(SEQ ID NO: 4500)
DVQLLESGGGLVQPGGSLRLSCAASGFTFDDIAMTWVRQAPGKGLEWVSSIYSYGPNTYYADSVKGRFTISTDSAKNTL

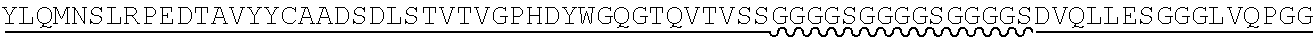

SLRLSCAASGFTFDDIAMTWVRQAPGKGLEWVSSIYSYGPNTYYADSVKGRFTISTDSAKNTLYLQMNSLRPEDTAVYY

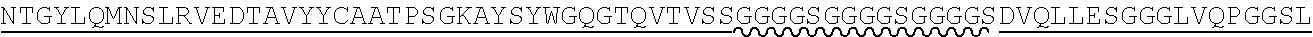

VVDVSQEDPEVQFNWYVDGVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGK
ODY-1098
(SEQ ID NO: 4501)
DVQLLESGGGLVOPGGSLRLSCAASGRTESDYGMGWFRQAPGKDSEFVAAINWSNARTNYADSVKGRETISRDNAK

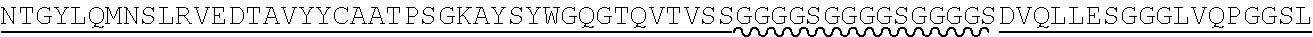

RLSCAASGRTFSDYGMGWFRQAPGKDSEFVAAINWSNARTNYADSVKGRFTISRDNAKNTGYLQMNSLRVEDTAVY

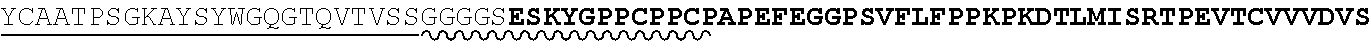

QEDPEVQFNWYVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1099
(SEQ ID NO: 4502)
DVQLLESGGGLVQPGGSLRLSCAASGRTFGSYTMGWFROAPGKEREFLASIRWTGGSTSYADSVKGRFTISRDDAKKT

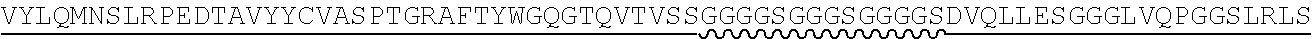

CAASGRTFGSYTMGWFRQAPGKEREFLASIRWTGGSTSYADSVKGRFTISRDDAKKTVYLQMNSLRPEDTAVYYCVAS

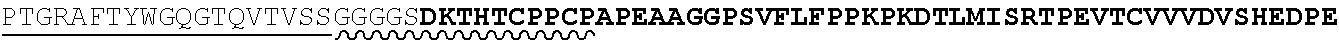

VKFNWYVDGVEVHNAKTKPREEQYNSTYRVVSVLTVLHQDWINGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-1100
(SEQ ID NO:4503)
DVQLLESGGGLVQPGGSLRLSCAASGFTEDDIAMTWVRQAPGKGLEWVSSIYSYGPNTYYADSVKGRFTISTDSÅKNTL

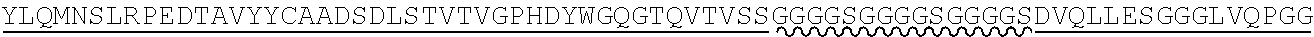

SLRLSCAASGFTFDDIAMTWVRQAPGKGLEWVSSIYSYGPNTYYADSVKGRFTISTDSAKNTLYLQMNSLRPEDTAVYY

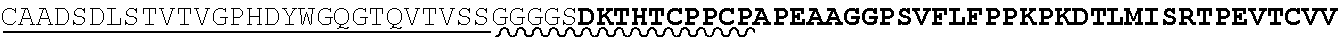

VDVSHEDPEVKFNWYVDGVEVHNAKTKPREEQYNSTYRVVSVLTVLHQDWLNGKEYKCKVSNKALAAPIEKTISKA
KGQPREPQVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
ODY-1101
(SEQ ID NO: 4504)
DVQLLESGGGLVQPGGSLRLSCAASGRTFSDYGMGWFRQAPGKDSEFVAAINWSNARTNYADSVKGRFTISRDNAK

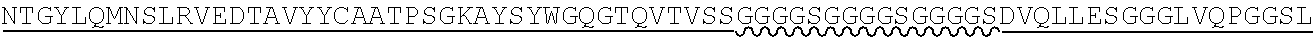

RLSCAASGRTFSDYGMGWFRQAPGKDSEFVAAINWSNARTNYADSVKGRFTISRDNAKNTGYLOMNSLRVEDTAVY

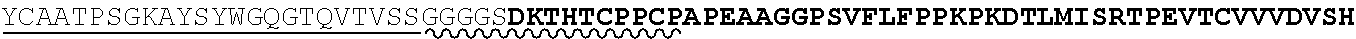

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-1102
(SEQ ID NO: 4505)
EVQLLESGGGLVQPGGSLRLSCAASGRTFGSYTMGWFRQAPGKEREFLASIRWTGGSTSYADSVKGRFTISRDDAKKT

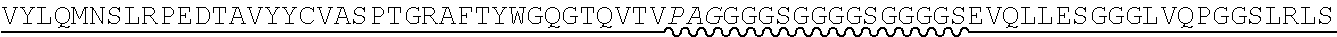

CAASGRTFGSYTMGWFRQAPGKEREFLASIRWTGGSTSYADSVKGRFTISRDDAKKTVYLQMNSLRPEDTAVYYCVAS

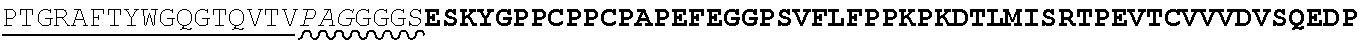

EVQFNWYVDGVEVHNAKTKPREEQFNSTYRVVSVLTVLHQDWLNGKEYKCKVSNKGLPSSIEKTISKAKGQPREPQ
VYTLPPSQEEMTKNQVSLTCLVKGFYPSDIAVEWESNGQPENNYKTTPPVLDSDGSFFLYSRLTVDKSRWQEGNVFS
CSVMHEALHNHYTQKSLSLSLGK
ODY-1103
(SEQ ID NO: 4506)
EVQLLESGGGLVQPGGSLRLSCAASGFTFDDIAMTWVRQAPGKGLEWVSSIYSYGPNTYYADSVKGRFTISTDSAKNTL

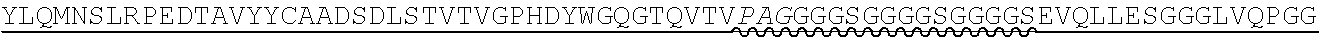

SLRLSCAASGFTFDDIAMTWVRQAPGKGLEWVSSIYSYGPNTYYADSVKGRFTISTDSAKNTLYLQMNSLRPEDTAVYY

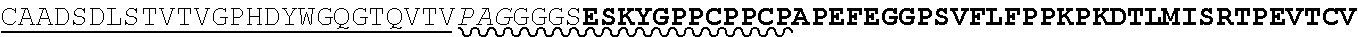

VVDVSQEDPEVQFNWYVDGVEVHNAKTKPREEQFNSTYRVVSVLTVLHQDWLNGKEYKCKVSNKGLPSSIEKTISK
AKGQPREPQVYTLPPSQEEMTKNQVSLTCLVKGFYPSDIAVEWESNGQPENNYKTTPPVLDSDGSFFLYSRLTVDKS
RWQEGNVFSCSVMHEALHNHYTQKSLSLSLGK
ODY-1105
(SEQ ID NO: 4507)
EVQLLESGGGLVQPGGSLRLSCAASGRTFGSYTMGWFRQAPGKEREFLASIRWTGGSTSYADSVKGRFTISRDDAKKT

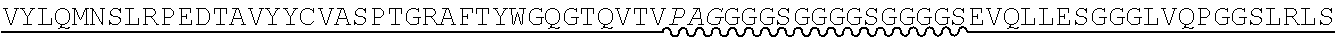

CAASGRTEGSYTMGWFRQAPGKEREFLASIRWTGGSTSYADSVKGRETISRDDAKKTVYLOMNSLRPEDTAVYYCVAS

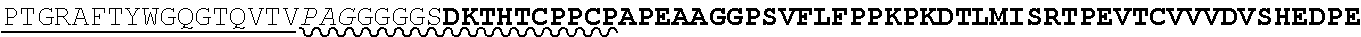

VKFNWYVDGVEVHNAKTKPREEQYNSTYRVVSVLTVLHQDWLNGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-1106
(SEQ ID NO: 4508)
EVQLLESGGGLVQPGGSLRLSCAASGFTFDDIAMTWVRQAPGKGLEWVSSIYSYGPNTYYADSVKGRFTISTDSAKNTL

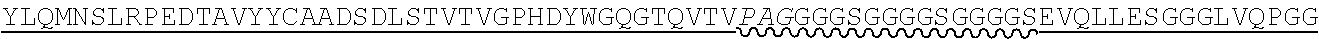

SLRLSCAASGETFDDIAMTWVRQAPGKGLEWVSSIYSYGPNTYYADSVKGRFTISTDSAKNTLYLOMNSLRPEDTAVYY

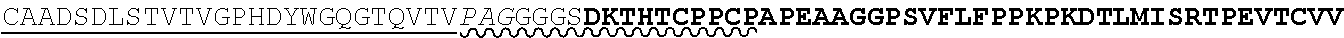

VDVSHEDPEVKFNWYVDGVEVHNAKTKPREEQYNSTYRVVSVLTVLHQDWLNGKEYKCKVSNKALAAPIEKTISKA
KGQPREPQVYTLPPSREEMTKNQVSLTCLVKGFYPSDIAVEWESNGQPENNYKTTPPVLDSDGSFFLYSKLTVDKSR
WQQGNVFSCSVMHEALHNHYTQKSLSLSPGK
ODY-1109
(SEQ ID NO: 4509)
DVQLLESGGGLVQPGGSLRLSCAASGFTFDDIAMTWVRQAPGKGLEWVSSIYSYGPNTYYADSVKGRFTISTDSAKNTL

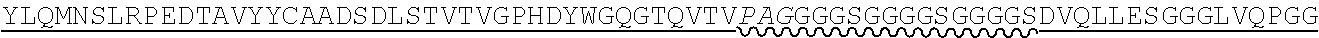

GSLRLSCAASGETEDDIAMTWVRQAPGKGLEWVSSIYSYGPNTYYADSVKGRETISTDSAKNTLYLOMNSLRPEDTAVY

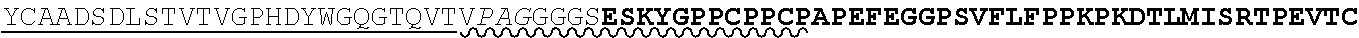

VVVDVSQEDPEVQFNWYVDGVEVHNAKTKPREEQFNSTYRVVSVLTVLHQDWINGKEYKCKVSNKGLPSSIEKTIS
KAKGQPREPQVYTLPPSQEEMTKNQVSLTCLVKGFYPSDIAVEWESNGQPENNYKTTPPVLDSDGSFFLYSRLTVDK
SRWQEGNVFSCSVMHEALHNHYTQKSLSLSLGK
ODY-1112
(SEQ ID NO: 4510)
DVQLLESGGGLVOPGGSLRLSCAASGFTEDDIAMTWVRQAPGKGLEWVSSIYSYGPNTYYADSVKGRFTISTDSAKNTL

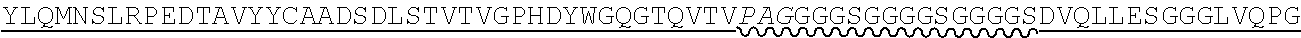

GSLRISCAASGFTFDDIAMTWVRQAPGKGLEWVSSIYSYGPNTYYADSVKGRFTISTDSAKNTLYLQMNSLRPEDTAVY

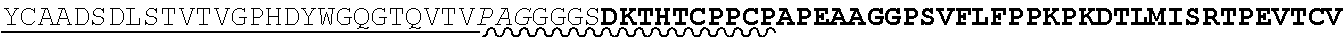

VVDVSHEDPEVKFNWYVDGVEVHNAKTKPREEQYNSTYRVVSVLTVLHQDWLNGKEYKCKVSNKALAAPIEKTISK
AKGQPREPQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPGK
ODY-1114
(SEQ ID NO: 4511)
DVQLLESGGGLVQPGGSLRLSCAASGRTFGSYTMGWFRQAPGKEREFLASIRWTGGSTSYADSVKGRETISRDDAKKT

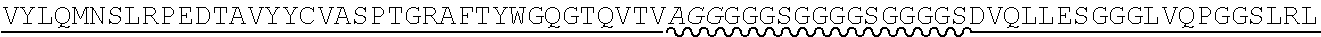

SCAASGRTFGSYTMGWFRQAPGKEREFLASIRWTGGSTSYADSVKGRETISRDDAKKTVYLOMNSLRPEDTAVYYCVA

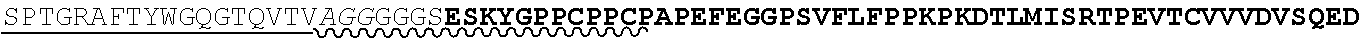

PEVQFNWYVDGVEVHNAKTKPREEQFNSTYRVVSVLTVLHQDWLNGKEYKCKVSNKGLPSSIEKTISKAKGQPREP
QVYTLPPSQEEMTKNQVSLTCLVKGFYPSDIAVEWESNGQPENNYKTTPPVLDSDGSFFLYSRLTVDKSRWQEGNV
FSCSVMHEALHNHYTQKSISISLGK
ODY-1115
(SEQ ID NO: 4512)
DVQLLESGGGLVQPGGSLRLSCAASGFTFDDIAMTWVRQAPGKGLEWVSSIYSYGPNTYYADSVKGRFTISTDSAKNTL

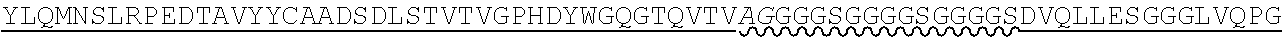

GSLRLSCAASGFTFDDIAMTWVRQAPGKGLEWVSSIYSYGPNTYYADSVKGRFTISTDSAKNTLYLOMNSLRPEDTAVY

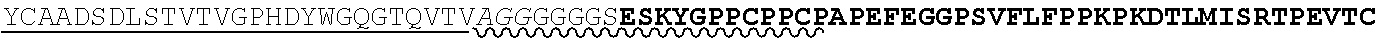

VVVDVSQEDPEVQFNWYVDGVEVHNAKTKPREEQFNSTYRVVSVLTVLHQDWINGKEYKCKVSNKGLPSSIEKTIS
KAKGQPREPQVYTLPPSQEEMTKNQVSLTCLVKGFYPSDIAVEWESNGQPENNYKTTPPVLDSDGSFFLYSRLTVDK
SRWQEGNVFSCSVMHEALHNHYTQKSLSLSLGK
ODY-1116
(SEQ ID NO: 4513)
DVQLLESGGGLVQPGGSLRLSCAASGRTESDYGMGWFRQAPGKDSEFVAAINWSNARTNYADSVKGRFTISRDNAK

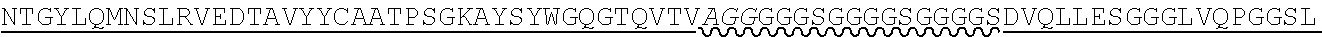

RLSCAASGRTFSDYGMGWFRQAPGKDSEFVAAINWSNARTNYADSVKGRETISRDNAKNTGYLQMNSLRVEDTAVY

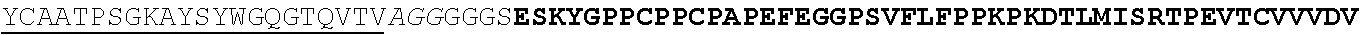

SQEDPEVQFNWYVDGVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSFFLYSRLTVDKSRWQE
GNVFSCSVMHEALHNHYTQKSLSLSLGK
ODY-1164
(SEQ ID NO: 4709)
EVQLLESGGGLVOPGGSLRLSCAASGRTFSDYGMGWFRQAPGKDSEFVAAINWSNARTNYADSVKGRFTISRDNAKN

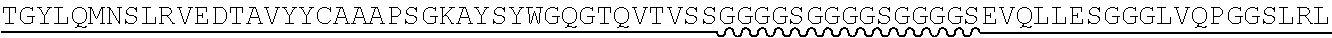

SCAASGRTFSDYGMGWFRQAPGKDSEFVAAINWSNARTNYADSVKGRFTISRDNAKNTGYLQMNSLRVEDTAVYYC

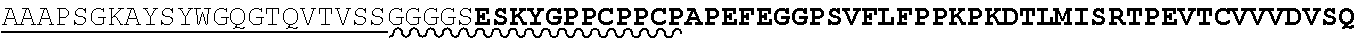

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1165
(SEQ ID NO: 4710)
EVQLLESGGGLVQPGGSLRLSCAASGRTFSDYGMGWFRQAPGKDSEFVAAINWSNARTNYADSVKGRFTISRDNAKN

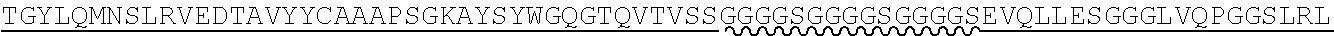

SCAASGRTFSDYGMGWFRQAPGKDSEFVAAINWSNARTNYADSVKGRFTISRDNAKNTGYLQMNSLRVEDTAVYYC

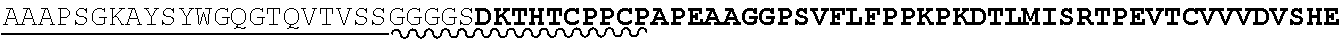

DPEVKFNWYVDGVEVHNAKTKPREEQYNSTYRVVSVLTVLHQDWLNGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1166
(SEQ ID NO: 4711)
DVQLLESGGGLVQPGGSLRLSCAASGRTESDYGMGWFROAPGKDSEFVAAINWSNARTNYADSVKGRFTISRDNAK

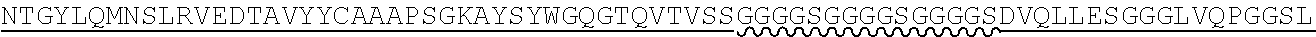

RLSCAASGRTESDYGMGWFRQAPGKDSEFVAAINWSNARTNYADSVKGRFTISRDNAKNTGYLQMNSLRVEDTAVY

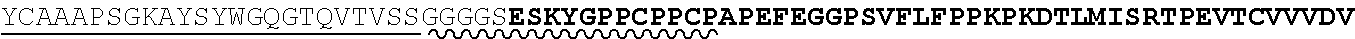

SQEDPEVQFNWYVDGVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSFFLYSRLTVDKSRWQE
GNVFSCSVMHEALHNHYTQKSLSLSLGK
ODY-1167
(SEQ ID NO: 4712)
DVQLLESGGGLVQPGGSLRLSCAASGRTFSDYGMGWFRQAPGKDSEFVAAINWSNARTNYADSVKGRFTISRDNAK

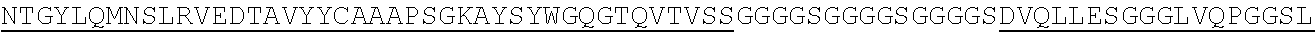

RLSCAASGRTFSDYGMGWFRQAPGKDSEFVAAINWSNARTNYADSVKGRFTISRDNAKNTGYLQMNSLRVEDTAVY
YCAAAPSGKAYSYWGQGTQVTVSSGGGGSDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGKEYKCKVSNKALAAPIEKTISKAKGQP
REPQVYTLPPSREEMTKNQVSLTCLVKGFYPSDIAVEWESNGQPENNYKTTPPVLDSDGSFFLYSKLTVDKSRWQQ
GNVFSCSVMHEALHNHYTQKSLSLSPGK
ODY-1168
(SEQ ID NO: 4713)
EVQLLESGGGLVQPGGSLRLSCAASGRTFSDYGMGWFROAPGKDSEFVAAINWSNARTNYADSVKGRFTISRDNAKN

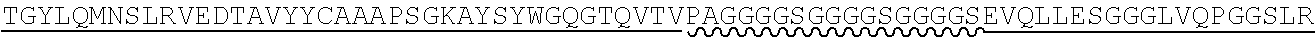

LSCAASGRIESDYGMGWFROAPGKDSEFVAAINWSNARTNYADSVKGRETISRDNAKNTGYLOMNSLRVEDTAVYY

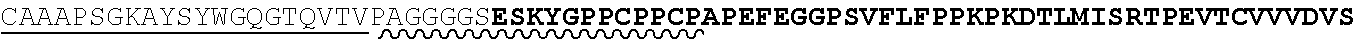

QEDPEVQFNWYVDGVEVHNAKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1169
(SEQ ID NO: 4714)
EVQLLESGGGLVOPGGSLRLSCAASGRTFSDYGMGWFRQAPGKDSEFVAAINWSNARTNYADSVKGRFTISRDNAKN

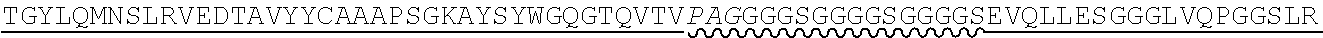

LSCAASGRTFSDYGMGWFRQAPGKDSEFVAAINWSNARTNYADSVKGRFTISRDNAKNTGYLQMNSLRVEDTAVYY

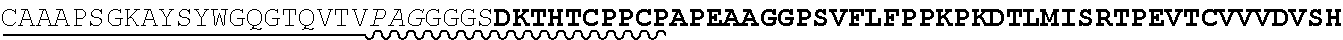

EDPEVKFNWYVDGVEVHNAKTKPREEQYNSTYRVVSVLTVLHQDWLNGKEYKCKVSNKALAAPIEKTISKAKGQPR
EPQVYTLPPSREEMTKNQVSLTCLVKGFYPSDIAVEWESNGQPENNYKTTPPVLDSDGSFFLYSKLTVDKSRWQQG
NVFSCSVMHEALHNHYTQKSLSLSPGK
ODY-1170
(SEQ ID NO: 4715)
DVQLLESGGGLVQPGGSLRLSCAASGRTESDYGMGWFRQAPGKDSEFVAAINWSNARTNYADSVKGRETISRDNAK

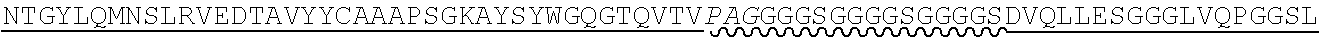

RLSCAASGRTFSDYGMGWFRQAPGKDSEFVAAINWSNARTNYADSVKGRFTISRDNAKNTGYLQMNSLRVEDTAVY

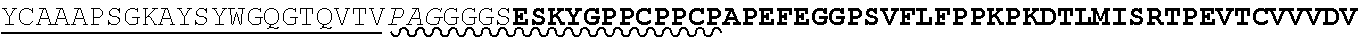

SQEDPEVQFNWYVDGVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSFFLYSRLTVDKSRWQE
GNVFSCSVMHEALHNHYTQKSLSLSLGK
ODY-1171
(SEQ ID NO: 4716)
DVQLLESGGGLVOPGGSLRLSCÅASGRTESDYGMGWFRQAPGKDSEFVAAINWSNARTNYADSVKGRETISRDNAK

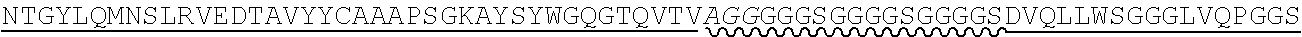

LRLSCAASGRTFSDYGMGWFRQAPGKDSEFVAAINWSNARTNYADSVKGRFTISRDNAKNTGYLQMNSLRVEDTAVY

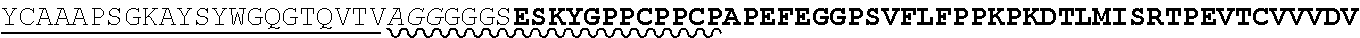

SQEDPEVQFNWYVDGVEVHNAKTKPREEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYKTTPPVLDSDGSFFLYSRLTVDKSRWQE
GNVFSCSVMHEALHNHYTQKSLSLSLGK
ODY-1213
(SEQ ID NO: 4735)
EVQLLESGGGLVQPGGSLRLSCAASGRTFSDYGMGWFRQAPGKEREFVATINWSNGRTTYADSVKGRFTISRDNAKN

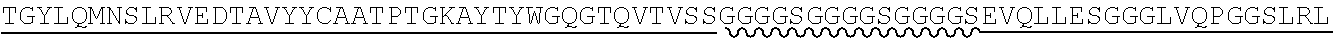

SCAASGRTFSDYGMGWFRQAPGKEREFVATINWSNGRTTYADSVKGRFTISRDNAKNTGYLOMNSLRVEDTAVYYCA

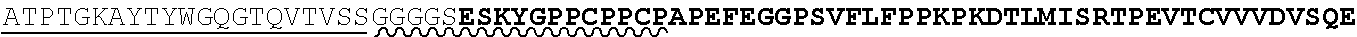

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
ODY-1214
(SEQ ID NO: 4736)
EVQLLESGGGLVQPGGSLRLSCAASGRTFSDYGMGWFRQAPGKEREFVATINWSNGRTTYADSVKGRETISRDNAKN

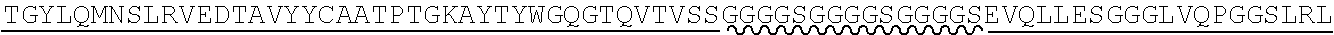

SCAASGRTESDYGMGWFRQAPGKEREFVATINWSNGRTTYADSVKGRETISRDNAKNTGYLQMNSLRVEDTAVYYCA

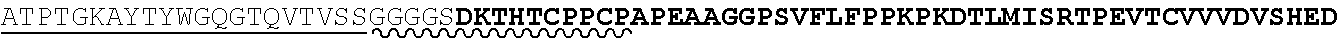

PEVKFNWYVDGVEVHNAKTKPREEQYNSTYRVVSVLTVLHQDWLNGKEYKCKVSNKALAAPIEKTISKAKGQPREP
QVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGK
ODY-1215
(SEQ ID NO: 4737)
DVQLLESGGGLVQPGGSLRLSCAASGRTFSDYGMGWFRQAPGKEREFVATINWSNGRTTYADSVKGRFTISRDNAKN

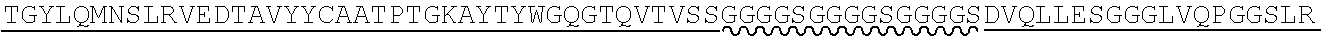

LSCAASGRTFSDYGMGWFRQAPGKEREFVATINWSNGRTTYADSVKGRFTISRDNAKNTGYLQMNSLRVEDTAVYYC

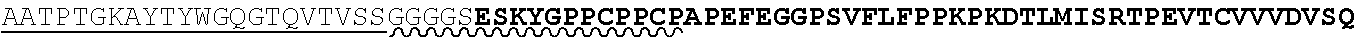

EDPEVQFNWYVDGVEVHNAKTKPREEQFNSTYRVVSVLTVLHQDWINGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1216
(SEQ ID NO: 4738)
DVQLLESGGGLVQPGGSLRLSCAASGRTESDYGMGWFRQAPGKEREFVATINWSNGRTTYADSVKGRFTISRDNAKN

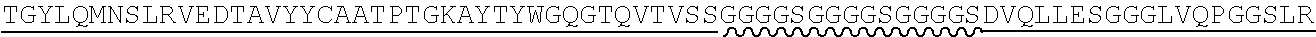

LSCAASGRTESDYGMGWFROAPGKEREFVATINWSNGRTTYADSVKGRETISRDNAKNTGYLQMNSLRVEDTAVYYC

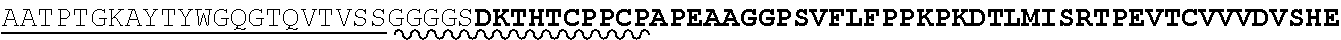

DPEVKFNWYVDGVEVHNAKTKPREEQYNSTYRVVSVLTVLHQDWLNGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1217
(SEQ ID NO: 4739)
EVQLLESGGGLVOPGGSLRLSCAÅSGRTESDYGMGWFRQAPGKEREFVATINWSNGRTTYADSVKGRETISRDNAKN

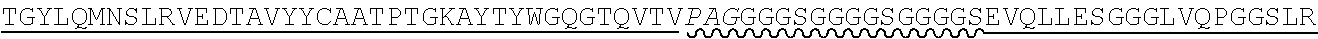

LSCAASGRTESDYGMGWFROAPGKEREFVATINWSNGRTTYADSVKGRFTISRDNAKNTGYLQMNSLRVEDTAVYYC

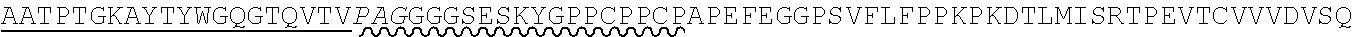

EDPEVQFNWYVDGVEVHNAKTKPREEQFNSTYRVVSVLTVLHQDWLNGKEYKCKVSNKGLPSSIEKTISKAKGQPR
EPQVYTIPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1218
(SEQ ID NO: 4740)
EVQLLESGGGLVQPGGSLRLSCAASGRTESDYGMGWFROAPGKEREFVATINWSNGRTTYADSVKGRFTISRDNAKN

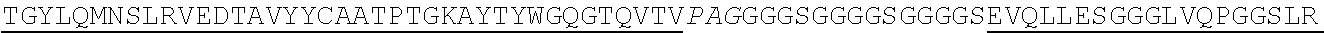

LSCAASGRTFSDYGMGWFRQAPGKEREFVATINWSNGRTTYADSVKGRFTISRDNAKNTGYLQMNSLRVEDTAVYC

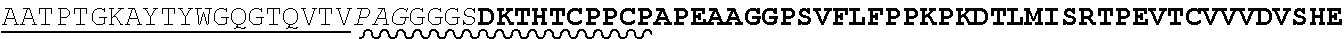

DPEVKFNWYVDGVEVHNAKTKPREEQYNSTYRVVSVLTVLHQDWLNGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1219
(SEQ ID NO: 4741)
DVQLLESGGGLVQPGGSLRLSCAASGRTESDYGMGWFROAPGKEREFVATINWSNGRTTYÅDSVKGRETISRDNAKN

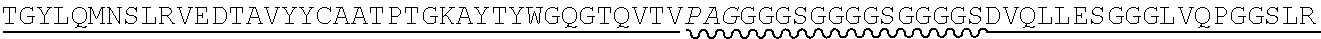

LSCAASGRTFSDYGMGWFRQAPGKEREFVATINWSNGRTTYADSVKGRFTISRDNAKNTGYLQMNSLRVEDTAVYYC

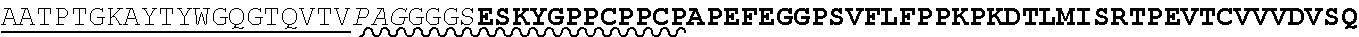

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1220
(SEQ ID NO: 4742)
DVQLLESGGGLVQPGGSLRLSCAASGRTFSDYGMGWFRQAPGKEREFVATINWSNGRTTYADSVKGRFTISRDNAKN

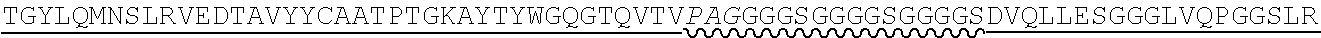

LSCAASGRTFSDYGMGWFRQAPGKEREFVATINWSNGRTTYADSVKGRFTISRDNAKNTGYLQMNSLRVEDTAVYYC

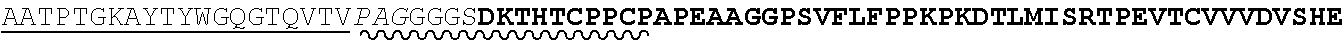

DPEVKFNWYVDGVEVHNAKTKPREEQYNSTYRVVSVLTVLHQDWINGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1221
(SEQ ID NO: 4743)
DVQLLESGGGLVQPGGSLRLSCAASGRTESDYGMGWFRQAPGKEREFVATINWSNGRTTYADSVKGRETISRDNAKN

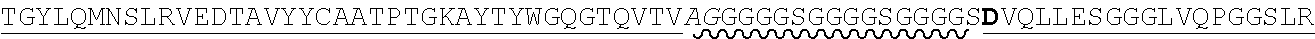

LSCAASGRTESDYGMGWFROAPGKEREFVATINWSNGRTTYADSVKGRFTISRDNAKNTGYLOMNSLRVEDTAVYYC

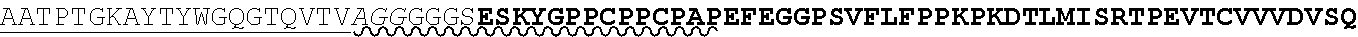

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1222
SEQ ID NO: (4744)
EVQLLESGGGLVOPGGSLRLSCAASGRTFSDYGMGWFROAPGKEREFVATINWSNARTTYADSVKGRFTISRDNAKN

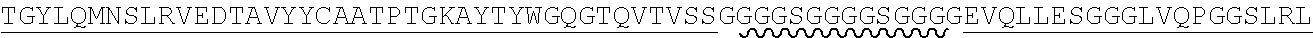

SCAASGRTFSDYGMGWFRQAPGKEREFVATINWSNARTTYADSVKGRFTISRDNAKNTGYLQMNSLRVEDTAVYYCA

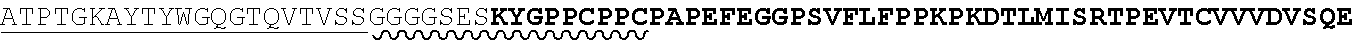

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
(ODY-1223)
(SEQ ID NO: 4745)
EVQLLESGGGLVQPGGSLRLSCAASGRTFSDYGMGWFRQAPGKEREFVATINWSNARTTYADSVKGRFTISRDNAKN

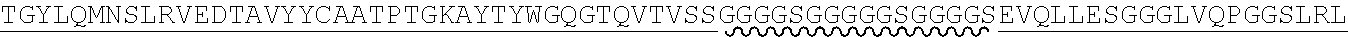

SCAASGRTFSDYGMGWFRQAPGKEREFVATINWSNARTTYADSVKGRFTISRDNAKNTGYLQMNSLRVEDTAVYYCA

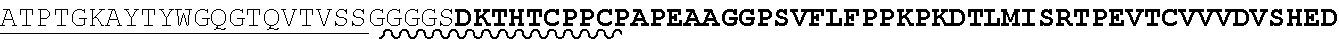

PEVKFNWYVDGVEVHNAKTKPREEQYNSTYRVVSVLTVLHQDWINGKEYKCKVSNKALAAPIEKTISKAKGQPREP
QVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGK
ODY-1224
(SEQ ID NO: 4746)
DVQLLESGGGLVQPGGSLRLSCAASGRTFSDYGMGWFRQAPGKEREFVATINWSNARTTYADSVKGRFTISRDNAKN

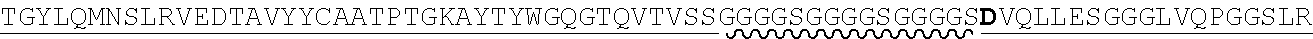

LSCAASGRTFSDYGMGWFRQAPGKEREFVATINWSNARTTYADSVKGRFTISRDNAKNTGYLOMNSLRVEDTAVYYC

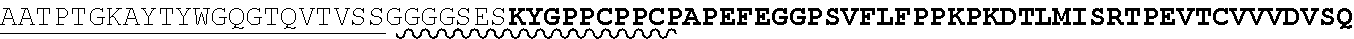

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1225
(SEQ ID NO: 4747)
DVQLLESGGGLVQPGGSLRLSCAASGRTESDYGMGWFRQAPGKEREFVATINWSNARTTYADSVKGRETISRDNAKN

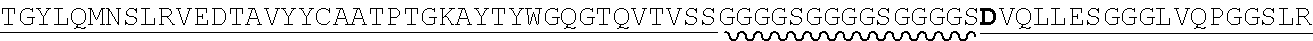

LSCAASGRTESDYGMGWFROAPGKEREFVATINWSNARTTYADSVKGRETISRDNAKNTGYLOMNSLRVEDTAVYYC

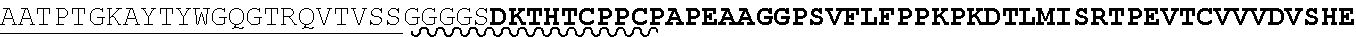

DPEVKFNWYVDGVEVHNAKTKPREEQYNSTYRVVSVLTVLHQDWLNGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1226
(SEQ ID NO: 4748)
EVQLLESGGGLVQPGGSLRLSCAASGRTFSDYGMGWFRQAPGKEREFVATINWSNARTTYADSVKGRFTISRDNAKN

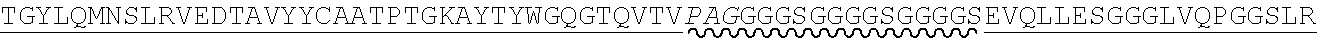

LSCAASGRTFSDYGMGWFRQAPGKEREFVATINWSNARTTYADSVKGRFTISRDNAKNTGYLQMNSLRVEDTAVYYC

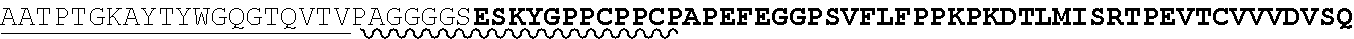

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1227
SEQ ID NO: 4749)
EVQLLESGGGLVQPGGSLRLSCAASGRTFSDYGMGWFRQAPGKEREFVATINWSNARTTYADSVKGRFTISRDNAKN

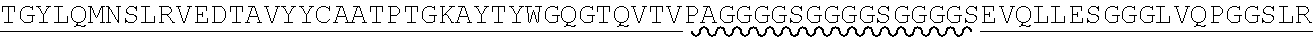

LSCAASGRTESDYGMGWFRQAPGKEREFVATINWSNARTTYADSVKGRETISRDNAKNTGYLQMNSLRVEDTAVYYC

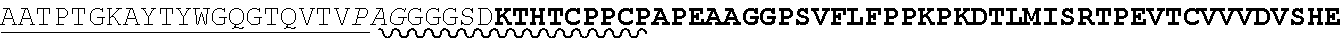

DPEVKFNWYVDGVEVHNAKTKPREEQYNSTYRVVSVLTVLHQDWLNGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
(ODY-1228)
(SEQ ID NO: 4750)
DVQLLESGGGLVQPGGSLRLSCAASGRTESDYGMGWFRQAPGKEREFVATINWSNARTTYADSVKGRFTISRDNAKN

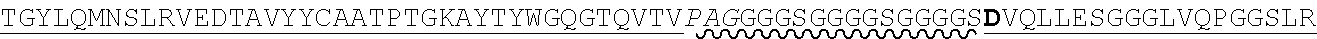

LSCAASGRTFSDYGMGWFROAPGKEREFVATINWSNARTTYADSVKGRFTISRDNAKNTGYLQMNSLRVEDTAVYYC

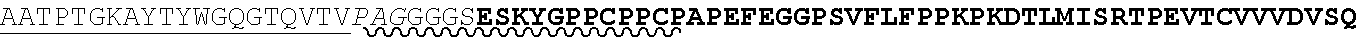

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1229
(SEQ ID NO: 4751)
DVQLLESGGGLVQPGGSLRLSCAASGRTFSDYGMGWFRQAPGKEREFVATINWSNARTTYADSVKGRFTISRDNAKN

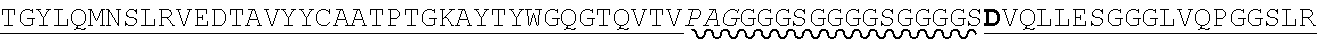

LSCAASGRTESDYGMGWFROAPGKEREFVATINWSNARTTYADSVKGRETISRDNAKNTGYLOMNSLRVEDTAVYYC

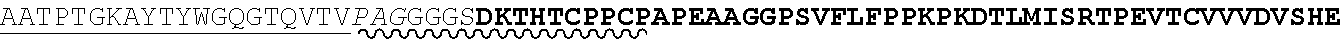

DPEVKFNWYVDGVEVHNAKTKPREEQYNSTYRVVSVLTVLHQDWINGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1230
(SEQ ID NO: 4752)
DVQLLESGGGLVOPGGSLRLSCAASGRTESDYGMGWERQAPGKEREFVATINWSNARTTYADSVKGRETISRDNAKN

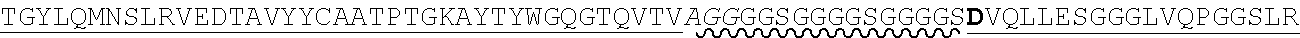

LSCAASGRTFSDYGMGWFRQAPGKEREFVATINWSNARTTYADSVKGRFTISRDNAKNTGYLQMNSLRVEDTAVYYC

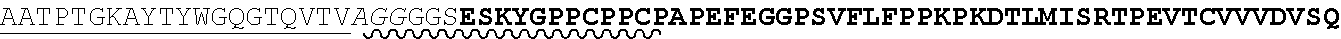

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1231
(SEQ ID NO: 4753)
EVQLLESGGGLVQPGGSLRLSCAASGRTFSDYGMGWFRQAPGKEHEFVASINWSNGRTSYADSAKGRIFTISRDNAKN

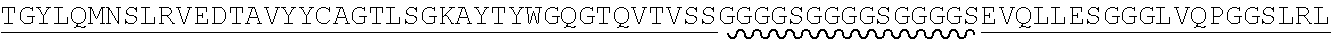

SCAASGRTESDYGMGWFRQAPGKEHEFVASINWSNGRTSYADSAKGRETISRDNAKNTGYLQMNSLRVEDTAVYYCA

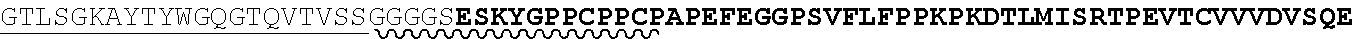

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
ODY-1232
(SEQ ID NO: 4754)
EVQLLESGGGLVQPGGSLRLSCAASGRTFSDYGMGWFRQAPGKEHEFVASINWSNGRTSYADSAKGRFTISRDNAKN

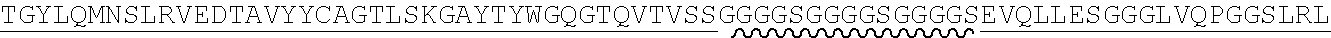

SCAASGRTFSDYGMGWFRQAPGKEHEFVASINWSNGRTSYADSAKGRFTISRDNAKNTGYLQMNSLRVEDTAVYYCA

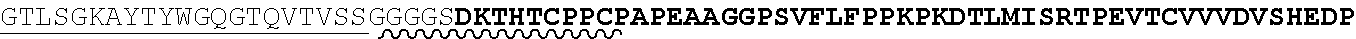

EVKFNWYVDGVEVHNAKTKPREEQYNSTYRVVSVLTVLHQDWLNGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-1233
(SEQ ID NO: 4755)
DVQLLESGGGLVQPGGSLRLSCAASGRTFSDYGMGWFRQAPGKEHEFVASINWSNGRTSYADSAKGRFTISRDNAKN

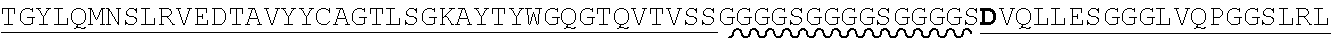

SCAASGRTESDYGMGWFROAPGKEHEFVASINWSNGRTSYADSAKGRETISRDNAKNTGYLQMNSLRVEDTAVYYCA

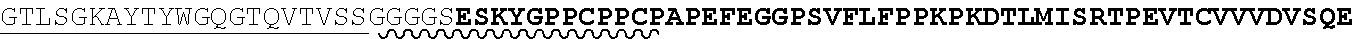

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
ODY-1234
(SEQ ID NO: 4756)
DVQLLESGGGLVQPGGSLRLSCAASGRTESDYGMGWFROAPGKEHEFVASINWSNGRTSYADSAKGRETISRDNAKN

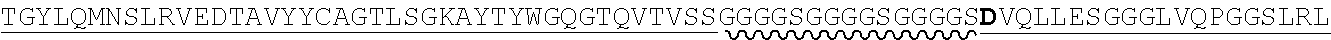

SCAASGRTESDYGMGWFRQAPGKEHEFVASINWSNGRTSYADSAKGRFTISRDNAKNTGYLQMNSLRVEDTAVYYCA

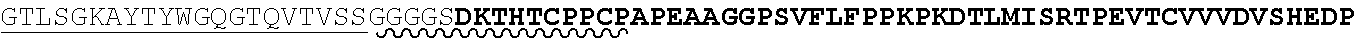

EVKFNWYVDGVEVHNAKTKPREEQYNSTYRVVSVLTVLHQDWLNGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-1235
(SEQ ID NO: 4757)
EVQLLESGGGLVQPGGSLRLSCAASGRTFSDYGMGWFRQAPGKEHEFVASINWSNGRTSYADSAKGRFTISRDNAKN

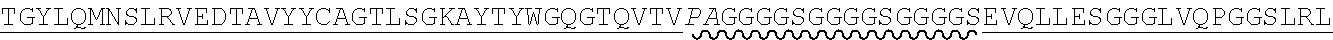

SCAASGRTESDYGMGWFRQAPGKEHEFVASINWSNGRTSYADSAKGRETISRDNAKNTGYLOMNSLRVEDTAVYYCA

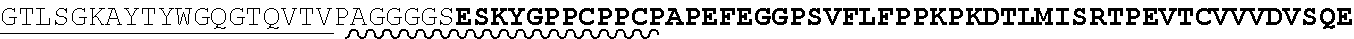

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
ODY-1236
(SEQ ID NO: 4758)
EVQLLESGGGLVQPGGSLRLSCAASGRTFSDYGMGWFRQAPGKEHEFVASINWSNGRTSYADSAKGRFTISRDNAKN

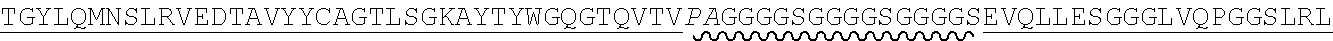

SCAASGRTESDYGMGWFRQAPGKEHEFVASINWSNGRTSYADSAKGRETISRDNAKNTGYLQMNSLRVEDTAVYYCA

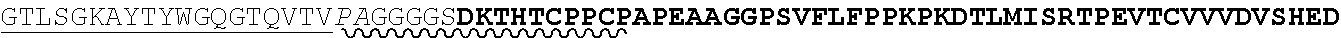

PEVKFNWYVDGVEVHNAKTKPREEQYNSTYRVVSVLTVLHQDWLNGKEYKCKVSNKALAAPIEKTISKAKGQPREP
QVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGK
ODY-1237
(SEQ ID NO: 4759)
DVQLLESGGGLVQPGGSLRLSCAASGRTFSDYGMGWFRQAPGKEHEFVASINWSNGRTSYADSAKGRFTISRDNAKN

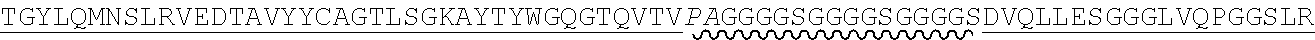

LSCAASGRTFSDYGMGWFRQAPGKEHEFVASINWSNGRTSYADSAKGRFTISRDNAKNTGYLQMNSLRVEDTAVYYC

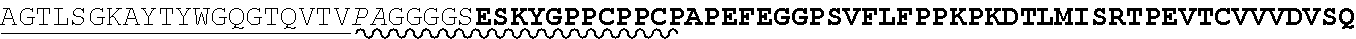

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1238
(SEQ ID NO: 4760)
DVQLLESGGGLVQPGGSLRLSCAASGRTFSDYGMGWFRQAPGKEHEFVASINWSNGRTSYADSAKGRETISRDNAKN

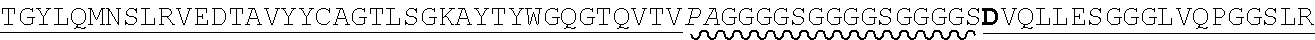

LSCAASGRTFSDYGMGWFRQAPGKEHEFVASINWSNGRTSYADSAKGRETISRDNAKNTGYLQMNSLRVEDTAVYYC

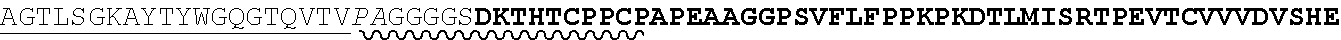

DPEVKFNWYVDGVEVHNAKTKPREEQYNSTYRVVSVLTVLHQDWLNGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1239
(SEQ ID NO: 4761)
DVQLLESGGGLVOPGGSLRLSCAASGRTESDYGMGWFRQAPGKEHEFVASINWSNGRTSYADSAKGRETISRDNAKN

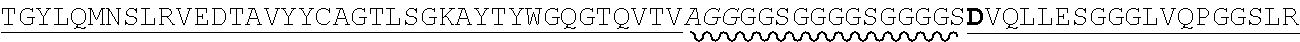

LSCAASGRTESDYGMGWFROAPGKEHEFVASINWSNGRTSYADSAKGRFTISRDNAKNTGYLQMNSLRVEDTAVYYC

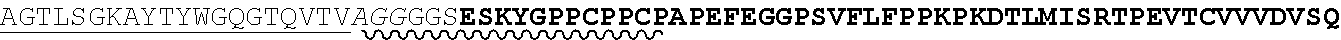

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1240
(SEQ ID NO: 4762)
EVQLLESGGGLVOPGGSLRLSCAASGRTFSDYGMGWFRQAPGKEHEFVASINWSNARTSYADSAKGRFTISRDNAKN

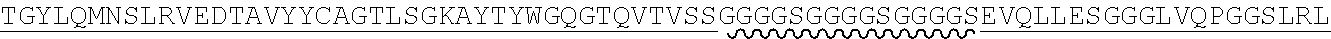

SCAASGRTFSDYGMGWFRQAPGKEHEFVASINWSNARTSYADSAKGRFTISRDNAKNTGYLOMNSLRVEDTAVYYCA

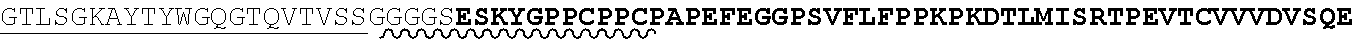

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
ODY-1241
(SEQ ID NO: 4763)
EVQLLESGGGLVQPGGSLRLSCAASGRTESDYGMGWFRQAPGKEHEFVASINWSNARTSYADSÅKGRFTISRDNÅKN

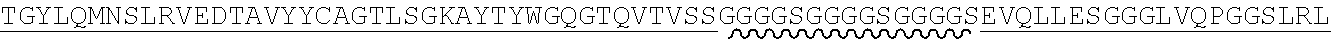

SCAASGRTESDYGMGWFRQAPGKEHEFVASINWSNARTSYADSAKGRFTISRDNAKNTGYLQMNSLRVEDTAVYYCA

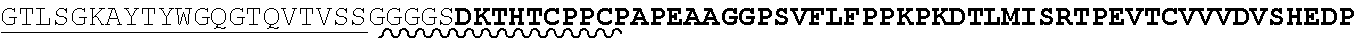

EVKFNWYVDGVEVHNAKTKPREEQYNSTYRVVSVLTVLHQDWLNGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-1242
(SEQ ID NO: 4764)
DVQLLESGGGLVQPGGSLRLSCAASGRTESDYGMGWFRQAPGKEHEFVASINWSNARTSYADSAKGRFTISRDNAKN

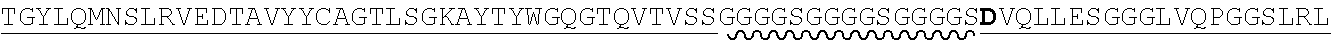

SCAASGRTFSDYGMGWFRQAPGKEHEFVASINWSNARTSYADSAKGRFTISRDNAKNTGYLQMNSLRVEDTAVYYCA

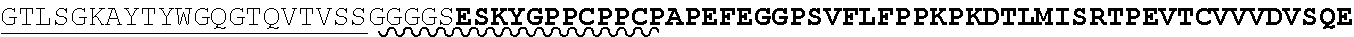

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
ODY-1243
(SEQ ID NO: 4765)
DVQLLESGGGLVQPGGSLRLSCAASGRTESDYGMGWFROAPGKEHEFVASINWSNARTSYADSAKGRETISRDNAKN

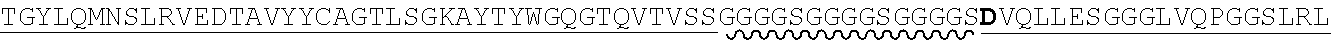

SCAASGRTESDYGMGWFRQAPGKEHEFVASINWSNARTSYADSAKGRETISRDNAKNTGYLQMNSLRVEDTAVYYCA

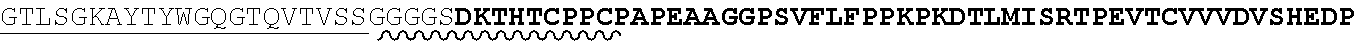

EVKFNWYVDGVEVHNAKTKPREEQYNSTYRVVSVLTVLHQDWLNGKEYKCKVSNKALAAPIEKTISKAKGQPREPQ
VYTLPPSREEMTKNQVSLTCLVKGFYPSDIAVEWESNGQPENNYKTTPPVLDSDGSFFLYSKLTVDKSRWQQGNVFS
CSVMHEALHNHYTQKSLSLSPGK
ODY-1244
(SEQ ID NO: 4766)
EVQLLESGGGLVQPGGSLRLSCAASGRTFSDYGMGWFRQAPGKEHEFVASINWSNARTSYADSAKGRFTISRDNAKN

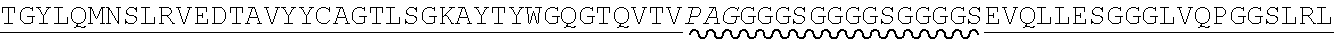

SCAASGRTFSDYGMGWFRQAPGKEHEFVASINWSNARTSYADSAKGRFTISRDNAKNTGYLQMNSLRVEDTAVYYCA

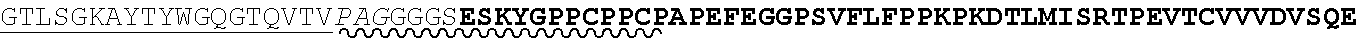

DPEVQFNWYVDGVEVHNAKTKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDGSFFLYSRLTVDKSRWQEGN
VFSCSVMHEALHNHYTQKSLSLSLGK
ODY-1245
(SEQ ID NO: 4767)
EVQLLESGGGLVQPGGSLRLSCAASGRTFSDYGMGWFROAPGKEHEFVASINWSNARTSYADSAKGRETISRDNAKN

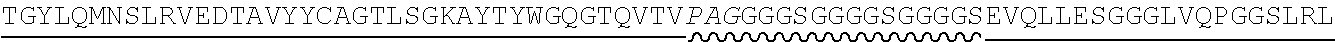

SCAASGRTESDYGMGWFRQAPGKEHEFVASINWSNARTSYADSAKGRETISRDNAKNTGYLQMNSLRVEDTAVYYCA

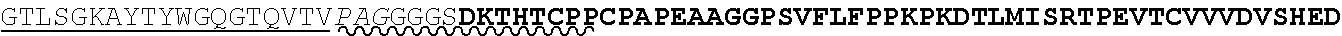

PEVKFNWYVDGVEVHNAKTKPREEQYNSTYRVVSVLTVLHQDWLNGKEYKCKVSNKALAAPIEKTISKAKGQPREP
QVYTLPPSREEMTKNQVSLTCLVKGFYPSDIAVEWESNGQPENNYKTTPPVLDSDGSFFLYSKLTVDKSRWQQGNV
FSCSVMHEALHNHYTQKSLSLSPGK
ODY-1246
(SEQ ID NO: 4768)
DVQLLESGGGLVOPGGSLRLSCAASGRTESDYGMGWFROAPGKEHEFVASINWSNARTSYADSAKGRETISRDNAKN

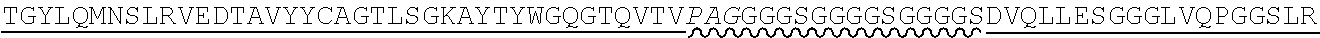

LSCAASGRTESDYGMGWFRQAPGKEHEFVASINWSNARTSYADSAKGRFTISRDNAKNTGYLQMNSLRVEDTAVYYC

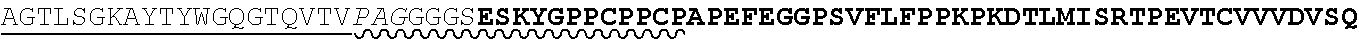

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK
ODY-1247
(SEQ ID NO: 4769)
DVQLLESGGGLVQPGGSLRLSCAASGRTESDYGMGWFRQAPGKEHEFVASINWSNARTSYADSAKGRFTISRDNAKN

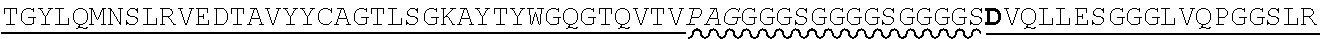

LSCAASGRTESDYGMGWFRQAPGKEHEFVASINWSNARTSYADSAKGRETISRDNAKNTGYLOMNSLRVEDTAVYYC

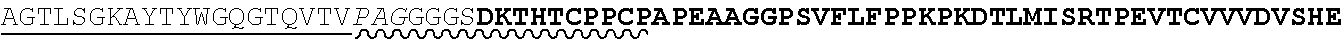

DPEVKFNWYVDGVEVHNAKTKPREEQYNSTYRVVSVLTVLHQDWINGKEYKCKVSNKALAAPIEKTISKAKGQPRE
PQVYTLPPSREEMTKNQVSLTCLVKGFYPSDIAVEWESNGQPENNYKTTPPVLDSDGSFFLYSKLTVDKSRWQQGN
VFSCSVMHEALHNHYTQKSLSLSPGK
ODY-1248
(SEQ ID NO: 4770)
DVQLLESGGGLVQPGGSLRLSCAASGRTESDYGMGWFROAPGKEHEFVASINWSNARTSYADSAKGRETISRDNAKN

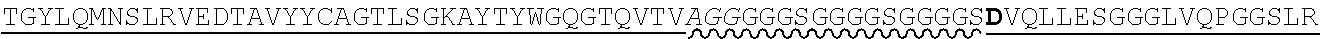

LSCAASGRTFSDYGMGWFROAPGKEHEFVASINWSNARTSYADSAKGRFTISRDNAKNTGYLQMNSLRVEDTAVYYC

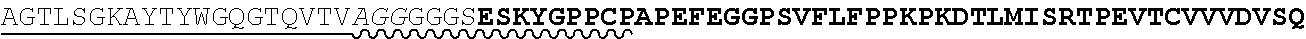

EDPEVQFNWYVDGVEVHNAKTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSDGSFFLYSRLTVDKSRWQEG
NVFSCSVMHEALHNHYTQKSLSLSLGK

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. An antigen-binding protein that specifically binds tumor necrosis factor receptor 2 (TNFR2), comprising a complementarity determining region 3 (CDR3) comprising an amino acid sequence selected from
a).
(SEQ ID NO: 60)
(Y/F)YQ(S/A)LS(T/S)(P/A)N(Y/F)GQ(V/T)F;
b).
(SEQ ID NO: 61)
AADSDL(S/R)TV(V/T)VGPHDY;
c).
(SEQ ID NO: 62)
AKDAG(S/G)WG(T/R)GPFG(Y/F)(E/D)YDY;
d).
(SEQ ID NO: 63)
AA(T/A)PSGKAY(T/S)Y;
e).
(SEQ ID NO: 64)
ATPGPY(T/S/M)YCAPYGSSWSRGYDY;
f).
(SEQ ID NO: 65)
ARV(R/G)G(T/S/A)PY(E/D)Y(N/G)Y;
g).
(SEQ ID NO: 66)
(T/A/V)A(S/A)PTGRAF(T/N/A)Y;
h).
(SEQ ID NO: 42)
AGSAFDF;
i).
(SEQ ID NO: 67)
S(V/M)(V/L)GRDM(M/V)TY;
j).
(SEQ ID NO: 4063)
AVGDFEGELVLKGDY;
k).
(SEQ ID NO: 4517)
AAD(L/V)G(F/V/Y)LY(A/T/V)DYV(P/R)LH(M/T)HHFGS;
and
l).
(SEQ ID NO: 4771)
A(A/G)(T/A)(P/L)(S/T)GKAY(T/S)Y.

2. The antigen-binding protein of claim 1, wherein the CDR3 comprises an amino acid sequence selected from SEQ ID NOs: 3, 6, 10, 14, 18, 22, 26, 30, 34, 38, 42, 46, 4063, 4067, 4071, 4524 4530, and 4727-4730.

3. The antigen-binding protein of claim 1 or 2, further comprising a CDR1 comprising an amino acid sequence selected from
a).
(SEQ ID NO: 68)
GSI(V/F)(R/S)(T/A)(N/D)(S/G/A);
b).
(SEQ ID NO: 69)
GFT(F/L)DD(I/Y)A;
c).
(SEQ ID NO: 70)
GFTFS(S/R/G)YA;
d).
(SEQ ID NO: 16)
GRTFSDYG;
e).
(SEQ ID NO: 71)
G(L/F)TLDYYA;
f).
(SEQ ID NO: 72)
GF(T/N)FSMYS;
g).
(SEQ ID NO: 73)
GRTF(G/R/S)(N/S)(Y/L)(T/F);
h).
(SEQ ID NO: 40)
GASLSRNA;
i).
(SEQ ID NO: 74)
GS(I/T)FRFPP;
j).
(SEQ ID NO: 4061)
GFTLDDYA;
and
k).
(SEQ ID NO: 4519)
G(F/V)(S/T)LD(D/Y)(H/Y)T.

4. The antigen-binding protein of claim 3, wherein the CDR1 comprises an amino acid sequence selected from SEQ ID NOs: 1, 5, 8, 12, 16, 20, 24, 28, 32, 36, 40, 44, 4061, 4065, 4069, 4520, and 4719-4722.

5. The antigen-binding protein of any one of claims 1-4, further comprising a CDR2 comprising an amino acid sequence selected from
a).
(SEQ ID NO: 75)
IRSDGF(T/I);
b).
(SEQ ID NO: 76)
I(Y/F)SY(S/G)(S/P)NT;
c).
(SEQ ID NO: 77)
I(Y/S)(S/D)DGS(E/D)T;
d).
(SEQ ID NO: 4699)
INWSN(G/A)RT;
e).
(SEQ ID NO: 78)
I(S/N)(V/T)(S/G)DGST;
f).
(SEQ ID NO: 79)
IDT(R/G)GST;
g).
(SEQ ID NO: 80)
IR(W/R/Y)(T/P)G(G/L)(S/I)T;
h).
(SEQ ID NO: 41)
IYDDGET;
i).
(SEQ ID NO: 45)
LTSGGST;
j).
(SEQ ID NO: 4062)
IFSYSSNT;
and
k).
(SEQ ID NO: 4518)
I(N/S)SNDG(S/T)(T/V).

6. The antigen-binding protein of claim 5, wherein the CDR2 comprises an amino acid sequence selected from SEQ ID NOs: 2, 9, 13, 17, 21, 25, 29, 33, 37, 41, 45, 4062, 4066, 4070, 4527, and 4723-4726.

7. The antigen-binding protein of any one of claims 1, 3, and 5, wherein the antigen-binding protein comprises
i) a CDR1 comprising an amino acid sequence of SEQ ID NO: 68, a CDR2 comprising an amino acid sequence of SEQ ID NO: 75, and a CDR3 comprising an amino acid sequence of SEQ ID NO: 60;
ii) a CDR1 comprising an amino acid sequence of SEQ ID NO: 69, a CDR2 comprising an amino acid sequence of SEQ ID NO: 76, a CDR3 comprising an amino acid sequence of SEQ ID NO: 61;
iii) a CDR1 comprising an amino acid sequence of SEQ ID NO: 70, a CDR2 comprising an amino acid sequence of SEQ ID NO: 77, a CDR3 comprising an amino acid sequence of SEQ ID NO: 62;
iv) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 63;
v) a CDR1 comprising an amino acid sequence of SEQ ID NO: 71, a CDR2 comprising an amino acid sequence of SEQ ID NO: 78, a CDR3 comprising an amino acid sequence of SEQ ID NO: 64;
vi) a CDR1 comprising an amino acid sequence of SEQ ID NO: 72, a CDR2 comprising an amino acid sequence of SEQ ID NO: 79, a CDR3 comprising an amino acid sequence of SEQ ID NO: 65;
vii) a CDR1 comprising an amino acid sequence of SEQ ID NO: 73, a CDR2 comprising an amino acid sequence of SEQ ID NO: 80, a CDR3 comprising an amino acid sequence of SEQ ID NO: 66;
viii) a CDR1 comprising an amino acid sequence of SEQ ID NO: 40, a CDR2 comprising an amino acid sequence of SEQ ID NO: 41, a CDR3 comprising an amino acid sequence of SEQ ID NO: 42; or
ix) a CDR1 comprising an amino acid sequence of SEQ ID NO: 74, a CDR2 comprising an amino acid sequence of SEQ ID NO: 45, a CDR3 comprising an amino acid sequence of SEQ ID NO: 67;
x) a CDR1 comprising an amino acid sequence of SEQ ID NO: 4061, a CDR2 comprising an amino acid sequence of SEQ ID NO: 4062, a CDR3 comprising an amino acid sequence of SEQ ID NO: 4063;
xi). a CDR1 comprising an amino acid sequence of SEQ ID NO: 4519, a CDR2 comprising an amino acid sequence of SEQ ID NO: 4518, a CDR3 comprising an amino acid sequence of SEQ ID NO: 4517; or
xii). a CDR1 comprising an amino acid sequence of SEQ ID NO: 16, a CDR2 comprising an amino acid sequence of SEQ ID NO: 4699, a CDR3 comprising an amino acid sequence of SEQ ID NO: 4771.

8. The antigen-binding protein of claim 7, wherein the antigen-binding protein comprises
a) a CDR1 comprising an amino acid sequence of SEQ ID NO: 69, a CDR2 comprising an amino acid sequence of SEQ ID NO: 76, a CDR3 comprising an amino acid sequence of SEQ ID NO: 61;
b) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 63;
c) a CDR1 comprising an amino acid sequence of SEQ ID NO: 73, a CDR2 comprising an amino acid sequence of SEQ ID NO: 80, a CDR3 comprising an amino acid sequence of SEQ ID NO: 66; or
d) a CDR1 comprising an amino acid sequence of SEQ ID NO: 16, a CDR2 comprising an amino acid sequence of SEQ ID NO: 4699, a CDR3 comprising an amino acid sequence of SEQ ID NO: 4771.

9. The antigen-binding protein of any one of claims 1, 3, 5, and 7, wherein the antigen-binding protein comprise comprises
i) a CDR1 with an amino acid sequence of GSI(V/F)(R/S)(A/T)(N/D)(G/A) (SEQ ID NO: 4700), a CDR2 comprising an amino acid sequence of IRSDGFT (SEQ ID NO: 2), and a CDR3 comprising an amino acid sequence of YYQ(S/A)LSSPNYGQ(V/T)F (SEQ ID NO: 4701);
ii) a CDR1 with an amino acid sequence of GFTFDDIA (SEQ ID NO: 8), a CDR2 comprising an amino acid sequence of IYSYGPNT (SEQ ID NO: 9), and a CDR3 comprising an amino acid sequence of AADSDLSTVV(V/T)GPHDY (SEQ ID NO: 4702);
iii) a CDR1 with an amino acid sequence of GFTFSRYA (SEQ ID NO: 12), a CDR2 comprising an amino acid sequence of ISDDGSDT (SEQ ID NO: 13), and a CDR3 comprising an amino acid sequence of AKDAGSWGTGPFGYEYDY (SEQ ID NO: 14);
iv) a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of AA(T/A)PSGKAYSY (SEQ ID NO: 4703);
v) a CDR1 with an amino acid sequence of GLTLDYYA (SEQ ID NO: 20), a CDR2 comprising an amino acid sequence of ISTSDGST (SEQ ID NO: 21), and a CDR3 comprising an amino acid sequence of ATPGPYTYCAPYGSSWSRGYDY (SEQ ID NO: 22);
vi) a CDR1 with an amino acid sequence of GF(T/N)FSMYS (SEQ ID NO: 72), a CDR2 comprising an amino acid sequence of IDT(R/G)GST (SEQ ID NO: 79), and a CDR3 comprising an amino acid sequence of ARV(G/R)G(T/A)PYEY(N/G)Y (SEQ ID NO: 4704);
vii) a CDR1 with an amino acid sequence of GRTF(G/S)S(Y/L)(T/F) (SEQ ID NO: 4705), a CDR2 comprising an amino acid sequence of IR(W/R/Y)(T/P)G(G/L)(S/I)T (SEQ ID NO: 80), and a CDR3 comprising an amino acid sequence of (A/V)A(A/S)PTGRAF(T/N)Y (SEQ ID NO: 4707);
viii) a CDR1 with an amino acid sequence of GASLSRNA (SEQ ID NO: 40), a CDR2 comprising an amino acid sequence of IYDDGET (SEQ ID NO: 41), and a CDR3 comprising an amino acid sequence of AGSAFDF (SEQ ID NO: 42);
ix) a CDR1 with an amino acid sequence of GS(T/I)FRFPP (SEQ ID NO: 4708), a CDR2 comprising an amino acid sequence of LTSGGST (SEQ ID NO: 45), and a CDR3 comprising an amino acid sequence of SVLGRDM(M/V)TY (SEQ ID NO: 4706);
x) a CDR1 with an amino acid sequence of GFTLDDYA (SEQ ID NO: 4061), a CDR2 comprising an amino acid sequence of IFSYSSNT (SEQ ID NO: 4062), and a CDR3 comprising an amino acid sequence of AVGDFEGELVLKGDY (SEQ ID NO: 4063);
xi) a CDR1 with an amino acid sequence of GFTLDYYT (SEQ ID NO: 4065), a CDR2 comprising an amino acid sequence of ISSNDGSV (SEQ ID NO: 4066), and a CDR3 comprising an amino acid sequence of AADLGYLYVDYVRLHTHHFGS (SEQ ID NO: 4067); or
xii). a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of A(A/G)(T/A)(P/L)(S/T)GKAY(T/S)Y (SEQ ID NO: 4771).

10. The antigen-binding protein of claim 8 or claim 9, wherein the antigen-binding protein comprise comprises
a) a CDR1 with an amino acid sequence of GFTFDDIA (SEQ ID NO: 8), a CDR2 comprising an amino acid sequence of IYSYGPNT (SEQ ID NO: 9), and a CDR3 comprising an amino acid sequence of AADSDLSTVV(V/T)GPHDY (SEQ ID NO: 4702);
b) a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of AA(T/A)PSGKAYSY (SEQ ID NO: 4703);
c) a CDR1 with an amino acid sequence of GRTF(G/S)S(Y/L)(T/F) (SEQ ID NO: 4705), a CDR2 comprising an amino acid sequence of IR(W/R/Y)(T/P)G(G/L)(S/I)T (SEQ ID NO: 80), and a CDR3 comprising an amino acid sequence of (A/V)A(A/S)PTGRAF (T/NJY (SEQ ID NO: 4707); or
d). a CDR1 with an amino acid sequence of GRTFSDYG (SEQ ID NO: 16), a CDR2 comprising an amino acid sequence of INWSN(G/A)RT (SEQ ID NO: 4699), and a CDR3 comprising an amino acid sequence of A(A/G)(T/A)(P/L)(S/T)GKAY(T/S)Y (SEQ ID NO: 4771).

11. The antigen-binding protein of any one of claims 1-7 and 9, wherein the antigen-binding protein comprise
i) a CDR1 comprising an amino acid sequence of SEQ ID NO: 1, a CDR2 comprising an amino acid sequence of SEQ ID NO: 2, and a CDR3 comprising an amino acid sequence of SEQ ID NO: 3;
ii) a CDR1 comprising an amino acid sequence of SEQ ID NO: 5, a CDR2 comprising an amino acid sequence of SEQ ID NO: 2, a CDR3 comprising an amino acid sequence of SEQ ID NO: 6;
iii) a CDR1 comprising an amino acid sequence of SEQ ID NO: 8, a CDR2 comprising an amino acid sequence of SEQ ID NO: 9, a CDR3 comprising an amino acid sequence of SEQ ID NO: 10;
iv) a CDR1 comprising an amino acid sequence of SEQ ID NO: 12, a CDR2 comprising an amino acid sequence of SEQ ID NO: 13, a CDR3 comprising an amino acid sequence of SEQ ID NO: 14;
v) a CDR1 comprising an amino acid sequence of SEQ ID NO: 16, a CDR2 comprising an amino acid sequence of SEQ ID NO: 17, a CDR3 comprising an amino acid sequence of SEQ ID NO: 18;
vi) a CDR1 comprising an amino acid sequence of SEQ ID NO: 20, a CDR2 comprising an amino acid sequence of SEQ ID NO: 21, a CDR3 comprising an amino acid sequence of SEQ ID NO: 22;
vii) a CDR1 comprising an amino acid sequence of SEQ ID NO: 24, a CDR2 comprising an amino acid sequence of SEQ ID NO: 25, a CDR3 comprising an amino acid sequence of SEQ ID NO: 26;
viii) a CDR1 comprising an amino acid sequence of SEQ ID NO: 28, a CDR2 comprising an amino acid sequence of SEQ ID NO: 29, a CDR3 comprising an amino acid sequence of SEQ ID NO: 30;
ix) a CDR1 comprising an amino acid sequence of SEQ ID NO: 32, a CDR2 comprising an amino acid sequence of SEQ ID NO: 33, a CDR3 comprising an amino acid sequence of SEQ ID NO: 34;
x) a CDR1 comprising an amino acid sequence of SEQ ID NO: 36, a CDR2 comprising an amino acid sequence of SEQ ID NO: 37, a CDR3 comprising an amino acid sequence of SEQ ID NO: 38;
xi) a CDR1 comprising an amino acid sequence of SEQ ID NO: 40, a CDR2 comprising an amino acid sequence of SEQ ID NO: 41, a CDR3 comprising an amino acid sequence of SEQ ID NO: 42;
xii) a CDR1 comprising an amino acid sequence of SEQ ID NO: 44, a CDR2 comprising an amino acid sequence of SEQ ID NO: 45, a CDR3 comprising an amino acid sequence of SEQ ID NO: 46;
xiii) a CDR1 comprising an amino acid sequence of SEQ ID NO: 4061, a CDR2 comprising an amino acid sequence of SEQ ID NO: 4062, a CDR3 comprising an amino acid sequence of SEQ ID NO: 4063;
xiv). a CDR1 comprising an amino acid sequence of SEQ ID NO: 4065, a CDR2 comprising an amino acid sequence of SEQ ID NO: 4066, a CDR3 comprising an amino acid sequence of SEQ ID NO: 4067;
xv). a CDR1 comprising an amino acid sequence of SEQ ID NO: 4069, a CDR2 comprising an amino acid sequence of SEQ ID NO: 4070, a CDR3 comprising an amino acid sequence of SEQ ID NO: 4071;
xvi). a CDR1 comprising an amino acid sequence of SEQ ID NO: 4520, a CDR2 comprising an amino acid sequence of SEQ ID NO: 45, a CDR3 comprising an amino acid sequence of SEQ ID NO: 46;
xvii). a CDR1 comprising an amino acid sequence of SEQ ID NO: 8, a CDR2 comprising an amino acid sequence of SEQ ID NO: 9, a CDR3 comprising an amino acid sequence of SEQ ID NO: 4524;
xviii). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 18;
xix). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 4530;
xx). a CDR1 comprising an amino acid sequence of SEQ ID NO: 4719, a CDR2 comprising an amino acid sequence of SEQ ID NO: 4723, a CDR3 comprising an amino acid sequence of SEQ ID NO: 4727;
xxi). a CDR1 comprising an amino acid sequence of SEQ ID NO: 4720, a CDR2 comprising an amino acid sequence of SEQ ID NO: 4724, a CDR3 comprising an amino acid sequence of SEQ ID NO: 4728;
xxii). a CDR1 comprising an amino acid sequence of SEQ ID NO: 4721, a CDR2 comprising an amino acid sequence of SEQ ID NO: 4725, a CDR3 comprising an amino acid sequence of SEQ ID NO: 4729; or
xxiii). a CDR1 comprising an amino acid sequence of SEQ ID NO: 4722, a CDR2 comprising an amino acid sequence of SEQ ID NO: 4726, a CDR3 comprising an amino acid sequence of SEQ ID NO: 4730.

12. The antigen-binding protein of any one of claims 1-11, wherein the antigen-binding protein comprises
a) a CDR1 comprising an amino acid sequence of SEQ ID NO: 16, a CDR2 comprising an amino acid sequence of SEQ ID NO: 17, a CDR3 comprising an amino acid sequence of SEQ ID NO: 18;
b). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 18;
c). a CDR1 comprising an amino acid sequence of SEQ ID NO: 16, a CDR2 comprising an amino acid sequence of SEQ ID NO: 4527, a CDR3 comprising an amino acid sequence of SEQ ID NO: 4530;
d) a CDR1 comprising an amino acid sequence of SEQ ID NO: 8, a CDR2 comprising an amino acid sequence of SEQ ID NO: 9, a CDR3 comprising an amino acid sequence of SEQ ID NO: 10;
e). a CDR1 comprising an amino acid sequence of SEQ ID NO: 8, a CDR2 comprising an amino acid sequence of SEQ ID NO: 9, a CDR3 comprising an amino acid sequence of SEQ ID NO: 4524;
f). a CDR1 comprising an amino acid sequence of SEQ ID NO: 4069, a CDR2 comprising an amino acid sequence of SEQ ID NO: 4070, a CDR3 comprising an amino acid sequence of SEQ ID NO: 4071;
g). a CDR1 comprising an amino acid sequence of SEQ ID NO: 4719, a CDR2 comprising an amino acid sequence of SEQ ID NO: 4723, a CDR3 comprising an amino acid sequence of SEQ ID NO: 4727;
h). a CDR1 comprising an amino acid sequence of SEQ ID NO: 4720, a CDR2 comprising an amino acid sequence of SEQ ID NO: 4724, a CDR3 comprising an amino acid sequence of SEQ ID NO: 4728;
i). a CDR1 comprising an amino acid sequence of SEQ ID NO: 4721, a CDR2 comprising an amino acid sequence of SEQ ID NO: 4725, a CDR3 comprising an amino acid sequence of SEQ ID NO: 4729; or
j). a CDR1 comprising an amino acid sequence of SEQ ID NO: 4722, a CDR2 comprising an amino acid sequence of SEQ ID NO: 4726, a CDR3 comprising an amino acid sequence of SEQ ID NO: 4730.

13. The antigen-binding protein of any one of claims 1-12, wherein the antigen-binding protein is a single-domain antibody.

14. The antigen-binding protein of claim 13, wherein the single-domain antibody is a VHH, a VNAR, or an engineered VH domain.

15. The antigen-binding protein of claim 14, wherein the VHH is a camelid VHH.

16. The antigen-binding protein of claim 15, wherein the VHH comprises an amino acid sequence selected from any one of SEQ ID NOs: 4, 7, 11, 15, 19, 23, 27, 31, 35, 39, 43, 47, 4064, 4068, 4072, 4521, 93-640, 4079-4125 2805-3363, 4359-4420, and 4605-4628, or a sequence having at least 75% identity thereto.

17. The antigen-binding protein of claim 15 or 16, wherein the VHH comprises an amino acid sequence selected from any one of SEQ ID NOs: 4, 7, 11, 15, 19, 23, 27, 31, 35, 39, 43, 47, 4064, 4068, 4072, and 4521, or a sequence having at least 75% identity thereto.

18. The antigen-binding protein of claim 14, wherein the VHH is a humanized VHH.

19. The antigen-binding protein of claim 18, wherein the humanized VHH comprises an amino acid sequence selected from any one of SEQ ID NOs: 81-92, 4076-4078, 4523, 4526, 4529, 4532, 4731-4734, 641-1127, and 4126-4172, or a sequence having at least 75% identity thereto.

20. The antigen-binding protein of claim 19, wherein the humanized VHH comprises an amino acid sequence selected from any one of SEQ ID NOs: 81-92, 4076-4078, 4523, 4526, 4529, 4731-4734, and 4532 or a sequence having at least 75% identity thereto.

21. The antigen-binding protein of any one of claims 1-20, wherein the antigen-binding protein has an agonist effect upon binding to TNFR2.

22. The antigen-binding protein of any one of claims 1-21, wherein the antigen-binding protein binds to human TNFR2.

23. The antigen-binding protein of claim 22, wherein the antigen-binding protein binds to human TNFR2 with a K_{D} of less than about 3×10⁻⁷ M.

24. The antigen-binding protein of claim 23, wherein the antigen-binding protein binds to human TNFR2 with a K_{D} of about 1×10⁻¹⁰ to 5×10⁻⁸ M.

25. The antigen-binding protein of any one of claims 1-10, wherein the antigen-binding protein binds to cyno TNFR2.

26. The antigen-binding protein of claim 25, wherein the antigen-binding protein binds to cyno TNFR2 with a K_{D} of less than about 3×10⁻⁷ M.

27. The antigen-binding protein of claim 26, wherein the antigen-binding protein binds to cyno TNFR2 with a K_{D} of about 1×10⁻⁹ to 2×10⁻⁷ M.

28. The antigen-binding protein of any one of claims 1-27, wherein the antigen-binding protein binds to the same epitope(s) as antibody clone MR2-1.

29. The antigen-binding protein of any one of claims 1-27, wherein the antigen-binding protein does not bind to the same epitope(s) as antibody clone MR2-1.

30. The antigen-binding protein of any one of claims 1-29, wherein the antigen-binding protein increases expression of one or more proteins selected from a protein in the NF-kB pathway, FOXP3, HELIOS, EZH2, HLA-DR, ICAM-1, OX-40, ICOS, and CCR8.

31. The antigen-binding protein of any one of claims 1-30, wherein the antigen-binding protein comprises one or more modifications that reduce binding of said antigen-binding protein by pre-existing antibodies found in human blood or serum.

32. A fusion protein that specifically binds tumor necrosis factor receptor 2 (TNFR2), comprising one or more of said antigen-binding proteins of any one of claims 1-31.

33. The fusion protein of claim 32, which comprises two said antigen-binding proteins.

34. The fusion protein of claim 32, which comprises four said antigen-binding proteins.

35. The fusion protein of any one of claims 32-34, wherein the one or more antigen-binding proteins bind to the same epitope on TNFR2.

36. The fusion protein of any one of claims 32-34, wherein the one or more antigen-binding proteins bind to different epitopes on TNFR2.

37. The fusion protein of any one of claims 32-36, wherein the one or more antigen-binding proteins are one or more single-domain antibodies.

38. The fusion protein of claim 37, wherein one or more single-domain antibodies are one or more VHHs.

39. The fusion protein of any one of claims 32-38, which further comprises an immunoglobulin Fc region.

40. The fusion protein of claim 39, wherein the immunoglobulin Fc region is an Fc region of a human immunoglobulin.

41. The fusion protein of claim 40, wherein the immunoglobulin Fc region is an Fc region of human IgG1, IgG2, IgG3 or IgG4, or a variant thereof.

42. The fusion protein of claim 41, wherein the immunoglobulin Fc region is an Fc region of human IgG1, or a variant thereof.

43. The fusion protein of claim 42, wherein the Fc region of human IgG1 comprises one or more mutations selected from L234A, L235A, G237A, D265A, N297A, and/or P329A according to EU numbering.

44. The fusion protein of claim 43, wherein the Fc region of human IgG1 comprises a set of mutations selected from
1. L234A and L235A;
2. L234A, L235A, and P329A;
3. D265A, N297A and P329A; and
4. L234A, L235A, and G237A.

45. The fusion protein of claim 41, wherein the immunoglobulin Fc region is an Fc region of human IgG4, or a variant thereof.

46. The fusion protein of claim 45, wherein the Fc region of human IgG4 comprises one or more mutations selected from S228P, L235E, L235A, and/or F234A according to EU numbering.

47. The fusion protein of claim 46, wherein the Fc region of human IgG4 comprises a set of mutations selected from
1. S228P and L235E;
2. S228P and L235A;
3. S228P, F234A, and L235E; and
4. S228P, F234A, and L235A.

48. The fusion protein of any one of claims 32-52, which further comprises a cytokine.

49. The fusion protein of claim 48, wherein the cytokine is IL-2, or a variant thereof.

50. The fusion protein of claim 49, wherein the cytokine is an IL-2 variant comprising a N88D mutation.

51. The fusion protein of any one of claims 32-50, which further comprises a moiety that binds to serum albumin.

52. The fusion protein of claim 32, which comprises the amino acid sequence of any one of SEQ ID Nos: 3933-3964, 4483-4513, 4686-4696, 4709-4716, and 4735-4770, or a sequence having at least 75% identity thereto.

53. A conjugate comprising the antigen-binding protein of any one of claims 1-31 or the fusion protein of any one of claims 32-52, wherein the antigen-binding protein or fusion protein is conjugated to a second moiety.

54. The conjugate of claim 53, wherein the second moiety is selected from a detectable label, a drug, a toxin, a radionuclide, an enzyme, an immunomodulatory agent, a cytokine, a cytotoxic agent, a chemotherapeutic agent, a diagnostic agent, or a combination thereof.

55. The conjugate of claim 54, wherein the second moiety is a cytokine.

56. The conjugate of claim 55, wherein the cytokine is IL-2, or a variant thereof.

57. The conjugate of claim 56, wherein the cytokine is IL-2 variant comprising a N88D mutation.

58. A polynucleotide molecule encoding the antigen-binding protein of any one of claims 1-31 or the fusion protein of any one of claims 32-52.

59. The polynucleotide molecule of claim 54, which comprises the nucleotide sequence of any one of SEQ ID NOs: 48-59, 4073-4075, 4522, 4525, 4528, 4531, 3364-3922, 4421-4482, and 4629-4652 or a sequence having at least 70% identity thereto.

60. The polynucleotide molecule of claim 59, which comprises the nucleotide sequence of any one of SEQ ID NOs: 48-59, 4073-4075, 4522, 4525, 4528, and 4531, or a sequence having at least 70% identity thereto.

61. A recombinant vector comprising the polynucleotide molecule of any one of claims 58-60.

62. A host cell comprising polynucleotide molecule of any one of claims 58-60, or the expression vector of claim 61.

63. A kit comprising the antigen-binding protein of any one of claims 1-31, the fusion protein of any one of claims 32-52, or the conjugate of any one of claims 53-57, the polynucleotide molecule of any one of claims 58-60, or the recombinant vector of claim 61, and optionally, instructions and/or packaging for the same.

64. A pharmaceutical composition comprising the antigen-binding protein of any one of claims 1-31, the fusion protein of any one of claims 32-52, or the conjugate of any one of claim 53-57, the polynucleotide molecule of any one of claims 58-60, or the recombinant vector of claim 61, and a pharmaceutically acceptable carrier and/or excipient.

65. A method for preparing an antigen-binding protein or a fusion protein that specifically binds tumor necrosis factor receptor 2 (TNFR2), comprising the steps of:
(a) culturing the host cell of claim 62 in a culture medium under conditions suitable for expression of the antigen-binding protein or fusion protein, and
(b) isolating the antigen-binding protein or fusion protein from the host cell and/or culture medium.

66. A method for promoting proliferation, activating and/or enhancing suppressive function, and/or stabilizing immunosuppressive phenotype of a population of regulatory T cells (Treg) comprising contacting the population of regulatory T cells with the antigen-binding protein of claim 1-31, the fusion protein of any one of claims 32-52, or the conjugate of any one of claim 53-57.

67. The method of claim 66, wherein said contacting occurs in vitro.

68. The method of claim 66, wherein said contacting occurs in vivo.

69. The method of claim 68, wherein the method further comprises administering the antigen-binding protein, the fusion protein, or the conjugate into a subject in need thereof.

70. A method of treating or preventing a disease or disorder in a subject in need thereof, said method comprising administering to the subject the antigen-binding protein of claim 1-31, the fusion protein of any one of claims 32-52, or the conjugate of any one of claim 53-57.

71. The method of claim 70, wherein the disease or disorder is an immunological disease, inflammatory disease, cancer, cardiovascular disease, or an infertility and pregnancy-associated disease.

72. The method of claim 71, wherein the immunological disease is selected from an autoimmune disease, a neurological condition, an allergy, asthma, macular degeneration, muscular atrophy, a disease related to miscarriage, atherosclerosis, bone loss, a musculoskeletal disease, obesity, a graft-versus-host disease, and an allograft rejection.

73. The method of claim 72, wherein the autoimmune disease is selected from lupus, alopecia areata, ankylosing spondylitis, antiphospholipid syndrome, autoimmune Addison's disease, autoimmune hemolytic anemia, autoimmune hepatitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, Crohn's disease, essential mixed cryoglobulinemia, fibromyalgia-fibromyositis, Goodpastures disease, Graves' disease, Guillain-Barré, Hashimoto's thyroiditis, hypothyroidism, idiopathic pulmonary fibrosis, idiopathic thrombocytopenia purpura (ITP), IgA nephropathy, juvenile arthritis, lichen planus, lichen sclerosis, IgG4-related disease, Meniere's disease, mixed connective tissue disease, multiple sclerosis, myasthenia gravis, neuromyelitis optica spectrum disease, pemphigus vulgaris or related blistering skin disease, pernicious anemia, polyarteritis nodosa, polychondritis, polyglandular syndromes, polymyalgia rheumatica, polymyositis and dermatomyositis, premature ovarian failure, primary agammaglobulinemia, primary biliary cirrhosis, psoriasis, primary ovarian insufficiency, Raynaud's phenomenon, Reiter's syndrome, rheumatic fever, rheumatoid arthritis, sarcoidosis, scleroderma, Sjögren's syndrome, spondyloarthritis, stiff-man syndrome, type I diabetes, Takayasu arteritis, temporal arteritis/giant cell arteritis, ulcerative colitis, uveitis, vasculitis, vitiligo, and Wegener's granulomatosis (Granulomatosis with polyanglitis) or other immune vasculitis.

74. The method of claim 73, wherein the lupus is systemic lupus erythematosus (SLE), cutaneous lupus, lupus nephritis, neonatal lupus, or drug-induced lupus.

75. The method of claim 74, wherein the cutaneous lupus is acute cutaneous lupus, chronic cutaneous lupus erythematosus, discoid lupus erythematosus (DLE), or subacute cutaneous lupus erythematosus.

76. The method of claim 72, wherein the neurological condition is selected from a brain tumor, a brain metastasis, a spinal cord injury, schizophrenia, epilepsy, Amyotrophic lateral sclerosis (ALS), Alzheimer's disease, Huntington's disease, Parkinson's disease, and stroke.

77. The method of claim 72, wherein the allergy is selected from food allergy, seasonal allergy, pet allergy, hives, hay fever, allergic conjunctivitis, poison ivy allergy oak allergy, mold allergy, drug allergy, dust allergy, cosmetic allergy, and chemical allergy.

78. The method of claim 72, wherein the allograft rejection is selected from skin graft rejection, bone graft rejection, vascular tissue graft rejection, ligament graft rejection, and organ graft rejection.

79. The method of claim 72, wherein the ligament graft rejection is selected from cricothyroid ligament graft rejection, caudal cruciate ligament graft rejection, periodontal ligament graft rejection, suspensory ligament of the lens graft rejection, palmar radiocarpal ligament graft rejection, dorsal radiocarpal ligament graft rejection, ulnar collateral ligament graft rejection, radial collateral ligament graft rejection, suspensory ligament of the breast graft rejection, anterior sacroiliac ligament graft rejection, posterior sacroiliac ligament graft rejection, sacrotuberous ligament graft rejection, sacrospinous ligament graft rejection, inferior pubic ligament graft rejection, superior pubic ligament graft rejection, anterior cruciate ligament graft rejection, lateral collateral ligament graft rejection, posterior cruciate ligament graft rejection, medial collateral ligament graft rejection, cranial cruciate ligament graft rejection, and patellar ligament graft rejection.

80. The method of claim 72, wherein the organ graft rejection is selected from heart graft rejection, lung graft rejection, kidney graft rejection, liver graft rejection, pancreas graft rejection, intestine graft rejection, and thymus graft rejection.

81. The method of claim 72, wherein the graft-versus-host disease arises from a bone marrow transplant or one or more blood cells selected from B-cells, T-cells, basophils, common myeloid progenitor cells, common lymphoid progenitor cells, dendritic cells, eosinophils, hematopoietic stem cells, neutrophils, natural killer cells, megakaryocytes, monocytes, or macrophages.

82. The method of claim 72, wherein the inflammatory disease is acute or chronic inflammation.

83. The method of claim 72, wherein the inflammatory disease is selected from osteoarthritis, atopic dermatitis, endometriosis, polycystic ovarian syndrome, inflammatory bowel disease, fibrotic lung disease, and cardiac inflammation.

84. The method of claim 72, wherein the cancer is selected from adenoid cystic carcinoma, adrenal gland tumor, amyloidosis, anal cancer, appendix cancer, astrocytoma, ataxia-telangiectasia, Beckwith-Wiedemann syndrome, bile duct cancer (cholangiocarcinoma), Birt-Hogg-Dubé syndrome, bladder cancer, bone cancer (sarcoma of bone), brain stem glioma, brain tumor, breast cancer, inflammatory breast cancer, metastatic breast cancer, male breast cancer, Carney complex, central nervous system tumors (brain and spinal cord), cervical cancer, childhood cancer, colorectal cancer, Cowden syndrome, craniopharyngioma, desmoid tumor, desmoplastic infantile ganglioglioma, childhood tumor, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor (GIST), germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS-related cancer, juvenile polyposis syndrome, kidney cancer, lacrimal gland tumor, laryngeal and hypopharyngeal cancer, acute lymphoblastic leukemia (ALL), acute myeloid leukemia (AML), B-cell prolymphocytic leukemia and hairy cell leukemia, chronic lymphocytic leukemia (CLL), chronic myeloid leukemia (CML), chronic T-cell lymphocytic leukemia, eosinophilic leukemia, Li-Fraumeni syndrome, liver cancer, lung cancer, non-small cell lung cancer, small cell lung cancer, hodgkin lymphoma, non-hodgkin lymphoma, lynch syndrome, mastocytosis, medulloblastoma, melanoma, meningioma, mesothelioma, multiple endocrine neoplasia type 1, multiple endocrine neoplasia type 2, multiple myeloma, MUTYH (or MYH)-associated polyposis, myelodysplastic syndromes (MDS), nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neuroendocrine tumors, neurofibromatosis type 1, neurofibromatosis type 2, nevoid basal cell carcinoma syndrome, oral and oropharyngeal cancer, osteosarcoma, ovarian, fallopian tube, and peritoneal cancer, pancreatic cancer, parathyroid cancer, penile cancer, Peutz-Jeghers syndrome, pheochromocytoma and paraganglioma, pituitary gland tumor, pleuropulmonary blastoma, prostate cancer, retinoblastoma, rhabdomyosarcoma, salivary gland cancer, Kaposi sarcoma, soft tissue sarcomas, skin cancer (non-melanoma), small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom macroglobulinemia (lymphoplasmacytic lymphoma), Werner syndrome, Wilms tumor, or xeroderma pigmentosum.

85. The method of claim 72, wherein the cardiovascular disease is selected from atherosclerosis, heart failure, left heart failure with reduced ejection fraction, left heart failure with preserved ejection fraction, right ventricular failure, congestive heart failure, restrictive cardiomyopathy, dilated cardiomyopathy, hypertrophic cardiomyopathy, ischemic cardiomyopathy, idiopathic cardiomyopathy, and hypertension.

86. The method of claim 72, wherein the infertility and pregnancy-associated diseases is selected from recurrent pregnancy loss, pre-eclampsia, preterm labor, fetal growth restriction, or intrauterine growth restriction.

87. A method of regenerating a tissue or organ comprising one or more TNFR2+ cells, said method comprising contacting the tissue or organ with an effective amount of the antigen-binding protein of claim 1-31, the fusion protein of any one of claims 32-52, or the conjugate of any one of claim 53-57.

88. The method of claim 87, wherein said tissue or organ is selected from pancreas, salivary gland, pituitary gland, kidney, heart, lung, hematopoietic system, cranial nerves, heart, aorta, olfactory gland, ear, nerve, eye, thymus, tongue, bone, liver, small intestine, large intestine, gastrointestinal, lung, brain, skin, peripheral nervous system, central nervous system, spinal cord, breast, embryonic structures, embryo, and testes tissue.

89. The method of claim 87 or 88, wherein said contacting occurs in vitro.

90. The method of claim 87 or 88, wherein said contacting occurs in vivo.

91. The method of claim 90, wherein the method further comprises administering the antigen-binding protein, the fusion protein, or the conjugate into a subject in need thereof.

92. A method for inducing tolerance to a foreign agent and/or preventing or reducing immune response to a foreign agent in a subject in need thereof, said method comprising administering to the subject the antigen-binding protein of claim 1-31, the fusion protein of any one of claims 32-52, or the conjugate of any one of claim 53-57.

93. The method of claim 92, wherein the foreign agent is a therapeutic protein or peptide, a viral vector, a bacterial vector, a fungal vector, a biochemical vector, a lipid, carbohydrate, a nucleic acid, a sperm, an oocyte, or an embryo.

94. The method of claim 93, wherein the viral vector is a DNA or RNA vector.

95. The method of any one of claims 69-83 and 91-94, wherein the subject is a mammal.

96. The method of claim 95, wherein the mammal is human.
